(19) United States
(12) Patent Application Publication
(10) Pub. No.: US 2025/0215508 A1
(43) Pub. Date: Jul. 3, 2025

(54) CRISPR-Cas9 AS A SELECTIVE AND SPECIFIC CELL KILLING TOOL

(71) Applicant: The Johns Hopkins University, Baltimore, MD (US)

(72) Inventors: James R. Eshleman, Baltimore, MD (US); Selina Shiqing K. Teh, Baltimore, MD (US); Kirsten D. Bowland, Baltimore, MD (US); Nicholas Roberts, Baltimore, MD (US)

(21) Appl. No.: 19/051,327

(22) Filed: Feb. 12, 2025

Related U.S. Application Data
(63) Continuation of application No. PCT/US2023/031039, filed on Aug. 24, 2023.
(60) Provisional application No. 63/438,300, filed on Jan. 11, 2023.
(60) Provisional application No. 63/401,375, filed on Aug. 26, 2022.

(51) Int. Cl. C12Q 1/6886; A61K 38/46; A61P 35/00; C12N 15/11

(52) U.S. Cl. CPC C12Q 1/6886; A61K 38/465; A61P 35/00; C12N 15/11; C12N 2310/20; C12N 2320/34; C12Q 2600/156

(57) ABSTRACT

A CRISPR-Cas9 system for treating a disease, disorder, or condition associated with one or more somatic mutations in a subject in need of treatment thereof is disclosed. The system comprises a sgRNA-guided Cas9, wherein the sgRNA targets between about 1 to about 50 mutations in a target cell. The CRISPR-Cas9 system can be used to treat diseases, disorders, or conditions associated with one or more somatic mutations, including cancers, autoimmune diseases, and/or neurodegenerative diseases. Additionally, the present disclosure relates to methods of identifying somatic mutations in a tumor that produce a protospacer adjacent motif (PAM) and methods of designing a CRISPR-Cas 9 system to target PAMs identified in a tumor sample obtained from a subject.

### RELATED APPLICATION INFORMATION

This application is a continuation application of International Application No. PCT/US2023/031039, filed on Aug. 24, 2023, which claims priority to U.S. Application No. 63/401,375 filed on Aug. 26, 2022, and U.S. Application No. 63/438,300 filed on Jan. 1, 2023, the contents of each of which are herein incorporated by reference.

### FEDERALLY SPONSORED RESEARCH OR DEVELOPMENT

This invention was made with government support under grant CA164592-01 awarded by the National Institutes of Health. The government has certain rights in the invention.

### SEQUENCE LISTING STATEMENT

The contents of the electronic sequence listing titled JHU_41220_601_ST26.xml (Size: 422,398 bytes; and Date of Creation: Feb. 11, 2025) is herein incorporated by reference in its entirety.

### TECHNICAL FIELD

The present disclosure relates to a CRISPR-Cas9 system for treating a disease, disorder, or condition associated with somatic mutations in a subject in need of treatment thereof. More specifically, the present disclosure relates to a CRISPR-Cas9 system comprising a sgRNA-guided Cas9, wherein the sgRNA targets between 1-50 mutations in a target cell in a subject. Additionally, the present disclosure relates to methods of identifying somatic mutations in a tumor that produce a protospacer adjacent motif (PAM) and methods of designing a CRISPR-Cas 9 system to target PAMs identified in a tumor sample obtained from a subject.

### BACKGROUND

Solid tumors arise from multistep carcinogenesis, produced by the accumulation of driver mutations in oncogenes and tumor suppressor genes (2, 3). However, the vast majority of mutations found in cancers are passengers (1, 4). Since cancer is a clonal disease, all malignant cells should contain the mutations present in the cancer initiating cell at the beginning of tumorigenesis.

Since its discovery, reduction to a two-component system, and demonstration of activity in human cells, the CRISPR-Cas9 system has been rapidly adopted by scientists as the tool of choice for gene editing (5-7). CRISPR-Cas9 works by introducing a double-strand break (DSB) as directed by a complementary single-guide RNA (sgRNA) sequence in the presence of a protospacter adjacent motif (PAM), where the break is then repaired by one of the three endogenous DSB repair systems. However, CRISPR-Cas9 has been associated with off-target activity and other toxicities, sometimes resulting in unintentional loss of whole chromosome arms (8, 9).

### SUMMARY

In one embodiment, the presently disclosed subject matter relates to a method of identifying somatic mutations in a tumor that produce a protospacer adjacent motif (PAM) in a subject. In some aspects, the method comprising the steps of:
a. obtaining from a subject having at least one tumor: i) at least one sample from the tumor; and ii) at least one non-tumor sample;
b. obtaining DNA from the tumor sample and from the non-tumor sample;
c. performing next generation sequencing of DNA obtained from the tumor sample and the normal sample to produce a tumor sequence and a normal sequence;
d. aligning the tumor sequence and the normal sequence; and
e. identifying one or more somatic mutations in the tumor sequence that produce one or more PAMs.

In some aspects of the above method, the tumor sample is a tissue sample, a blood sample, a plasma sample, a serum sample, an urine sample, cerebrospinal fluid, stool or feces, saliva, ascites fluid, sputum, synovial fluid, or any combination thereof.

In other aspects of the above method, the non-tumor sample is a tissue sample, a blood sample, a plasma sample, a serum sample, an urine sample, cerebrospinal fluid, stool or feces, saliva, ascites fluid, sputum, synovial fluid, or any combination thereof.

In still further aspects of the above method, the identifying of one or more somatic mutations in the tumor sequence involves identifying one or more single somatic base substitutions (BS), one or more structural variants (SV), or one or more BS and SVs that produce one or more PAMs.

In still further aspects, the tumor is cancer. In yet further aspects, the cancer is pancreatic cancer, lung cancer, esophageal cancer, or any combinations thereof.

In still further aspects of the above method, the next generation sequencing is whole genome sequencing.

In yet another embodiment, the presently disclosed subject matter relates to a method of designing a CRISPR-Cas 9 system to target protospacer adjacent motifs (PAMs) identified in a tumor sample obtained from a subject. The method comprises the steps of:
a. obtaining from a subject having a tumor: i) at least one sample from the tumor; and ii) at least one non-tumor sample;
b. obtaining DNA from the tumor sample and from the non-tumor sample;
c. performing next generation sequencing of DNA obtained from the tumor cell line and the normal cell line to produce a tumor sequence and a normal sequence;
d. aligning the tumor sequence and the normal sequence;
e. identifying one or more somatic mutations in the tumor sequence that produce one or more PAMs; and
f. designing one or more CRISPR-Cas9 systems, wherein the CRISPR-Cas9 system comprises one or more sgRNAs that target a sequence adjacent to one or more PAMs.

In some aspects of the above method, the tumor sample is a tissue sample, a blood sample, a plasma sample, a serum sample, an urine sample, cerebrospinal fluid, stool or feces, saliva, ascites fluid, sputum, synovial fluid, or any combination thereof.

In other aspects of the above method, the non-tumor sample is a tissue sample, a blood sample, a plasma sample, a serum sample, an urine sample, cerebrospinal fluid, stool or feces, saliva, ascites fluid, sputum, synovial fluid, or any combination thereof.

In still further aspects of the above method, the identifying of one or more somatic mutations in the tumor sequence involves identifying one or more single somatic base substitutions (BS), one or more structural variants (SV), or one or more BS and SVs that produce one or more PAMs.

In still further aspects, the tumor is cancer. In yet further aspects, the cancer is pancreatic cancer, lung cancer, esophageal cancer, or any combinations thereof.

In still further aspects of the above method, the next generation sequencing is whole genome sequencing.

In still other aspects, the presently disclosed subject matter relates to a method of treating a subject suffering from pancreatic cancer, lung cancer, esophageal cancer, or any combination thereof, the method comprising administering to the subject a therapeutically effective amount of the CRISPR-Cas9 system designed according to the above method.

In another embodiment, the presently disclosed subject matter provides a CRISPR-Cas9 system for treating a disease, disorder, or condition associated with one or more somatic mutations, the system comprising a single-guide RNA or sgRNA-guided Cas9 (collectively, “sgRNA”), wherein the sgRNA targets between about 1 to about 50 mutations in a target cell.

In some aspect, the CRISPR-Cas9 system comprises a sgRNA, wherein the sgRNA is designed as a multi-target sgRNA that are both patient-specific and cancer-specific. In certain aspects, the CRISPR-Cas9 system comprises a sgRNA, wherein the sgRNA is selected from the group consisting of NT, NT2, HPRTc.80, HPRTc.465, 531F(2), 52F(3), 715F(5), 451F(6), 176R(7), 551R(8), 230F(12), 164R(14), 676F(16), AGGn, L1.4_209F, and ALU_112a. In one aspect, the NT has the sequence of SEQ ID NO:1. SEQ ID NO: 1 is GTATTACTGATATTGGTGGG. In another aspect, the NT2 has the sequence of SEQ ID NO:2. SEQ ID NO:2 is GCGAGGTATTCGGCTCCGCG. In yet another aspect, the HPRTc.80 has the sequence of SEQ ID NO:3. SEQ ID NO:3 is ATTATGCTGAGGATTTGGAA. In still yet another aspect, the HPRTc.465 has the sequence of SEQ ID NO:4. SEQ ID NO:4 is TGGATTATACTGCCTGACCA. In yet another aspect, the 531F(2) has the sequence of SEQ ID NO:5. SEQ ID NO:5 is CACTCAGCATCGACTTACGA. In still yet a further aspect, the 52F(3) has the sequence of SEQ ID NO:6. SEQ ID NO:6 is TAATTACTGCACGATGCGCA. In yet another aspect, the 715F(5) has the sequence of SEQ ID NO:7. SEQ ID NO:7 is ATATATATGCGATCGAGCCC. In yet a further aspect, the 451F(6) has the sequence of SEQ ID NO:8. SEQ ID NO:8 is ACTAGTGTGCGTATGATTTG. In still yet another aspect, the 176R(7) has the sequence of SEQ ID NO:9. SEQ ID NO:9 is TCGATGTTCTACATCGATGT. In still yet a further aspect, the 551R(8) has the sequence of SEQ ID NO:10. SEQ ID NO:10 is TTGAATTGAGTTGCAACCGA. In yet another aspect, the 230F(12) has the sequence of SEQ ID NO:11. SEQ ID NO:11 is TTGTCCCACAATGATACTTG. In still yet another aspect, the 164R(14) has the sequence of SEQ ID NO:12. SEQ ID NO:12 is GGATATTTCACTACAGACTT. In still yet a further aspect, the 676F(16) has the sequence of SEQ ID NO:13. SEQ ID NO:13 is CTCCGAACTTAACTTGCCCT. In still a further aspect, the AGGn has the sequence of SEQ ID NO:14. SEQ ID NO:14 is AGGAGGAGGAGGAGGAGGAG. In another aspect, the L1.4_209F has the sequence of SEQ ID NO:15. SEQ ID NO:15 is TGCCTCACCTGGGAAGCGCA. In still another aspect, the ALU_112a has the sequence of SEQ ID NO:16. SEQ ID NO:16 is TTGCCCAGGCTGGAGTGCAG.

In one aspect, the CRISPR-Cas9 system comprises an sgRNA, wherein the sgRNA targets between about 1 to about 50 mutations in a target cell. In particular aspects, the sgRNA targets at least 50 mutations, at least 49 mutations, at least 48 mutations, at least 47 mutations, at least 46 mutations, at least 45 mutations, at least 44 mutations, at least 43 mutations, at least 42 mutations, at least 41 mutations, at least 40 mutations, at least 39 mutations, at least 38 mutations, at least 37 mutations, at least 36 mutations, at least 35 mutations, at least 34 mutations, at least 33 mutations, at least 32 mutations, at least 31 mutations, at least 30 mutations, at least 29 mutations, at least 28 mutations, at least 27 mutations, at least 26 mutations, at least 25 mutations, at least 24 mutations, at least 23 mutations, at least 22 mutations, at least 21 mutations, at least 20 mutations, at least 19 mutations, at least 18 mutations, at least 17 mutations, at least 16 mutations, at least 15 mutations, at least 14 mutations, at least 13 mutations, at least 12 mutations, at least 11 mutations, at least 10 mutations, at least 9 mutations, at least 8 mutations, at least 7 mutations, at least 6 mutations, at least 5 mutations, at least 4 mutations, at least 3 mutations, at least 2 mutations or at least 1 mutation. In some aspects, the targeting mutations are within non-coding regions in the target cell.

In other embodiments, the presently disclosed subject matter provides an sgRNA defined in Table 2. In some aspects, the sgRNA is selected from the group consisting of NT, NT2, HPRTc.80, HPRTc.465, 531F(2), 52F(3), 715F(5), 451F(6), 176R(7), 551R(8), 230F(12), 164R(14), 676F(16), AGGn, L1.4_209F, and ALU_112a. In one aspect, the NT has the sequence of SEQ ID NO:1. SEQ ID NO:1 is GTATTACTGATATTGGTGGG. In another aspect, the NT2 has the sequence of SEQ ID NO:2. SEQ ID NO:2 is GCGAGGTATTCGGCTCCGCG. In yet another aspect, the HPRTc.80 has the sequence of SEQ ID NO:3. SEQ ID NO:3 is ATTATGCTGAGGATTTGGAA. In still yet another aspect, the HPRTc.465 has the sequence of SEQ ID NO:4. SEQ ID NO:4 is TGGATTATACTGCCTGACCA. In yet another aspect, the 531F(2) has the sequence of SEQ ID NO:5. SEQ ID NO:5 is CACTCAGCATCGACTTACGA. In still yet a further aspect, the 52F(3) has the sequence of SEQ ID NO:6. SEQ ID NO:6 is TAATTACTGCACGATGCGCA. In yet another aspect, the 715F(5) has the sequence of SEQ ID NO:7. SEQ ID NO:7 is ATATATATGCGATCGAGCCC. In yet a further aspect, the 451F(6) has the sequence of SEQ ID NO:8. SEQ ID NO:8 is ACTAGTGTGCGTATGATTTG. In still yet another aspect, the 176R(7) has the sequence of SEQ ID NO:9. SEQ ID NO:9 is TCGATGTTCTACATCGATGT. In still yet a further aspect, the 551R(8) has the sequence of SEQ ID NO:10. SEQ ID NO:10 is TTGAATTGAGTTGCAACCGA. In yet another aspect, the 230F(12) has the sequence of SEQ ID NO:11. SEQ ID NO:11 is TTGTCCCACAATGATACTTG. In still yet another aspect, the 164R(14) has the sequence of SEQ ID NO:12. SEQ ID NO:12 is GGATATTTCACTACAGACTT. In still yet a further aspect, the 676F(16) has the sequence of SEQ ID NO:13. SEQ ID NO:13 is CTCCGAACTTAACTTGCCCT. In still a further aspect, the AGGn has the sequence of SEQ ID NO:14. SEQ ID NO:14 is AGGAGGAGGAGGAGGAGGAG. In another aspect, the L1.4_209F has the sequence of SEQ ID NO:15. SEQ ID NO:15 is TGCCTCACCTGGGAAGCGCA. In still another aspect, the ALU_112a has the sequence of SEQ ID NO:16. SEQ ID NO:16 is TTGCCCAGGCTGGAGTGCAG.

In other aspects, the presently disclosed subject matter provides a method for treating a disease, disorder, or condition associated with one or more somatic mutations in a subject in need of treatment thereof, the method comprising administering an effective amount of the presently disclosed CRISPR-Cas9 system to a target cell of the subject in need of treatment thereof. In certain aspects, the disease, disorder, or condition comprises a cancer. In particular aspects, the cancer is pancreatic cancer. In certain aspects, the cancer is a metastatic cancer.

In yet another embodiment, the present disclosure relates to a method for identifying novel protospacer adjacent motifs (PAMs), novel target sites, or novel PAMs and novel target sites in cells of a sample obtained from a subject. The method comprises:
a) analyzing sequencing data from one or more cells obtained from the subject for one or more somatic single base substitutions (SBS), one or more structural variants (SV), or one or more SBS and SVs that produce a PAM, a target site, or a PAM and a target site; and
b) identifying one or more PAMs, target sites, or PAMs and target sites in the cells based on the analysis in step a).

In the above method, the disease, disorder, or condition can be cancer.

In the above method, the cell is a cancer cell, a B-cell, a T-cell, a nerve cell, or combinations thereof. In some aspects, the one or more cells is a cancer cell. When the one or more cells is a cancer cell, the cancer cell is a cancer initiating cell.

In some aspects, the sequencing data is whole genome sequencing data.

In another embodiment, the present disclosure relates to a method of treating a disease, disorder or a condition in a subject. The method comprises:
a) analyzing sequencing data from one or more cells of a sample obtained from a subject suffering from a disease, disorder, or a condition, for one or more somatic single base substitutions (SBS), one or more structural variants (SV), or one or more SBS and SVs that produce a PAM, a target site, or a PAM and a target site;
b) identifying one or more PAMs, target sites, or PAMs and target sites in the cells based on the analysis in step a); and
c) administering to the subject an effective amount of a CRISPR-Cas9 system comprising a sgRNA, wherein the sgRNA targets (i) a sequence adjacent to the PAM; (ii) the target site; or (iii) combinations of (i) and (ii).

In the above method, the disease, disorder, or condition can be cancer.

In the above method, the cell is a cancer cell, a B-cell, a T-cell, a nerve cell, or combinations thereof. In some aspects, the one or more cells is a cancer cell. When the one or more cells is a cancer cell, the cancer cell is a cancer initiating cell.

In some aspects, the sequencing data is whole genome sequencing data.

In still other aspects of the above method, the method further comprises monitoring the subject receiving treatment with the CRISPR-Cas9 system.

In yet another embodiment, the present disclosure relates to a method of treating a subject suffering from a disease, disorder or a condition. The method comprises:
a) identifying one or more single somatic single base substitutions (SBS), one or more structural variants (SV), or one or more SBS and SVs that produce a PAM, a target site, or a PAM and a target site in one or more cells of a sample obtained from a subject suffering from a disease, disorder, or a condition; and
b) administering to the subject an effective amount of a CRISPR-Cas9 system comprising a sgRNA, wherein the sgRNA targets (i) a sequence adjacent to the PAM; (ii) the target site; or (iii) combinations of (i) and (ii).

In the above method, the disease, disorder, or condition can be cancer.

In the above method, the cell is a cancer cell, a B-cell, a T-cell, a nerve cell, or combinations thereof. In some aspects, the one or more cells is a cancer cell. When the one or more cells is a cancer cell, the cancer cell is a cancer initiating cell.

In still other aspects of the above method, the method further comprises monitoring the subject receiving treatment with the CRISPR-Cas9 system.

In still another embodiment, the present disclosure relates to a method of treating a subject suffering from a disease, disorder, or condition. The method comprises:
a) obtaining a sample from a subject suffering from a disease, disorder, or condition that is receiving treatment with a CRISPR-Cas system comprising a sgRNA that has developed resistance to said treatment;
b) identifying one or more single somatic single base substitutions (SBS), one or more structural variants (SV), or one or more SBS and SVs that were not previously identified in the subject and that produce a PAM, a target site, or a PAM and a target site in one or more cells of a sample obtained from the subject and that is different than the PAM and/or target site previously identified in the subject; and
c) administering to the subject an effective amount of a CRISPR-Cas9 system comprising a sgRNA, wherein the sgRNA targets (i) a sequence adjacent to the PAM; (ii) the target site; or (iii) combinations of (i) and (ii) identified in step b).

In the above method, the disease, disorder, or condition can be cancer.

In the above method, the cell is a cancer cell, a B-cell, a T-cell, a nerve cell, or combinations thereof. In some aspects, the one or more cells is a cancer cell. When the one or more cells is a cancer cell, the cancer cell is a cancer initiating cell.

In still other aspects of the above method, the method further comprises monitoring the subject receiving treatment with the CRISPR-Cas9 system.

In certain aspects, administering the CRISPR-Cas9 system to the target cell induces multiple double-strand breaks (DSBs). In one aspect, the CRISPR-Cas9 system targets at least 1 site in the target cell. In another aspect, In one aspect, the CRISPR-Cas9 system targets at least 2 sites, at least 3 sites, at least 4 sites, at least 5 sites, at least 6 sites, at least 7 sites, at least 8 sites, at least 9 sites, at least 10 sites, at least 11 sites, at least 12 sites, at least 13 sites, at least 14 sites, at least 15 sites, at least 16 sites, at least 17 sites, at least 18 sites, at least 19 sites, at least 20 sites, at least 21 sites, at least 22 sites, at least 23 sites, at least 24 sites, at least 25 sites, at least 26 sites, at least 27 sites, at least 28 sites, at least 29 sites, at least 30 sites, at least 31 sites, at least 32 sites, at least 33 sites, at least 34 sites, at least 35 sites, at least 36 sites, at least 37 sites, at least 38 sites, at least 39 sites, at least 40 sites, ta least 41 sites, at least 42 sites, at least 43 sites, at least 44 sites, at least 45 sites, at least 46 sites, at least 47 sites, at least 48 sites, at least 49 sites, or at least 50 sites in the target cell.

In certain aspects, the CRISPR-Cas9 system is delivered via a viral vector or one or more nanoparticles. In particular aspects, the viral vector is selected from an adenovirus, adeno-associated virus, retrovirus, lentivirus, Newcastle disease virus (NDV), and lymphocytic choriomeningitis virus (LCMV).

In certain aspects, the subject is a mammalian subject. In particular aspects, the mammalian subject is a human subject.

In other aspects, the presently disclosed subject matter provides a kit comprising the presently disclosed CRISPR-Cas9 system.

In other aspects, the presently disclosed subject matter provides a method for identifying novel protospacer adjacent motifs (PAMs), the method comprising analyzing whole genome sequencing (WGS) data of somatic single base substitutions (SBSs) for non-coding SBSs that create novel PAMs.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1A-1D show shows cytotoxicity as a function of the number of target sites. Growth inhibition as a function of the number of target sites in the human genome for two pancreatic cancer (PC) cell lines constitutively expressing Cas9 as detected by (FIG. 1A) alamarBlue cell viability reagent (R² Panc10.05=0.7424, TS0111=0.7685) and (FIG. 1B) phase microscopy (R² Panc10.05=0.7072, TS0111=0.6340) in 1:1000 dilution cultures. The assays were highly concordant (Pearson correlation coefficient=0.981) and cell line responses qualitatively similar (Pearson correlation coefficient ≥0.79). Data exclusion is based on criteria detailed in FIG. 11C. FIG. 1C shows the growth inhibition in the two PC cell lines for various sgRNAs. Note that the 12- and 14-target sgRNAs (230F(12) and 164R(14), respectively) show inhibition comparable to the positive control sgRNAs (AGGn, L1.4_209F, ALU_112a). FIG. 1D shows sgRNA tag survival of various sgRNAs as a function of time. All data with three biological replicates; error bars indicate mean±SEM.

FIG. 2A-2F show the genomic instability detected by cytogenetics and WGS. TS0111-Cas9-EGFP cells transduced with 164R(14) harvested on (FIG. 2A) day 1 and (FIG. 2B) day 10 after transduction. FIG. 2C shows the cytogenetic change (events per 100 metaphase cells) as a function of time. FIG. 2D shows the breakpoints on dicentric, tricentric, and ring chromosomes categorized by whether at targeted or non-targeted sites. FIG. 2E shows the break-apart FISH probe results for one of the target sites on 1q41 analyzed on day 14. FIG. 2F shows the WGS of Panc10.05-Cas9-EGFP surviving clones after treatment with multi-target sgRNAs bioinformatically analyzed to identify structural variants (SVs). SVs were categorized by whether they resulted from 2 sites targeted (green), 1 site targeted (red) or whether they were completely novel (no sites targeted, blue). Error bars indicate mean±SEM. 2 colonies each except 164R(14) (n=1).

FIG. 3A-3E show the polyploidization and apoptosis after treatment with 164R(14). FIG. 3A shows that Panc10.05-Cas9-EGFP cells transduced with NT2 or 164R(14), and stained with wheat germ agglutinin (WGA; green) and Hoechst (blue) 14 days after transduction. White arrow indicates a large nucleus and yellow arrows indicate multiple nuclei in a single cell. Metaphase images of cells on (FIG. 3B) day 0 and (FIG. 3C) day 10 after transduction of TS0111-Cas9-EGFP cells with 164R(14). FIG. 3D shows the number of cells with >6 X chromosomes over time using XY FISH. FIG. 3E shows the apoptosis of Panc10.05-Cas9-EGFP after treatment with 164R(14) or control (NT2), showing an increase on days 7 (Welch t test, two-tailed, p=0.046) and 14 (p=0.025) compared to pre-transduction, and decreased by day 21 (p=0.148). 3 biological replicates are shown.

FIG. 4A-4D show selective cell killing. FIG. 4A shows that co-cultures of Cas9-expressing human pancreatic cancer (Panc10.05) and mouse fibroblast (NIH 3T3) cell lines transduced with human-specific 230F(12) sgRNA, and monitored over time using flow cytometry and a human-mouse polymorphism NGS assay. Error bars indicate mean±SEM; 3 biological replicates. FIG. 4B shows the mutation frequency at 7 Panc480-specific target sites in parental Panc480, Cas9 expressing Panc480, 480 lymphoblasts (Onc3286), or a negative control Panc1002 cell line after treatment with the NT (−) or MT7 (+) multiplex sgRNA vector. FIG. 4C shows flow cytometry analysis of Panc480-Cas9-mApple and Panc10.05-Cas9-EGFP cell mixtures after treatment with NT, or the multiplex sgRNA vectors, MT7 and Top7. Error bars indicate mean±SEM; 3 biological replicates with 2 technical replicates each. FIG. 4D shows STR analysis of Panc480 (parental)/Panc10.05-Cas9-EGFP (−Cas9) or Panc480-Cas9-mApple/Panc10.05-Cas9-EGFP (+Cas9) cell line mixtures after treatment with MT7 or Top7. Error bars indicate mean±SEM; 3 biological replicates with 2 technical replicates each for +Cas9, 1 technical replicate each for −Cas9.

FIG. 5A-5C show that novel PAMs are conserved as we age, and targeting multiple sites causes genomic instability that leads to delayed cancer cell death. FIG. 5A shows Novel PAMs arising from mutations in two primary tumors were confirmed in regional lymph node metastases. FIG. 5B shows cancer initiation cell (CIC) mutations occur at approximately 40 mutations/year/cell during the time between the zygote and the birth of the CIC. CIC mutations and initiating driver mutations are expected to be in all cancer cells (light red cells). Other driver mutations and passenger mutations that arise during the time between the CIC and diagnosis should be subclonal (dark red cells). These mutations produce an average of 488 novel PAMs (absent in normal lymphs) when a patient reaches around 59 years old. The figure is created with BioRender.com. FIG. 5C shows toxicity in multi-target sgRNA-transduced PC cells occurred following the induction of multiple DSBs and their repair resulting in polyploidization, chromosomal rearrangement, and ultimately cell death.

FIG. 6A-6F show that both Cas9 and sgRNA have to be present to achieve maximal toxicity, and most mutations came from perfect target sites. FIG. 6A shows the functional Cas9 activities of four PC cell lines (Panc10.05, TS0111, Panc480, and Panc1002) labeled with Cas9-EGFP or Cas9-mApple are shown. Error bars indicate mean±SEM; 3 biological replicates. FIG. 6B shows that two PC cell lines (Panc10.05 and TS0111), labeled with dCas9-EGFP or Cas9-EGFP, were transduced with non-targeting sgRNAs (indicated as “multitarget sgRNA −”) or sgRNAs targeting repetitive elements (indicated as “multitarget sgRNA +”). Cells were then plated at 1:10 dilution, and toxicity was quantified via alamarBlue cell viability assay. Error bars indicate mean±SEM; 3 biological replicates. FIG. 6C shows the WGS of Panc10.05 resistant colonies showed number of predicted target sites highly correlates with the number of Cas9-induced mutated sites in Panc10.05 (Pearson r=0.9875), in which the number of mutated sites were determined by copy number of each target site in Panc10.05. FIG. 6D shows that the total Cas9-induced mutation frequency of all target sites in each clone was plotted against alamarBlue growth inhibition data from the clonogenicity experiment (R-squared of Panc10.05 and TS0111 are 0.846 and 0.764, respectively). The predicted number of target site which assumes 100% VAF at all perfect target sites were also plotted against the same inhibition data (R-squared of Panc10.05 and TS0111 are 0.728 and 0.687, respectively). FIG. 6E shows that the correlation between total mutation frequency of perfect target site and all mutated sites. Dotted lines indicate only perfect target sites are mutated at a 100% mutation frequency. Pearson r correlation coefficient of Panc10.05 and TS0111 are 0.994 and 0.997, respectively. FIG. 6F shows that the WGS data of 40 resistant colonies were analyzed to interrogate the effect of single nucleotide variant (SNV) present on perfect target site on their respective mutation frequencies. Most colonies with <25% perfect target sites containing SNV (x-axis) exhibited >50% mutation frequency on their perfect target sites, except for 2 colonies.

FIG. 7A-7D show a dose-response of target sites vs toxicity is observed across different PC cell lines, and significant sgRNA reduction is mostly observed after day 7 of sgRNA transduction. FIG. 7A shows sgRNA tag survival at day 21 after transduction for sgRNAs targeting different numbers of sites in the human genome. Error bars indicate mean±SEM. FIG. 7B shows sgRNA tag survival directly correlated with growth inhibition, especially when the growth inhibition exceeded 70% (alamarBlue, Pearson correlation coefficient: −0.811, p=0.0004). FIG. 7C shows the results of treating five PC cell lines with Cas9 and multi-target sgRNAs that have 0-16 predicted perfect target sites in the human genome. FIG. 7D shows the results of treating two PC cell lines that express Cas9-EGFP constitutively, after transduction with multi-target sgRNAs that have 0-16 predicted perfect target sites in the human genome. Cells were plated at 1:10 dilution, and toxicity was quantified via alamarBlue cell viability assay in a 96-well plate. All data shown in this figure consists of 3 biological replicates.

FIG. 8A-8E show the mutation frequency peaks at around day 3-5 post transduction of a 14-cutter sgRNA, and the sgRNA expression leads to genomic instability over time. FIG. 8A shows the mutation frequency at 8 different target loci of Panc10.05-Cas9-EGFP cells at 8 different target loci transduced with a 14-cutter sgRNA, 164R(14) at various time points. FIG. 8B shows the karyotype of TS0111-Cas9-EGFP without sgRNA transduction. Chromosome breakage analysis of transduced cells on day (FIG. 8C) 3, (FIG. 8D) 14, and (FIG. 8E) 16 were shown with genomic instability features indicated. FIG. 8F shows a total of 90 dicentric and tricentric chromosomes were analyzed to characterize the location of breakpoints to determine if the breakpoint is present at a target region of 164R(14) or a non-target region, and whether it is located at the telomeric end of chromosomes or non-telomeric regions.

FIG. 9A-9D show a demonstration of translocations as a result of CRISPR-Cas9 cuts, and SV identification and quantification using Trellis. FIG. 9A shows an illustration of the break-apart FISH strategy at the 1q41 cut site. Abnormal FISH patterns were shown using cells collected at various timepoints. FIG. 9B shows that complex rearrangements are observed with cells on day 16 post transduction of sgRNA. FIG. 9C shows the percentage of cells with rearrangements at 1q41 as a function of time is shown. FIG. 9D shows WGS of Panc10.05-Cas9-EGFP surviving clones were bioinformatically analyzed using Trellis to identify SVs. The BAM files are bowtie2-aligned and showed higher sensitivity and less specificity than bwa-aligned files used in FIG. 2F with a different SV caller (Manta). Error bars indicate mean±SEM; 2 resistant colonies each, except 164R(14) (1 colony).

FIG. 10A-10D show expression of a 14-cutter sgRNA, 164R(14), in Panc10.05-Cas9-EGFP cells leads to polyploidy and apoptosis. Shown are the cells on day 14 post-transduction of either a (FIG. 10A) non-targeting sgRNA, NT2, or (FIG. 10B) a 14-cutter sgRNA, 164R(14). Cells membranes were stained with wheat germ agglutinin (WGA; green fluorescence) and genomic content with Hoechst (blue). FIG. 10C shows annexin V flow cytometry assay was performed to quantify proportion of live cells (Welch t tests; two-tailed; p-values for day 7=0.046, day 14=0.025, and day 21=0.151) compared to non-targeting (NT2) sgRNA control over time. FIG. 10D shows that TUNEL staining was also performed to quantify apoptotic cells. For both assays, error bars indicate mean±SEM; three biological replicates were shown.

FIG. 11A-11B show strategies to target somatic mutations in cancer. Three methods were implemented to design sgRNAs based on somatic PAMs and novel breakpoints found in three PC cell lines: FIG. 11A shows WES-based base substitution identification, WGS-based base substitution identification, and FIG. 11B shows structural variant identification. For example, (FIG. 11A) some base substitution mutations (C→G) can create a novel PAM site; (FIG. 11B) with a deletion, novel DNA sequences (green) are juxtaposed next to a pre-existing NGG site. SVs could also theoretically generate a novel NGG (not shown). Numbers shown are the averages of three PC cell lines.

FIG. 12A-12F show human cell line-specific toxicity is reproducible across different combinations of mouse-human co-cultures, and this toxicity is a result of the presence of both Cas9 and human-specific sgRNA. FIG. 12A shows a comparison of number of target sites of NT (SEQ ID NO:1) and 230F(12) (SEQ ID NO:11) sgRNAs in both mouse (mm10) and human (hg38) genomes. “mm” refers to mismatch. FIG. 12B shows an alignment of the mouse and human RC3H2 orthologs shows differences of a 3 bp indel and 3 SNPs between the two species, highlighted by red boxes. PCR primer sequences are underlined. FIG. 12C shows the sensitivity and accuracy of the mouse-human NGS assay was validated by deep sequencing known mixes of mouse and human DNA. Pearson r=0.9941, p<0.0001. FIG. 12D shows TS0111 and NIH 3T3 Cas9-expressing cell lines were co-cultured and transduced with 230F(12). Shown are the changes in TS0111 cell population over time by flow cytometry and human-mouse NGS assay. FIG. 12E shows Panc10.05 and Panc02, a KPC-derived mouse cell line, were also co-cultured and transduced with the same sgRNA, in which the change in Panc10.05 cell population was measured by flow cytometry. FIG. 12F shows NIH 3T3-Cas9 was co-cultured with Panc10.05 parental, dCas9-expressing cell line, and Cas9-expressing cell line, separately, and transduced with 230F, in which the change in NIH 3T3 cell population was measured by flow cytometry. For FIG. 12D-FIG. 12F, error bars indicate mean±SEM; three biological replicates were shown.

FIG. 13A-FIG. 13B show lentiGuide-puro_Panc480-MT7 and -Top7, and dose-response of the STR profiling assay. FIG. 13A shows tandem CRISPR array with U6 promoter, sgRNA sequence (red line), and gRNA scaffold targeting 7 novel PAMs in the Panc480 cell line. Cartoon courtesy of SnapGene. FIG. 13B shows the locus and guide sequence for each of the 7 targets in MT7 and Top7 (Targets: chr8_201457-SEQ ID NO: 455; chr17_5377742-SEQ ID NO:456; chr3_537601-SEQ ID NO:457; chr3_59525282-SEQ ID NO:458; chrX_3982448-SEQ ID NO:459; chr8_29032916-SEQ ID NO:460; chr18_1819017-SEQ ID NO:461; chr19_58564841-SEQ ID NO:462; chr6_124767224-SEQ ID NO:463). FIG. 13C shows the sensitivity and accuracy of the STR profiling assay was validated using known mixes of Panc480 and Panc10.05 cells. Pearson r=0.9803, p=0.0006.

FIG. 14 is schematic showing a representative clinical trial workflow demonstrating implementation of the claimed methods of the present disclosure.

FIG. 15A-15E show that somatic PAM discovery yielded hundreds of novel PAMs in pancreatic cancers (PCs). FIG. 15A shows somatic NGG PAMs can arise through SBS that creates a novel G from A/T/C (indicated as X), and this novel G is adjacent to an existing G one nucleotide downstream (SBS 1) or upstream (SBS 2) of the novel G. Examples of T>G are shown. The same concept applies to the complementary strand, in which SBS produces a novel CCN sequence. FIG. 15B shows IGV screenshots of two novel PAMs found in Panc480 tumor which are absent in their corresponding normal. FIG. 15C shows mutational signatures of two pancreatic cancer cell lines (Panc480 and Panc504), showing the proportion of mutations created novel Gs and Cs that could potentially form novel PAMs (highlighted in red boxes). Y-axis is the percentage of SBS. FIG. 15D shows the workflow of somatic PAM discovery. Whole genome sequencing was performed on both tumor cell line and corresponding normal cell line to obtain somatic SBSs via tumor-normal subtraction. An average of 4548 somatic SBSs were found. A somatic PAM discovery software, PAMfinder, was employed to identify SBSs that produced novel PAMs, resulting in an average of 417 somatic PAMs per cell line, which was 9.2% of the SBSs discovered. After applying a variant allele frequency (VAF) cutoff of 95% and inspecting the potential sgRNAs for risk of off-target activity, we shortlisted an average of 33 sgRNAs per cell line for downstream testing. FIG. 15E shows the proportions of novel PAMs discovered in Panc480 (left) and Panc504 (middle), and Panc1002 (right) that were located in different regions of the genome. Others include non-coding RNAs, untranslated regions, and 1-kb regions upstream/downstream of transcription start/end sites. VAF cutoff=30%. For Panc480, no novel PAMs were found in exons.

FIG. 16A-16E show hundreds to thousands of somatic PAMs were found in different adult solid tumor types. FIG. 16A shows the workflow of PAM discovery in 591 tumor samples using tumor-normal subtracted variant call files from ICGC. All analyses were corrected based on the tumor purity of individual sample. Samples from four cohorts were included: APGI-AU (Pancreas (AU); N=44), PACA-CA (Pancreas (CA); N=130), LUCA-KR (Lung (KR); N=29), and OCCAMS-GB (esophagus (GB); N=388). (B-C) Truncated violin plots present the total number of (FIG. 16B) base substitutions (log scale) and (FIG. 16C) novel PAMs (log scale) in each cohort. (FIG. 16D) Truncated violin plots present the percentage of base substitutions that contributed to somatic PAM. Kolmogorov-Smirnov tests were performed. ns indicates non-significant; **** indicates P<0.0001. (E) Mutational spectra analysis in each cohort.

FIG. 17A-17F shows that selective cell killing was achieved with low number of targets discovered from our novel PAM approach. FIG. 17A shows novel PAMs arising from mutations in two primary tumors were confirmed of their presence in metastatic sites via Sanger sequencing. FIG. 17B shows co-cultures of Cas9-expressing human PC (Panc10.05) and mouse fibroblast (NIH 3T3) cell lines transduced with human-specific 230F(12) sgRNA were monitored over time using flow cytometry and a human-mouse polymorphism NGS assay. Error bars indicate mean±SEM; N=3. FIG. 17C shows a tandem CRISPR array with U6 promoter, sgRNA sequence (red line), and sgRNA scaffold targeting 7 novel PAMs in the Panc480 cell line. Diagram was generated by SnapGene. FIG. 17D shows the mutation frequency at 7 Panc480-specific target sites in parental Panc480, Cas9-expressing Panc480, Panc480 patient's Cas9-expressing lymphoblasts (Onc3286), and Panc1002 (negative control) cell lines after treatment with NT (−) or MT7 (+) multiplex sgRNA vector. FIG. 17E show flow cytometry analysis of Panc480-Cas9-mApple and Panc10.05-Cas9-EGFP cell mixtures after treatment with NT or MT7 on day 1 and day 21 post transduction of sgRNAs. Paired t tests were performed; ns indicates p>0.05; ** indicates p<0.01. Error bars indicate mean±SEM; 3 biological replicates with 2 technical replicates each. FIG. 17F shows the STR analysis of Panc480 (parental)/Panc10.05-Cas9-EGFP (−Cas9) or Panc480-Cas9-mApple/Panc10.05-Cas9-EGFP (+Cas9) cell line mixtures after treatment with MT7 on day 21. Paired t tests were performed; * indicates p<0.05; ** indicates p<0.01. Error bars indicate mean±SEM; 3 biological replicates with 2 technical replicates each for +Cas9, 1 technical replicate each for −Cas9.

FIG. 18A-FIG. 18C shows the structural variants create novel CRISPR-Cas9 target sites. Structural variants, such as (FIG. 18A) deletion and (FIG. 18B) translocation, could give rise to novel target sequence if the new junction is in proximity of an existing NGG PAM (shown) or creates a new PAM (not shown). For example, (FIG. 18C) a chr1: chr9 translocation in Panc480 gave rise to a novel breakpoint that is in proximity of an existing AGG PAM (labeled in green). This breakpoint is characterized by a 5 bp GGAGC (SEQ ID NO:17) microhomology at its junction (labeled in red).

FIG. 19A-19C shows that mutational signatures indicate clock-like signatures for most SBSs. Mutational signatures of SBSs found in (FIG. 19A) Panc480, (FIG. 19B) Panc504, and (FIG. 19C) Panc1002 suggest that most mutations arose from aging. The only exception is SBS18 found in Panc1002, which is linked to possible damage by reactive oxygen species. Y-axis is the percentage of SBS.

FIG. 20 shows that human cell line-specific toxicity was reproducible across different combinations of mouse-human co-cultures, and this selective cell elimination required the presence of both Cas9 and human-specific sgRNA. (FIG. 20A-FIG. 20B) Cas9 activity assay was performed on (FIG. 20A) four PC cell lines (Panc10.05, TS0111, Panc480, and Panc1002) and (FIG. 20B) two mouse cell lines (NIH3T3 and Panc02), all labeled with Cas9-EGFP or Cas9-mApple, to quantify mutation frequency at the HPRT1 gene locus. FIG. 20C shows the alignment of the mouse and human RC3H2 orthologs shows differences of a 3 bp indel and 3 SNPs between the two species, highlighted by red boxes. PCR primer sequences are underlined. FIG. 20D shows the sensitivity and accuracy of the mouse-human NGS assay was validated by deep sequencing known mixes of mouse and human DNA. Pearson r=0.9941, p<0.0001, N=3. FIG. 20E shows that TS0111 and NIH 3T3 Cas9-expressing cell lines were co-cultured and transduced with 230F(12). Shown are the changes in TS0111 cell population over time by flow cytometry and human-mouse NGS assay. FIG. 20F shows the Panc10.05 and Panc02, a KPC-derived mouse cell line, were also co-cultured and transduced with the same sgRNA, in which the change in Panc10.05 cell population was measured by flow cytometry. FIG. 20G shows the NIH 3T3-Cas9 was co-cultured with Panc10.05 parental, dCas9-expressing cell line, and Cas9-expressing cell line, separately, and transduced with 230F(12), in which the change in NIH 3T3 cell population was measured by flow cytometry. For FIG. 20E-FIG. 20G, error bars indicate mean±SEM; N=3.

FIG. 21 shows the dose-response of the STR profiling assay. Sensitivity and accuracy of the STR profiling assay was validated using known mixes of Panc480 and Panc10.05 cells. Pearson r=0.9803, p=0.0006.

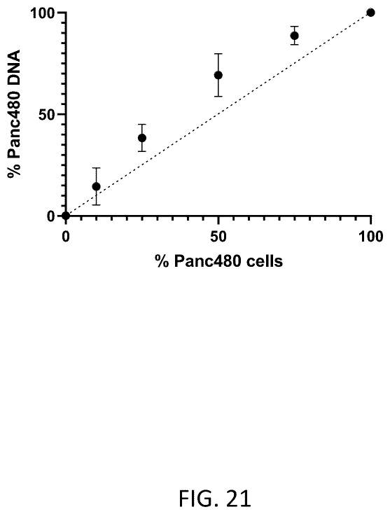

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. Likewise, the term “include” and its grammatical variants are intended to be non-limiting, such that recitation of items in a list is not to the exclusion of other like items that can be substituted or added to the listed items. The present disclosure contemplates other embodiments “comprising,” “consisting of” and “consisting essentially of,” the embodiments or elements presented herein, whether explicitly set forth or not.

The singular forms “a,” “and” and “the” include plural references unless the context clearly dictates otherwise. Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Units, prefixes, and symbols are denoted in their Systeme International de Unites (SI) accepted form. Unless otherwise indicated, nucleic acids are written left to right in 5′ to 3′ orientation; amino acid sequences are written left to right in amino to carboxy orientation.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

As used herein, the term “administering” means the actual physical introduction of a CRISPR-Cas9 system into or onto (as appropriate) a target cell. Any and all methods of introducing the composition into the target cell are contemplated according to the disclosure; the method is not dependent on any particular means of introduction and is not to be so construed. Means of introduction are well-known to those skilled in the art, and also are exemplified herein.

“Vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

As used herein, the term “treating,” “treat,” or “treatment” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed CRISPR-Cas9 systems can be administered prophylactically to prevent or reduce the incidence or recurrence of the disease, disorder, or condition.

As used herein, the term “inhibit” or “inhibits” means to decrease, suppress, attenuate, diminish, arrest, or stabilize an activity associated with a disease or a disease-related pathway or the development or progression of a disease, disorder, or condition, e.g. cancer, by at least 10%, 20%, 30%, 40%, 50%, 60%, 70%, 80%, 90%, 95%, 98%, 99%, or even 100% compared to an untreated control subject, cell, biological pathway, or biological activity.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a CRISPR-Cas9 system described herein and at least one other therapeutic agent, such as a chemotherapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, the term “CRISPR-Cas9” is a molecular scissor that can induce a double strand break (DSB) at a specific genomic location as determined by the sgRNA sequence. In one embodiment, DSBs are known to be toxic to cells and lead to cell death, which is the driving mechanism behind many cytotoxic therapies, such as radiation therapies. In one embodiment, the CRISPR-Cas9 is known as a gene-editing technology for modifying, deleting, correcting, or inserting precise regions of DNA. In some embodiments, the CRISPR/Cas9 edits genes by precisely cutting DNA and then letting natural DNA repair processes to take over.

As used herein, the term “sgRNAs” or “sgRNA-guided Cas 9” as used interchangeably herein, refers to a single guide RNA, which is a single RNA molecule that contains both the custom-designed short crRNA sequence fused to the scaffold tracrRNA sequences. In some embodiments, sgRNA is synthetically made in vitro or in vivo from a DNA template.

As used herein, the term “cancer” refers to a disease caused by an uncontrolled division of abnormal cells in a part of the body. Examples of cancer include, but are not limited to, anal cancer, bile duct cancer, bladder cancer, bone cancer, brain tumor and/or cancer, breast cancer, bronchial tumors, Burkitt lymphoma, cardiac tumors, cervical cancer, leukemia, colorectal cancer, uterine cancer, esophageal cancer, ewing sarcoma, fallopian tube cancer, gallbladder cancer, gastric cancer, gastrointestinal carcinoid tumor, head and neck cancer, kidney cancer, liver cancer, lip and oral cavity cancer, lung cancer, lymphoma, melanoma, skin cancer, metastatic cancer, mouth cancer, ovarian cancer, pancreatic cancer, prostate cancer, rectal cancer, salivary gland cancer, throat cancer, thyroid cancer or any combinations thereof.

As used herein, the term “pancreatic cancer” refers to a type of cancer that starts in the pancreas. Pancreatic cancer types include, but are not limited to, exocrine pancreatic cancer, neuroendocrine pancreatic cancer. The most common type of pancreatic cancer, adenocarcinoma of the pancreas, starts when exocrine cells in the pancreas start to grow out of control.

As used herein, the term “benign pancreatic disease” and “pancreatic disease” as used herein interchangeably refer to pancreatic disease which is not cancer or has become cancer. Benign pancreatic disease includes pancreatitis, various types of cysts and tumors, pancreatic intraepithelial neoplasia (PanIN) and intraductal papillary mucinous neoplasm (IPMN) lesions, and mucinous cystic neoplasm (MCN).

As used herein, the term “early-stage pancreatic cancer” as used herein refers to pancreatic cancer which is limited to the pancreas, outside the pancreas or nearby lymph nodes, but has not expanded into nearby major blood vessels or nerves or distant organs. Early-stage pancreatic cancer includes stage 0, stage I and stage II pancreatic cancers. See Yachida et al. (2010) Nature 467:1114-1119; see also National Comprehensive Cancer Network (NCCN) Guidelines Version 2.2012 Pancreatic Adenocarcinoma.

As used herein, the term “late-stage pancreatic cancer” as used herein refers to pancreatic cancer which has expanded into nearby major blood vessels, nerves or distant organs. Late-stage pancreatic cancer includes stage III or stage IV pancreatic cancer.

As used herein, the term “stage 0 pancreatic cancer” as used herein refers to pancreatic cancer limited to a single layer of cells in the pancreas. The pancreatic cancer is not visible on imaging tests or to the naked eye. The tumor is confined to the top layers of pancreatic duct cells and has not invaded deeper tissues or spread outside of the pancreas. Stage 0 tumors are sometimes referred to as pancreatic carcinoma in situ or pancreatic intraepithelial neoplasia III (PanIn III).

As used herein, the term “stage I pancreatic cancer” as used herein refers to cancer confined or limited to the pancreas and has not spread to nearby lymph nodes. “Stage IA” refers to a tumor confined to the pancreas and is less than 2 cm in size. “Stage IB” refers to a tumor confined to the pancreas and is greater than 2 cm in size.

As used herein, the term “stage II pancreatic cancer” as used herein refers to local spread cancer that has grown outside the pancreas or has spread to nearby lymph nodes. “Stage IIA” refers to a tumor growing outside the pancreas but not into large blood vessels, nearby lymph nodes or distant sites. “Stage IIB” refers to a tumor either confined to the pancreas or growing outside the pancreas but has not spread into nearby large blood vessels or major nerves. Stage IIB may spread to nearby lymph nodes but has not spread to distant sites.

As used herein, the term “stage III pancreatic cancer” as used herein refers to wider spread cancer that has expanded into nearby major blood vessels or nerves but has not metastasized. The tumor is growing outside the pancreas into nearby large blood vessels or major nerves and may or may not have spread to nearby lymph nodes. It has not spread to distant sites.

As used herein, the term “stage IV pancreatic cancer” as used herein refers to confirmed spread cancer that has spread to distant organs or sites. Stage IVA pancreatic cancer is locally confined, but involves adjacent organs or blood vessels, thereby hindering surgical removal. Stage IVA pancreatic cancer is also referred to as localized or locally advanced. Stage IVB pancreatic cancer has spread to distant organs, most commonly the liver. Stage IVB pancreatic cancer is also called metastatic.

As used herein, the term “metastasis cancer” refers to a cancer that spreads from where it started to a distant part of the body is called metastatic cancer. For many types of cancer, it is also called stage IV (4) cancer.

As used herein, the term “target cell” refers to a cell selectively affected, identified by, attacked and/or targeted by the CRISPR-Cas9 system as described herein. In some embodiments, the target cells are, but not limited to, one or more cells having one or more somatic mutations, such as, cancer cells, particularly pancreatic, lung, and esophageal cancer. In some aspects, the one or more somatic mutations produce one or more protospacer adjacent motifs (PAMs) and/or target sites (e.g., sequences).

As used herein, the term “protospacer adjacent motifs (PAMs)” refers to a short DNA sequence (typically 2-6 base pairs in length) that follows the DNA region targeted for cleavage by the CRISPR system, such as CRISPR-Cas9. The PAM is generally required for a Cas nuclease to cut and is typically found 3-4 nucleotides downstream from the cut site.

### 2. Methods of Designing CRISPR-Cas9 Systems for Treating Disease

In some embodiments, the present disclosure relates to methods of identifying somatic mutations in one or more tumors that produces one or more protospacer adjacent motifs (PAMs) and/or novel target sites (e.g., sequences) in a subject. As used herein, the term “somatic mutation(s)” refers to any alteration at the cellular level in somatic tissues occurring after fertilization. Examples of somatic mutations include, but are not limited to, cancer and noncancerous disease (such as autoimmune and/or neurodegenerative diseases). The methods described herein can be used on any subject or patient that is suffering or believed to be suffering from a disease, disorder, a condition, or any combination thereof. In some aspects, the subject is suspected of having a tumor. In other aspects, the subject is confirmed or known to have a tumor. In some further aspects, the tumor is cancer.

The first step of the method involves obtaining two samples from the subject. The first sample is a sample from the tumor in the subject. The second sample is a non-tumor (e.g., normal) sample from the (same) subject. The sample can be obtained from the subject using routine techniques in the art. For example, the one or more tumor samples can be a tissue sample, a blood sample, a plasma sample, a serum sample, an urine sample, cerebrospinal fluid, stool or feces, saliva, ascites fluid, sputum, synovial fluid, or any combination thereof. In some further aspects, the tumor sample can be a cell, such as, for example, a cancer initiating cell (CIC). The one or more non-tumor samples can be a tissue sample, a blood sample, a plasma sample, a serum sample, an urine sample, cerebrospinal fluid, stool or feces, saliva, ascites fluid, sputum, synovial fluid, or any combination thereof. In some aspects, once the tumor sample and non-tumor samples (e.g., normal sample) are obtained from the subject, at least one tumor cell line is prepared from the tumor sample and at least one non-tumor or normal cell line is produced from the non-tumor (e.g., normal) sample. The tumor and normal cell lines can be produced using routine techniques known in the art. After the tumor and normal cell lines are produced, DNA from each of the tumor and normal cell lines is obtained using routine techniques known in the art.

In other aspects, DNA is obtained from the tumor and normal samples, without generating cell lines, using routine techniques known in the art.

Once DNA from each of the tumor and normal cell lines or from the tumor and normal cells is obtained, then next generation sequencing, such as whole genome sequencing (e.g., whole genome sequencing-based base substitution identification), whole exome sequencing (e.g., whole exome sequencing-based base substitution identification), structural variant identification, Sanger sequencing, etc.) of each of the DNA is performed using routine techniques known in the art to produce a tumor sequence and a normal sequence.

Once the tumor and normal sequences are obtained, a tumor-normal subtraction can be performed using one or more bioinformatics pipelines known in the art to obtain tumor only somatic mutations and to exclude germline mutations that exist in both the tumor and normal samples. After the subtraction is performed, somatic mutations in the tumor sequence that produce one or more PAMs and/or target sites are identified using next generation sequencing, such as, for example, whole genome sequencing (e.g., whole genome sequencing-based base substitution identification), whole exome sequencing (e.g., whole exome sequencing-based base substitution identification), structural variant identification, Sanger sequencing, etc.). Specifically, the tumor sequence is analyzed to identify one or more somatic base substitutions (BS), such as single base substitutions (SBS), one or more structural variants (SV), or one or more BS and SVs that produce a novel (e.g., new) PAM, a novel (e.g., new) target site, or a novel PAM and a novel target site (which can be in the coding region of the subject's genome or the non-coding region of the subject's genome). Once the one or more BS and/or SVs are identified, one or more novel PAMs and/or target sites are identified. In some aspects, the novel PAM and/or novel target site will have a variant allele frequency (VAF) of at least 1%, at least 2%, at least 3%, at least 4%, at least 5%, at least 6%, at least 7%, at least 8%, at least 9% or at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, or at least 99% depending on the method used (e.g., next generation sequencing, such as, for example, whole genome sequencing-based base substitution identification, whole exome sequencing-based base substitution identification, structural variation identification, Sanger sequencing, etc.).

Once the one or more novel PAMs and/or target sites are identified, then one or more sgRNAs can be designed using routine techniques known in the art. Generally, the sgRNAs will have a VAF greater than 50%, greater than 60%, greater than 70%, greater than 75%, greater than 80%, greater than 85%, greater than 90%, or greater than 95%. Additionally, once the one or more novel PAMs and/or target sites are identified, then PCR, Sanger sequencing, or other techniques known in the art can be used to confirm that the designed sgRNAs target the somatic mutations that produce the one or more PAMs and/or target sites.

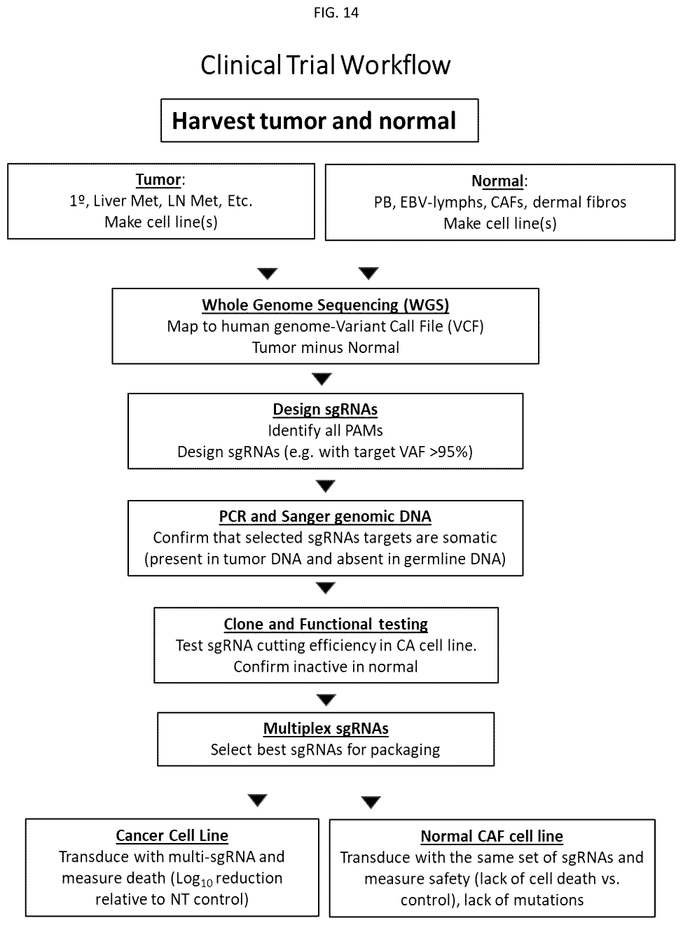

A flow chart providing a method of the present disclosure is shown in FIG. 14.

Once the PAM and/or target site is identified, the subject can be administered an effective amount of a CRISPR-Cas9 system comprising a sgRNA which has been designed to target the novel PAM and/or novel target site. Specifically, the sgRNA targets a sequence adjacent to the novel PAM and/or directly targets the novel target site in proximity to an existing PAM. As used herein, the term “adjacent” means a sequence that is next to the PAM.

The sgRNAs contained in the CRISPR-Cas9 system are designed to be both patient-specific and cancer-specific by identifying novel structural variants or base substitutions that lead to novel target site and/or novel PAMs as a result of base substitutions. In some aspects, the sgRNAs are designed to have multiple (e.g., 1-50) target sites for the effect of multiple double-stranded breaks (DSBs). In other words, the sgRNAs are designed as multi-target sgRNAs. In another aspect, the sgRNAs are designed to cut in non-coding regions of the genome. In still another aspect, the sgRNAs are designed to have low numbers of off-target sites and high targeting efficiencies. In a further aspect, the sgRNA determines a specific genomic location for a double-strand break. In certain aspects, the sgRNA is selected from the group consisting of NT, NT2, HPRTc.80, HPRTc.465, 531F(2), 52F(3), 715F(5), 451F(6), 176R(7), 551R(8), 230F(12), 164R(14), 676F(16), AGGn, L1.4_209F, and ALU_112a. In one aspect, the NT has the sequence of SEQ ID NO: 1. SEQ ID NO:1 is GTATTACTGATATTGGTGGG. In another aspect, the NT2 has the sequence of SEQ ID NO:2. SEQ ID NO:2 is GCGAGGTATTCGGCTCCGCG. In yet another aspect, the HPRTc.80 has the sequence of SEQ ID NO:3. SEQ ID NO:3 is ATTATGCTGAGGATTTGGAA. In still yet another aspect, the HPRTc.465 has the sequence of SEQ ID NO:4. SEQ ID NO:4 is TGGATTATACTGCCTGACCA. In yet another aspect, the 531F(2) has the sequence of SEQ ID NO:5. SEQ ID NO:5 is CACTCAGCATCGACTTACGA. In still yet a further aspect, the 52F(3) has the sequence of SEQ ID NO:6. SEQ ID NO:6 is TAATTACTGCACGATGCGCA. In yet another aspect, the 715F(5) has the sequence of SEQ ID NO:7. SEQ ID NO:7 is ATATATATGCGATCGAGCCC. In yet a further aspect, the 451F(6) has the sequence of SEQ ID NO:8. SEQ ID NO:8 is ACTAGTGTGCGTATGATTTG. In still yet another aspect, the 176R(7) has the sequence of SEQ ID NO:9. SEQ ID NO:9 is TCGATGTTCTACATCGATGT. In still yet a further aspect, the 551R(8) has the sequence of SEQ ID NO:10. SEQ ID NO:10 is TTGAATTGAGTTGCAACCGA. In yet another aspect, the 230F(12) has the sequence of SEQ ID NO:11. SEQ ID NO:11 is TTGTCCCACAATGATACTTG. In still yet another aspect, the 164R(14) has the sequence of SEQ ID NO:12. SEQ ID NO:12 is GGATATTTCACTACAGACTT. In still yet a further aspect, the 676F(16) has the sequence of SEQ ID NO:13. SEQ ID NO:13 is CTCCGAACTTAACTTGCCCT. In still a further aspect, the AGGn has the sequence of SEQ ID NO:14. SEQ ID NO:14 is AGGAGGAGGAGGAGGAGGAG. In another aspect, the L1.4_209F has the sequence of SEQ ID NO:15. SEQ ID NO:15 is TGCCTCACCTGGGAAGCGCA. In still another aspect, the ALU_112a has the sequence of SEQ ID NO:16. SEQ ID NO:16 is TTGCCCAGGCTGGAGTGCAG.

### 3. CRISPR-Cas9 System

In another embodiment, the present disclosure relates to using the CRISPR-Cas9 system designed according to the methods described above in Section 2, as a selective cell killing tool by identifying PAMs and/or other target sites (e.g., sequences) specific to a tumor cell, designing sgRNAs targeting the PAMs and/or other target sites, and introducing the CRISPR-Cas9 system into the cell of a subject to induce multiple DSBs. In other embodiments, the presently disclosed subject matter provides the CRISPR-Cas9 system for treating a disease, disorder, or condition associated with one or more somatic mutations in a subject in need of treatment thereof, the system comprising an sgRNA-guided Cas9, wherein the sgRNA targets between about 1 to about 50 somatic mutations in a target cell.

More specifically the presently disclosed CRISPR-Cas9 system is capable of cancer-specific selective toxicity in subjects suffering from one or more types of cancer. In still another embodiment, the CRISPR-Cas9 system allows for customized targeting from treatment of one or more cancers. In one aspect, the present disclosure is not limited to the coding regions of the human genome (i.e., since all of the mutations targeted in the disclosed approach fall within non-coding regions, which make up 99% of the human genome), but include other vertebrates as well.

In some aspects, the CRISPR-Cas9 system can be used in any disease in which somatic mutations are present and elimination of diseased cells would be beneficial to the health of the subject. The presently disclosed CRISPR-Cas9 system, in particular, can advantageously be used to treat cancers, since cancers are inherently genetically unstable with one or more somatic mutations. Examples of cancer include, but are not limited to, anal cancer, bile duct cancer, bladder cancer, bone cancer, brain tumor and/or cancer, breast cancer, bronchial tumors, Burkitt lymphoma, cardiac tumors, cervical cancer, leukemia, colorectal cancer, uterine cancer, esophageal cancer, ewing sarcoma, fallopian tube cancer, gallbladder cancer, gastric cancer, gastrointestinal carcinoid tumor, head and neck cancer, kidney cancer, liver cancer, lip and oral cavity cancer, lung cancer, lymphoma, melanoma, skin cancer, metastatic cancer, mouth cancer, ovarian cancer, pancreatic cancer, prostate cancer, rectal cancer, salivary gland cancer, throat cancer, thyroid cancer or any combinations thereof. In one aspect, pancreatic cancer, which is the third leading cancer death with limited treatment efficacy, has more than 400 mutations per cell line that can be targeted by the presently disclosed CRISPR-Cas9 system.

In one particular aspect, the presently disclosed subject matter provides the CRISPR-Cas9 system for treating pancreatic cancer. In one aspect, the pancreatic cancer is benign pancreatic disease. In another aspect, the pancreatic cancer is early-stage pancreatic cancer. In yet another aspect, the pancreatic cancer is late-stage pancreatic cancer. In yet still another aspect, the pancreatic cancer is stage 0 pancreatic cancer. In a further another aspect, the pancreatic cancer is stage I pancreatic cancer. In yet still a further aspect, the pancreatic cancer is stage II pancreatic cancer. In still a further aspect, the pancreatic cancer is stage III pancreatic cancer. In still a further aspect, the pancreatic cancer is stage IV pancreatic cancer. In another particular aspects, the presently disclosed subject matter provides the CRISPR-Cas9 system for treating metastatic cancer. In a representative example involving pancreatic cancer cells, simultaneous targeting of at least 12 sites in the human genome leads to greater than 99% cell death. This toxicity is specific to the target cell and absent in non-target cells.

In some aspects, the target cells are, but not limited to, associated with one or more somatic mutations, such as, cancer cells, particularly pancreatic cancer, and metastatic cancer. In another aspect, the target cells are B-cells, T-cells and/or nerve cells. The somatic mutations have been described previously herein. In some aspects, the targeting mutations are not limited to the coding regions of the human genome. More specifically, in other aspects, the targeting mutations are within non-coding regions of the human genome.

In certain embodiments, the somatic mutations in cancer produce novel PAM sites targetable by CRISPR-Cas9. Therefore, in some aspects, the CRISPR-Cas9 system targets novel PAMs to kill the cancer or other disease causing cells (e.g., B-cells, T-cells, and/or nerve cells).

In certain embodiments, the present disclosure provides a CRISPR-Cas9 system comprising a sgRNA. As discussed above in section 2, the sgRNAs are designed to be both patient-specific and cancer-specific by identifying novel structural variants or base substitutions that lead to novel target site and/or novel PAMs as a result of base substitutions. In some aspects, the sgRNAs are designed to have multiple (e.g., 1-50) target sites for the effect of multiple DSBs. In other words, the sgRNAs are designed as multi-target sgRNAs. In another aspect, the sgRNAs are designed to cut in non-coding regions of the genome. In still another aspect, the sgRNAs are designed to have low numbers of off-target sites and high targeting efficiencies. In a further aspect, the sgRNA determines a specific genomic location for a double-strand break. In certain aspects, the sgRNA is selected from the group consisting of NT, NT2, HPRTc.80, HPRTc.465, 531F(2), 52F(3), 715F(5), 451F(6), 176R(7), 551R(8), 230F(12), 164R(14), 676F(16), AGGn, L1.4_209F, and ALU_112a. In one aspect, the NT has the sequence of SEQ ID NO:1. SEQ ID NO:1 is GTATTACTGATATTGGTGGG. In another aspect, the NT2 has the sequence of SEQ ID NO: 2. SEQ ID NO:2 is GCGAGGTATTCGGCTCCGCG. In yet another aspect, the HPRTc.80 has the sequence of SEQ ID NO:3. SEQ ID NO:3 is ATTATGCTGAGGATTTGGAA. In still yet another aspect, the HPRTc.465 has the sequence of SEQ ID NO:4. SEQ ID NO:4 is TGGATTATACTGCCTGACCA. In yet another aspect, the 531F(2) has the sequence of SEQ ID NO:5. SEQ ID NO:5 is CACTCAGCATCGACTTACGA. In still yet a further aspect, the 52F(3) has the sequence of SEQ ID NO:6. SEQ ID NO:6 is TAATTACTGCACGATGCGCA. In yet another aspect, the 715F(5) has the sequence of SEQ ID NO:7. SEQ ID NO:7 is ATATATATGCGATCGAGCCC. In yet a further aspect, the 451F(6) has the sequence of SEQ ID NO:8. SEQ ID NO:8 is ACTAGTGTGCGTATGATTTG. In still yet another aspect, the 176R(7) has the sequence of SEQ ID NO:9. SEQ ID NO:9 is TCGATGTTCTACATCGATGT. In still yet a further aspect, the 551R(8) has the sequence of SEQ ID NO:10. SEQ ID NO:10 is TTGAATTGAGTTGCAACCGA. In yet another aspect, the 230F(12) has the sequence of SEQ ID NO:11. SEQ ID NO:11 is TTGTCCCACAATGATACTTG. In still yet another aspect, the 164R(14) has the sequence of SEQ ID NO:12. SEQ ID NO:12 is GGATATTTCACTACAGACTT. In still yet a further aspect, the 676F(16) has the sequence of SEQ ID NO:13. SEQ ID NO:13 is CTCCGAACTTAACTTGCCCT. In still a further aspect, the AGGn has the sequence of SEQ ID NO:14. SEQ ID NO:14 is AGGAGGAGGAGGAGGAGGAG. In another aspect, the L1.4_209F has the sequence of SEQ ID NO:15. SEQ ID NO:15 is TGCCTCACCTGGGAAGCGCA. In still another aspect, the ALU_112a has the sequence of SEQ ID NO:16. SEQ ID NO:16 is TTGCCCAGGCTGGAGTGCAG.

In some embodiments, the multi-target sgRNA transduction leads to genomic instability and toxicity, and the accumulation of genomic instability events ultimately leads to cell death.

In certain embodiments, the present disclosure provides a CRISPR-Cas9 system comprising a sgRNA, wherein the sgRNA targets between about 1 to about 50 somatic mutations in a target cell. In some embodiments, the sgRNAs of the CRISPR-Cas9 system are designed as multi-target sgRNAs. In one aspect, the sg RNA targets at least 50 mutations in the target cell. In yet another aspect, the sgRNA targets at least 49 mutations in the target cell. In yet another aspect, the sgRNA targets at least 48 mutations in the target cell. In yet another aspect, the sgRNA targets at least 47 mutations in the target cell. In yet another aspect, the sgRNA targets at least 46 mutations in the target cell. In yet another aspect, the sgRNA targets at least 45 mutations in the target cell. In yet another aspect, the sgRNA targets at least 44 mutations in the target cell. In yet another aspect, the sgRNA targets at least 43 mutations in the target cell. In yet another aspect, the sgRNA targets at least 42 mutations in the target cell. In yet another aspect, the sgRNA targets at least 41 mutations in the target cell. In yet another aspect, the sgRNA targets at least 40 mutations in the target cell. In yet another aspect, the sgRNA targets at least 39 mutations in the target cell. In yet another aspect, the sgRNA targets at least 38 mutations in the target cell. In yet another aspect, the sgRNA targets at least 37 mutations in the target cell. In yet another aspect, the sgRNA targets at least 36 mutations in the target cell. In yet another aspect, the sgRNA targets at least 35 mutations in the target cell. In yet another aspect, the sgRNA targets at least 34 mutations in the target cell. In yet another aspect, the sgRNA targets at least 33 mutations in the target cell. In yet another aspect, the sgRNA targets at least 32 mutations in the target cell. In yet another aspect, the sgRNA targets at least 31 mutations in the target cell. In yet another aspect, the sgRNA targets at least 30 mutations in the target cell. In yet another aspect, the sgRNA targets at least 29 mutations in the target cell. In yet another aspect, the sgRNA targets at least 28 mutations in the target cell. In yet another aspect, the sgRNA targets at least 27 mutations in the target cell. In yet another aspect, the sgRNA targets at least 26 mutations in the target cell. In yet another aspect, the sgRNA targets at least 25 mutations in the target cell. In yet another aspect, the sgRNA targets at least 24 mutations in the target cell. In yet another aspect, the sgRNA targets at least 23 mutations in the target cell. In yet another aspect, the sgRNA targets at least 22 mutations in the target cell. In yet another aspect, the sgRNA targets at least 21 mutations in the target cell. In yet another aspect, the sgRNA targets at least 20 mutations in the target cell. In yet another aspect, the sgRNA targets at least 19 mutations in the target cell. In yet another aspect, the sgRNA targets at least 18 mutations in the target cell. In yet another aspect, the sgRNA targets at least 17 mutations in the target cell. In yet another aspect, the sgRNA targets at least 16 mutations in the target cell. In yet another aspect, the sgRNA targets at least 15 mutations in the target cell. In yet another aspect, the sgRNA targets at least 14 mutations in the target cell. In still yet another aspect, the sgRNA targets at least 13 mutations in the target cell. Instill yet another aspect, the sgRNA targets at least 12 mutations in the target cell. In yet a further aspect, the sgRNA targets at least 11 mutations in the target cell. In still yet a further aspect, the sgRNA targets at least 10 mutations in the target cell. In another aspect, the sgRNA targets at least 9 mutations in the target cell. In still another aspect, the sgRNA targets at least 8 mutations in the target cell. In yet another aspect, the sgRNA targets at least 7 mutations in the target cell. In still yet another aspect, the sgRNA targets at least 6 mutations in the target cell. In a further aspect, the sgRNA targets at least 5 mutations in the target cell. In yet a further aspect, the sgRNA targets at least 4 mutations in the target cell. In still yet a further aspect, the sgRNA targets at least 3 mutations in the target cell. In still yet a further aspect, the sgRNA targets at least 2 mutations in the target cell. In still yet a further aspect, the sgRNA targets at least 1 mutation in the target cell. In a representative example involving pancreatic cancer cells, sgRNA targets simultaneously at least 12 sites in the human genome. The simultaneous targeting of at least 12 sites in the human genome leads to greater than 99% cell death. This toxicity is specific to the target cell and absent in non-target cells.

In some embodiments, the formation of novel structural variants (SVs) is originated from CRISPR-Cas9 cutting at sgRNA target sites. The formation of novel SVs is a direct result of CRISPR-Cas9 cut, and these genomic rearrangements or chromosomal rearrangements are observed in the target sites. The toxicity following the induction of multiple DSBs that resulted in ongoing genomic rearrangements, chromosomal rearrangements, and/or polyploidization ultimately leads to cell death.

### 4. Multi-Target sgRNAs

In some embodiments, the presently disclosed subject matter provides an approach to identify and design sgRNAs that are both patient-specific and cancer-specific by identifying novel structural variants or base substitutions that lead to novel target sites and/or novel PAMs as a result of base substitutions. In one embodiment, the sgRNA determines a specific genomic location for a double-strand break. In another embodiment, the multi-target sgRNA transduction leads to genomic instability and toxicity and the accumulation of genomic instability events ultimately leads to cell death. Without wishing to be bound to any particular theory, it is believed that this same principle can be applied to all cancers, since mutations are a hallmark of cancer.

In some embodiments, the presently disclosed subject matter provides sgRNAs designed to have multiple (e.g., 1-50) target sites for the effect of multiple DSBs. In other words, the sgRNAs are designed as multi-target sgRNAs. In another aspect, the sgRNAs are designed to cut in non-coding regions of the genome. In still another aspect, the sgRNAs are designed to have low numbers of off-target sites and high targeting efficiencies. In some aspects, the sgRNA is selected from the group consisting of NT, NT2, HPRTc.80, HPRTc.465, 531F(2), 52F(3), 715F(5), 451F(6), 176R(7), 551R(8), 230F(12), 164R(14), 676F(16), AGGn, L1.4_209F, and ALU_112a. In one aspect, the NT has the sequence of SEQ ID NO:1. SEQ ID NO:1 is GTATTACTGATATTGGTGGG. In another aspect, the NT2 has the sequence of SEQ ID NO:2. SEQ ID NO:2 is GCGAGGTATTCGGCTCCGCG. In yet another aspect, the HPRTc.80 has the sequence of SEQ ID NO:3. SEQ ID NO:3 is ATTATGCTGAGGATTTGGAA. In still yet another aspect, the HPRTc.465 has the sequence of SEQ ID NO:4. SEQ ID NO:4 is TGGATTATACTGCCTGACCA. In yet another aspect, the 531F(2) has the sequence of SEQ ID NO:5. SEQ ID NO:5 is CACTCAGCATCGACTTACGA. In still yet a further aspect, the 52F(3) has the sequence of SEQ ID NO:6. SEQ ID NO:6 is TAATTACTGCACGATGCGCA. In yet another aspect, the 715F(5) has the sequence of SEQ ID NO:7. SEQ ID NO:7 is ATATATATGCGATCGAGCCC. In yet a further aspect, the 451F(6) has the sequence of SEQ ID NO:8. SEQ ID NO:8 is ACTAGTGTGCGTATGATTTG. In still yet another aspect, the 176R(7) has the sequence of SEQ ID NO:9. SEQ ID NO:9 is TCGATGTTCTACATCGATGT. In still yet a further aspect, the 551R(8) has the sequence of SEQ ID NO:10. SEQ ID NO:10 is TTGAATTGAGTTGCAACCGA. In yet another aspect, the 230F(12) has the sequence of SEQ ID NO:11. SEQ ID NO:11 is TTGTCCCACAATGATACTTG. In still yet another aspect, the 164R(14) has the sequence of SEQ ID NO:12. SEQ ID NO:12 is GGATATTTCACTACAGACTT. In still yet a further aspect, the 676F(16) has the sequence of SEQ ID NO:13. SEQ ID NO:13 is CTCCGAACTTAACTTGCCCT. In still a further aspect, the AGGn has the sequence of SEQ ID NO:14. SEQ ID NO:14 is AGGAGGAGGAGGAGGAGGAG. In another aspect, the L1.4_209F has the sequence of SEQ ID NO:15. SEQ ID NO:15 is TGCCTCACCTGGGAAGCGCA. In still another aspect, the ALU_112a has the sequence of SEQ ID NO:16. SEQ ID NO:16 is TTGCCCAGGCTGGAGTGCAG.

In one embodiment, the multi-target sgRNA transduction leads to genomic instability and toxicity. In one aspect, the mechanism of cell death is caused by the accumulation of genomic instability events, that ultimately led to cell death.

### 5. Method of Treating a Disease, Disorder, or Condition Associated with One or More Somatic Mutations

In some embodiments, the presently disclosed subject matter provides a method for treating a disease, disorder, or condition associated with one or more somatic mutations in a subject in need of treatment thereof, the method comprising administering an effective or therapeutically effective amount of the presently disclosed CRISPR-Cas9 system to a target cell of the subject in need of treatment thereof. The CRISPR-Cas9 system to be administered to a subject is designed according to the methods described above in Section 2. In one aspect, the CRISPR-Cas9 system is a selective cell killing tool capable of identifying mutations specific to one or more target cells. In another aspect, the CRISPR-Cas9 system of the present disclosure allows sgRNAs to be designed that target one or more somatic mutations (namely, 1-50 somatic mutations), such as those that produce one or more PAMs and/or target sites (e.g., sequences). In still yet a further aspect, the present disclosure provides for the introduction of a CRISPR-Cas9 system into one or more cells to induce multiple DSBs.

In another aspect, the CRISPR-Cas9 system comprises a sgRNA, wherein the sgRNA targets between about 1 to about 50 somatic mutations in a target cell. In still another aspect, the CRISPR-Cas9 system customizes the targeting. In still a further aspect, the mutations targeted as described in the present disclosure fall within non-coding regions. The CRISPR-Cas9 system has been described previously herein in section 3.

While not wishing to be bound by any theory, it is believed that administering to a subject suffering from a disease, disorder, a condition, or a combination thereof, a CRISPR-Cas9 system comprising a sgRNA which has been designed to target a sequence adjacent to the novel PAM and/or novel target site in one or more cells that cause or is associated with the disease, disorder or condition will cause a DSB in the one or more cells thereby resulting in the death of the cell. For example, targeting a sequence adjacent to a novel PAM and/or novel target site in cancer cells will result in the death of the cells and treatment of the cancer.

In yet other aspects, the presently disclosed method is applicable to any disease, disorder, or condition that is associated with one or more somatic mutations. In some aspects, the disease, disorder or condition comprises any disease in which one or more somatic mutations are present and elimination of diseased cells containing such mutations would be beneficial to health. Examples of somatic mutations include, but are not limited to, cancer and noncancerous disease. The presently disclosed CRISPR-Cas9 system, in particular, can advantageously be used to treat cancers, since cancers are inherently genetically unstable with one or more somatic mutations. In some aspects, one or more somatic mutations include a cancer. In particular aspects, the cancer is pancreatic cancer. In one aspect, the pancreatic cancer is benign pancreatic disease. In another aspect, the pancreatic cancer is early-stage pancreatic cancer. In yet another aspect, the pancreatic cancer is late-stage pancreatic cancer. In yet still another aspect, the pancreatic cancer is stage 0 pancreatic cancer. In a further another aspect, the pancreatic cancer is stage I pancreatic cancer. In yet still a further aspect, the pancreatic cancer is stage II pancreatic cancer. In still a further aspect, the pancreatic cancer is stage III pancreatic cancer. In still a further aspect, the pancreatic cancer is stage IV pancreatic cancer. In certain aspects, the cancer is metastatic cancer.

In some embodiments, the target cells are, but not limited to, associated with one or more somatic mutations, such as, cancer cells (such as, for example, a cancer initiating cell (CIC)), particularly pancreatic cancer, and metastatic cancer. However, any cell that causes a disease, disorder or condition (e.g., B-cells, T-cells, and/or nerve cells, etc.) can be targeted. The somatic mutations have been described previously herein. In some aspects, the targeting mutations are not limited to the coding regions of the human genome. More specifically, in other aspects, the targeting mutations are within non-coding regions of the human genome.

In some embodiments, sgRNAs are designed to have multiple (e.g., 1-50) target sites for the effect of multiple DSBs. In other words, the sgRNAs are designed as multi-target sgRNAs. In another aspect, the sgRNAs are designed to cut in one or more non-coding regions of the genome. In still another aspect, the sgRNAs are designed to have low numbers of off-target sites and high targeting efficiencies. In one aspect, the sg RNA targets at least 50 mutations in the target cell. In yet another aspect, the sgRNA targets at least 49 mutations in the target cell. In yet another aspect, the sgRNA targets at least 48 mutations in the target cell. In yet another aspect, the sgRNA targets at least 47 mutations in the target cell. In yet another aspect, the sgRNA targets at least 46 mutations in the target cell. In yet another aspect, the sgRNA targets at least 45 mutations in the target cell. In yet another aspect, the sgRNA targets at least 44 mutations in the target cell. In yet another aspect, the sgRNA targets at least 43 mutations in the target cell. In yet another aspect, the sgRNA targets at least 42 mutations in the target cell. In yet another aspect, the sgRNA targets at least 41 mutations in the target cell. In yet another aspect, the sgRNA targets at least 40 mutations in the target cell. In yet another aspect, the sgRNA targets at least 39 mutations in the target cell. In yet another aspect, the sgRNA targets at least 38 mutations in the target cell. In yet another aspect, the sgRNA targets at least 37 mutations in the target cell. In yet another aspect, the sgRNA targets at least 36 mutations in the target cell. In yet another aspect, the sgRNA targets at least 35 mutations in the target cell. In yet another aspect, the sgRNA targets at least 34 mutations in the target cell. In yet another aspect, the sgRNA targets at least 33 mutations in the target cell. In yet another aspect, the sgRNA targets at least 32 mutations in the target cell. In yet another aspect, the sgRNA targets at least 31 mutations in the target cell. In yet another aspect, the sgRNA targets at least 30 mutations in the target cell. In yet another aspect, the sgRNA targets at least 29 mutations in the target cell. In yet another aspect, the sgRNA targets at least 28 mutations in the target cell. In yet another aspect, the sgRNA targets at least 27 mutations in the target cell. In yet another aspect, the sgRNA targets at least 26 mutations in the target cell. In yet another aspect, the sgRNA targets at least 25 mutations in the target cell. In yet another aspect, the sgRNA targets at least 24 mutations in the target cell. In yet another aspect, the sgRNA targets at least 23 mutations in the target cell. In yet another aspect, the sgRNA targets at least 22 mutations in the target cell. In yet another aspect, the sgRNA targets at least 21 mutations in the target cell. In yet another aspect, the sgRNA targets at least 20 mutations in the target cell. In yet another aspect, the sgRNA targets at least 19 mutations in the target cell. In yet another aspect, the sgRNA targets at least 18 mutations in the target cell. In yet another aspect, the sgRNA targets at least 17 mutations in the target cell. In yet another aspect, the sgRNA targets at least 16 mutations in the target cell. In another aspect, the sgRNA targets at least 15 mutations in the target cell. In yet another aspect, the sgRNA targets at least 14 mutations in the target cell. In still yet another aspect, the sgRNA targets at least 13 mutations in the target cell. In particular aspects, the sgRNA targets at least 12 mutations in the target cell. In yet a further aspect, the sgRNA targets at least 11 mutations in the target cell. In still yet a further aspect, the sgRNA targets at least 10 mutations in the target cell. In another aspect, the sgRNA targets at least 9 mutations in the target cell. In still another aspect, the sgRNA targets at least 8 mutations in the target cell. In yet another aspect, the sgRNA targets at least 7 mutations in the target cell. In still yet another aspect, the sgRNA targets at least 6 mutations in the target cell. In a further aspect, the sgRNA targets at least 5 mutations in the target cell. In yet a further aspect, the sgRNA targets at least 4 mutations in the target cell. In still yet a further aspect, the sgRNA targets at least 3 mutations in the target cell. In still yet a further aspect, the sgRNA targets at least 2 mutations in the target cell. In still yet a further aspect, the sgRNA targets at least 1 mutation in the target cell. In a representative example involving pancreatic cancer cells, sgRNA targets simultaneously at least 12 sites in the human genome. The simultaneous targeting of at least 12 sites in the human genome leads to greater than 99% cell death. This toxicity is specific to the target cell and absent in non-target cells.

In certain embodiments, the CRISPR-Cas9 system is administered to the subject to induce one or more DSBs in the target cell, at a location adjacent to the novel PAM and/or novel target site as previously described herein. In certain aspects, the CRISPR-Cas9 system is administered to the subject to induce one or more DSBs in the target cell such as one or more cancer cells, at a location adjacent to the novel PAM and/or novel target site. In other aspects, the CRISPR-Cas9 system induced DSBs is selectively toxic (e.g., causes the death of the cell) to target cells, such as malignant cells. In certain embodiments, the CRISPR-Cas9 system is administered to the subject to induce one or more DSBs in the target cell such as one or more B and/or T-cells, at a location adjacent to the novel PAM and/or novel target site identified as previously described herein.

In certain embodiments, passenger mutations in cancer produce novel PAM sites targetable by CRISPR-Cas9. Therefore, in some aspects, the CRISPR-Cas9 system is administered to the novel PAMs to kill one or more cancer cells.

In some embodiments, the methods described herein involve monitoring the subject being treated with the CRISPR-Cas9 system for recurrence of the disease, disorder, or conditions. For example, a subject suffering from cancer and being treated with a CRISPR-Cas9 system prepared as described herein can be monitored for recurrence or relapse of the disease, disorder, or condition. Alternatively, the subject can be monitored for the development of resistance to the particular CRISPR-Cas9 treatment being employed. In the instance where a subject develops resistance to the particular CRISPR-Cas9 treatment, a sample is obtained from the subject in which such resistance has developed. Sequence data is obtained and analyzed from these cells to identify one or more somatic new (e.g., previously unidentified) base substitutions (BS), such as single base substitutions (SBS), one or more new (e.g., previously unidentified) structural variants (SV), or one or more BS and SVs that produce a novel (e.g., new) PAM, a novel (e.g., new) target site, or a novel PAM and a novel target site. Once the PAM and/or target site is identified, a new CRISPR-Cas9 system can be designed to target the novel PAM and/or novel target site using the methods described previously herein.

In some embodiments, the CRISPR-Cas9 system described herein and at least one other therapeutic agent, such as a chemotherapeutic agent, an autoimmune drug (e.g., immunosuppressant), an anti-inflammatory agent, etc., can be administered. In one aspect of the presently disclosed subject matter, the active agents are combined and administered in a single dosage form. In another aspect, the active agents are administered in separate dosage forms (e.g., wherein it is desirable to vary the amount of one but not the other) alternately or sequentially on the same or separate days. The single dosage form may include additional active agents for the treatment of the disease state.

Further, the CRISPR-Cas9 systems described herein can be administered alone or in combination with adjuvants that enhance stability of the CRISPR-Cas9 systems, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

In certain embodiments, the CRISPR-Cas9 system is delivered via a viral vector or one or more nanoparticles. In some aspects, the vector is a multiple sgRNA expression vector. In particular aspects, the viral vector is selected from an adenovirus, adeno-associated virus, retrovirus, lentivirus, Newcastle disease virus (NDV), and lymphocytic choriomeningitis virus (LCMV).

In certain embodiments, the subject is a mammalian subject. In particular embodiments, the mammalian subject is a human subject.

The timing of administration of a CRISPR-Cas9 system described herein and at least one additional therapeutic agent can be varied so long as the beneficial effects of the combination of these agents are achieved. Accordingly, the phrase “in combination with” refers to the administration of a CRISPR-Cas9 system described herein and at least one additional therapeutic agent either simultaneously, sequentially, or a combination thereof. Therefore, a subject administered a combination of a CRISPR-Cas9 system described herein and at least one additional therapeutic agent can receive a CRISPR-Cas9 system and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the CRISPR-Cas9 system described herein and at least one additional therapeutic agent are administered simultaneously, they can be administered to the subject as separate pharmaceutical compositions, each comprising either a CRISPR-Cas9 system or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a CRISPR-Cas9 system described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### 6. Kit

In one embodiment, the presently disclosed subject matter provides a kit comprising the CRISPR-Cas9 system described above in section 3. Additionally, in another embodiment, the kit comprises the CRISPR-Cas9 system in combination at least one other therapeutic agent, such as a chemotherapeutic agent, an autoimmune drug (e.g., immunosuppressant), an anti-inflammatory agent, etc., can be administered. In still another embodiment, the kit comprises the CRISPR-Cas9 system in combination with adjuvants that enhance stability of the CRISPR-Cas9 systems, alone or in combination with one or more therapeutic agents.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The descriptions and specific examples that follow are only intended for the purposes of illustration and are not to be construed as limiting in any manner.

### Example 1: Materials and Methods for Use in Example 2

### Study Design

A dose-response of number of double strand breaks to cell death was performed.

The timing and mechanism of cell death was next determined. Then, it was determined how many somatic PAMs could be found in 3 different cancer cell lines using 3 different approaches, and finally showed that targeting them could result in selective cell death.

Multitarget sgRNA Design

Chromosome range was entered into CRISPOR (35) 2 kb at a time starting at chr1:0-2000 and ending at chr1:100,248,000-100,250,000 based on hg19 and hg38, respectively. sgRNAs that have 2-16 perfect target sites were selected from the pool of sgRNA options generated by CRISPOR based on the following criteria: (1) none of the perfect target sites and potential off-target sites target exons; (2) Doench′16 (36) efficiency score is >50%, and (3) the number of off-targets that have no mismatches in the 12 bp adjacent to the PAM (SEED region) is <10. Sequences of non-targeting control sgRNAs were obtained from Doench et al (36) (NT) and Chiou et al (37) (NT2). HPRT1 sgRNAs (1-cutters) were designed using CRISPOR. Positive control sgRNAs were designed by either putting together a trinucleotide sequence (AGGn) or by inserting LINE-1 and Alu element sequences to CRISPOR.

### Cell Viability and Clonogenicity Assay

Cells were seeded for 24 hours before the media was replaced to contain 10 ug/mL of polybrene. Lentivirus of MOI 10 was added into the media and transduction took place for 18-20 hours. The media was then removed, washed once with PBS, and replaced with media that contained 5 ug/mL blasticidin. After 48 hours, the cells were split into two 96-well plates (one with 1:10 dilution and one with 1:1000 dilution of the original cultures) with media that contained both 5 ug/mL blasticidin and 1 ug/mL puromycin for selection. When cells in non-targeting controls reached full confluence, colonies were counted based on phase microscopy observation in 1:1000 dilution cultures. Then, 10 μL of alamarBlue Cell Viability Reagent (ThermoFisher) was added to 90 uL cell culture medium per well on 96-well plates. The plates were incubated at 37° C. for 3 or 24 hours, depending on cell lines, and transferred to BMG POLARstar Optima microplate reader for fluorescence reading. Excitation was set at 544 nm and emission at 590 nm, with a gain of 1000 and required value of 90%.

### Whole Genome Sequencing (WGS) of Surviving Colonies

Genomic DNA was extracted from surviving colonies of clonogenicity assay using QIAamp UCP DNA Micro Kit (QIAGEN) by following manufacturer's protocol. SKCCC Experimental and Computational Genomics Core sent the samples to New York Genome Center (NYGC) for WGS with an Illumina HiSeq 2000 using the TruSeq DNA prep kit. Sequencing was carried out so as to obtain 30× coverage from 2×100 bp paired-end reads. FASTQ files were aligned to both hg19 and hg38 using bwa v0.7.7 (mem, https://github.com/lh3/bwa) to create BAM files. The default parameters were used. Picard-tools1.119 (http://broadin ub.io/picard/) was used to add read groups as well as remove duplicate reads. GATK v3.6.0 (38) base call recalibration steps were used to create a final alignment file.

Cut Site Determination and Off-Target Analysis from WGS

BAM files were put into Integrated Genome Viewer (IGV(39)) to inspect all perfect and potential off-target sites (up to 4 mismatches). Actual cut site was determined by presence of mutation (insertion, deletion, or structural variant) at the sgRNA target region. Quantification of mutation frequency of all target sites were done using CRISPResso2 pipeline. For mutations that are SVs, quantification was manually done on IGV.

To identify potential off-target sites more objectively, MuTect2 v3.6.0 (38) was used to call somatic variants between the sample-control pairs. The default parameters and SnpEff (v4.1)(40) were used to annotate the passed variant calls and to create a clean tab separated table of variants. Manta v0.29.6 (15) was used to call somatic structural variants and indels between the sample-control pairs. The default parameters were used. Variants were annotated according to UCSC refseq annotations using an in-house script. From the list of results generated, for loci within the Excel files were looked for that closely matched our sgRNA sequence. This was performed with R script that performed the following steps: 1) Read in an Excel file containing one mutation per row. 2) Obtain the forward and reverse strand sequences from the hg19 genome between the start −50 bp and stop +50 bp positions of the locus. 3) Align each locus's forward and reverse sequences to the target sgRNA with no gaps using the Smith-Waterman algorithm. 4) Determine the number of mismatches between the sgRNA and the nearest matching piece of DNA within each junctions. Output the original information along with new columns displaying the mismatches between each junction and the sgRNA into a new Excel file. From the list of outputs, potential target sites were only considered that had <5 bp homology to the sgRNA sequence.

### Copy Number Calculation Based on WGS Data

Genome-wide copy number variants from the WGS data were generated using NxClinical software version 5.2 (BioDiscovery Inc., El Segundo, CA), which was described previously(41). Briefly, two algorithms were utilized including the “Self-reference” algorithm and the “Multi-Scale Reference” algorithm. Copy number variants were detected using the hidden Markov model based on NxClinical SNP-FASST2 algorithm, with autosomal log 2 ratio thresholds set at 0.7, 0.35, −0.35, and −1.5 for the detection of high-copy gains, duplications, monoallelic deletions, and biallelic deletions, respectively. Both sequencing read depths (the relative coverage) and B-allele frequencies were used to confirm copy number variant status.

sgRNA Tag Survival Assay

Cells were seeded for 24 hours before the media was replaced to contain 10 ug/mL of polybrene. Lentivirus of MOI 1 was added into the media and transduction took place for 18-20 hours. The media was then removed, washed once with PBS, and replaced with media that contained 5 ug/mL blasticidin. After 24 hours, approximately 1 million cells were collected for day 1 timepoint, and the remaining cells were subjected to both 5 ug/mL blasticidin and 1 ug/mL puromycin selections simultaneously. Cells were collected on day 7, 14, and 21 post-transduction, and along with day 1 cells, genomic extractions were performed using QIAamp UCP DNA Micro Kit (QIAGEN) by following manufacturer's protocol. sgRNA library was prepared by amplifying the sgRNA target region from gDNAs using NGS primers provided by Joung et al. (42), based on the protocol outlined in the paper, and sent for NGS (Supplemental Table 7). Read counts of each sgRNA were extracted from FASTQ files and were put through the MAGeCK (43) pipeline to obtain sgRNA fold change.

### Next Generation Sequencing (NGS) of Amplicons

PCR was performed with primers containing partial Illumina adapter sequences to generate amplicons. Either NEBNext High-Fidelity 2×PCR Master Mix (NEB) or Platinum SuperFi II PCR Master Mix (Thermo Fisher) was used for PCR preparations, and thermocycling conditions were set based on manufacturers' suggestions. Amplicons were purified using QIAGEN MinElute PCR purification kit based on manufacturer's protocol. Purified PCR products were sent to Azenta for Amplicon-EZ service, in which 2×250 bp sequencing was performed to provide ˜50,000 reads per sample. FASTQ files were obtained for further analysis.

### Chromosome Breakage Assay

The TS0111-Cas9-EGFP cells plated at 5×10⁵/ml were treated with a 14-cutter sgRNA and harvested at 0, 1, 3, 7, 10, 14, 16 and 21 days. Colcemid (0.01 μg/ml) was added 20 hours before harvesting. Cells were then exposed to 0.075 M KCl hypotonic solution for 30 minutes, fixed in 3:1 methanol: acetic acid and stained with Leishman's for 3 minutes. For each treatment, one hundred consecutive analyzable metaphases were analyzed for induction of chromosome abnormalities including chromosome/chromatid breaks and exchanges.

### 1q41 Break-Apart FISH Assay

FISH was performed on the TSO111-Cas9-EGFP cells before and after a 14-cutter sgRNA treatment (from 0, 1, 3, 7, 10, 14, 16 and 21 days) using RP11-14B15 and RP11-120E23 probes flanking a 1q41 sgRNA cut according to the manufacturer's protocol (Empiregenomics Inc., Williamsville, NY). The RP11-14B15 probe is for the 5′ (centromeric) side of the 1q41 sgRNA cut and in Spectrum Orange. The RP11-120E23 probe is for the 3′ (telomeric) side of the 1q41 sgRNA cut and in Spectrum Green. For these probes, an overlapping red/green or fused yellow signal represents the normal pattern, and separate red and green signals indicate the presence of a rearrangement. The normal cutoff was calculated based on the scoring of the TSO111-Cas9-EGFP cells before sgRNA treatment (day 0). The normal cutoff for an analysis of 500 cells with the 1q41 break-apart probe set is calculated using the Microsoft Excel β inverse function, =BETAINV (confidence level, false-positive cells plus 1, number of cells analyzed). This formula calculates a one-sided upper confidence limit for a specified percentage proportion based on an exact computation for a binomial distribution assessment. The normal cutoff for the 1q41 break-apart probe set is 0.6% (for a 95% confidence level). For each time point, a total of 500 nuclei were visually evaluated with fluorescence microscopy using a Zeiss Axioplan 2, with MetaSystems imaging software (MetaSystems, Medford, MA), to determine percentages of abnormal cells.

### SV Identification and Quantification

From the WGS BAM files of surviving colonies, Manta v0.29.6 was used to call somatic SVs and between the sample and the control, in which the control is the Panc10.05-Cas9-EGFP non-transduced cell line. The default parameters were used. Variants were annotated according to UCSC refseq annotations using an in-house script. The list of SVs generated were then individually, visually inspected on IGV to validate its presence in sample and absence in control. Novel SVs were quantified using SVs that have passed the manual screening.

### Cell Membrane and Genomic Staining

Alexa Fluor 488 conjugate of wheat germ agglutinin (WGA; ThermoFisher) was used to stain cell membrane on fixed cells according to manufacturer's protocol. Hoechst stain was used to stain genomic content by incubating the cells in Hoechst for 10 minutes in room temperature before covering the cell with mounting media.

### XY FISH Assay

Fluorescence in situ hybridization (FISH) was performed on the TS0111-Cas9-EGFP cells before and after a 14-cutter sgRNA treatment (from 0, 1, 3, 7, 10, 14, 16 and 21 days) using X/Y centromere FISH probes according to the manufacturer's protocol (Abbott Molecular Inc., Des Plaines, IL). For each time point, a total of 200 nuclei were visually evaluated with fluorescence microscopy using a Zeiss Axioplan 2, with MetaSystems imaging software (MetaSystems, Medford, MA), to determine copy number of the X chromosome.

### Apoptosis Assays

Cells were detached using Accutase and stained with Annexin V binding antibodies and propidium iodide using BioLegend's APC Annexin V Apoptosis Detection Kit, according to manufacturer's protocol. Fluorescence were quantified using Attune NxT Flow Cytometer. Cells were also platted on black with clear flat bottom 96-well plates and stained with both TUNEL and Hoechst using Cell Meter Live Cell TUNEL Apoptosis Assay Kit (Red Fluorescence), according to manufacturer's protocol (AAT Bioquest). BMG POLARstar Optima microplate reader for fluorescence reading. For TUNEL measurement, excitation was set at 544 nm and emission at 590 nm, with a gain of 1000 and required value of 90%. For Hoechst, excitation was set at 490 nm and emission at 520 nm, with a gain of 1700 and required value of 90%. Final calculation was done based on a formula used by Daniel and DeCoster (44).

SV Target Validation and sgRNA Design

A list of SVs were compiled from SVs previously published in Norris et al. (2015) and SVs generated by Trellis (16). SVs that were present in germline based on IGV visual inspection were eliminated from the list. Primers were designed to PCR amplify across breakpoints and sent for Sanger sequencing (See below Table 1).

TABLE 1
Primers for PCR and Sanger validation of novel structural variants

| Forward | | | |
|---|---|---|---|
| primer* | Sequence# | Reverse primer* | Sequence |
| PANC480_Chr1: | GTAAAACGACGGCCAGCTC | PANC480_Chr1: | CAGGAAACAGCTATGACTCTG |
| 174M_td Fwd | TTTGGCTGATGTTCC (SEQ | 174M_td Rev | CACATAACGGTGGA |
| | ID NO: 18) | | (SEQ ID NO: 108) |
| PANC480_chr1_ | GTAAAACGACGGCCAGAAG | PANC480_chr1_ | GCCTGTCCCTTGTTTCCTTG |
| 154d_st1_fwd | AATCGCCTGAACCTGGG | 154d_st1_rev | (SEQ ID NO: 109) |
| | (SEQ ID NO: 19) | | |
| PANC480_Chr1: | GTAAAACGACGGCCAGTCT | PANC480_Chr1: | CAGGAAACAGCTATGACAGTA |
| 222M_t Fwd | CAAAGTTACACGTCA (SEQ | 222M_t Rev | GAGAAGCTTGAAAT |
| | ID NO: 20) | | (SEQ ID NO: 110) |
| PANC480_chr1_ | GTAAAACGACGGCCAGACT | PANC480_chr1_ | TGCACACATCACAAAGAAGTT |
| 248t_st1_fwd | ACCACTCCTTCATCCCC | 248t_st1_rev | TC |
| | (SEQ ID NO: 21) | | (SEQ ID NO: 111) |
| PANC480_chr2_ | GTAAAACGACGGCCAGGTT | PANC480_chr2_ | CCCAGGCTGTTCTCGAAAAC |
| 26d_st1_fwd | CACCATCTTAGCCACAGG | 26d_st1_rev | (SEQ ID NO: 112) |
| | (SEQ ID NO: 22) | | |
| PANC480_Chr2: | GTAAAACGACGGCCAGAAA | PANC480_Chr2: | CAGGAAACAGCTATGACATGA |
| 149M_D_FWD | GAGTGTGACGGAGGG (SEQ | 149M_D_REV | AAACAGTGAAATAT |
| | ID NO: 23) | | (SEQ ID NO: 113) |
| PANC480_chr2_ | GTAAAACGACGGCCAGTAT | PANC480_chr2_ | GGAACCTCTGCTCTTCATGAC |
| 221d_st1_fwd | TTGATGAGGGCCAGTGC | 221d_st1_rev | (SEQ ID NO: 114) |
| | (SEQ ID NO: 24) | | |
| PANC480_Chr2: | GTAAAACGACGGCCAGAGT | PANC480_Chr2: | CAGGAAACAGCTATGACTGAA |
| 164M_td Fwd | GGCATGGAACAGATT (SEQ | 125M_td Rev | AATCAAAAGTATCT |
| | ID NO: 25) | | (SEQ ID NO: 115) |
| PANC480_chr2_ | GTAAAACGACGGCCAGTTA | PANC480_chr2_ | CACTTGATTGGGATGAATCG |
| 164tf_jt2_Fwd | CCAAAGTTCCCCAGGTG | 164tf_jt2_Rev | (SEQ ID NO: 116) |
| | (SEQ ID NO: 26) | | |
| PANC480_chr2_ | GTAAAACGACGGCCAGGAG | PANC480_chr2_ | CCCAGAAGGAATGAAGTCCA |
| 210tf_jt1_Fwd | GCAGGCATGGAAAGTTA | 210tf_jt1_Rev | (SEQ ID NO: 117) |
| | (SEQ ID NO: 27) | | |
| PANC480_chr2_ | GTAAAACGACGGCCAGAGC | PANC480_chr2_ | GGGAAAAGTCTCCCTGGTTC |
| 221tf18_jt1_Fwd | AGGCTTTATGCCACATC | 221tf18_jt1_Rev | (SEQ ID NO: 118) |
| | (SEQ ID NO: 28) | | |
| PANC480_chr2_ | GTAAAACGACGGCCAGGCC | PANC480_chr2_ | ATCTGACACAAAGGCCCAAG |
| 221tf17_jt1_Fwd | ACATCTTTCCCATTCAA | 221tf17_jt1_Rev | (SEQ ID NO: 119) |
| | (SEQ ID NO: 29) | | |
| PANC480_Chr2: | GTAAAACGACGGCCAGTTA | PANC480_Chr2: | CAGGAAACAGCTATGACCTGT |
| 209M_t Fwd | AAGCTTTTGGACTTT (SEQ | 209M_t Rev | ACTCTGAAAGGATG |
| | ID NO: 30) | | (SEQ ID NO: 120) |
| PANC480_chr2_ | GTAAAACGACGGCCAGATT | PANC480_chr2_ | TGTTCAGAGAAGTCTTTGCTCA |
| 214t_st1_fwd | CTACCTGTTCAGGGCCC | 214t_st1_rev | (SEQ ID NO: 121) |
| | (SEQ ID NO: 31) | | |
| PANC480_chr2: | GTAAAACGACGGCCAGTTC | PANC480_chr2: | CAGGAAACAGCTATGACTAGC |
| 221M_t Fwd | AACTAGGTAGGTCTC (SEQ | 221M_t Rev | TGGATCTAGGGATT |
| | ID NO: 32) | | (SEQ ID NO: 122) |
| PANC480_chr4_ | GTAAAACGACGGCCAGTGA | PANC480_chr4_ | CCTCCTCCTGAATTCCTCCT |
| 106tf_jt2_Fwd | AAGATGCAATGCTCCTG | 106tf_jt2_Rev | (SEQ ID NO: 123) |
| | (SEQ ID NO: 33) | | |
| PANC480_Chr4: | GTAAAACGACGGCCAGCTG | PANC480_Chr4: | CAGGAAACAGCTATGACTTCC |
| 57M_t_FWD | AGCTTATTCTCAGAC (SEQ | 57M_t_REV | AACTTCTTTACATC |
| | ID NO: 34) | | (SEQ ID NO: 124) |
| PANC480_chr4: | GTAAAACGACGGCCAGCGA | PANC480_chr4: | CAGGAAACAGCTATGACGCTA |
| 106M_t Fwd | TCTCAAATCAAACTC (SEQ | 106M_t Rev | CACATATTTCATAA |
| | ID NO: 35) | | (SEQ ID NO: 125) |
| PANC480_chr5_ | GTAAAACGACGGCCAGGGG | PANC480_chr5_ | CCCACCAACCAGAGAGAACT |
| 81t_st1_fwd | CATACAGGGACAATTCAC | 81t_st1_rev | (SEQ ID NO: 126) |
| | (SEQ ID NO: 36) | | |
| PANC480_chr5_ | GTAAAACGACGGCCAGGGT | PANC480_chr5_ | CTGTGTGGCTGCTTTCACTG |
| 43tf_jt1_Fwd | TCCACAGTAACCCAGCA | 43tf_jt1_Rev | (SEQ ID NO: 127) |
| | (SEQ ID NO: 37) | | |
| PANC480_chr5_ | GTAAAACGACGGCCAGGGG | PANC480_chr5_ | TGTAAGATGGAGCAGGGACC |
| 81t_st2_fwd | CATACAGGGACAATTCAC | 81t_st2_rev | (SEQ ID NO: 128) |
| | (SEQ ID NO: 38) | | |
| PANC480_Chr6: | GTAAAACGACGGCCAGTTT | PANC480_Chr6: | CAGGAAACAGCTATGACCCTG |
| 28M_d Fwd | TCTGCTGATAATTTC (SEQ | 28M_d Rev | GATGACATATTTGT |
| | ID NO: 39) | | (SEQ ID NO: 129) |
| PANC480_chr6: | GTAAAACGACGGCCAGAGA | PANC480_chr6: | CAGGAAACAGCTATGACCTGA |
| 25M_td Fwd | AAGAAAAGGTAGGAA (SEQ | 25M_td Rev | ATTTACAAATTCGT |
| | ID NO: 40) | | (SEQ ID NO: 130) |
| PANC480_chr6_ | GTAAAACGACGGCCAGCCA | PANC480_chr6_ | GTATGAGGGCCAATTTGTGG |
| 25id_jt2_Fwd | CTCCTGGCTTCAAGAAC | 25id_jt2_Rev | (SEQ ID NO: 131) |
| | (SEQ ID NO: 41) | | |
| PANC480_chr6_ | GTAAAACGACGGCCAGAGG | PANC480_chr6_ | TGCGCGTGTTTTAAGAGAGG |
| 27id_fwd1 | GACATGTCATAAGCCTCT | 27id_rev2 | (SEQ ID NO: 132) |
| | (SEQ ID NO: 42) | | |
| PANC480_chr8_ | GTAAAACGACGGCCAGTAG | PANC480_chr8_ | TAACAGGAGAATTGGGCGGT |
| 127tf_fwd1 | CTTGATGGGGATGGCAT | 127tf_rev1 | (SEQ ID NO: 133) |
| | (SEQ ID NO: 43) | | |
| PANC480_Chr9: | GTAAAACGACGGCCAGAAA | PANC480_Chr9: | CAGGAAACAGCTATGACCCAA |
| 14M_d Fwd | GAAGGAAGGAACCAC (SEQ | 14M_d Rev | CAAGAGTAAAGGTT |
| | ID NO: 44) | | (SEQ ID NO: 134) |
| PANC480_chr9_ | GTAAAACGACGGCCAGGGA | PANC480_chr9_ | AGGCTCCTTTTGAACACCTTC |
| 78d_st1_fwd | ACCTCACAAAGTAACTCTG | 78d_st1_rev | (SEQ ID NO: 135) |
| | G (SEQ ID NO: 45) | | |
| PANC480_chr9_ | GTAAAACGACGGCCAGACA | PANC480_chr9_8 | AATGAACCACCCTGTCCCAT |
| 84t_st2_fwd | CATTCGAAGGAGGCTCA | 84t_st1_rev | (SEQ ID NO: 136) |
| | (SEQ ID NO: 46) | | |
| PANC480_chr18_ | GTAAAACGACGGCCAGCCA | PANC480_chr18_ | GGCCCAGATGTCTCACTACA |
| 75i_fwd1 | CTAGCCTGGCATATCTGA | 75i_rev2 | (SEQ ID NO: 137) |
| | (SEQ ID NO: 47) | | |
| PANC480_chr18_ | GTAAAACGACGGCCAGTTC | PANC480_chr18_ | CTCCCATCCGAAGAGACAGC |
| 76i_fwd1 | ATCTATGTCTTTGGTGGCT | 76i_rev2 | (SEQ ID NO: 138) |
| | (SEQ ID NO: 48) | | |
| PANC504_chr3_ | GTAAAACGACGGCCAGACA | PANC504_chr3_ | GGCTATACATACCTGCACAGC |
| 60d_jt1_Fwd | CCCCCACCAACTGTAGA | 60d_jt1_Rev | A |
| | (SEQ ID NO: 49) | | (SEQ ID NO: 139) |
| PANC504_chr4_ | GTAAAACGACGGCCAGAGG | PANC504_chr4_ | TGCATGGCTTCTTCTACAAGTG |
| 21d_st1_fwd | ATATGTGGAAAGCGCTCT | 21d_st1_rev | (SEQ ID NO: 140) |
| | (SEQ ID NO: 50) | | |
| PANC504_chr4_ | GTAAAACGACGGCCAGCAC | PANC504_chr4_ | GGAACATTGCTCCCCATTCC |
| 21td_fwd2 | ATCACATTTGCAGGGGA | 21td_rev1 | (SEQ ID NO: 141) |
| | (SEQ ID NO: 51) | | |
| PANC504_chr4_ | GTAAAACGACGGCCAGCGT | PANC504_chr4_ | TCTTGGGATCATCCTTGACA |
| 66td_fwd1 | TTCCCAACTAAATGCAGA | 66td_rev1 | (SEQ ID NO: 142) |
| | (SEQ ID NO: 52) | | |
| PANC504_chr4_ | GTAAAACGACGGCCAGTGG | PANC504_chr4_ | CGACCTCCTTCCAATCCAGT |
| 59i_fwd1 | CCCTTATCCCTTCTTTT | 59i_rev1 | (SEQ ID NO: 143) |
| | (SEQ ID NO: 53) | | |
| PANC504_chr4_ | GTAAAACGACGGCCAGGGG | PANC504_chr4_ | CTCGTCAGAACCAACGGTCT |
| 2t_fwd1 | GACTTGGCTATTTCACA | 2t_rev1 | (SEQ ID NO: 144) |
| | (SEQ ID NO: 54) | | |
| PANC504_chr4_ | GTAAAACGACGGCCAGACT | PANC504_chr4_ | GCAGGCAAACAGGAACAGAA |
| 59t_st1_fwd | TCCCAGTCAGTGTGTACA | 59t_st1_rev | (SEQ ID NO: 145) |
| | (SEQ ID NO: 55) | | |
| PANC504_chr6_ | GTAAAACGACGGCCAGAAG | PANC504_chr6_ | GTGACAGCGAGTCAGACGTT |
| 26d_jt2_Fwd | CCCAGGAATTCAAGACC | 26d_jt1_Rev | (SEQ ID NO: 146) |
| | (SEQ ID NO: 56) | | |
| PANC504_chr7_ | GTAAAACGACGGCCAGTGG | PANC504_chr7_ | AAGTGGAAGAGGTGAAGGGT |
| 68d_fwd1 | TACAGTTGGTTGATAACAC | 68d_rev1 | (SEQ ID NO: 147) |
| | A (SEQ ID NO: 57) | | |
| PANC504_chr7_ | GTAAAACGACGGCCAGGAG | PANC504_chr7_ | GGTTTTGTGGCTTCTTGCAT |
| 96d_fwd1 | TCCGGGCATTGTACAAG | 96d_rev1 | (SEQ ID NO: 148) |
| | (SEQ ID NO: 58) | | |
| PANC504_chr8_ | GTAAAACGACGGCCAGTGC | PANC504_chr8_ | AAGACGATCGAGACCATCCC |
| 64d_st1_fwd | ATTTGACGCGCTTGATA | 64d_st1_rev | (SEQ ID NO: 149) |
| | (SEQ ID NO: 59) | | |
| PANC504_chr8_ | GTAAAACGACGGCCAGCCC | PANC504_chr8_ | GCTTTGTTTTCCAGTGCCTG |
| 145tf_fwd1 | CTGATCAGCGTCAAATT | 145tf_rev1 | (SEQ ID NO: 150) |
| | (SEQ ID NO: 60) | | |
| PANC504_chr9_ | GTAAAACGACGGCCAGGGG | PANC504_chr9_ | TCTTGAGGAAGGGAGAAACAC |
| 20t_st1_fwd | AGGACGCTTCAGAGAAA | 20t_st1_rev | A |
| | (SEQ ID NO: 61) | | (SEQ ID NO: 151) |
| PANC504_Chr9: | GTAAAACGACGGCCAGACT | PANC504_Chr9: | CAGGAAACAGCTATGACCTAA |
| 24M t Fwd | TTAGTAATATGTTT | 24M_t_Rev | GGCAAACAACACTG |
| | (SEQ ID NO: 62) | | (SEQ ID NO: 152) |
| PANC504_chr11_ | GTAAAACGACGGCCAGGTC | PANC504_chr11_ | TCCATGGGCACTAGAAGAGC |
| 42t_fwd1 | TGTGCTGTCCCTCCTGT | 42t_rev1 | (SEQ ID NO: 153) |
| | (SEQ ID NO: 63) | | |
| PANC504_chr12_ | GTAAAACGACGGCCAGAAC | PANC504_chr12_ | GCCCTGAGCAATCCTATCTG |
| 96td_jt1_Fwd | CCCAACGATCAATTCAC | 96td_jt1_Rev | (SEQ ID NO: 154) |
| | (SEQ ID NO: 64) | | |
| PANC504_chr12_ | GTAAAACGACGGCCAGCAC | PANC504_chr12_ | ACGGGTTGAATGGATTGGTG |
| 88t_st1_fwd | AAAGCCCACACCATGAA | 88t_st1_rev | (SEQ ID NO: 155) |
| | (SEQ ID NO: 65) | | |
| PANC504_chr14_ | GTAAAACGACGGCCAGGGC | PANC504_chr14_ | GGAGGAATCAGTCTACCCAAT |
| 59t_st1_fwd | TCATTCGACTCACTTCC | 59t_st1_rev | T |
| | (SEQ ID NO: 66) | | (SEQ ID NO: 156) |
| PANC504_chr16_ | GTAAAACGACGGCCAGGCC | PANC504_chr16_ | CCAGAAAGGTGAATGCTGTCA |
| 73t_st1_fwd | ACACATTGTCTCATCCA | 73t_st1_rev | (SEQ ID NO: 157) |
| | (SEQ ID NO: 67) | | |
| PANC504_chr16_ | GTAAAACGACGGCCAGGGG | PANC504_chr16_ | TCAAACTTCAGCTGGGAACC |
| 75t_fwd2 | TTCAAGCAGTTCTCCTG | 75t_rev2 | (SEQ ID NO: 158) |
| | (SEQ ID NO: 68) | | |
| PANC504_chr17_ | GTAAAACGACGGCCAGAAT | PANC504_chr17_ | CATGGAGAAACAGGCGAGTG |
| 63t_st1_fwd | GCAGTGGGGTGAACAAC | 63t_st1_rev | (SEQ ID NO: 159) |
| | (SEQ ID NO: 69) | | |
| PANC504_chr17_ | GTAAAACGACGGCCAGCAC | PANC504_chr17_ | CTGGAGAGGCATGGAGAGTT |
| 64t_st1_fwd | CCATTTCTAGTGCTGCC | 64t_st1_rev | (SEQ ID NO: 160) |
| | (SEQ ID NO: 70) | | |
| PANC504_Chr17: | GTAAAACGACGGCCAGAGT | PANC504_Chr17: | CAGGAAACAGCTATGACTGTG |
| 39M_d Fwd | AGGGGTAGAGGACAG | 39M_d Rev | TGGTTCAGTATATC |
| | (SEQ ID NO: 71) | | (SEQ ID NO: 161) |
| PANC504_chr17_ | GTAAAACGACGGCCAGGGA | PANC504_chr17_ | TAGCAAGCACCACCTCCTCT |
| 50id_fwd1 | AGTGCAGGCAAAATGAT | 50id_rev1 | (SEQ ID NO: 162) |
| | (SEQ ID NO: 72) | | |
| PANC504_chr17_ | GTAAAACGACGGCCAGTGG | PANC504_chr17_ | ATAGGTGGTCATTCGAGGGC |
| 66i_fwd1 | TCTTCTTTCAAGGTTTGCC | 66i_rev1 | (SEQ ID NO: 163) |
| | (SEQ ID NO: 73) | | |
| PANC504_Chr18: | GTAAAACGACGGCCAGAAG | PANC504_Chr18: | CAGGAAACAGCTATGACATTC |
| 50M-1_n1 Fwd | CTCTTGAAGACATAA | 50-1_n1_Rev | CAAAGCCATGCTAA |
| | (SEQ ID NO: 74) | | (SEQ ID NO: 164) |
| PANC504_Chr18: | GTAAAACGACGGCCAGAGT | PANC504_Chr18: | CAGGAAACAGCTATGACTCCA |
| 50M Fwd | CAAAGGCCCTCCTCT | 50M Rev | GCCTCAGACAGAAC |
| | (SEQ ID NO: 75) | | (SEQ ID NO: 165) |
| PANC504_Chr18: | GTAAAACGACGGCCAGTAC | PANC504_Chr18: | CAGGAAACAGCTATGACTTCA |
| 48M Fwd | CATAGGATGCTTAAC | 48M_Rev | GCCCAGATCCCTAA |
| | (SEQ ID NO: 76) | | (SEQ ID NO: 166) |
| PANC504_Chr22: | GTAAAACGACGGCCAGGTC | PANC504_Chr22: | CAGGAAACAGCTATGACAAGT |
| 30M Fwd | CCAGCTACTTGGGAG | 50M Rev | CAGATCACCTTCAT |
| | (SEQ ID NO: 77) | | (SEQ ID NO: 167) |
| PANC1002Chr1: | GTAAAACGACGGCCAGGGA | PANC1002Chr1: | CAGGAAACAGCTATGACGTAT |
| 74M_d Fwd | AACTTCATAAACATT | 74M_d Rev | TTCTCCAACCTATA |
| | (SEQ ID NO: 78) | | (SEQ ID NO: 168) |
| PANC1002_chr1_ | GTAAAACGACGGCCAGTTA | PANC1002_chr1_ | TTTGCTGCAGCTAGCCATTT |
| 72d_jt2_Fwd | GGGAGGCAAATCAACCA | 72d_jt2_Rev | (SEQ ID NO: 169) |
| | (SEQ ID NO: 79) | | |
| PANC1002_chr1_ | GTAAAACGACGGCCAGAAT | PANC1002_chr1_ | GAGAGACAGAGACAGAGGTG |
| 72id_fwd2 | TGTGCCCTGACCATGC | 72id_rev2 | A |
| | (SEQ ID NO: 80) | | (SEQ ID NO: 170) |
| PANC1002Chr2: | GTAAAACGACGGCCAGGGC | PANC1002Chr2: | CAGGAAACAGCTATGACTCAT |
| 5M_d Fwd | GTTCCTTGGGGTTCA | 5M_d Rev | CCAAATCTACTTTC |
| | (SEQ ID NO: 81) | | (SEQ ID NO: 171) |
| PANC1002Chr2: | GTAAAACGACGGCCAGGAA | PANC1002Chr2: | CAGGAAACAGCTATGACTGAG |
| 74M_d Fwd | ATGATGTCTGGAGGA | 74M_d Rev | GAAGTGAAAACATT |
| | (SEQ ID NO: 82) | | (SEQ ID NO: 172) |
| PANC1002Chr2: | GTAAAACGACGGCCAGTTC | PANC1002Chr2: | CAGGAAACAGCTATGACGCTC |
| 156M_d Fwd | TCTGTTGAGGTTGAC | 156M_d Rev | TTTTCTTTTTCTTT |
| | (SEQ ID NO: 83) | | (SEQ ID NO: 173) |
| PANC1002_Chr3: | GTAAAACGACGGCCAGGTC | PANC1002_Chr3: | CAGGAAACAGCTATGACACCC |
| 69M Fwd | AATATTGAAAGAAGG | 69M Rev | AGTTAACATCACAA |
| | (SEQ ID NO: 84) | | (SEQ ID NO: 174) |
| PANC1002 Chr4: | GTAAAACGACGGCCAGTAT | PANC1002 Chr4: | CAGGAAACAGCTATGACGCAC |
| 178M Fwd | AGCCATCATAGCATA | 178M Rev | CTACCTCACCTGCA |
| | (SEQ ID NO: 85) | | (SEQ ID NO: 175) |
| PANC1002Chr5: | GTAAAACGACGGCCAGAAG | PANC1002Chr5: | CAGGAAACAGCTATGACTTCT |
| 27439M_d Fwd | CTGCAGATCTTCACG | 27439M_d Rev | GTAATTCTACAAGA |
| | (SEQ ID NO: 86) | | (SEQ ID NO: 176) |
| PANC1002Chr5: | GTAAAACGACGGCCAGGTA | PANC1002Chr5: | CAGGAAACAGCTATGACAAGA |
| 27824M_d Fwd | ATATATTTAAAGATT | 27824M_d Rev | TGGTGAAGAATTAG |
| | (SEQ ID NO: 87) | | (SEQ ID NO: 177) |
| PANC1002Chr5: | GTAAAACGACGGCCAGCTC | PANC1002Chr5: | CAGGAAACAGCTATGACGAAG |
| 115M_Hd Fwd | TAGATCTGGATGAGG | 115M_Hd Rev | CAGGGTTTTCTGCA |
| | (SEQ ID NO: 88) | | (SEQ ID NO: 178) |
| PANC1002Chr5: | GTAAAACGACGGCCAGAAT | PANC1002Chr5: | CAGGAAACAGCTATGACGTAA |
| 26M_d Fwd | ATGGAAGATACTAAT | 26M_d Rev | ATGTCATATTGTGA |
| | (SEQ ID NO: 89) | | (SEQ ID NO: 179) |
| PANC1002_chr5_ | GTAAAACGACGGCCAGCCA | PANC1002_chr5_ | GGGGTTCAGAACTTCAGTGG |
| 22t_st1_fwd | AATATGAAAGCCCCAAA | 22t_st1_rev | (SEQ ID NO: 180) |
| | (SEQ ID NO: 90) | | |
| PANC1002 Chr6: | GTAAAACGACGGCCAGTCT | PANC1002 Chr6: | CAGGAAACAGCTATGACTATG |
| 81M Fwd | TCTGTGTCGCTCACG | 81M_n1 Rev | ATCACCTTGTATAA |
| | (SEQ ID NO: 91) | | (SEQ ID NO: 181) |
| PANC1002_chr7_ | GTAAAACGACGGCCAGGTG | PANC1002_chr7_ | ATGGATTGGGTGTCCAGAAA |
| 3d_jt1_Fwd | AATTTCCTGGGGTTCAG | 3d_jt1_Rev | (SEQ ID NO: 182) |
| | (SEQ ID NO: 92) | | |
| PANC1002Chr7: | GTAAAACGACGGCCAGTGA | PANC1002Chr7: | CAGGAAACAGCTATGACAATG |
| 344M_d Fwd | TGGCACAAAGGAAAA | 34M_d Rev | GGAAAGATATATAA |
| | (SEQ ID NO: 93) | | (SEQ ID NO: 183) |
| PANC1002_chr7_ | GTAAAACGACGGCCAGGGG | PANC1002_chr7_ | TGGGAGAAGACCCAGCTAAA |
| 111d_st1_fwd | TTGCAGTCTTCCTTGTC | 111d_st1_rev | (SEQ ID NO: 184) |
| | (SEQ ID NO: 94) | | |
| PANC1002 Chr8: | GTAAAACGACGGCCAGTAC | PANC1002 Chr8: | CAGGAAACAGCTATGACCCTC |
| 123M Fwd | CAATTACATGTGAGG | 123M Rev | CAAATACCATCCCA |
| | (SEQ ID NO: 95) | | (SEQ ID NO: 185) |
| PANC1002 Chr8: | GTAAAACGACGGCCAGTGT | PANC1002_Chr8: | CAGGAAACAGCTATGACTTCC |
| 138M_n1 Fwd | GATAGGCTAAATAAT | 138M_n1 Rev | TGTCCAGCATTCAC |
| | (SEQ ID NO: 96) | | (SEQ ID NO: 186) |
| PANC1002_chr8_ | GTAAAACGACGGCCAGAGA | PANC1002 chr8_ | TGCGTTGTTATCATACTGTGC |
| 51d_st1_fwd | TGGAGAAGGGAATGCAA | 51d_st1_rev | (SEQ ID NO: 187) |
| | (SEQ ID NO: 97) | | |
| PANC1002_chr9_ | GTAAAACGACGGCCAGATT | PANC1002 chr9_ | ACATGCCGTACAAGTCATCC |
| 21t_fwd1 | AGCCCCTGGAAAGCAGT | 21t_rev2 | (SEQ ID NO: 188) |
| | (SEQ ID NO: 98) | | |
| PANC1002_chr9_ | GTAAAACGACGGCCAGATT | PANC1002 chr9_ | GGGATGGGGAAAGAGAAGTC |
| 21995t_st1_fwd | GTGCAGAAGCCAGTCCT | 21995t_st1_rev | (SEQ ID NO: 189) |
| | (SEQ ID NO: 99) | | |
| PANC1002 chr12_ | GTAAAACGACGGCCAGCCC | PANC1002 chr12_ | TCCCTGAGAAAGTCCTGGTTT |
| 28i_jt1_Fwd | ATTGCAAGCCTACAGTT | 28i_jt1 Rev | (SEQ ID NO: 190) |
| | (SEQ ID NO: 100) | | |
| PANC1002Chr12: | GTAAAACGACGGCCAGATC | PANC1002Chr12: | CAGGAAACAGCTATGACTGTT |
| 86M_d Fwd | TTTCTCTTACCCTAC | 86M_d Rev | AACTAGAATAA |
| | (SEQ ID NO: 101) | | (SEQ ID NO: 191) |
| PANC1002_chr13_ | GTAAAACGACGGCCAGGGG | PANC1002 chr13_ | GACAAAGTGGCATGGCATGA |
| 53d_fwd1 | ACAGTAGAGGCATCAGA | 53d_rev2 | (SEQ ID NO: 192) |
| | (SEQ ID NO: 102) | | |
| PANC1002Chr13: | GTAAAACGACGGCCAGAAA | PANC1002Chr13: | CAGGAAACAGCTATGACTTCC |
| 82M_d Fwd | TGTTTTTGAAGTTCA | 82M_d Rev | CTGCAATGGAGGGC |
| | (SEQ ID NO: 103) | | (SEQ ID NO: 193) |
| PANC1002Chr13: | GTAAAACGACGGCCAGATC | PANC1002Chr13: | CAGGAAACAGCTATGACGAAA |
| 95M_d Fwd | ATTTTATCTTCAATT | 95M_d Rev | AGGCAAAACCACAA |
| | (SEQ ID NO: 104) | | (SEQ ID NO: 194) |
| PANC1002 chr17_ | GTAAAACGACGGCCAGGCT | PANC1002 chr17_ | CACCAAGCCATTCATGAGGG |
| 11tf_fwd1 | TGTGGGAAATGCAGAAT | 11tf_rev2 | (SEQ ID NO: 195) |
| | (SEQ ID NO: 105) | | |
| PANC1002 chr17_ | GTAAAACGACGGCCAGCTT | PANC1002 chr17_ | GAAGGGGGAAAAGGGTGATA |
| 12t_st1_fwd | CCCCTCCCTAGTTGACC | 12t_st1_rev | (SEQ ID NO: 196) |
| | (SEQ ID NO: 106) | | |
| PANC1002 Chr18: | GTAAAACGACGGCCAGGCA | PANC1002 Chr18: | CAGGAAACAGCTATGACATTG |
| 48M Fwd | TTGTAGATTCATACA | 48M_n1 Rev | GCTGGTGGGCACAC |
| | (SEQ ID NO: 107) | | (SEQ ID NO: 197) |

*Primers were named by their target cell line (e.g. “Panc480”), chromosome location (e.g. “chr1”) followed by either the first few numbers of the coordinates in the thousands (e.g. “550”) or the millions (e.g. “53M”).
#M13F sequence was adapted to forward primers for Sanger sequencing.

Among the validated ones, potential sgRNA sequences were selected in which either the PAM spans across the breakpoint junction or at least 4 bases of the sgRNA sequence cross the junction. Then, the sequence was put into CRISPOR and selected for candidates that have >50 specificity score.

WES Target Identification and sgRNA Design

1 ug of genomic DNA was used to prepare the genomic DNA library, then human exome capture was performed following a modified protocol from Agilent's SureSelect Paired-End Version 2.0 Human Exome Kit as previously described (32, 45). Captured DNA libraries were sequenced with a Genome Analyzer IIx System to 200× coverage, yielding 2×150 bp reads. FASTQ files were aligned to human genome hg18 with the Eland algorithm in CASAVA 1.7 software (Illumina), and the Database of Single Nucleotide Polymorphisms (dbSNP) was used in the analysis of the WES data. Mutations were inspected to include novel Cs that are adjacent to an existing C or novel Gs that are adjacent to an existing G, and visually confirmed on IGV. The resulting list of mutations was put through CRISPOR and the ones that can produce sgRNAs with >50 specificity score in CRISPOR are subsequently examined for their VAFs.

WGS Target Validation and sgRNA Design

DNA from tumor and non-tumor tissue for Panc480, Panc504, and Panc1002 were whole genome sequenced, aligned to the human genome (hg19), and variants called as previously described (46). Putative somatic mutations with a quality score of “PASS”, a distinct coverage (DP)>10, and a genotype quality score (GQ)>20 were identified using BEDTools (47). Somatic mutations were annotated with region-based (Func.refGene) and gene-based (Gene.refGene) identifications using ANNOVAR (48). Flanking sequences 2 base pairs 5′ and 3′ to somatic mutation positions were obtained from UCSC table browser (49). The following inclusion criteria are implemented: (1) novel Cs that are adjacent to an existing C, or novel Gs that are adjacent to an existing G; (2) VAF of at least 5% in tumor; (3) a minimum of 18× read depth (50) in both germline and tumor. These mutations were then visually inspected and confirmed on IGV. Somatic mutations with VAF >95% were chosen to put through CRISPOR. Somatic mutations that can produce sgRNAs with >50 specificity score in CRISPOR are subsequently validated by PCR and Sanger sequencing (See Supplemental Table 2, below).

TABLE 2
Primers for PCR and Sanger validation of novel base substitutions
discovered from WGS approach

| Primer name | Purpose | Sequence |
|---|---|---|
| Panc480_chr3: 537601_Fwd | Panc480 mutation | TGAGACTGTATTTGTGGGCCA |
| | validation | (SEQ ID NO: 198) |
| Panc480_chr3: 59525282_Fwd | Panc480 mutation | GGCCCTCACCATGTAAAAGG |
| | validation | (SEQ ID NO: 199) |
| Panc480_chr18: 1819017_Fwd | Panc480 mutation | ACTGGGAAGTTGGGTCTTCA |
| | validation | (SEQ ID NO: 200) |
| Panc480_chrX: 3982448_Fwd | Panc480 mutation | TGGAGGTAGGATATTACAGGGAA |
| | validation | (SEQ ID NO: 201) |
| Panc480_chr19: 58564841_Fwd | Panc480 mutation | GCCATCCACTCACTACAGGT |
| | validation | (SEQ ID NO: 202) |
| Panc480_chr8: 29032916_Fwd | Panc480 mutation | TGGAAGGCTAGAGGAAGCTG |
| | validation | (SEQ ID NO: 203) |
| Panc480_chr6: 124767224_Fwd | Panc480 mutation | TGTGTGCCTTCAAAATGGGG |
| | validation | (SEQ ID NO: 204) |
| Panc480_chr6: 55808003_Fwd | Panc480 mutation | TGAAGCATACATTCTGGAGGTT |
| | validation | (SEQ ID NO: 205) |
| Panc480_chr11: 64364029_Fwd | Panc480 mutation | TGGATGAACTGGATGGATGA |
| | validation | (SEQ ID NO: 206) |
| Panc480_chr6: 92757856_Fwd | Panc480 mutation | TGCCTAGTCCAGTAATGCGA |
| | validation | (SEQ ID NO: 207) |
| Panc480_chr17: 5377742_Fwd | Panc480 mutation | ACACCATGGCCTCATCTATCA |
| | validation | (SEQ ID NO: 208) |
| Panc480_chr4: 131074842_Fwd | Panc480 mutation | TGCTCTCAACTTTCCCTGGA |
| | validation | (SEQ ID NO: 209) |
| Panc480_chr8: 201457_Fwd | Panc480 mutation | GGGGGATGGTCATGAGATTT |
| | validation | (SEQ ID NO: 210) |
| Panc480_chr3: 86665957_Fwd | Panc480 mutation | CCTGCCCCAGTGAAATCAGT |
| | validation | (SEQ ID NO: 211) |
| Panc480_chr9: 15347394_Fwd | Panc480 mutation | AGGCAGCTAGAGTTCACAGG |
| | validation | (SEQ ID NO: 212) |
| Panc480_chr9: 110569399_Fwd | Panc480 mutation | GCAGAGGGGAGCTCTTTTCT |
| | validation | (SEQ ID NO: 213) |
| Panc480_chr1: 34085551_Fwd | Panc480 mutation | CCATTCCTCTCCACACTCCA |
| | validation | (SEQ ID NO: 214) |
| Panc480_chr3: 537601_rev | Panc480 mutation | AGCACGCAATATTACTGGGAAC |
| | validation | (SEQ ID NO: 215) |
| Panc480_chr3: 59525282_rev | Panc480 mutation | TGACCACCACATCCAGGAT |
| | validation | (SEQ ID NO: 216) |
| Panc480_chr18: 1819017_rev | Panc480 mutation | CACTCCCAAGAACGCAGAAT |
| | validation | (SEQ ID NO: 217) |
| Panc480_chrX: 3982448_rev | Panc480 mutation | ACCATCGTTTTAAAAGGTGCAA |
| | validation | (SEQ ID NO: 218) |
| Panc480_chr19: 58564841_rev | Panc480 mutation | GCTCGAGATCACAGTCCCTT |
| | validation | (SEQ ID NO: 219) |
| Panc480_chr8: 29032916_rev | Panc480 mutation | ATGTGCGGTGGTAGGAGAAG |
| | validation | (SEQ ID NO: 220) |
| Panc480_chr6: 124767224_rev | Panc480 mutation | AGCAATATGGAGGAACAAAAGCA |
| | validation | (SEQ ID NO: 221) |
| Panc480_chr6: 55808003_rev | Panc480 mutation | GTCATCCACTTCATCCACTTCA |
| | validation | (SEQ ID NO: 222) |
| Panc480_chr11: 64364029_rev | Panc480 mutation | AGGAGTGGCTGCAAATTGTT |
| | validation | (SEQ ID NO: 223) |
| Panc480_chr6: 92757856_rev | Panc480 mutation | CGGTATAGTTTCCACAGCAGG |
| | validation | (SEQ ID NO: 224) |
| Panc480_chr17: 5377742_rev | Panc480 mutation | CAGTTTGCCAGTGGTTCCTC |
| | validation | (SEQ ID NO: 225) |
| Panc480_chr4: 131074842_rev | Panc480 mutation | CACCGAGTTTGAGATGCCTG |
| | validation | (SEQ ID NO: 226) |
| Panc480_chr8: 201457_rev | Panc480 mutation | TGATCCAGTGTGGGTGAGAA |
| | validation | (SEQ ID NO: 227) |
| Panc480_chr3: 86665957_rev | Panc480 mutation | GGAGAGTGTACCCTGTTGCT |
| | validation | (SEQ ID NO: 228) |
| Panc480_chr9: 15347394_rev | Panc480 mutation | GCCCCGCTACTGAGAGAATA |
| | validation | (SEQ ID NO: 229) |
| Panc480_chr9: 110569399_rev | Panc480 mutation | ACCTCATCTCCCTGCTATGC |
| | validation | (SEQ ID NO: 230) |
| Panc480_chr1: 34085551_rev | Panc480 mutation | TCAGCCTCATCTTTCTCCCA |
| | validation | (SEQ ID NO: 231) |
| Panc1002_chr3: 41255526_fwd | Panc1002 mutation | ACTTGACATGTATGGTGGGG |
| | validation | (SEQ ID NO: 232) |
| Panc1002_chr3: 76569799_fwd | Panc1002 mutation | GGATTTTACAGCTGGAAGGGATC |
| | validation | (SEQ ID NO: 233) |
| Panc1002_chr4: 32408343_fwd | Panc1002 mutation | GCAACATTGCATGTTCAGAAA |
| | validation | (SEQ ID NO: 234) |
| Panc1002_chr4: 117677347_fwd | Panc1002 mutation | CGGTAGCTTGGATGACAGAA |
| | validation | (SEQ ID NO: 235) |
| Panc1002_chr4: 180416652_fwd | Panc1002 mutation | GGCCCTACCCATACCTACTG |
| | validation | (SEQ ID NO: 236) |
| Panc1002_chr4: 180746369_fwd | Panc1002 mutation | TAGGACTACAGCAGCACACC |
| | validation | (SEQ ID NO: 237) |
| Panc1002_chr6: 123690025_fwd | Panc 1002 mutation | TCCATTCCTTGTTCTTGCCAC |
| | validation | (SEQ ID NO: 238) |
| Panc1002_chr6: 153579209_fwd | Panc 1002 mutation | CCAAGCAACATAAAGCAGCA |
| | validation | (SEQ ID NO: 239) |
| Panc1002_chrX: 28266415_fwd | Panc 1002 mutation | TCTTTCTCCTAGATCTGGACACT |
| | validation | (SEQ ID NO: 240) |
| Panc1002_chrX: 56623848_fwd | Panc1002 mutation | GCTGCCTTTCTTCCAGTGAT |
| | validation | (SEQ ID NO: 241) |
| Panc1002_chrX: 116828813_fwd | Panc 1002 mutation | AGGCTCCACTGCTTCTGTGT |
| | validation | (SEQ ID NO: 242) |
| Panc1002_chr8: 12552195_fwd | Panc1002 mutation | TCCTGGGGCAATTTTACTTTT |
| | validation | (SEQ ID NO: 243) |
| Panc1002_chr8: 47456593_fwd | Panc 1002 mutation | GCTCACCCACTTTCCATTCA |
| | validation | (SEQ ID NO: 244) |
| Panc1002_chr8: 81741154_fwd | Panc1002 mutation | TCTGCCCCAACATGAGACTT |
| | validation | (SEQ ID NO: 245) |
| Panc1002_chr9: 23649543_fwd | Panc1002 mutation | TGTCCACACCTACAATCCTGA |
| | validation | (SEQ ID NO: 246) |
| Panc1002_chr11: 55366717_fwd | Panc1002 mutation | TCAGTTGTTTCACAGATCTGCA |
| | validation | (SEQ ID NO: 247) |
| Panc1002_chr12: 47771504_fwd | Panc1002 mutation | GTGCAGCTTCACTCCTCACA |
| | validation | (SEQ ID NO: 248) |
| Panc1002_chr18: 58907286_fwd | Panc1002 mutation | CAATTGCAACGGGAATTCTT |
| | validation | (SEQ ID NO: 249) |
| Panc1002_chrY: 17028622_fwd | Panc 1002 mutation | GCAGATAATGACCTTCCTATTGC |
| | validation | (SEQ ID NO: 250) |
| Panc1002_chr3: 15793085_fwd | Panc1002 mutation | GGTAGAGAAAAGCCCTGAGGA |
| | validation | (SEQ ID NO: 251) |
| Panc1002_chr3: 27365096_fwd | Panc 1002 mutation | GAGAACGGGAGGATTCTGG |
| | validation | (SEQ ID NO: 252) |
| Panc1002_chr4: 45316432_fwd | Panc 1002 mutation | TGCATCACAAGGGTTATTGC |
| | validation | (SEQ ID NO: 253) |
| Panc1002_chr4: 58746119_fwd | Panc1002 mutation | ATGCAACCTTTTGTGTTCCA |
| | validation | (SEQ ID NO: 254) |
| Panc1002_chr4: 63298774_fwd | Panc1002 mutation | TGTGGCACAGATTTATTAGCAGA |
| | validation | (SEQ ID NO: 255) |
| Panc1002_chr7: 158427297_fwd | Panc1002 mutation | ACAGGCACAACCATCCATTT |
| | validation | (SEQ ID NO: 256) |
| Panc1002_chrX: 9204373_fwd | Panc1002 mutation | ATGCCTGCATTTACCACCAT |
| | validation | (SEQ ID NO: 257) |
| Panc1002_chrX: 99446566_fwd | Panc1002 mutation | CCAATTTTAGGCATGCAGGT |
| | validation | (SEQ ID NO: 258) |
| Panc1002_chr8: 88685752_fwd | Panc1002 mutation | GGCAAATGTTCCCTGATGTT |
| | validation | (SEQ ID NO: 259) |
| Panc1002_chr9: 15744747_fwd | Panc1002 mutation | GCCAATCATGTGCCTCTCTT |
| | validation | (SEQ ID NO: 260) |
| Panc1002_chr17: 876863_fwd | Panc1002 mutation | TTTCCCAGGCTTCGTCGAT |
| | validation | (SEQ ID NO: 261) |
| Panc1002_chr18: 39354909_fwd | Panc1002 mutation | GCGGGGATTTGCACAGAATT |
| | validation | (SEQ ID NO: 262) |
| Panc1002_chr18: 51635625_fwd | Panc1002 mutation | GCACTCGAAGGCTTCTCC |
| | validation | (SEQ ID NO: 263) |
| Panc1002_chr19: 5559720_fwd | Panc1002 mutation | TCAATCAAGTGAGACAGGGCT |
| | validation | (SEQ ID NO: 264) |
| Panc1002_chr21: 24912568_fwd | Panc1002 mutation | CATGGGAGGCTGGATTCATT |
| | validation | (SEQ ID NO: 265) |
| Panc1002_chr3: 41255526_rev | Panc 1002 mutation | CTCCCCATAGCTAAGGACCA |
| | validation | (SEQ ID NO: 266) |
| Panc1002_chr3: 76569799_rev | Panc1002 mutation | GTCAAGATGTGGACTACTAGCA |
| | validation | (SEQ ID NO: 267) |
| Panc1002_chr4: 32408343_rev | Panc1002 mutation | GCCAAATCGGAAACAAAGAA |
| | validation | (SEQ ID NO: 268) |
| Panc1002_chr4: 117677347_rev | Panc1002 mutation | CAATGTAAGTGGGCAGCAGA |
| | validation | (SEQ ID NO: 269) |
| Panc1002_chr4: 180416652_rev | Panc1002 mutation | ACCAAGGCTAAAGATCAGTGAT |
| | validation | (SEQ ID NO: 270) |
| Panc1002_chr4: 180746369_rev | Panc1002 mutation | TCATTGGTATTTGGAGCTTTGC |
| | validation | (SEQ ID NO: 271) |
| Panc1002_chr6: 123690025_rev | Panc1002 mutation | CCAGCCTCTAGAACTGTGGA |
| | validation | (SEQ ID NO: 272) |
| Panc1002_chr6: 153579209_rev | Panc1002 mutation | ATGGTGTGTCAGACGCTGTT |
| | validation | (SEQ ID NO: 273) |
| Panc1002_chrX: 28266415_rev | Panc 1002 mutation | GGTAAATAACTTTGTCCTGGGTG |
| | validation | (SEQ ID NO: 274) |
| Panc1002_chrX: 56623848_rev | Panc 1002 mutation | GAAATTCTTCCTGCCAGCAC |
| | validation | (SEQ ID NO: 275) |
| Panc1002_chrX: 116828813_rev | Panc 1002 mutation | TGGTGGTGTTGGTGATTCAG |
| | validation | (SEQ ID NO: 276 - Same as SEQ ID |
| | | NO: 267) |
| Panc1002_chr8: 12552195_rev | Panc1002 mutation | TGGTGGTGTTGGTGATTCAG |
| | validation | (SEQ ID NO: 277) |
| Panc1002_chr8: 47456593_rev | Panc1002 mutation | TGCTTGCTTAAACTCCTCAGT |
| | validation | (SEQ ID NO: 278) |
| Panc1002_chr8: 81741154_rev | Panc1002 mutation | GGGTGACAATCTTCCTGTGG |
| | validation | (SEQ ID NO: 279) |
| Panc1002_chr9: 23649543_rev | Panc1002 mutation | GTTCCTTCAATTGCCGATGT |
| | validation | (SEQ ID NO: 280) |
| Panc1002_chr11: 55366717_rev | Panc1002 mutation | CAGCTCATCCAGAACCCAGA |
| | validation | (SEQ ID NO: 281) |
| Panc1002_chr12: 47771504_rev | Panc1002 mutation | ATGCTGCTGTGATCGTTTTG |
| | validation | (SEQ ID NO: 282) |
| Panc1002_chr18: 58907286_rev | Panc1002 mutation | GGAAAGTGGTGTCCAGGATG |
| | validation | (SEQ ID NO: 283) |
| Panc1002_chrY: 17028622_rev | Panc1002 mutation | CATGAATTACAAGGGCAGCAA |
| | validation | (SEQ ID NO: 284) |
| Panc1002_chr3: 15793085_rev | Panc 1002 mutation | ATAGGCGTACCCCTGAATCC |
| | validation | (SEQ ID NO: 285) |
| Panc1002_chr3: 27365096_rev | Panc1002 mutation | AAAGACCTTTGAAGGATGCAA |
| | validation | (SEQ ID NO: 286) |
| Panc1002_chr4: 45316432_rev | Panc1002 mutation | TGGATTCCAGAAATTGTTTTTGA |
| | validation | (SEQ ID NO: 287) |
| Panc1002_chr4: 58746119_rev | Panc1002 mutation | GCTATTCATTAGCGGGGACA |
| | validation | (SEQ ID NO: 288) |
| Panc1002_chr4: 63298774_rev | Panc1002 mutation | AAAGGCTTAGTGCTGACCTTACA |
| | validation | (SEQ ID NO: 289) |
| Panc1002_chr7: 158427297_rev | Panc1002 mutation | CATGGGCAGTTTGCTTTACC |
| | validation | (SEQ ID NO: 290) |
| Panc1002_chrX: 9204373_rev | Panc 1002 mutation | TTTCCAAGGTGATGACCACA |
| | validation | (SEQ ID NO: 291) |
| Panc1002_chrX: 99446566_rev | Panc1002 mutation | AGAAGGCCCTTTCATCATCA |
| | validation | (SEQ ID NO: 292) |
| Panc1002_chr8: 88685752_rev | Panc1002 mutation | AACTGGATTGGTTGCTGCTT |
| | validation | (SEQ ID NO: 293) |
| Panc1002_chr9: 15744747_rev | Panc1002 mutation | ACACTGTATTTCGCTTACATGCA |
| | validation | (SEQ ID NO: 294) |
| Panc1002_chr17: 876863_rev | Panc1002 mutation | TGGGTGACAGAGCAAGACT |
| | validation | (SEQ ID NO: 295) |
| Panc1002_chr18: 39354909_rev | Panc1002 mutation | GGCTCCTCCTCCCTACAAAT |
| | validation | (SEQ ID NO: 296) |
| Panc1002_chr18: 51635625_rev | Panc1002 mutation | TCATCCCTTTGTCCAGCAGA |
| | validation | (SEQ ID NO: 297) |
| Panc1002_chr19: 5559720_rev | Panc1002 mutation | TGTCCTCATTTCCCTGTGCA |
| | validation | (SEQ ID NO: 298) |
| Panc1002_chr21: 24912568_rev | Panc1002 mutation | AGACACGTAACGGCAGATGT |
| | validation | (SEQ ID NO: 299) |
| Panc504_chr1: 90925384_fwd | Panc504 mutation | TCTTTGTCTTGTGCATGGCG |
| | validation | (SEQ ID NO: 300) |
| Panc504_chr1: 109094826_fwd | Panc504 mutation | CTTAGAAAAGGCACAGCATAGG |
| | validation | (SEQ ID NO: 301) |
| Panc504_chr4: 96761136_fwd | Panc504 mutation | GCTCCAGGGTTTAACAGGGA |
| | validation | (SEQ ID NO: 302) |
| Panc504_chr4: 147513098_fwd | Panc504 mutation | GCCAGCCTTGAAGTGTGTC |
| | validation | (SEQ ID NO: 303) |
| Panc504_chrX: 10649926_fwd | Panc504 mutation | GCACATCCAAATTTATTCACACG |
| | validation | (SEQ ID NO: 304) |
| Panc504_chrX: 137303674_fwd | Panc504 mutation | GAACAACACCAGGCACATAGT |
| | validation | (SEQ ID NO: 305) |
| Panc504_chrX: 141322626_fwd | Panc504 mutation | GGAATTCCTGACTCCAAAACA |
| | validation | (SEQ ID NO: 306) |
| Panc504_chr9: 10209960_fwd | Panc504 mutation | CTGGTGCTTTTGTTTTGATTAGG |
| | validation | (SEQ ID NO: 307) |
| Panc504_chr9: 77440886_fwd | Panc504 mutation | AGGCAACAGGACATTTCAGG |
| | validation | (SEQ ID NO: 308) |
| Panc504_chr9: 105373293_fwd | Panc504 mutation | GCTGTTCCAATACAAGCCCC |
| | validation | (SEQ ID NO: 309) |
| Panc504_chr9: 133876782_fwd | Panc504 mutation | TCTGGTCCCATAACTGCACA |
| | validation | (SEQ ID NO: 310) |
| Panc504_chr10: 4171262_fwd | Panc504 mutation | TCTGGAGAACAAAGGCATTCC |
| | validation | (SEQ ID NO: 311) |
| Panc504_chr13: 107175748_fwd | Panc504 mutation | GGTTCCTGACTTCCATACGG |
| | validation | (SEQ ID NO: 312) |
| Panc504_chr18: 39014688_fwd | Panc504 mutation | GGGAGGGAGGGAAGAAACAA |
| | validation | (SEQ ID NO: 313) |
| Panc504_chr18: 48358086_fwd | Panc504 mutation | TGCATTTCTTATTTCCCAGCAAC |
| | validation | (SEQ ID NO: 314) |
| Panc504_chr18: 63239834_fwd | Panc504 mutation | AGCTGTGCAGGATTGAATTCT |
| | validation | (SEQ ID NO: 315) |
| Panc504_chr21: 23671417_fwd | Panc504 mutation | ATGACCAAAATGAGAAATTATTAGC |
| | validation | (SEQ ID NO: 316) |
| Panc504_chr1: 25383677_fwd | Panc504 mutation | GTATGCCAGGAGCCAGGTT |
| | validation | (SEQ ID NO: 317) |
| Panc504_chr1: 30192392_fwd | Panc504 mutation | CTTGGGTATGTGCCTTGCTC |
| | validation | (SEQ ID NO: 318) |
| Panc504_chr1: 73167766_fwd | Panc504 mutation | GCATGTGTTTACCTGGCCTAC |
| | validation | (SEQ ID NO: 319) |
| Panc504_chr1: 82861966_fwd | Panc504 mutation | CCTAAGGGTGTGACTCCAGA |
| | validation | (SEQ ID NO: 320) |
| Panc504_chr4: 32481045_fwd | Panc504 mutation | CATCACGCCCGGCTAATTTT |
| | validation | (SEQ ID NO: 321) |
| Panc504_chr4: 98124868_fwd | Panc504 mutation | GAGCTTTTGAATGGTGACTGGA |
| | validation | (SEQ ID NO: 322) |
| Panc504_chr4: 146038680_fwd | Panc504 mutation | CAAGCGCCTATGGAGTTGTC |
| | validation | (SEQ ID NO: 323) |
| Panc504_chr4: 177915089_fwd | Panc504 mutation | AGAAACCAGTGAAGGATCTCC |
| | validation | (SEQ ID NO: 324) |
| Panc504_chr4: 189873183_fwd | Panc504 mutation | GGGCAATAAACATGAAAAGTGGT |
| | validation | (SEQ ID NO: 325) |
| Panc504_chr5: 50335067_fwd | Panc504 mutation | ACAGCCCCAATCTGTTTCAC |
| | validation | (SEQ ID NO: 326) |
| Panc504_chr5: 76384387_fwd | Panc504 mutation | TAGAGGAGTTGGGGGAAGGT |
| | validation | (SEQ ID NO: 327) |
| Panc504_chr5: 117548593_fwd | Panc504 mutation | TCATCCCGAGAGTTATATCCCC |
| | validation | (SEQ ID NO: 328) |
| Panc504_chr7: 97304833_fwd | Panc504 mutation | AAGATCAAGCCAGCCACAAT |
| | validation | (SEQ ID NO: 329) |
| Panc504_chr7: 110208712_fwd | Panc504 mutation | CATCAACTCACTCACAGGCAG |
| | validation | (SEQ ID NO: 330) |
| Panc504_chr7: 137081417_fwd | Panc504 mutation | GATGTGCTGGCATGTGGAC |
| | validation | (SEQ ID NO: 331) |
| Panc504_chrX: 19715766_fwd | Panc504 mutation | GCTGCGGGACATAGAACTGT |
| | validation | (SEQ ID NO: 332) |
| Panc504_chrX: 22650252_fwd | Panc504 mutation | TGACCCTGGAATTCACCTGC |
| | validation | (SEQ ID NO: 333) |
| Panc504_chrX: 27834613_fwd | Panc504 mutation | TGTATCTGCGCCAAGGGAAA |
| | validation | (SEQ ID NO: 334) |
| Panc504_chrX: 105633682_fwd | Panc504 mutation | TTTTGAGTGAACGTGGCAGC |
| | validation | (SEQ ID NO: 335) |
| Panc504_chrX: 113360530_fwd | Panc504 mutation | AGGATTACTGATTGGGCCACT |
| | validation | (SEQ ID NO: 336) |
| Panc504_chr8: 15708017_fwd | Panc504 mutation | AGGTTTGTTCTCCCATAGTTGA |
| | validation | (SEQ ID NO: 337) |
| Panc504_chr9: 128664573_fwd | Panc504 mutation | AGATGTTTGCTCCAAGAACCT |
| | validation | (SEQ ID NO: 338) |
| Panc504_chr13: 67584092_fwd | Panc504 mutation | ACAAAGACATGCAACAGATCACA |
| | validation | (SEQ ID NO: 339) |
| Panc504_chr13: 70467817_fwd | Panc504 mutation | AGCAAACAAAAGAACCACTAGCT |
| | validation | (SEQ ID NO: 340) |
| Panc504_chr13: 92785652_fwd | Panc504 mutation | AGGGTGTCGTACTAAATGGGA |
| | validation | (SEQ ID NO: 341) |
| Panc504_chr18: 69135730_fwd | Panc504 mutation | CCAAGGTTAGGTGTGGGGAA |
| | validation | (SEQ ID NO: 342) |
| Panc504_chr22: 34609948_fwd | Panc504 mutation | GCTAAGGTGATCAACAAGTTTCC |
| | validation | (SEQ ID NO: 343) |
| Panc504_chr21: 29359027_fwd | Panc504 mutation | AGATCTCCCTTTTGTTGGTTGA |
| | validation | (SEQ ID NO: 344) |
| Panc504_chr1: 90925384_rev | Panc504 mutation | CAGGGATGTGTGGGAGATGA |
| | validation | (SEQ ID NO: 345) |
| Panc504_chr1: 109094826_rev | Panc504 mutation | GGTACGCACTCAATAGCTGG |
| | validation | (SEQ ID NO: 346) |
| Panc504_chr4: 96761136_rev | Panc504 mutation | GGGTGATAGAGGCAGGTCC |
| | validation | (SEQ ID NO: 347) |
| Panc504_chr4: 147513098_rev | Panc504 mutation | CCTTTACCCTCAAGTGCTTTCC |
| | validation | (SEQ ID NO: 348) |
| Panc504_chrX: 10649926_rev | Panc504 mutation | TGAGTGTCTATTAAGTGCCAGTG |
| | validation | (SEQ ID NO: 349) |
| Panc504_chrX: 137303674_rev | Panc504 mutation | CAGACCACCTATGACTAGAGCA |
| | validation | (SEQ ID NO: 350) |
| Panc504_chrX: 141322626_rev | Panc504 mutation | GTCCCCCTTCCTCAATCAAT |
| | validation | (SEQ ID NO: 351) |
| Panc504_chr9: 10209960_rev | Panc504 mutation | TGTTTTCAGAAATAAACTTTTTCACC |
| | validation | (SEQ ID NO: 352) |
| Panc504_chr9: 77440886_rev | Panc504 mutation | CTCTGGGAATTGTGGTCGTT |
| | validation | (SEQ ID NO: 353) |
| Panc504_chr9: 105373293_rev | Panc504 mutation | GGTGCTACTTGTCTCTCAGC |
| | validation | (SEQ ID NO: 354) |
| Panc504_chr9: 133876782_rev | Panc504 mutation | CATGAAATGGGAACGGTAGG |
| | validation | (SEQ ID NO: 355) |
| Panc504_chr10: 4171262_rev | Panc504 mutation | CCACAGACAGAGTAGGACAGA |
| | validation | (SEQ ID NO: 356) |
| Panc504_chr13: 107175748_rev | Panc504 mutation | CAGCACATCCTCCTTCCTCC |
| | validation | (SEQ ID NO: 357) |
| Panc504_chr18: 39014688_rev | Panc504 mutation | TCCCACCGTTCTCTGATCAT |
| | validation | (SEQ ID NO: 358) |
| Panc504_chr18: 48358086_rev | Panc504 mutation | AGTTGCTGTGGAGACCTTCA |
| | validation | (SEQ ID NO: 359) |
| Panc504_chr18: 63239834_rev | Panc504 mutation | ACTTGTTTCATGCCCTTGTTTT |
| | validation | (SEQ ID NO: 360) |
| Panc504_chr21: 23671417_rev | Panc504 mutation | TTGGTTGTGCTTCTTGTTGAA |
| | validation | (SEQ ID NO: 361) |
| Panc504_chr1: 25383677_rev | Panc504 mutation | TCGAGAAGGGAAAGATTGGA |
| | validation | (SEQ ID NO: 362) |
| Panc504_chr1: 30192392_rev | Panc504 mutation | TGGTGATGGAGGCAATGACT |
| | validation | (SEQ ID NO: 363) |
| Panc504_chr1: 73167766_rev | Panc504 mutation | ATAGGAGGGAGGCACAAGTG |
| | validation | (SEQ ID NO: 364) |
| Panc504_chr1: 82861966_rev | Panc504 mutation | GGTGATAAAGCGACCTTGAGT |
| | validation | (SEQ ID NO: 365) |
| Panc504_chr4: 32481045_rev | Panc504 mutation | GTACAGAGTCTCGGATGCTTTT |
| | validation | (SEQ ID NO: 366) |
| Panc504_chr4: 98124868_rev | Panc504 mutation | CACACCACTCCATTTGTCTGT |
| | validation | (SEQ ID NO: 367) |
| Panc504_chr4: 146038680_rev | Panc504 mutation | TGCTCAGTGATTAAATTCCAAGG |
| | validation | (SEQ ID NO: 368) |
| Panc504_chr4: 177915089_rev | Panc504 mutation | ATGCTATCATCATGGGCCCC |
| | validation | (SEQ ID NO: 369) |
| Panc504_chr4: 189873183_rev | Panc504 mutation | TGGACAGACATTTGGGGTGA |
| | validation | (SEQ ID NO: 370) |
| Panc504_chr5: 50335067_rev | Panc504 mutation | TCCAGGTGACTTGATGTAGCA |
| | validation | (SEQ ID NO: 371) |
| Panc504_chr5: 76384387_rev | Panc504 mutation | CAGCAGCAAAAGATGAGCAG |
| | validation | (SEQ ID NO: 372) |
| Panc504_chr5: 117548593_rev | Panc504 mutation | TCTGTCCTAATGCCCTTCCA |
| | validation | (SEQ ID NO: 373) |
| Panc504_chr7: 97304833_rev | Panc504 mutation | AGCTCTGGAAGTAGGCATTGA |
| | validation | (SEQ ID NO: 374) |
| Panc504_chr7: 110208712_rev | Panc504 mutation | CCACTGAGGGTATTGGGACA |
| | validation | (SEQ ID NO: 375) |
| Panc504_chr7: 137081417_rev | Panc504 mutation | TGAGTTGGTGTGGAGAGGAA |
| | validation | (SEQ ID NO: 376) |
| Panc504_chrX: 19715766_rev | Panc504 mutation | TAGCACCCCAGATCTCAGTG |
| | validation | (SEQ ID NO: 377) |
| Panc504_chrX: 22650252_rev | Panc504 mutation | GATTGAACCCTCATCATTTGCC |
| | validation | (SEQ ID NO: 378) |
| Panc504_chrX: 27834613_rev | Panc504 mutation | CCCCGCTGCACTCAATAAC |
| | validation | (SEQ ID NO: 379) |
| Panc504_chrX: 105633682_rev | Panc504 mutation | GCATTCTCTCACTCAAGCACA |
| | validation | (SEQ ID NO: 380) |
| Panc504_chrX: 113360530_rev | Panc504 mutation | TGGCTGTTCAGATATTGGATTCA |
| | validation | (SEQ ID NO: 381) |
| Panc504_chr8: 15708017_rev | Panc504 mutation | GGGGAAAGAGATGAGAAGAGAGA |
| | validation | (SEQ ID NO: 382) |
| Panc504_chr9: 128664573_rev | Panc504 mutation | AGAGTCATTGTCTACGATCCCA |
| | validation | (SEQ ID NO: 383) |
| Panc504_chr13: 67584092_rev | Panc504 mutation | TGCTCTTCACATTTCCTGAACA |
| | validation | (SEQ ID NO: 384) |
| Panc504_chr13: 70467817_rev | Panc504 mutation | GCCATTTCCAGAATTGAGACCA |
| | validation | (SEQ ID NO: 385) |
| Panc504_chr13: 92785652_rev | Panc504 mutation | TGCCTCCTTGAATGAACTGTG |
| | validation | (SEQ ID NO: 386) |
| Panc504_chr18: 69135730_rev | Panc504 mutation | AGAGAGAAACACTAGTAGCCTGA |
| | validation | (SEQ ID NO: 387) |
| Panc504_chr22: 34609948 rev | Panc504 mutation | GCGTAACTGCTAGAAGAAGAGA |
| | validation | (SEQ ID NO: 388) |
| Panc504_chr21: 29359027_rev | Panc504 mutation | AAGTCACTGGGAAGCAGTCA |
| | validation | (SEQ ID NO: 389) |

### Co-Culture Assays

Cells that expressed either mApple or EGFP fluorescence were co-cultured at different ratios. Proportion of mApple-expressing cells post-transduction of sgRNAs were measured at different time points using Attune NxT Flow Cytometer (ThermoFisher). FCS Express 7 (De Novo Software) was used to analyze the flow cytometry data.

### Mouse-Human NGS Assay

The RC3H2 gene was selected as the mouse and human orthologs differ by a 3 bp indel follow by 3 SNPs. Primers for unbiased PCR amplification of the locus in mouse and human DNA were previously developed by Lin et. al.(17), designated as primer pair 45 (See, Table 3 below)

TABLE 3
Primers used for mouse-human NGS assay

| Primer name | Sequence |
|---|---|
| NGS-RC3H2-45- | AATGATACGGCGACCACCGAGATCTACACTCTTTCCCTACACGACGCTCT |
| Lib-Fwd-1 | TCCGATCTTAAGTAGAGactaagtcaaggctactgtg |
| | (SEQ ID NO: 390) |
| NGS-RC3H2-45- | AATGATACGGCGACCACCGAGATCTACACTCTTTCCCTACACGACGCTCT |
| Lib-Fwd-2 | TCCGATCTATCATGCTTAactaagtcaaggctactgtg |
| | (SEQ ID NO: 391) |
| NGS-RC3H2-45- | AATGATACGGCGACCACCGAGATCTACACTCTTTCCCTACACGACGCTCT |
| Lib-Fwd-3 | TCCGATCTGATGCACATCTactaagtcaaggctactgtg |
| | (SEQ ID NO: 392) |
| NGS-RC3H2-45- | AATGATACGGCGACCACCGAGATCTACACTCTTTCCCTACACGACGCTCT |
| Lib-Fwd-4 | TCCGATCTCGATTGCTCGACactaagtcaaggctactgtg |
| | (SEQ ID NO: 393) |
| NGS-RC3H2-45- | AATGATACGGCGACCACCGAGATCTACACTCTTTCCCTACACGACGCTCT |
| Lib-Fwd-5 | TCCGATCTTCGATAGCAATTCactaagtcaaggctactgtg |
| | (SEQ ID NO: 394) |
| NGS-RC3H2-45- | CAAGCAGAAGACGGCATACGAGATTC |
| Lib-KO-Rev-1 | GCCTTGGTGACTGGAGTTCAGACGTGTGCTCTTCCGATCTttctggtgtcagtatgga |
| | ag |
| | (SEQ ID NO: 395) |
| NGS-RC3H2-45- | CAAGCAGAAGACGGCATACGAGATAT |
| Lib-KO-Rev-2 | AGCGTCGTGACTGGAGTTCAGACGTGTGCTCTTCCGATCTttctggtgtcagtatgg |
| | aag |
| | (SEQ ID NO: 396) |
| NGS-RC3H2-45- | CAAGCAGAAGACGGCATACGAGATGA |
| Lib-KO-Rev-3 | AGAAGTGTGACTGGAGTTCAGACGTGTGCTCTTCCGATCTttctggtgtcagtatgg |
| | aag |
| | (SEQ ID NO: 397) |
| NGS-RC3H2-45- | CAAGCAGAAGACGGCATACGAGATAT |
| Lib-KO-Rev-4 | TCTAGGGTGACTGGAGTTCAGACGTGTGCTCTTCCGATCTttctggtgtcagtatgga |
| | ag |
| | (SEQ ID NO: 398) |
| NGS-RC3H2-45- | CAAGCAGAAGACGGCATACGAGATCG |
| Lib-KO-Rev-5 | TTACCAGTGACTGGAGTTCAGACGTGTGCTCTTCCGATCTttctggtgtcagtatgga |
| | ag |
| | (SEQ ID NO: 399) |

For this assay, a 101 bp amplicon in the RC3H2 gene was amplified with primers containing Illumina adaptor sequences. Amplicons were subjected to NGS, and FASTQ files were aligned to the hg19 genome using bwa 0.7.17 (51) and visualized in IGV. Human and mouse reads were quantified as reads, and deletions, respectively, as the 3 bp-shorter mouse sequence maps as a deletion in the human genome. The assay was validated by sequencing 3 replicates of known mixtures of mouse and human DNA. For validation, mouse DNA was obtained from the liver of a nude mouse, and human DNA from human splenic tissue.

### CRISPR Multiplex Plasmid Functional Testing

To test the efficacy of multiplex CRISPR arrays expressing multiple sgRNA cassettes, the targeted cell line Panc480 was transduced at a 10:1 MOI with lentivirus expressing a non-targeting sgRNA (NT) or the multiplexed CRISPR array in a lentiGuide-puro backbone. Fourteen days after transduction and selection with puromycin, cells were harvested and gDNA (Table 2) with NGS adaptors and sent to Azenta for NGS. The sequencing data was analyzed for the percent of edited reads by CRISPResso2. Functional testing was performed in parallel for a non-targeted cell line, Panc1002, and a patient-matched EBV lymph normal cell line for Panc480, Onc3286. All targeted loci in the Panc480 cell line were found to be edited at varying efficiencies but no editing was detected in Panc1002 or Onc3286.

### STR Analysis

Mixed human DNA samples were PCR amplified using the AmpFLSTR Identifiler PCR Amplification Kit that amplifies 15 microsatellites (Applied Biosystems, Foster City, CA) per manufacturer's instructions, and amplicons resolved on a 3130 capillary electrophoresis instrument (Applied Biosystems). Percentage of a given individual was calculated from on-scale informative peak heights using chimeranalyzer (https://github.com/young-jon/chimeranalyzer).

### Confirmation of PAMs in Regional Lymph Nodes

FFPE preserved lymph nodes for Panc1002 and Panc504 were sectioned, deparaffinized, and macrodissected, and DNA was extracted by QIAamp DNA Mini Kit

(QIAGEN). Novel PAMs previously discovered in WGS of the primary tumor cell lines were PCR amplified with M13-tagged primers (Panc1002/504 mutation validation primers under “WGS target validations”) and Sanger sequenced. Sequence traces were compared to Sanger of the tumor cell line and patient-matched normal DNA to confirm the presence or absence of the mutation leading to the novel PAM.

### Statistical Analysis

The appropriate statistical tests were performed in GraphPad Prism (Version 9.2.0). The statistical models used were stated in results and in the Brief Description of the Figures. For all statistically significant results, * indicates p<0.05, ** indicates p<0.01, *** indicates p<0.001, and **** indicates p<0.0001.

dCas9 Plasmid Construction

pLentiCas9-T2A-GFP was a gift from Roderic Guigo & Rory Johnson {Pulido-Quetglas, 2017 #51} (Addgene plasmid #78548) and pZLCv2-3×FLAG-dCas9-HA-2×NLS {Campbell, 2018 #52} was a gift from Stephen Tapscott (Addgene plasmid #106357). Primers were designed to amplify the vector from pLentiCas9-T2A-GFP and dCas9 insert from pZLCv2-3×FLAG-dCas9-HA-2×NLS using Q5 Hot Start High-Fidelity polymerase (NEB) according to the manufacturer's protocol (Table 4, below).

TABLE 4
Primers for dCas9-EGFP plasmid construction and validation

| Name | Sequence | Purpose |
|---|---|---|
| Vector forward | Gtacgagacacggatcgacctgtctcagctgggaggcgacaagc | Gibson assembly |
| | gacctgccgccacaaa | |
| | (SEQ ID NO: 400) | |
| Vector reverse | Ctgtgttctggcggcaaacccgttgcgaaaaagaacgttcacggc | |
| | gactactgcacttat | |
| | (SEQ ID NO: 401) | |
| Insert forward | Gaacgttctttttcgcaacgggtttgccgccagaacacaggaccgg | |
| | tgccgcccaccatg | |
| | (SEQ ID NO: 402) | |
| Insert Reverse | Gtcgcctcccagctgagacaggtcgatccgtgtctcgtacaggcc | |
| | ggtgatgctctggtg | |
| | (SEQ ID NO: 403) | |
| D10 Forward | Tggctccgcctttttcccga | Validation (primers |
| | (SEQ ID NO: 404) | amplify across both |
| D10 Reverse | Ctcggctgtttctccgctgt | nuclease domains) |
| | (SEQ ID NO: 405) | |
| H840 Forward | Gagctgggcagccagatcct | |
| | (SEQ ID NO: 406) | |
| H840 Reverse | Cttggcattcagcagctggc | |
| | (SEQ ID NO: 407) | |

PCR products were subjected to gel electrophoresis with 0.8% agarose gel at 150V for 2 hours. Gel extraction was performed with QIAquick Gel Extraction Kit (QIAGEN) according to the manufacturer's protocol to purify the vectors and inserts. Then, Gibson assembly was performed with a 3:1 ratio of insert:vector using Gibson Assembly Master Mix (NEB) and an incubation time of 1 hour at 50° C. The Gibson product was transformed into NEB 5-alpha Competent E. coli according to the manufacturer's protocol and were selected by both carbenicillin and ampicillin. Plasmids were extracted from ampicillin-resistant clones using QIAprep Spin Miniprep kit (QIAGEN) according to the manufacturer's protocol. Analytical digestion with restriction enzymes (NEB) was performed to verify the identity of the plasmid. Primers were designed to PCR and Sanger sequence regions spanning D10 and H840 of dCas9 to validate the mutations on dCas9.

### Cas9-mApple Plasmid Construction

mApple-N1 {Shaner, 2008 #53} was a gift from Michael Davidson (Addgene plasmid #54567). Primers were designed to amplify the vector from pLentiCas9-T2A-GFP and mApple insert from mApple-N1 using Q5 Hot Start High-Fidelity polymerase (NEB) according to the manufacturer's protocol (Table 5, below).

TABLE 5
Primers for Cas9-mApple plasmid construction and validation

| Name | Sequence | Purpose |
|---|---|---|
| Vector forward | Ctccaccggcggcatggacgagctgtacaagcatcatcac | Gibson |
| | (SEQ ID NO: 408) | assembly |
| Vector reverse | Ccatgttattctcctcgcccttgctcaccatggtggcgac | |
| | (SEQ ID NO: 409) | |
| Insert forward | Gggcgaggagaataacatggccatcatcaaggagttcatg | |
| | (SEQ ID NO: 410) | |
| Insert Reverse | Cgtccatgccgccggtggagtggcggccctcggcgcgttc | |
| | (SEQ ID NO: 411) | |
| mCherry-F | CCCCGTAATGCAGAAGAAGA | Insertion |
| | (SEQ ID NO: 412) | validation |
| WPRE-R | CATAGCGTAAAAGGAGCAACA | |
| | (SEQ ID NO: 413) | |

PCR products were subjected to gel electrophoresis with 0.8% agorose gel at 150V for 2 hours. Gel extraction was performed with QIAquick Gel Extraction Kit (QIAGEN) according to the manufacturer's protocol to purify the vectors and inserts. Then, Gibson assembly was performed with a 2:1 ratio of insert:vector using Gibson Assembly Master Mix (NEB) and an incubation time of 1 hour at 50° C. The Gibson product was transformed into NEB 5-alpha Competent E. coli according to the manufacturer's protocol and were selected by both carbenicillin and ampicillin. Plasmids were extracted from ampicillin-resistant clones using QIAprep Spin Miniprep kit (QIAGEN) according to the manufacturer's protocol. Analytical digestion with restriction enzymes (NEB) was performed to verify the identity of the plasmid. Primers were designed to confirm insertion. The plasmid was then transfected into 293T cells with Invitrogen Lipofectamine 3000 reagent and P3000 reagent (ThermoFisher) according to manufacturer's protocol, and observe under fluorescence microscope for functional validation.

sgRNA-Expressing Plasmid Construction

lentiGuide-Puro {Sanjana, 2014 #54} was a gift from Feng Zhang (Addgene plasmid #52963) and lentiCRISPRv2 puro {Stringer, 2019 #56} was a gift from Brett Stringer (Addgene plasmid #98290). Oligonucleotides of sgRNA sequences were ordered from IDT for cloning into both lentiGuide-Puro and lentiCRISPRv2 puro backbones according to Feng Zhang's Lab Target Guide Sequence Cloning protocol. The resulting product was transformed into One Shot Stb13 chemically competent E. coli (ThermoFisher) according to the manufacturer's protocol and selected with both carbenicillin and ampicillin. Plasmids were extracted from ampicillin-resistant clones using QIAprep Spin Miniprep kit (QIAGEN) according to the manufacturer's protocol. Analytical digestion with restriction enzymes (NEB) was performed to verify the identity of the plasmids and Sanger sequencing was performed to validate the insertion of sgRNA sequence.

### Cell Culture

Panc10.05, TS0111, Panc480, Panc1002, A10.7, A6L, A32.1, NIH3T3, Panc02, Onc3286, and their derivative cell lines were STR profiled and mycoplasma tested before the start of experiments. All cells, except for Onc3286, were maintained in monolayer cultures at 37° C. and 5% CO₂. The culture medium consists of 1×DMEM, 10% fetal bovine serum, 2 mM L-glutamine, and 1× antibiotic antimycotic solution (Sigma; contains 100u penicillin, 100 ug streptomycin, and 0.25 ug amphotericin B). Onc3286 was maintained in a suspension culture at 37° C. and 5% CO₂. The culture medium consists of 1×RPMI 1640, 20% heat-inactivated bovine calf serum, 2 mM L-glutamine, and 1× antibiotic antimycotic solution (Sigma).

### Lentivirus Titer Preparation and Quantification

pCMV-VSV-G {Stewart, 2003 #57} was a gift from Dr. Bob Weinberg (Addgene plasmid #8454), pMDLg/pRRE and pRSV-Rev were gifts from Dr. Didier Trono {Dull, 1998 #58} (Addgene plasmid #12251 & #12253). 2.5 ug pCMV-VSV-G, 5 ug pMDLg/pRRE, 5 ug pRSV-Rev, and 7.5 ug transfer plasmids were used along with 50 uL Invitrogen Lipofectamine 3000 reagent and 40 uL P3000 reagent (ThermoFisher) for transfection into 293T cells on a 10-cm plate (95-99% confluent at transfection). Cell culture and transfection workflows were the same as the manufacturer's protocol. Upon harvesting and pooling the lenvirus-containing supernatant, the clarified supernatant was concentrated with Lenti-X Concentrator (Takara Bio) by following the manufacturer's protocol. Lenti-X qRT-PCR titration kit (Takara Bio) was used to quantify an aliquot of the clarified lentiviral supernatant according to the manufacturer's protocol.

### Fluorescent Cell Line Construction

Cells were seeded at 50% confluence for 24 hours before the media was replaced to contain 10 ug/mL of polybrene. Lentivirus of MOI 0.01 was added into the media and transduction took place for 18-20 hours. The media was then removed, washed once with PBS, and replaced with normal media. After 24 hours, the media was replaced with media that contained 5 ug/mL blasticidin for a 7-day selection. The cells were then sent to the SKCCC Flow Cytometry Core or SKCCC High Parameter Flow Core for fluorescence activated cell sorting using BD FACSAria II or BD Fusion sorter, respectively, to sort for cells with the optimal fluorescence intensity. The sorted cells were cultured in the presence of blasticidin selection and subjected to STR profiling and mycoplasma testing. Fluorescence microscopy was performed to verify the presence of fluorescent marker before experiments were carried out on these cell lines.

### Cas9 Activity Assay

Cells were transduced with sgRNAs targeting HPRT1 gene to induce mutations, which could be functionally screened via 6-thioguanine (6-TG) positive selection. For human, the sgRNA used is HPRTc.465 and non-targeting control is NT2; for mouse, it is mchrX:52M with mchrX:53M as an off-target control (Table 6, below).

TABLE 6
sgRNAs and primers for Cas9 activity assay

| Name | Sequence | Purpose |
|---|---|---|
| NT2 | GCGAGGTATTCGGCTCCGCG (SEQ ID NO: 2) | sgRNAs for |
| HPRTc.465 | TGGATTATACTGCCTGACCA (SEQ ID NO: 4) | human cells |
| mchrX:52M | TGCTCCACTTTGAAACAGCTG (SEQ ID NO: 414) | sgRNAs for |
| mchrX:53M | GGGGACTGACATTACCTCTGC (SEQ ID NO: 415) | mouse cells |
| i_HPRTc.465_ | AATGATACGGCGACCACCGAGATCTACACTCTTT | NGS primers |
| Fwd-2 | CCCTACACGACGCTCTTCCGATCTATCATGCTTA | for human cell |
| | GAGGGCCAGATGATATAGATTCC | lines |
| | (SEQ ID NO: 416) | |
| ib_HPRTc.465_ | CAAGCAGAAGACGGCATACGAGATATAGCGTCG | |
| Rev-2 | TGACTGGAGTTCAGACGTGTGCTCTTCCGATCTG | |
| | GCAAGGAAGTGACTGTAATTATG | |
| | (SEQ ID NO: 417) | |
| mchrX_52M_ | AATGATACGGCGACCACCGAGATCTACACTCTTT | NGS primers |
| Fwd | CCCTACACGACGCTCTTCCGATCTTAAGTAGAGT | for mouse cell |
| | GCTCCACTTTGAAACAGCTG | lines |
| | (SEQ ID NO: 418) | |
| mchrX_52M_ | CAAGCAGAAGACGGCATACGAGATTCGCCTTGG | |
| Rev | TGACTGGAGTTCAGACGTGTGCTCTTCCGATCTA | |
| | CACATGCCTCTCCTCTCTCT | |
| | (SEQ ID NO: 419) | |

Target site was PCR amplified and sent for NGS (Table 6). Mutation frequency of target site is quantified using CRISPResso2 pipeline {Clement, 2019 #59}. Alternatively, cells that survive 2 weeks of 3 ug/mL 6-TG indicate mutation at the HPRT1 gene.

### Single Nucleotide Variant (SNV) on Perfect Target Site Vs Mutation Frequency

To interrogate the effect of SNV present on perfect target site on the mutation frequencies calculated from each resistant colony sent for WGS, percentage of perfect target site with SNV was calculated by dividing the number of perfect target sites present with SNV based on WGS data by the number of perfect target sites predicted in each sgRNA; percentage of mutation frequency of each sgRNA was obtained by dividing total mutation frequency of all perfect target sites found in each colony by the number of predicted perfect target sites. Colonies with >25% perfect target sites containing SNV were excluded from the analysis to prevent the sgRNA sequence mismatch from confounding the toxicity analysis. Resistant colonies that exhibited <50% mutation frequency overall were also excluded from the toxicity analysis.

### Time-Course PCR

Panc10.05-Cas9-EGFP cells were transduced with 164R(14) sgRNA and cultured over the course of 2 weeks without antibiotic selection. Cell pellets were collected at various time points for gDNA extraction using QIAamp UCP DNA Micro Kit (QIAGEN) by following manufacturer's protocol (Table 7, below).

TABLE 7
NGS primers for time course PCR

| Locus | Primer | | |
|---|---|---|---|
| coordinate* | name | Forward primer | Reverse primer |
| chr1:224, 171, | 164R12_chr1_ | AATGATACGGCGACCACC | CAAGCAGAAGACGGCATAC |
| 172-224, 171, 194 | 224M_1 | GAGATCTACACTCTTTCCC | GAGATTC |
| | | TACACGACGCTCTTCCGAT | GCCTTGGTGACTGGAGTTCA |
| | | CTTAAGTAGAGGGGATCA | GACGTGTGCTCTTCCGATCT |
| | | TCACCAGACCTTTG | CACCACGCCTGCCTAATTTT |
| chr1:164, 976-164, | 164R12_chr1_ | AATGATACGGCGACCACC | CAAGCAGAAGACGGCATAC |
| 998 | 164_1 | GAGATCTACACTCTTTCCC | GAGATTC |
| | | TACACGACGCTCTTCCGAT | GCCTTGGTGACTGGAGTTCA |
| | | CTTAAGTAGAGGGGATCA | GACGTGTGCTCTTCCGATCT |
| | | TCACCGGACCTTT | CACCACGCCTGCCTAATTTT |
| | | (SEQ ID NO: 420; Same | (SEQ ID NO: 427) |
| | | as SEQ ID NO: 421) | |
| chr11:160, | 164R12_chr11_ | AATGATACGGCGACCACC | CAAGCAGAAGACGGCATAC |
| 165-160, 187 | 160_1 | GAGATCTACACTCTTTCCC | GAGATTC |
| | | TACACGACGCTCTTCCGAT | GCCTTGGTGACTGGAGTTCA |
| | | CTTAAGTAGAGGGGATCA | GACGTGTGCTCTTCCGATCT |
| | | TCACCGGACCTTT | TTTCATCATGTTGGCCAGGC |
| | | (SEQ ID NO: 421) | (SEQ ID NO: 428) |
| chr1:222, 684, | 164R12_chr1_ | AATGATACGGCGACCACC | CAAGCAGAAGACGGCATAC |
| 185-222, 684, 207 | 222M_2 | GAGATCTACACTCTTTCCC | GAGATAT |
| | | TACACGACGCTCTTCCGAT | AGCGTCGTGACTGGAGTTCA |
| | | CTATCATGCTTATCACCAG | GACGTGTGCTCTTCCGATCT |
| | | ACCTTCGGCTTTT | CACCACGCCTGCCTAATTTT |
| | | (SEQ ID NO: 422) | (SEQ ID NO: 429) |
| chr3:197, 916, | 164R12_chr3_ | AATGATACGGCGACCACC | CAAGCAGAAGACGGCATAC |
| 501-197, 916, 523 | 197M_1 | GAGATCTACACTCTTTCCC | GAGATTC |
| | | TACACGACGCTCTTCCGAT | GCCTTGGTGACTGGAGTTCA |
| | | CTTAAGTAGAGCACCACG | GACGTGTGCTCTTCCGATCT |
| | | CCTGCCTAATTTT | GGGATCATCACCGGACCTTT |
| | | (SEQ ID NO: 423; Same | (SEQ ID NO: 430; Same as |
| | | as SEQ ID NO: 424) | SEQ ID NO: 431) |
| chr16:90, 203, | 164R12_chr16_ | AATGATACGGCGACCACC | CAAGCAGAAGACGGCATAC |
| 887-90, 203, 909 | 90M_1 | GAGATCTACACTCTTTCCC | GAGATTC |
| | | TACACGACGCTCTTCCGAT | GCCTTGGTGACTGGAGTTCA |
| | | CTTAAGTAGAGCACCACG | GACGTGTGCTCTTCCGATCT |
| | | CCTGCCTAATTTT | GGGATCATCACCGGACCTTT |
| | | (SEQ ID NO: 424) | (SEQ ID NO: 431) |
| chr1:243, 251, | 164R12_chr1_ | AATGATACGGCGACCACC | CAAGCAGAAGACGGCATAC |
| 719-243, 251, 741 | 243M_2 | GAGATCTACACTCTTTCCC | GAGATAT |
| | | TACACGACGCTCTTCCGAT | AGCGTCGTGACTGGAGTTCA |
| | | CTATCATGCTTAGATCATC | GACGTGTGCTCTTCCGATCT |
| | | ACCGGACCTTTGG | GCCTCAGCCTCCTAAGTAGC |
| | | (SEQ ID NO: 425) | (SEQ ID NO: 432) |
| chr5:180, 721, | 164R12_chr5_ | AATGATACGGCGACCACC | CAAGCAGAAGACGGCATAC |
| 841-180, 721, 863 | 180M_1 | GAGATCTACACTCTTTCCC | GAGATTC |
| | | TACACGACGCTCTTCCGAT | GCCTTGGTGACTGGAGTTCA |
| | | CTTAAGTAGAGCACCACG | GACGTGTGCTCTTCCGATCT |
| | | CCTGCCTAATTTT | GGGATCATCACCGGACCTTT |
| | | (SEQ ID NO: 426) | (SEQ ID NO: 433) |

*Primers were designed for 8 loci of 164R(14) perfect target sites based on hg19.

Primers were designed for 8 perfect target regions of the 164R(14) for PCR and NGS. Quantification of mutation frequency of all target sites were done using CRISPResso2 pipeline.

### Karyotyping

Chromosome analyses were performed using the G-banding technique on TS0111-Cas9-EGFP cell line before and after treatment of a 14-cutter sgRNA using standard techniques. The abnormal karyotypes were described using the International System for Human Cytogenetic Nomenclature (ISCN 2020).

### SV Identification and Quantification Using Trellis

From the WGS BAM files of surviving colonies, Manta v0.29.6 was used to call somatic SVs and between the sample and the control, in which the control is the Panc10.05-Cas9-EGFP non-transduced cell line. The default parameters were used. Variants were annotated according to UCSC refseq annotations using an in-house script. The list of SVs generated were then individually, visually inspected on IGV to validate its presence in sample and absence in control. Novel SVs were quantified using SVs that have passed the manual screening.

For SV identification using Trellis {Langmead, 2012 #75}, we performed analysis on the Joint High Performance Computing Exchange, a 64 bit Linux Red Hat cluster, hosted at the Johns Hopkins Bloomberg School of Public Health. Bowtie2 {Langmead, 2012 #75} was used, with default settings, to align the paired end, 2×151 bp, Fastq files to Hg19. We indexed the aligned files with samtools version 1.14 {Li, 2009 #4} and used the resulting bam files as input to the R program Trellis for rearrangement detection {Papp, 2018 #33}. The Trellis code was customized to prevent removal of aligned read-pairs containing at least one read with a map quality below 30. This modification enabled rearrangements to be detected within low complexity reference sequence, a change necessary to detect rearrangements overlapping our target loci, all of which comprised sequences that were repeated multiple times within the reference genome. Trellis input settings included five minimum tags per cluster, 100 bp gap width between reads within a cluster, 10 k bp maximum cluster size, and 10 k bp minimum read-pair separation, and no automatic removal of genomic loci with previous annotation of publicly available samples indicating germline rearrangements. A secondary set of filters was applied to the primary Trellis results to remove likely artifacts. The secondary filters removed candidate rearrangements with mean map quality scores <1, read-pair count 40, at least one junction in the Y chromosome, Trellis annotation indicating a copy number change (either an amplification or deletion) and rearrangements junctions appearing in at least one of the two negative controls.

### Multiplex Cloning

Individual sgRNA targeting novel PAMs were obtained as ssDNA oligos from IDT and cloned into lentiGuide-puro (Addgene #52963) and lentiCRISPRv2-puro (Addgene #98290) lentiviral expression vectors per the protocol previously published by the Zhang Lab (Sanjana et al. 2014, Shalem et al. 2014). The U6 promoter, guide sequence, and sgRNA scaffold, referred to here as cassettes, were then PCR amplified off each lentiGuide-puro-sgRNA construct for each locus targeted (Table 8, below).

TABLE 8
Primers involved in multiplex sgRNA vector construction

| Primer name | Sequence | Purpose |
|---|---|---|
| Multi_lenti_frag_ | Cccacctcccaaccccgaggggacccagagagggcctatttc | amplification of |
| fwd1 | (SEQ ID NO: 434) | sgRNA cassettes |
| Multi_lenti_rev_2 | Gggaaataggccctctctgggtcgaaaaaagcaccgactcggtgccactt | |
| | (SEQ ID NO: 435) | |
| multiplex-BsrGI-fwd | tatcgttgTGTACAaggcagggatattcaccatt | amplification of |
| | (SEQ ID NO: 436) | LOH array out of |
| multiplex-MreI-rev | tatcgttgCGCCGGCGaattgtggatgaatactgcc | lentiGuide |
| | (SEQ ID NO: 437) | |
| lentiC_vecfwd-MreI | tatcgttgCGCCGGCGgaattcgctagctaggtcttg | linearization of |
| | (SEQ ID NO: 438) | lentiCRISPRv2- |
| lentiC_vecrev-BsrGI | tatcgttgTGTACAccaaactggatctctgc | puro |
| | (SEQ ID NO: 439) | |
| lentiG_vecfwd-MreI | tatcgttgCGCCGGCGgagacaaatggcagtattcatc | linearization of |
| | (SEQ ID NO: 440) | lentiGuide-puro |
| lentiG_vecrev-BsrGI | tatcgttgTGTACActctattcactatagaaagtacagcaaaaactattctt | |
| | aaacc | |
| | (SEQ ID NO: 441) | |
| Stitch_fragFwd | Agggatattcaccattatcgtcgtttcagacccacct | Gibson Assembly |
| | (SEQ ID NO: 442) | of LOH-7 partial |
| Stitch_fragRev | Gggttgggaggtgggtctgactcaagatctagttacgccaagct | assemblies |
| | (SEQ ID NO: 443) | |
| Stitch_vectorFwd | Tggcgtaactagatcttgagtcagacccacctcccaaccc | |
| | (SEQ ID NO: 444) | |
| Stitch_vectorRev | Gggaggtgggtctgaaacgacgataatggtgaa | |
| | (SEQ ID NO: 445) | |
| Mulitplex_lenti_ | Aggcagggatattcaccatt | Construct |
| fwd1 | (SEQ ID NO: 446) | validation |
| Mulitplex_lenti_ | Aattgtggatgaatactgcc | |
| rev2 | (SEQ ID NO: 447) | |
| 480LOHG1_fwd | GGAATCATCTTCACAGTTGT | |
| | (SEQ ID NO: 448) | |
| 480LOHG1_Rev | ACAACTGTGAAGATGATTCC | |
| | (SEQ ID NO: 449) | |
| 480LOHG4_fwd | CTAATGTATGACTGAAAGCT | |
| | (SEQ ID NO: 450) | |
| 480LOHG4_Rev | AGCTTTCAGTCATACATTAG | |
| | (SEQ ID NO: 451) | |
| 480LOHG5_fwd | GAGGTGTCTAAACCATGACA | |
| | (SEQ ID NO: 452) | |
| 480LOHG5_Rev | TGTCATGGTTTAGACACCTC | |
| | (SEQ ID NO: 453) | |
| pFH6-seq_fwd | Ctgcaggtcgaccatatggg | |
| | (SEQ ID NO: 454) | |

For multiplexing, the lentiGuide-puro construct containing the first guide was linearized by PpuMI digestion (NEB) and cassettes were serially added by Gibson assembly with PpuMI linearization of the growing array for each cycle (Table 8). The final multitarget-7 (MT7) construct was then back-cloned into the original species of lentiGuide-puro and verified by analytical digestion and Sanger sequencing (Table 8).

### Example 2: Increased Numbers of CRISPR-Cas9 Induced DSBs Inhibit Cell Growth

It was hypothesized that toxicity would increase with the number of simultaneously induced DSBs. To test this, sgRNAs were designed that were predicted to have multiple (2-16) target sites in the human genome, and designated them multi-target sgRNAs (Table 9, below)

TABLE 9:
sgRNAs used to perform clonogenicity and sgRNA survival assays

| | | | Number of | | Number of | |
|---|---|---|---|---|---|---|
| | | Number of | potential | Number of | potential | Doench |
| | | perfect | off- | perfect | off- | ‘16 |
| | | target | target | target | target | predicted |
| | | sites | sites | sites | sites | efficiency |
| sgRNA | Sequence¹ | (hg19)² | (hg19)² | (GRCh38)³ | (GRCh38)³ | score⁵ |
| NT | GTATTACTGATATTGGTGGG | 0 | 0-1-12-111 | 0 | 0-1-12-111 | NA |
| | (SEQ ID NO: 1) | | | | | |
| NT2 | GCGAGGTATTCGGCTCCGCG | 0 | 0-0-2-10 | 0 | 0-0-2-10 | NA |
| | (SEQ ID NO: 2) | | | | | |
| HPRTc.80 | ATTATGCTGAGGATTTGGAA | 1 | 0-2-34-228 | 1 | 0-2-35-231 | 65 |
| | (SEQ ID NO: 3) | | | | | |
| HPRTc.465 | TGGATTATACTGCCTGACCA | 1 | 0-2-8-70 | 1 | 0-2-8-70 | 64 |
| | (SEQ ID NO: 4) | | | | | |
| 531F(2) | CACTCAGCATCGACTTACGA | 2 | 4-1-0-17 | 2 | 4-1-0-17 | 66 |
| | (SEQ ID NO: 5) | | | | | |
| 52F(3) | TAATTACTGCACGATGCGCA | 3 | 0-0-2-13 | 3 | 0-0-2-13 | 59 |
| | (SEQ ID NO: 6) | | | | | |
| 715F(5) | ATATATATGCGATCGAGCCC | 5 | 2-1-5-28 | 5 | 2-1-5-28 | 54 |
| | (SEQ ID NO: 7) | | | | | |
| 451F(6)⁴ | ACTAGTGTGCGTATGATTTG | 6 | 0-1-4-65 | 6 | 0-1-4-65 | 57 |
| | (SEQ ID NO: 8) | | | | | |
| 176R(7) | TCGATGTTCTACATCGATGT | 6 | 1-1-6-168 | 7 | 2-1-6-168 | 60 |
| | (SEQ ID NO: 9) | | | | | |
| 551R(8) | TTGAATTGAGTTGCAACCGA | 8 | 2-1-4-47 | 8 | 2-1-4-49 | 61 |
| | (SEQ ID NO: 10) | | | | | |
| 230F(12)⁴ | TTGTCCCACAATGATACTTG | 12 | 7-1-8-94 | 12 | 8-1-8-94 | 61 |
| | (SEQ ID NO: 11) | | | | | |
| 164R(14)⁴ | GGATATTTCACTACAGACTT | 12 | 5-2-15-141 | 14 | 5-2-15-144 | 53 |
| | (SEQ ID NO: 12) | | | | | |
| 676F(16) | CTCCGAACTTAACTTGCCCT | 14 | 2-6-17-56 | 16 | 2-6-17-60 | 55 |
| | (SEQ ID NO: 13) | | | | | |
| AGGn | AGGAGGAGGAGGAGGAGGAG | Repeat | | Repeat | | 37 |
| | (SEQ ID NO: 14) | | | | | |
| L1.4_209F | TGCCTCACCTGGGAAGCGCA | 600 | 935-1723- | 604 | 939-1710- | 55 |
| | (SEQ ID NO: 15) | | 2210-1897 | | 2213-1908 | |
| ALU_112a | TTGCCCAGGCTGGAGTGCAG | Repeat | | Repeat | | 58 |
| | (SEQ ID NO: 16) | | | | | |

¹Sequences are followed in the genome by either canonical (NGG) and non-canonical (NGA/NAG) PAMs. CRISPOR analysis of the sgRNAs to identify the potential perfect and off-target sites (1-2-3-4 mismatches) in both ²hg19/GRCh37 and ²GRCh38 human reference genome.
⁴sgRNA is labeled as inefficient by CRISPOR.
⁵Cutting efficiency score based on data trained by Doench et al. 2016. Recommended for sgRNAs expressed with U6 promoter. The higher the efficiency score, the more likely is cleavage at this position.

To focus exclusively on the effect of multiple DSBs and exclude toxicity due to inactivation of specific gene functions, sgRNAs predicted to cut in non-coding regions of the genome were selected. (10). Two non-targeting (NT) sgRNAs were picked as negative controls, and sgRNAs that target repetitive elements as positive controls. Finally, as a functional test for Cas9 activity, two sgRNAs predicted to cut once in the HPRT1 gene were designed, due to the ability to select cells that have undergone gene inactivation using 6-thioguanine.

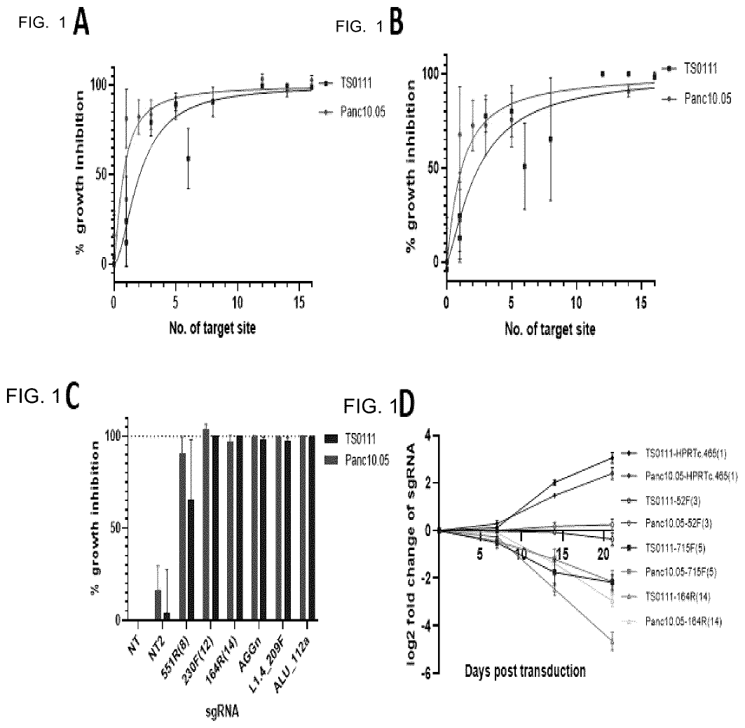

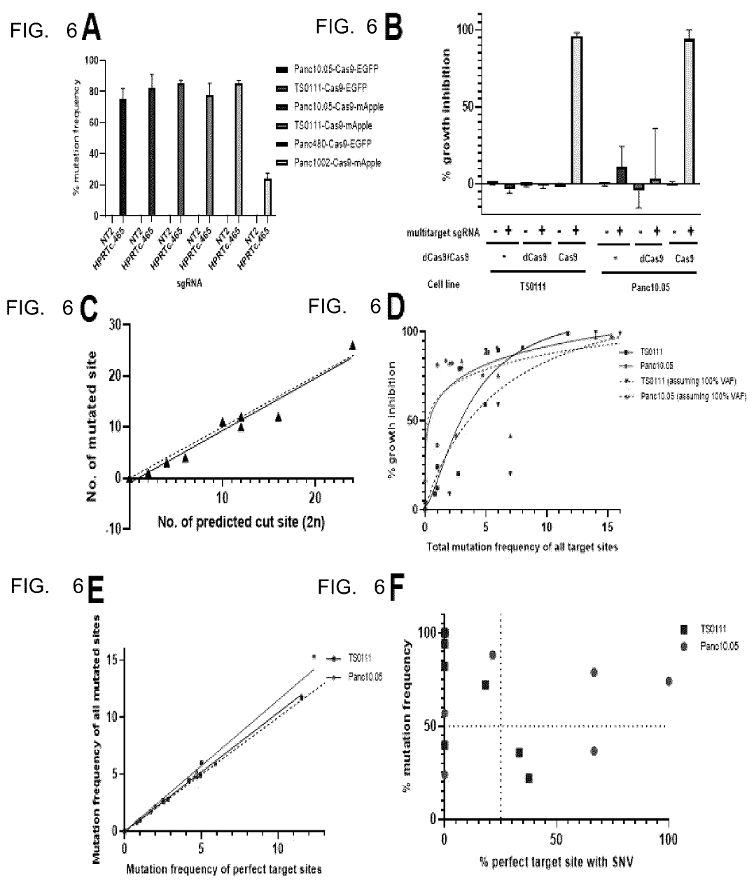

Two PC cell lines (Panc10.05 and TS0111) were constructed to constitutively express Cas9, documented functional activity (FIG. 6A), and confirmed that both Cas9 and sgRNA were required for toxicity (FIG. 6B). These were then transduced with the multi-target sgRNAs and measured growth inhibition using alamarBlue (FIG. 1A) and clonogenicity (FIG. 1B). Toxicity varied only slightly between the assays and cell lines though was qualitatively similar between them. The sgRNAs that targeted 3 sites corresponded to 73% growth inhibition (FIG. 1A and FIG. 1B), while those with 12 or more sites consistently showed >99% elimination for both cell lines (FIG. 1A-1C). While cell elimination increased as a function of the number of sites targeted, some variability was noted in this relationship (e.g., the 6-cutter showed less toxicity than the 5-cutter), which may be due to sgRNA targeting efficiency or other factors (11).

Due to concern that cutting might occur at off-target mismatched sites, whole genome sequencing (WGS) of surviving colonies from the multi-target treated cells was examined. When they could be obtained, two resistant colonies after single cell cloning for each sgRNA from both cell lines were studied by examining perfectly matched sites and those containing 1-4 mismatches. Notably, colonies for the 12-cutter or 16-cutter, and 8- to 14-cutters for the Panc10.05 and TS0111 cell lines respectively could not be obtained. From a total of 40 surviving colonies (21 from Panc10.05 and 19 from TS0111), >95% of mutations came from perfect target sites (84 out of 88 perfect target sites were mutated). Of 25 sites with 1 mismatch only 7 (28%) were targeted, and 0/27 for 2, 0/184 for 3, and 0/1688 for 4 mismatch sites were targeted (See Tables 10-13 shown below.

TABLE 10
Number of Cas9-induced cuts from WGS of surviving TS0111 and Panc10.05 colonies

| | Number of | | | | |
|---|---|---|---|---|---|
| | predicted | Number of | Number of | Number of | Total number of |
| | perfect target | potential off- | mutated sites | Panc10.05 mutated | Cas9-induced cuts |
| sgRNA | sites¹ | target sites² | in TS0111³ | sites in Panc10.05³ | in Panc10.05⁵ |
| NT | 0 | 0-1 | 0-0-0 | 0-0-0 | 0-0-0 |
| NT2 | 0 | 0-0 | 0-0-0 | 0-0-0 | 0-0-0 |
| HPRTc.80 | 1 | 0-2 | 1-0-0 | 1-0-0 | 1-0-0 |
| HPRTc.465 | 1 | 0-2 | 1-0-0 | 1-0-0 | 1-0-0 |
| 531F(2) | 2 | 4-1 | 2-0-0 | 2-0-0 | 3-0-0 |
| 52F(3) | 3 | 0-0 | 3-0-0 | 3-0-0 | 4-0-0 |
| 715F(5) | 5 | 2-1 | 5-1-0⁴ | 5-1-0 | 9-2-0 |
| 451F(6) | 6 | 0-1 | 6-0-0 | 6-0-0 | 12-0-0 |
| 176R(7) | 7 | 2-1 | 6-1-0 | 6-0-0 | 10-0-0 |
| 551R(8) | 8 | 2-1 | NA | 7-0-0 | 12-0-0 |
| 230F(12) | 12 | 8-1 | NA | NA | NA |
| 164R(14) | 14 | 5-2 | NA | 13-3-0⁴ | 21-5-0 |
| 676F(16) | 16 | 2-6 | 16-1-0 | NA | NA |

¹Number of perfect matches in CRISPOR using the GRCh38 human reference genome, including both canonical (NGG) and non-canonical (NGA/NAG) PAMs.
²From CRISPOR, 1 and 2 mismatches (mms).
³Matched or mismatched sites that are used from analysis of two resistant colonies for each sgRNA, using a VAF cutoff of 10%. Numbers are shown as 0 mm-1 mm-2 mm.
⁴Only one colony could be obtained.
⁵The number of sites cut that incorporates copy number of the target for Panc10.05 cell line based on hg19.
NA: not available since no resistant colonies could be obtained.

TABLE 11
List of predicted on- and off-target sites (1 and 2 mismatches) generated by CRISPOR
based on hg19; mutation analysis is performed for Panc10.05 surviving colonies

| | | Up_{—} | Down_{—} | Site_{—} | No_{—} | Pos_{—} | Copy_{—} | Mut_{—} | Mut_{—} | | |
|---|---|---|---|---|---|---|---|---|---|---|---|
| sgRNA | Chr | coord | coord | type | mm * | mm^{#} | no^{$} | freq ^{&} | type** | PAM | Note |
| NT_1 | chr2 | 157494340 | 157494362 | intergenic | 2 | 17, 18 | 2 | 0.00 | NA | AAG | |
| NT_2 | chr2 | 157494340 | 157494362 | intergenic | 2 | 17, 18 | 2 | 0.00 | NA | AAG | |
| HPRTc.80_1 | chrX | 133607441 | 133607463 | exon | 0 | NA | 1 | 1.00 | del | AGG | |
| | chr4 | 113190663 | 113190685 | exon | 2 | 2, 17 | 3 | 0.00 | NA | TGA | |
| | chr9 | 98907092 | 98907114 | intergenic | 2 | 8, 11 | 2 | 0.00 | NA | TAG | |
| HPRTc.80_2 | chrX | 133607441 | 133607463 | exon | 0 | NA | 1 | 1.00 | del | AGG | |
| | chr4 | 113190663 | 113190685 | exon | 2 | 2, 17 | 3 | 0.00 | NA | TGA | |
| | chr9 | 98907092 | 98907114 | intergenic | 2 | 8, 11 | 2 | 0.00 | NA | TAG | |
| HPRTc.465_1 | chrX | 133627578 | 133627600 | exon | 0 | NA | 1 | 1.00 | SV | AGG | |
| | chr20 | 1481410 | 1481432 | intergenic | 2 | 14, 19 | 2 | 0.00 | NA | TGG | |
| | chr13 | 51975960 | 51975982 | intron | 2 | 5, 18 | 2 | 0.00 | NA | GGA | |
| HPRTc.465_2 | chrX | 133627578 | 133627600 | exon | 0 | NA | 1 | 0.69 | indel | AGG | |
| | chr20 | 1481410 | 1481432 | intergenic | 2 | 14, 19 | 2 | 0.00 | NA | TGG | |
| | chr13 | 51975960 | 51975982 | intron | 2 | 5, 18 | 2 | 0.00 | NA | GGA | |
| 531F(2)_1 | chr1 | 531155 | 531177 | intron | 0 | NA | 1 | 1.00 | indel | TGG | |
| | chr8 | 30445 | 30467 | intergenic | 0 | NA | 2 | 1.00 | del | TGG | |
| | chr1 | 452604 | 452626 | intergenic | 1 | 18 | 1 | 0.16 | indel | TGG | |
| | chr17 | 81167615 | 81167637 | intergenic | 1 | 18 | 2 | 0.08 | indel | TGG | |
| | chr5 | 180880662 | 180880684 | intergenic | 1 | 18 | 2 | 0.02 | del | TGG | |
| | chr6 | 171035978 | 171036000 | intron | 1 | 18 | 1 | 0.10 | del | TGG | |
| | chr9 | 100967000 | 100967022 | intron | 2 | 3, 12 | 2 | 0.00 | NA | AGG | |
| 531F(2)_2 | chr1 | 531155 | 531177 | intron | 0 | NA | 1 | 1.00 | indel | TGG | |
| | chr8 | 30445 | 30467 | intergenic | 0 | NA | 2 | 1.00 | indel | TGG | |
| | chr1 | 452604 | 452626 | intergenic | 1 | 18 | 1 | 0.03 | indel | TGG | |
| | chr17 | 81167615 | 81167637 | intergenic | 1 | 18 | 2 | 0.06 | indel | TGG | |
| | chr5 | 180880662 | 180880684 | intergenic | 1 | 18 | 2 | 0.00 | NA | TGG | |
| | chr6 | 171035978 | 171036000 | intron | 1 | 18 | 1 | 0.00 | NA | TGG | |
| | chr9 | 100967000 | 100967022 | intron | 2 | 3, 12 | 2 | 0.00 | NA | AGG | |
| 52F(3)_1 | chr1 | 52017 | 52039 | intergenic | 0 | NA | 1 | 0.33 | del | TGG | |
| | chr15 | 102479109 | 102479131 | intergenic | 0 | NA | 2 | 0.00 | NA | TGG | |
| | chr19 | 93623 | 93645 | intergenic | 0 | NA | 1 | 0.39 | indel | TGG | |
| 52F(3)_2 | chr1 | 52017 | 52039 | intergenic | 0 | NA | 1 | 1.00 | indel | TGG | |
| | chr15 | 102479109 | 102479131 | intergenic | 0 | NA | 2 | 0.83 | indel | TGG | |
| | chr19 | 93623 | 93645 | intergenic | 0 | NA | 1 | 0.86 | indel | TGG | |
| 715F(5)_1 | chr1 | 715022 | 715044 | intron | 0 | NA | 1 | 1.00 | del | GGG | |
| | chr1 | 224181302 | 224181324 | intergenic | 0 | NA | 2 | 1.00 | del | GGG | |
| | chr10 | 38690926 | 38690948 | intron | 0 | NA | 2 | 0.39 | del | AGG | |
| | chr4 | 120376841 | 120376863 | intergenic | 0 | NA | 2 | 1.00 | del | GGG | |
| | chr7 | 56183073 | 56183095 | intron | 0 | NA | 2 | 1.00 | del | GGG | |
| | chr7 | 45807684 | 45807706 | intron | 1 | 15 | 2 | 1.00 | del | GGG | |
| | chr7 | 65959577 | 65959599 | intron | 1 | 7 | 1 | 0.03 | NA | GGG | |
| | chr14 | 45102271 | 45102293 | intergenic | 2 | 6, 10 | 3 | 0.00 | NA | AGG | |
| 715F(5)_2 | chr1 | 715022 | 715044 | intron | 0 | NA | 1 | 1.00 | del | GGG | |
| | chr1 | 224181302 | 224181324 | intergenic | 0 | NA | 2 | 1.00 | del + SV | GGG | |
| | chr10 | 38690926 | 38690948 | intron | 0 | NA | 2 | 1.00 | del | AGG | |
| | chr4 | 120376841 | 120376863 | intergenic | 0 | NA | 2 | 1.00 | SV | GGG | |
| | chr7 | 56183073 | 56183095 | intron | 0 | NA | 2 | 1.00 | indel + SV | GGG | |
| | chr7 | 45807684 | 45807706 | intron | 1 | 15 | 2 | 1.00 | del + SV | GGG | |
| | chr7 | 65959577 | 65959599 | intron | 1 | 7 | 1 | 0.00 | NA | GGG | |
| | chr14 | 45102271 | 45102293 | intergenic | 2 | 6, 10 | 3 | 0.00 | NA | AGG | |
| 451F(6)_1 | chr1 | 532400 | 532422 | intron | 0 | NA | 2 | 1.00 | del | AGG | sgRNA labeled as |
| | | | | | | | | | | | inefficient by CRISPOR |
| | chr1 | 451348 | 451370 | intergenic | 0 | NA | 2 | 0.81 | indel | GGG | SNV in PAM |
| | chr17 | 81166382 | 81166404 | intergenic | 0 | NA | 2 | 0.76 | indel | GGG | SNV in PAM |
| | chr5 | 180879406 | 180879428 | intergenic | 0 | NA | 2 | 0.63 | indel | GGG | SNV in PAM |
| | chr6 | 171034742 | 171034764 | intron | 0 | NA | 2 | 0.94 | indel | GGG | SNV in PAM; SNV on |
| | | | | | | | | | | | 4th base |
| | chr8 | 31585 | 31607 | intergenic | 0 | NA | 2 | 1.00 | indel | GGG | |
| | chr6 | 129467692 | 129467714 | intron | 2 | 18, 19 | 2 | 0.00 | NA | TGA | |
| 451F(6)_2 | chr1 | 532400 | 532422 | intron | 0 | NA | 2 | 1.00 | indel | AGG | sgRNA labeled as |
| | | | | | | | | | | | inefficient by CRISPOR |
| | chr1 | 451348 | 451370 | intergenic | 0 | NA | 2 | 0.67 | indel | GGG | SNV in PAM |
| | chr17 | 81166382 | 81166404 | intergenic | 0 | NA | 2 | 0.68 | indel | GGG | SNV in PAM |
| | chr5 | 180879406 | 180879428 | intergenic | 0 | NA | 2 | 0.56 | indel | GGG | SNV in PAM |
| | chr6 | 171034742 | 171034764 | intron | 0 | NA | 2 | 0.54 | del | GGG | SNV in PAM; SNV on |
| | | | | | | | | | | | 4th base |
| | chr8 | 31585 | 31607 | intergenic | 0 | NA | 2 | 0.86 | indel | GGG | |
| | chr6 | 129467692 | 129467714 | intron | 2 | 18, 19 | 2 | 0.00 | NA | TGA | |
| 176R(7)_1 | chr1 | 176766 | 176788 | intergenic | 0 | NA | 1 | 0.37 | indel | TGG | SNV on 9th base |
| | chr11 | 171957 | 171979 | intergenic | 0 | NA | 1 | 0.53 | indel | TGG | |
| | chr16 | 90192115 | 90192137 | intergenic | 0 | NA | 2 | 0.22 | indel | TGG | SNV on 9th base |
| | chr19 | 242211 | 242233 | intergenic | 0 | NA | 2 | 0.48 | indel | TGG | SNV on 18th base |
| | chr3 | 197904699 | 197904721 | intron | 0 | NA | 2 | 0.26 | indel | TGG | |
| | chr9 | 141131157 | 141131179 | intron | 0 | NA | 2 | 0.11 | indel | TGG | SNV on 9th base |
| | chr7 | 13063 | 13085 | intergenic | 1 | 18 | 2 | 0.00 | NA | TGG | Mutated sequence found |
| | | | | | | | | | | | in control |
| | chr8 | 151923 | 151945 | intergenic | 2 | 12, 18 | 1 | 0.00 | NA | TGG | SNV on 12th base (G−>C) |
| | | | | | | | | | | | turns sequence into 1 mm |
| 176R(7)_2 | chr1 | 176766 | 176788 | intergenic | 0 | NA | 1 | 0.40 | indel | TGG | SNV on 9th base |
| | chr11 | 171957 | 171979 | intergenic | 0 | NA | 1 | 0.44 | indel | TGG | |
| | chr16 | 90192115 | 90192137 | intergenic | 0 | NA | 2 | 0.25 | indel | TGG | SNV on 9th base |
| | chr19 | 242211 | 242233 | intergenic | 0 | NA | 2 | 0.72 | indel | TGG | SNV on 18th base |
| | chr3 | 197904699 | 197904721 | intron | 0 | NA | 2 | 0.76 | indel | TGG | |
| | chr9 | 141131157 | 141131179 | intron | 0 | NA | 2 | 0.39 | indel | TGG | SNV on 9th base |
| | chr7 | 13063 | 13085 | intergenic | 1 | 18 | 2 | 0.00 | NA | TGG | Mutated sequence found |
| | | | | | | | | | | | in control |
| | chr8 | 151923 | 151945 | intergenic | 2 | 12, 18 | 1 | 0.09 | NA | TGG | SNV on 12th base (G−>C) |
| | | | | | | | | | | | turns sequence into 1 mm |
| 551R(8)_1 | chr1 | 243156062 | 243156084 | intergenic | 0 | NA | 2 | 0.79 | indel + SV | AGG | SNV on 15th base |
| | chr1 | 433575 | 433597 | intergenic | 0 | NA | 1 | 0.75 | del | AGG | SNV on 15th base |
| | chr1 | 551010 | 551032 | intergenic | 0 | NA | 1 | 0.87 | del + SV | AGG | SNV on 15th base |
| | chr4 | 119363705 | 119363727 | intergenic | 0 | NA | 1 | 0.00 | NA | AGA | |
| | chr5 | 180860129 | 180860151 | intergenic | 0 | NA | 2 | 0.68 | del | AGG | SNV on 15th base |
| | chr6 | 171017812 | 171017834 | intergenic | 0 | NA | 2 | 0.82 | del | AGG | SNV on 15th base |
| | chr1 | 224077593 | 224077615 | intergenic | 0 | NA | 2 | 0.92 | del + SV | AGG | SNV on 15th base |
| | chr8 | 49474 | 49496 | intergenic | 0 | NA | 2 | 0.78 | del | AGG | SNV on 15th base |
| | chrY | 27471903 | 27471925 | intergenic | 1 | 2 | 0 | NA | NA | AGG | chrY doesn't exist |
| | chrY | 26490519 | 26490541 | intergenic | 1 | 2 | 0 | NA | NA | AGG | chrY doesn't exist |
| | chr1 | 32931999 | 32932021 | intergenic | 2 | 7 | 1 | 0.00 | NA | GGG | |
| 551R(8)_2 | chr1 | 243156062 | 243156084 | intergenic | 0 | NA | 2 | 0.94 | indel | AGG | SNV on 15th base |
| | chr1 | 433575 | 433597 | intergenic | 0 | NA | 1 | 0.94 | indel | AGG | SNV on 15th base |
| | chr1 | 551010 | 551032 | intergenic | 0 | NA | 1 | 0.90 | indel | AGG | SNV on 15th base |
| | chr4 | 119363705 | 119363727 | intergenic | 0 | NA | 1 | 0.00 | NA | AGA | |
| | chr5 | 180860129 | 180860151 | intergenic | 0 | NA | 2 | 1.00 | indel | AGG | SNV on 15th base |
| | chr6 | 171017812 | 171017834 | intergenic | 0 | NA | 2 | 0.67 | indel | AGG | SNV on 15th base |
| | chr1 | 224077593 | 224077615 | intergenic | 0 | NA | 2 | 0.93 | indel | AGG | SNV on 15th base |
| | chr8 | 49474 | 49496 | intergenic | 0 | NA | 2 | 0.87 | indel | AGG | SNV on 15th base |
| | chrY | 27471903 | 27471925 | intergenic | 1 | 2 | 0 | NA | NA | AGG | chrY doesn't exist |
| | chrY | 26490519 | 26490541 | intergenic | 1 | 2 | 0 | NA | NA | AGG | chrY doesn't exist |
| | chr1 | 32931999 | 32932021 | intergenic | 2 | 7 | 1 | 0.00 | NA | GGG | |
| 164R(14)_1 | chr1 | 224171172 | 224171194 | intron | 0 | NA | 1 | 1.00 | del + SV | TGG | sgRNA labeled as |
| | | | | | | | | | | | inefficient by CRISPOR |
| | chr1 | 164976 | 164998 | intron | 0 | NA | 1 | 1.00 | indel | CGG | SNV on 5th base |
| | chr11 | 160165 | 160187 | intergenic | 0 | NA | 1 | 1.00 | indel | CGG | |
| | chr1 | 222684185 | 222684207 | intergenic | 0 | NA | 2 | 1.00 | del + SV | TGG | |
| | chr3 | 197916501 | 197916523 | intron | 0 | NA | 2 | 1.00 | indel | CGG | |
| | chr19 | 230349 | 230371 | intergenic | 0 | NA | 2 | 1.00 | indel + SV | CGG | |
| | chr9 | 141142932 | 141142954 | intron | 0 | NA | 2 | 1.00 | indel | CGG | |
| | chr16 | 90203887 | 90203909 | intron | 0 | NA | 2 | 1.00 | indel + SV | CGG | |
| | chr1 | 243251719 | 243251741 | intron | 0 | NA | 2 | 1.00 | del + SV | TGG | |
| | chr5 | 180721841 | 180721863 | intergenic | 0 | NA | 2 | 1.00 | indel | CGG | |
| | chr7 | 45822037 | 45822059 | intron | 0 | NA | 2 | 0.37 | del | TAG | |
| | chr7 | 56477072 | 56477094 | intergenic | 0 | NA | 1 | 0.00 | NA | TGA | |
| | chr7 | 66320452 | 66320474 | intron | 1 | 8 | 1 | 0.00 | NA | TGA | |
| | chr1 | 700812 | 700834 | intergenic | 1 | 13 | 1 | 1.00 | indel + SV | CGG | |
| | chr10 | 38705403 | 38705425 | intergenic | 1 | 19 | 2 | 1.00 | del | TGG | |
| | chr4 | 120362394 | 120362416 | intron | 1 | 6 | 2 | 1.00 | indel | TGG | |
| | chr7 | 65948048 | 65948070 | intergenic | 1 | 8 | 1 | 0.00 | NA | TGA | |
| | chr2 | 113024328 | 113024350 | intergenic | 2 | 1, 10 | 2 | 0.00 | NA | CAG | |
| | chr14 | 84127138 | 84127160 | intergenic | 2 | 7, 14 | 2 | 0.00 | NA | TGG | |

* No_mm: Number of mismatches.
^{#}Pos_mm: Position of mismatch from PAM.
^{$}Copy_no: Copy number of target site.
^{&} Mut_freq: Mutation frequency is generated by CRISPResso WGS.
**Mut_type: “del” indicates deletions; “indel” indicates small insertions and deletions; “SV” indicates structural variants; “NA” indicates that a mutation is not found or the target site doesn't exist in controls.

TABLE 12
List of predicted on- and off-target sites (1 and 2 mismatches) generated by CRISPOR
based on hg38; mutation analysis is performed for Panc10.05 surviving colonies

| | | Up_{—} | Down_{—} | Site_{—} | No_{—} | Pos_{—} | Mut_{—} | Mut_{—} | | |
|---|---|---|---|---|---|---|---|---|---|---|
| sgRNA* | Chr | coord | coord | type | mm^{#} | mm^{$} | freq ^{&} | type** | PAM | Note |
| 176R(7)_1 | chr19 | 242211 | 242233 | intergenic | 0 | NA | 0.46 | indel | TGG | SNV on 18th base |
| | chr1 | 176766 | 176788 | intergenic | 0 | NA | 0.20 | indel | TGG | SNV on 9th base |
| | chr16 | 90125707 | 90125729 | intron | 0 | NA | 0.30 | indel | TGG | SNV on 9th base |
| | chr9 | 138240707 | 138240729 | intron | 0 | NA | 0.29 | indel | TGG | SNV on 9th base |
| | chr3 | 198177828 | 198177850 | intergenic | 0 | NA | 0.43 | indel | TGG | |
| | chr11 | 171957 | 171979 | intergenic | 0 | NA | 0.39 | indel | TGG | SNV on 18th base |
| | chr17 | 109767 | 109789 | intron | 0 | NA | NA | NA | TGG | No reads mapped to this region |
| | chr7 | 13063 | 13085 | intergenic | 1 | 18 | NA | NA | TGG | Mutated sequence found in control |
| | chr1 | 535353 | 535375 | intergenic | 1 | 9 | 0.00 | NA | TGG | |
| | chr8 | 201923 | 201945 | intergenic | 2 | 12, 18 | 0.00 | NA | TGG | |
| 176R(7)_2 | chr19 | 242211 | 242233 | intergenic | 0 | NA | 0.82 | indel | TGG | SNV on 18th base |
| | chr1 | 176766 | 176788 | intergenic | 0 | NA | 0.36 | indel | TGG | SNV on 9th base |
| | chr16 | 90125707 | 90125729 | intron | 0 | NA | 0.30 | indel | TGG | SNV on 9th base |
| | chr9 | 138240707 | 138240729 | intron | 0 | NA | 0.44 | indel | TGG | SNV on 9th base |
| | chr3 | 198177828 | 198177850 | intergenic | 0 | NA | 0.60 | indel | TGG | |
| | chr11 | 171957 | 171979 | intergenic | 0 | NA | 0.55 | indel | TGG | SNV on 18th base |
| | chr17 | 109767 | 109789 | intron | 0 | NA | NA | NA | TGG | No reads mapped to this region |
| | chr7 | 13063 | 13085 | intergenic | 1 | 18 | NA | NA | TGG | Mutated sequence found in control |
| | chr1 | 535353 | 535375 | intergenic | 1 | 9 | 0.00 | NA | TGG | |
| | chr8 | 201923 | 201945 | intergenic | 2 | 12, 18 | 0.00 | NA | TGG | |
| 164R(14)_1 | chr7 | 45782438 | 45782460 | intergenic | 0 | NA | 0.33 | indel | TAG | |
| | chr9 | 138252482 | 138252504 | intron | 0 | NA | 1.00 | indel | CGG | |
| | chr3 | 198189630 | 198189652 | intergenic | 0 | NA | 1.00 | indel | CGG | |
| | chr17 | 97982 | 98004 | intron | 0 | NA | 1.00 | indel | CGG | |
| | chr5 | 181294840 | 181294862 | intergenic | 0 | NA | 1.00 | indel | CGG | |
| | chr1 | 243088417 | 243088439 | intergenic | 0 | NA | 1.00 | del + SV | TGG | |
| | chr1 | 223983470 | 223983492 | intergenic | 0 | NA | 1.00 | del + SV | TGG | |
| | chr11 | 160165 | 160187 | intergenic | 0 | NA | 1.00 | indel | CGG | |
| | chr1 | 222510843 | 222510865 | intergenic | 0 | NA | 1.00 | del | TGG | |
| | chr1 | 523572 | 523594 | intergenic | 0 | NA | 1.00 | indel | CGG | |
| | chr16 | 90137479 | 90137501 | intergenic | 0 | NA | 1.00 | indel + SV | CGG | |
| | chr1 | 164,976 | 164,998 | intergenic | 0 | NA | 1.00 | indel | CGG | |
| | chr7 | 56409379 | 56409401 | intergenic | 0 | NA | 0.00 | NA | TGA | |
| | chr19 | 230349 | 230371 | intergenic | 0 | NA | 1.00 | indel + SV | CGG | |
| | chr1 | 765432 | 765454 | intergenic | 1 | 14 | 1.00 | del | CGG | |
| | chr10 | 38416475 | 38416497 | intergenic | 1 | 19 | 1.00 | del | TGG | |
| | chr4 | 119441239 | 119441261 | intergenic | 1 | 6 | 1.00 | indel | TGG | |
| | chr7 | 66855465 | 66855487 | intergenic | 1 | 8 | 0.00 | NA | TGA | |
| | chr7 | 66483061 | 66483083 | intergenic | 1 | 8 | 0.00 | NA | TGA | |
| | chr14 | 83660794 | 83660816 | intergenic | 2 | 7 | 0.00 | NA | TGG | |
| | chr2 | 112266751 | 112266773 | intergenic | 2 | 1, 10 | 0.00 | NA | CAG | |

*Only 176R(7) and 164R(14) are included as the number of predicted target sites for these two sgRNAs differ between hg19 and hg38. Refer to table S2 for the rest of the sgRNAs.
^{#}No_mm: Number of mismatches.
^{$}Pos_mm: Position of mismatch from PAM.
^{&} Mut_freq: Mutation frequency is generated by CRISPRessoWGS.
**Mut_type: “del” indicates deletions; “indel” indicates small insertions and deletions; “SV” indicates structural variants; “NA” indicates that a mutation is not found or the target site doesn't exist in controls.

TABLE 13
List of predicted on- and off-target sites (1 and 2 mismatches) generated by CRISPOR
based on hg38; mutation analysis is performed for TS0111 surviving colonies

| | | Up_{—} | Down_{—} | Site_{—} | No_{—} | Pos_{—} | Mut_{—} | Mut_{—} | | |
|---|---|---|---|---|---|---|---|---|---|---|
| sgRNA | Chr | coord | coord | type | mm* | mm^{#} | freq ^{$} | type^{&} | PAM | Note |
| NT_1 | chr2 | 156637828 | 156637850 | intergenic | 2 | 17, 18 | 0.00 | NA | AAG | |
| NT_2 | chr2 | 156637828 | 156637850 | intergenic | 2 | 17, 18 | 0.00 | NA | AAG | |
| HPRTc.80_1 | chrX | 134473411 | 134473433 | exon | 0 | NA | 1.00 | indel | AGG | |
| | chr4 | 112269507 | 112269529 | exon | 2 | 2, 17 | 0.00 | NA | TGA | |
| | chr9 | 96144810 | 96144832 | intergenic | 2 | 8, 11 | 0.00 | NA | TAG | |
| HPRTc.80_2 | chrX | 134473411 | 134473433 | exon | 0 | NA | 1.00 | del | AGG | |
| | chr4 | 112269507 | 112269529 | exon | 2 | 2, 17 | 0.00 | NA | TGA | |
| | chr9 | 96144810 | 96144832 | intergenic | 2 | 8, 11 | 0.00 | NA | TAG | |
| HPRTc.465_1 | chrX | 134493548 | 134493570 | exon | 0 | NA | 1.00 | indel | AGG | |
| | chr20 | 1500764 | 1500786 | intergenic | 2 | 14, 19 | 0.00 | NA | TGG | |
| | chr13 | 51401824 | 51401846 | intron | 2 | 5, 18 | 0.00 | NA | GGA | |
| HPRTc.465_2 | chrX | 134493548 | 134493570 | exon | 0 | NA | 0.95 | indel | AGG | |
| | chr20 | 1500764 | 1500786 | intergenic | 2 | 14, 19 | 0.00 | NA | TGG | |
| | chr13 | 51401824 | 51401846 | intron | 2 | 5, 18 | 0.00 | NA | GGA | |
| 531F(2)_1 | chr1 | 595775 | 595797 | intron | 0 | NA | 0.43 | indel | TGG | |
| | chr8 | 80445 | 80467 | intergenic | 0 | NA | 0.38 | indel | TGG | |
| | chr1 | 366711 | 366733 | intergenic | 1 | 18 | 0.00 | NA | TGG | |
| | chr17 | 83219846 | 83219868 | intergenic | 1 | 18 | 0.00 | NA | TGG | |
| | chr5 | 181453661 | 181453683 | intergenic | 1 | 18 | 0.00 | NA | TGG | |
| | chr6 | 170726890 | 170726912 | intron | 1 | 18 | 0.00 | NA | TGG | |
| | chr9 | 98204718 | 98204740 | intron | 2 | 3, 12 | 0.00 | NA | AGG | |
| 531F(2)_2 | chr1 | 595775 | 595797 | intron | 0 | NA | 0.45 | indel | TGG | |
| | chr8 | 80445 | 80467 | intergenic | 0 | NA | 0.33 | del | TGG | |
| | chr1 | 366711 | 366733 | intergenic | 1 | 18 | 0.00 | NA | TGG | |
| | chr17 | 83219846 | 83219868 | intergenic | 1 | 18 | 0.00 | NA | TGG | |
| | chr5 | 181453661 | 181453683 | intergenic | 1 | 18 | 0.00 | NA | TGG | |
| | chr6 | 170726890 | 170726912 | intron | 1 | 18 | 0.00 | NA | TGG | |
| | chr9 | 98204718 | 98204740 | intron | 2 | 3, 12 | 0.00 | NA | AGG | |
| 52F(3)_1 | chr1 | 52017 | 52039 | intergenic | 0 | NA | 1.00 | SV | TGG | |
| | chr15 | 101938906 | 101938928 | intergenic | 0 | NA | 1.00 | indel | TGG | |
| | chr19 | 93623 | 93645 | intergenic | 0 | NA | 1.00 | indel + SV | TGG | |
| 52F(3)_2 | chr1 | 52017 | 52039 | intergenic | 0 | NA | 1.00 | SV | TGG | |
| | chr15 | 101938906 | 101938928 | intergenic | 0 | NA | 0.64 | indel | TGG | |
| | chr19 | 93623 | 93645 | intergenic | 0 | NA | 1.00 | indel | TGG | |
| 715F(5)_1 | chr1 | 779642 | 779664 | intergenic | 0 | NA | 1.00 | indel + SV | GGG | |
| | chr1 | 223993600 | 223993622 | intergenic | 0 | NA | 1.00 | del + SV | GGG | |
| | chr10 | 38401998 | 38402020 | intergenic | 0 | NA | 1.00 | indel + SV | AGG | |
| | chr4 | 119455686 | 119455708 | intergenic | 0 | NA | 1.00 | del + SV | GGG | |
| | chr7 | 56115380 | 56115402 | intron | 0 | NA | 1.00 | SV | GGG | |
| | chr7 | 45768085 | 45768107 | intergenic | 1 | 15 | 1.00 | del + SV | GGG | |
| | chr7 | 66494590 | 66494612 | intron | 1 | 7 | 0.00 | NA | GGG | |
| | chr14 | 44633068 | 44633090 | intergenic | 2 | 6, 10 | 0.00 | NA | AGG | |
| 451F(6)_1 | chr1 | 597020 | 597042 | intergenic | 0 | NA | 0.87 | indel | AGG | sgRNA labeled as inefficient by |
| | | | | | | | | | | CRISPOR |
| | chr1 | 367966 | 367988 | intergenic | 0 | NA | 0.87 | indel | GGG | |
| | chr17 | 83218613 | 83218635 | intergenic | 0 | NA | 0.82 | indel | GGG | |
| | chr5 | 181452405 | 181452427 | intergenic | 0 | NA | 0.76 | indel | GGG | |
| | chr6 | 170725654 | 170725676 | intron | 0 | NA | 0.80 | indel | GGG | |
| | chr8 | 81585 | 81607 | intergenic | 0 | NA | 0.80 | indel | GGG | |
| | chr6 | 129146547 | 129146569 | intron | 2 | 18, 19 | NA | NA | TGA | No reads mapped to this region |
| 451F(6)_2 | chr1 | 597020 | 597042 | intergenic | 0 | NA | 0.68 | indel + SV | AGG | sgRNA labeled as inefficient by |
| | | | | | | | | | | CRISPOR |
| | chr1 | 367966 | 367988 | intergenic | 0 | NA | 0.93 | indel | GGG | |
| | chr17 | 83218613 | 83218635 | intergenic | 0 | NA | 0.77 | indel | GGG | |
| | chr5 | 181452405 | 181452427 | intergenic | 0 | NA | 0.85 | indel | GGG | |
| | chr6 | 170725654 | 170725676 | intron | 0 | NA | 0.60 | indel | GGG | |
| | chr8 | 81585 | 81607 | intergenic | 0 | NA | 0.60 | indel | GGG | |
| | chr6 | 129146547 | 129146569 | intron | 2 | 18, 19 | NA | NA | TGA | No reads mapped to this region |
| 176R(7)_1 | chr19 | 242211 | 242233 | intergenic | 0 | NA | 0.38 | indel | TGG | |
| | chr1 | 176766 | 176788 | intergenic | 0 | NA | 0.26 | indel | TGG | |
| | chr16 | 90125707 | 90125729 | intron | 0 | NA | 0.26 | indel | TGG | SNV on 9th base |
| | chr9 | 138240707 | 138240729 | intron | 0 | NA | 0.26 | indel | TGG | SNV on 9th base |
| | chr3 | 198177828 | 198177850 | intergenic | 0 | NA | 0.51 | indel | TGG | |
| | chr11 | 171957 | 171979 | intergenic | 0 | NA | 0.31 | indel | TGG | |
| | chr17 | 109767 | 109789 | intron | 0 | NA | NA | NA | TGG | Mutated sequence found in |
| | | | | | | | | | | control |
| | chr7 | 13063 | 13085 | intergenic | 1 | 18 | 0.40 | indel | TGG | SNV on 18th base |
| | chr1 | 535353 | 535375 | intergenic | 1 | 9 | 0.00 | NA | TGG | |
| | chr8 | 201923 | 201945 | intergenic | 2 | 12, 18 | NA | NA | TGG | Mutated sequence found in |
| | | | | | | | | | | control |
| 176R(7)_2 | chr19 | 242211 | 242233 | intergenic | 0 | NA | 0.61 | indel | TGG | |
| | chr1 | 176766 | 176788 | intergenic | 0 | NA | 0.37 | indel | TGG | |
| | chr16 | 90125707 | 90125729 | intron | 0 | NA | 0.44 | indel | TGG | SNV on 9th base |
| | chr9 | 138240707 | 138240729 | intron | 0 | NA | 0.49 | indel | TGG | SNV on 9th base |
| | chr3 | 198177828 | 198177850 | intergenic | 0 | NA | 0.51 | indel | TGG | |
| | chr11 | 171957 | 171979 | intergenic | 0 | NA | 0.60 | indel | TGG | |
| | chr17 | 109767 | 109789 | intron | 0 | NA | NA | NA | TGG | Mutated sequence found in |
| | | | | | | | | | | control |
| | chr7 | 13063 | 13085 | intergenic | 1 | 18 | 1.00 | indel | TGG | SNV on 18th base; poorly |
| | | | | | | | | | | mapped region |
| | chr1 | 535353 | 535375 | intergenic | 1 | 9 | 0.00 | NA | TGG | |
| | chr8 | 201923 | 201945 | intergenic | 2 | 12, 18 | 0.00 | NA | TGG | Mutated sequence found in |
| | | | | | | | | | | control |
| 676F(16)_1 | chr4 | 118623185 | 118623207 | intron | 0 | NA | 0.28 | indel | GGG | |
| | chr5 | 181319056 | 181319078 | intron | 0 | NA | 0.46 | indel | GGG | |
| | chr1 | 222484377 | 222484399 | intron | 0 | NA | 0.88 | indel | GGG | |
| | chr1 | 223959499 | 223959521 | intergenic | 0 | NA | 0.59 | indel | GGG | |
| | chr7 | 39784471 | 39784493 | intron | 0 | NA | 0.41 | indel | GGG | |
| | chr1 | 499872 | 499894 | intron | 0 | NA | 0.51 | indel | GGG | |
| | chr1 | 741603 | 741625 | intergenic | 0 | NA | 0.33 | indel | GGG | |
| | chr1 | 141264 | 141286 | intergenic | 0 | NA | 0.51 | indel | GGG | SNV on 5th base |
| | chr7 | 128643944 | 128643966 | intergenic | 0 | NA | 0.74 | indel | GGG | |
| | chr4 | 119417377 | 119417399 | intergenic | 0 | NA | 0.35 | indel | GGG | |
| | chr11 | 136364 | 136386 | intron | 0 | NA | 0.26 | indel | GGG | |
| | chr3 | 198213471 | 198213493 | intergenic | 0 | NA | 0.44 | indel | GGG | SNV on 5th base |
| | chr1 | 243064500 | 243064522 | intergenic | 0 | NA | 0.38 | indel | GGG | |
| | chr10 | 38440574 | 38440596 | intergenic | 0 | NA | 0.22 | indel | GAG | SNV on 2nd base of PAM |
| | chr17 | 74131 | 74153 | intron | 0 | NA | 0.60 | ins | GGG | |
| | chr9 | 138276155 | 138276177 | intergenic | 0 | NA | 0.25 | ins | GGG | Mutated sequence found in half |
| | | | | | | | | | | of sequence in control |
| | chr19 | 206644 | 206666 | intergenic | 1 | 5 | 0.06 | del | GGG | |
| | chr16 | 90161158 | 90161180 | intron | 1 | 5 | 0.12 | del | GGG | SNV on 5th base |
| | chr7 | 55755509 | 55755531 | intergenic | 2 | 9, 16 | 0.00 | NA | GGG | |
| | chr11 | 50085686 | 50085708 | intergenic | 2 | 9, 16 | 0.00 | NA | GGG | |
| | chr7 | 45800255 | 45800277 | intergenic | 2 | 9, 16 | 0.00 | NA | GGG | |
| | chr7 | 56385734 | 56385756 | intergenic | 2 | 9, 16 | 0.00 | NA | GGG | |
| | chr7 | 63730827 | 63730849 | intergenic | 2 | 9, 16 | 0.00 | NA | GGG | |
| | chr7 | 56846141 | 56846163 | intron | 2 | 9, 16 | 0.00 | NA | GGG | |
| 676F(16)_2 | chr4 | 118623185 | 118623207 | intron | 0 | NA | 1.00 | SV | GGG | |
| | chr5 | 181319056 | 181319078 | intron | 0 | NA | 0.96 | indel | GGG | |
| | chr1 | 222484377 | 222484399 | intron | 0 | NA | 1.00 | del | GGG | |
| | chr1 | 223959499 | 223959521 | intergenic | 0 | NA | 1.00 | del | GGG | |
| | chr7 | 39784471 | 39784493 | intron | 0 | NA | 1.00 | del + SV | GGG | |
| | chr1 | 499872 | 499894 | intron | 0 | NA | 1.00 | indel | GGG | |
| | chr1 | 741603 | 741625 | intergenic | 0 | NA | 0.96 | del | GGG | |
| | chr1 | 141264 | 141286 | intergenic | 0 | NA | 0.96 | indel + SV | GGG | SNV on 5th base |
| | chr7 | 128643944 | 128643966 | intergenic | 0 | NA | 1.00 | del + SV | GGG | |
| | chr4 | 119417377 | 119417399 | intergenic | 0 | NA | 1.00 | del + SV | GGG | |
| | chr11 | 136364 | 136386 | intron | 0 | NA | 1.00 | indel | GGG | |
| | chr3 | 198213471 | 198213493 | intergenic | 0 | NA | 1.00 | indel | GGG | SNV on 5th base |
| | chr1 | 243064500 | 243064522 | intergenic | 0 | NA | 1.00 | del + SV | GGG | |
| | chr10 | 38440574 | 38440596 | intergenic | 0 | NA | 1.00 | del | GAG | SNV on 2nd base of PAM |
| | chr17 | 74131 | 74153 | intron | 0 | NA | 1.00 | indel + SV | GGG | |
| | chr9 | 138276155 | 138276177 | intergenic | 0 | NA | NA | NA | GGG | Mutated sequence found in half |
| | | | | | | | | | | of sequence in control |
| | chr19 | 206644 | 206666 | intergenic | 1 | 5 | 0.04 | del + SV | GGG | |
| | chr16 | 90161158 | 90161180 | intron | 1 | 5 | 0.11 | del | GGG | SNV on 5th base |
| | chr7 | 55755509 | 55755531 | intergenic | 2 | 9, 16 | 0.00 | NA | GGG | |
| | chr11 | 50085686 | 50085708 | intergenic | 2 | 9, 16 | 0.00 | NA | GGG | |
| | chr7 | 45800255 | 45800277 | intergenic | 2 | 9, 16 | 0.00 | NA | GGG | |
| | chr7 | 56385734 | 56385756 | intergenic | 2 | 9, 16 | 0.00 | NA | GGG | |
| | chr7 | 63730827 | 63730849 | intergenic | 2 | 9, 16 | 0.00 | NA | GGG | |
| | chr7 | 56846141 | 56846163 | intron | 2 | 9, 16 | 0.00 | NA | GGG | |

^{*}No_mm: Number of mismatches.
^{#}Pos_mm: Position of mismatch from PAM.
^{$} Mut_freq: Mutation frequency is generated by CRISPRessoWGS.
^{&}Mut_type: “del” indicates deletions; “indel” indicates small insertions and deletions; “ins” indicates insertions; “SV” indicates structural variants; “NA” indicates that a mutation is not found or the target site doesn't exist in controls.

Considering the copy number of each mutated site, it was found that the total number of mutated sites in each resistant colony highly correlated with the predicted number of target sites (FIG. 6C). Since only 28% of 1 mismatch sites and none with 2 or more mismatches were targeted, the number of perfectly matched target sites predicted is a good approximation of the number of functional target sites.

To assess the impact of DSBs on toxicity, the mutation frequency at each target site was quantified, including both on- and off-targets, and the possible factors were examined that could have influenced the mutation frequency at each site. It was found that the total mutation frequency (combined variant allele frequency, VAF) of each colony correlated better with cell elimination compared to predicted number of target sites (FIG. 6D, Tables 11-13). In general, most mutations came from perfect target sites, and most sgRNAs produced >80% mutation frequency at all perfect target sites (FIG. 6E, Tables 11-13). For the colonies with lower mutation frequencies, most could be explained by cell line specificity, such as single nucleotide polymorphisms (SNPs) within the target sites (FIG. 6F). The data suggests that the number of DSBs produced directly correlated with cell growth inhibition.

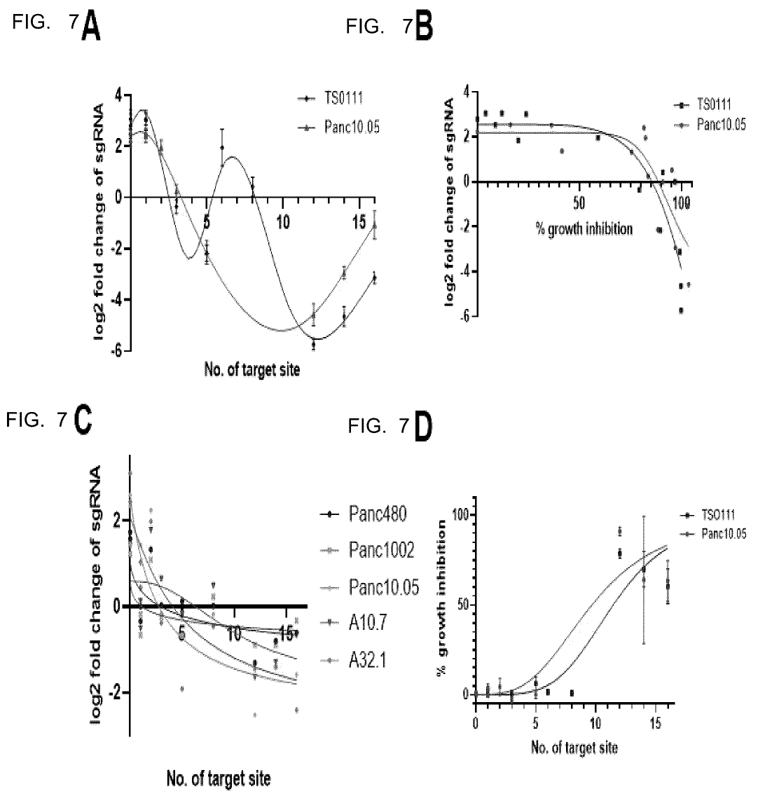

As an independent measure of cell death, sgRNA tag survival was assessed in the same two cell lines as a function of time, on the assumption that sgRNAs that were lethal to cells would be eliminated from the pool of tags, while sgRNAs with little or no toxicity should be well-represented in the pool at later time points (12, 13). All the multi-target sgRNAs were transduced together at low multiplicity of infection (MOI) and determined their baseline prevalence at day 1. The survival of the sgRNA tags in the pool were measured at 7, 14 and 21 days after transduction and compared the change of sgRNAs in the pool to the number of predicted target sites for the two cell lines (FIG. 7A). This confirmed a correlation between the number of predicted target sites in the human genome and degree of sgRNA tag loss in the surviving cell population. The sgRNA tag loss was compared to the results obtained from growth inhibition based on clonogenicity, where the correlation of the two was especially good when the growth inhibition exceeded 70% (FIG. 7B). This finding was also confirmed using sgRNA tag survival in 4 additional PC cell lines (FIG. 7C). Temporally, most of the reduction in sgRNA tag counts did not occur in the first 7 days, but rather occurred between days 7 and 21 (FIG. 1D). Clonogenicity assays performed with different dilutions also showed a similar temporal delay (FIG. 1A, FIG. 7D). Overall, cell elimination increased directly with the number of sites targeted in the human genome and was delayed compared to the time that the sgRNAs were introduced.

### Multiple DSBs Cause Genomic Instability and Delayed Cancer Cell Death

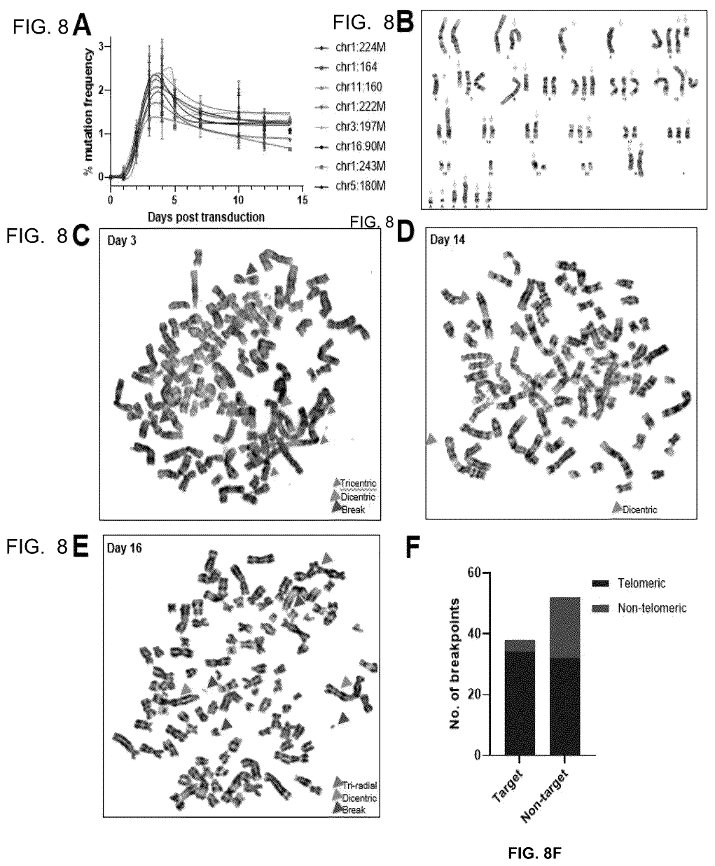

To assess the timing of DSB production, the 14-target sgRNA was transduced and quantified the mutation frequency at the target sites as a function of time. It was found that scission occurs over the course of days and peaked at days 3-5, consistent with other recent observations (FIG. 8A)(14). Because of the cell elimination, it was observed in the sgRNA tag survival experiments occurred over subsequent weeks, it was hypothesized that the mechanism of cell death was likely not due to DNA damage repair that was immediately and directly triggered by the multiple scission events, but rather was caused by a slower process such as genomic instability, which then ultimately led to cell death.

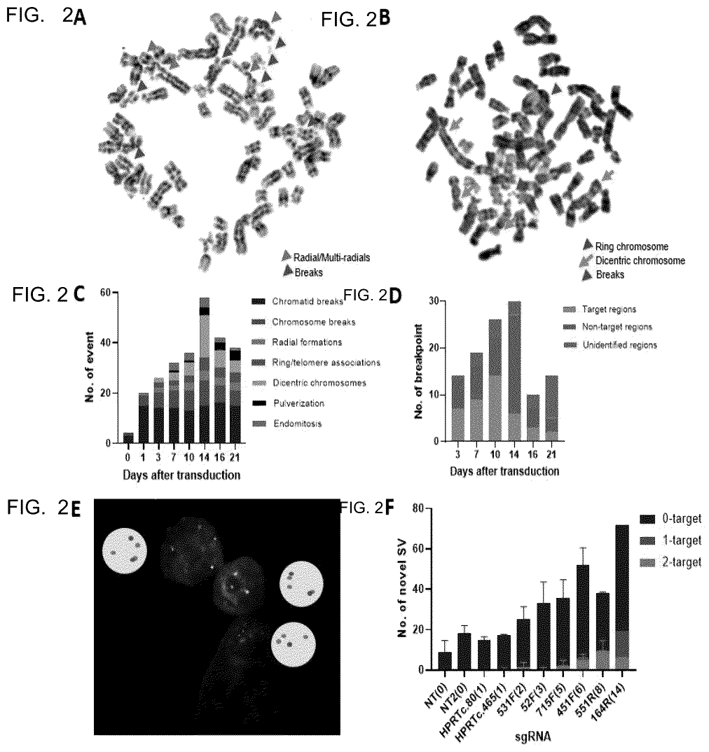

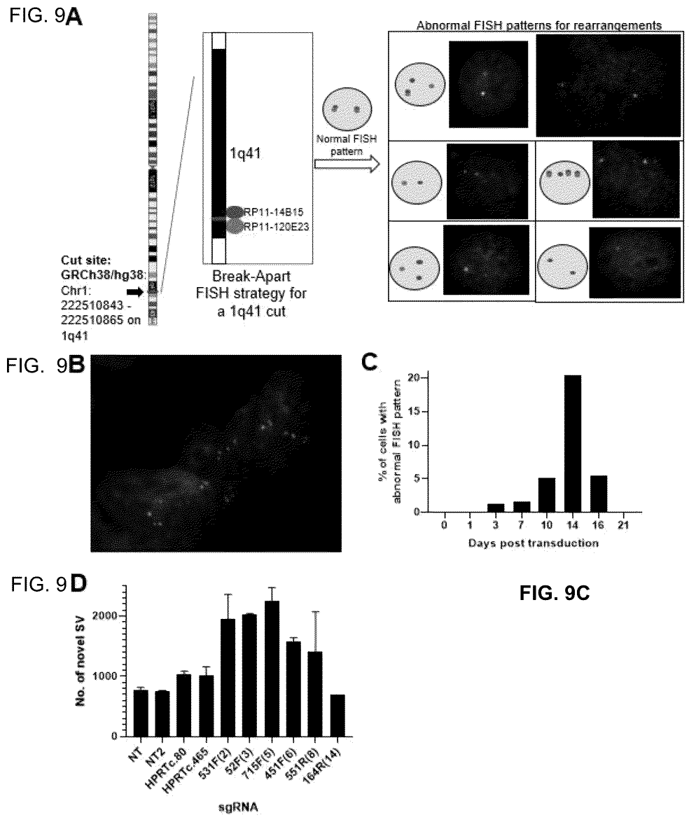

To test this hypothesis, the TS0111 Cas9-expressing cell line was selected, based on its simpler karyotype of the Cas9 cell lines at baseline (FIG. 8B), and it was treated with the 14-target sgRNA. Cytogenetic analysis was performed on cells harvested from 0-21 days at 3-4 day intervals using a chromosome breakage assay (FIG. 2A-2C, FIG. 8C-8E). At day 1, multiple chromosome and chromatid breaks were detected, along with radial formation that increased over time (FIG. 2A, 2C). Other karyotypic alterations also accumulated over time, including formation of ring, dicentric and tricentric chromosomes, telomere-telomere association, chromosome pulverization, and endomitosis (FIG. 2B-2C, FIG. 8C-8E). Most of these aberrations peaked at day 14, except for the chromatid and chromosome breaks where the frequency was maintained through day 21, suggesting ongoing occurrence of breakage events. The breakpoints on dicentric and tricentric chromosomes were also analyzed to examine whether they occurred at targeted or non-targeted regions based on chromosomal band locations of the sgRNA target sequences. Although targeted regions predominated at early time points and decreased as a function of time after transduction, non-targeted regions increased and peaked at day 14 (FIG. 2D). While most target regions were located at telomeric regions, 61.5% of novel structural variants (SVs) identified at non-targeted regions were also located at telomeric regions (FIG. 8F). To visually confirm that these SVs were a direct result of CRISPR-Cas9 cut, a break-apart fluorescence in situ hybridization (FISH) assay was performed on one of the target sites to observe for genomic rearrangements (FIG. 9A). The number of cells with abnormal FISH patterns increased over time and peaked at day 14 (FIG. 2E, FIG. 9B-9C), demonstrating that the formation of novel SVs indeed originated from CRISPR-Cas9 cutting at sgRNA target sites. These results indicate that targeting multiple regions at telomeric ends led to ongoing chromosomal rearrangements, which led to more SVs found near telomeric regions. In summary, treatment with the multi-target sgRNAs resulted in karyotypic abnormalities and SVs that mostly peaked at 14 days after introduction, rather than at the time of initial induction of the DSBs.

As a second method to study the effects of DSBs induced by multi-target sgRNAs, the WGS data of surviving colonies were analyzed to identify novel SVs. This approach was chosen because it would allow us to see the effects of repair at the sites directly targeted, but also look for evidence of off-target sites, which might include SVs that resulted from CRISPR-Cas9 targeting as well as SVs that arose at non-targeted sites. The SV detection software, Manta, was used to identify SVs in samples treated with multitarget sgRNAs, followed by visual inspection of all identified SVs using IGV for validation and quantification (15). The data showed that novel SVs increased as a function of the number of sgRNA target sites (FIG. 2F). and this finding has been corroborated by using a different SV caller, Trellis (FIG. 9D) (16). For the 14-cutter, only 7.7% of SVs were produced from two sites that were directly targeted, and 2.9% were produced where one site was targeted, while the majority (89.4%) were at non-targeted sites, consistent with ongoing genomic instability.

Further, comparisons between individual colonies transduced with the same sgRNA revealed that SVs in non-targeted regions were unique to each colony, supporting the concept that these are not a result of off-target effects. One instance of a shared novel SV was found, but the breakpoint differed from the guide sequence by 13 mismatches and was therefore likely present in the bulk cell line at a low level prior to selection by cloning. In summary, sequencing showed that the majority of SVs arose at non-targeted sites, and SVs in resistant colonies from the same sgRNA differed from each other, both supporting the concept of ongoing genomic instability.

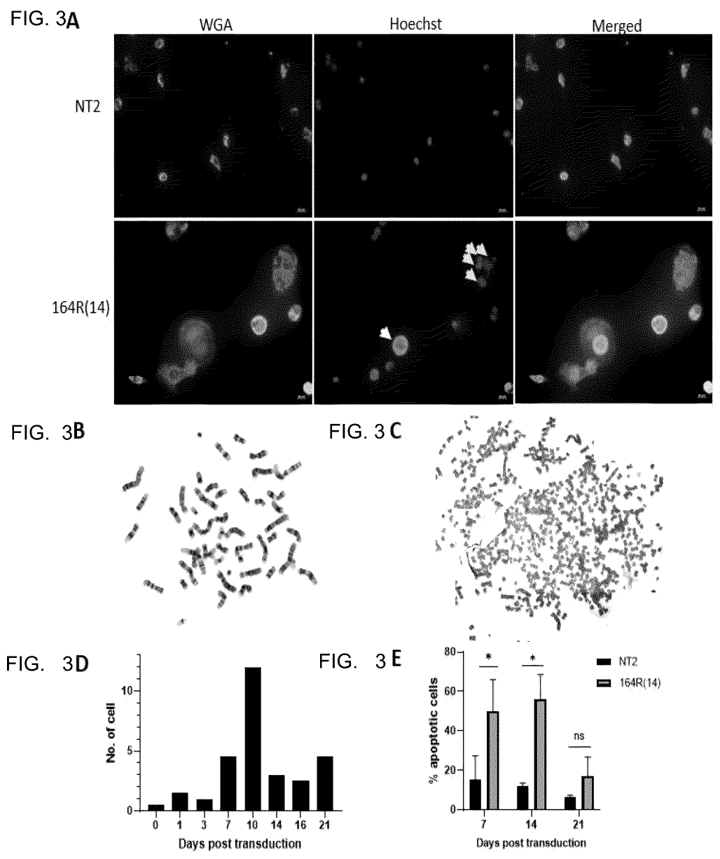

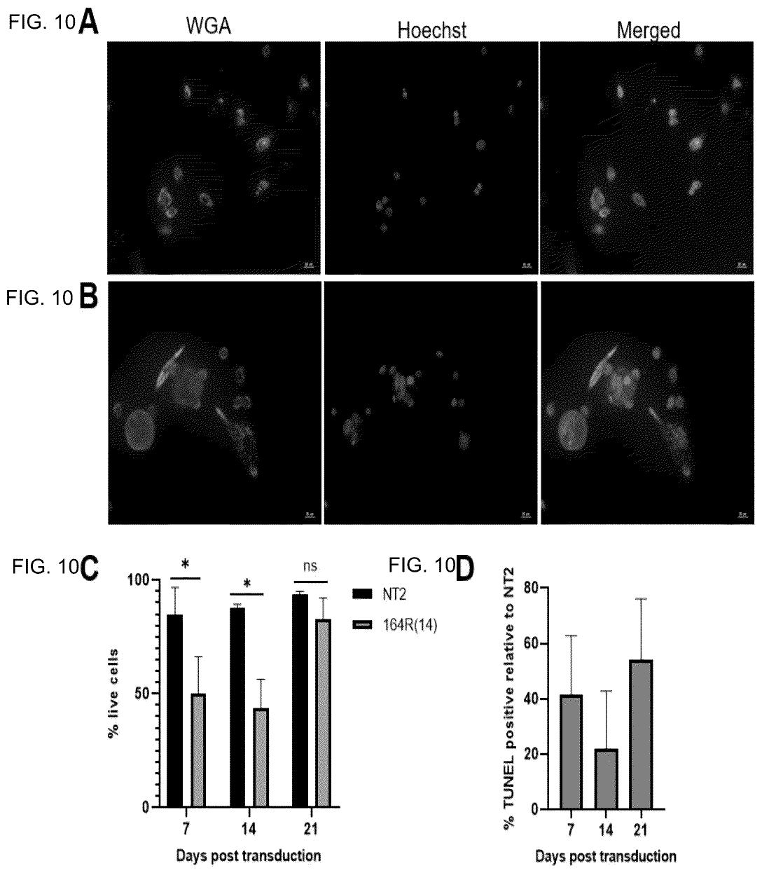

It was found that cells responded to the 14-cutter by becoming polyploid, manifesting as extremely large nuclei or multinucleated giant cells (FIG. 3A, FIG. 10A-10B). Metaphase images of transduced cells also showed that chromosome number increased after transduction and that the cells were clearly polyploid by day 10 (FIG. 3B-3C), with cells commonly containing >100 chromosomes. As this cell line is female, we confirmed polyploidization using XY FISH, counting cells with >6 copies of X chromosomes (FIG. 3D). Polyploidy peaked at day 10 and decreased by day 21. Additionally, apoptosis was assayed for and which was found to increase on days 7 and 14 compared to pre-transduction, and decreased by day 21 (FIG. 3D, FIG. 10C-10D). These data suggest that toxicity occurred following the induction of multiple DSBs that resulted in ongoing chromosomal rearrangements and polyploidization, ultimately leading to cell death via apoptosis and possibly other mechanisms.

Somatic single base substitutions in cancers create hundreds of novel PAMs

Having established the number of DSBs that resulted in cytotoxicity, this was compared to the number of sites in individual cancer cell lines that could be targeted. Somatic mutations in 3 PC cell lines for CRISPR targets were analyzed by searching for 5′-NGG-3′ PAMs that are recognized by the most commonly used Cas9, S. pyogenes Cas9. Three different approaches were used to identify PAMs. The first approach identified somatic mutations creating new CRISPR-Cas9 targets in exons, the second in SVs, and finally those in non-coding DNA.

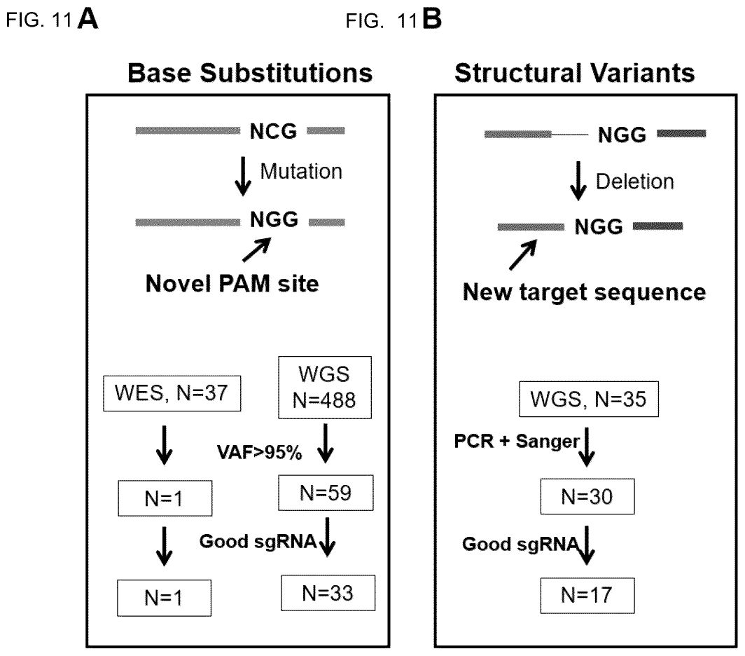

Exons for somatic mutations that created novel PAMs were first looked at under the hypothesis that disrupting these genes might be particularly toxic, especially if the gene were essential (Table 14 below, FIG. 11A).

TABLE 14
Novel PAMs discovered using WES, SV, and WGS

| | | | | | | | No. of | |
|---|---|---|---|---|---|---|---|---|
| | | Total no. of | | No. of PAM | | | good sgRNAs | |
| | | somatic | No. of | confirmed | | No. of good | with PAM | |
| Method | Cell line | mutations | novel PAM | in IGV* | | sgRNAs^{#} | of VAF >95% | |
| WES | Panc480 | 44 | 8 | 7 | (15.9%) | 5 | 2 | (28.6%) |
| | Panc504 | 38 | 3 | 1 | (2.6%) | 0 | 0 | (0%) |
| | Panc1002 | 30 | 4 | 4 | (13.3%) | 2 | 0 | (0%) |

| | | No. of | | | | | |
|---|---|---|---|---|---|---|---|
| | | somatic SVs | No. of | Total no. of | No. of | | |
| | | discovered | somatic SVs | somatic SVs | Sanger- | No. of | No. of |
| | | via SNP | discovered | (confirmed | validated | SVs with | good |
| Method | Cell line | microarray** | via WGS | on IGV) | SVs | PAM | sgRNAs^{#} |
| SV | Panc480 | 7 | 37 | 38 | 31 (81.6%) | 24 | 17 (54.8%) |
| | Panc504 | 8 | 33 | 37 | 29 (78.4%) | 18 | 15 (51.7%) |
| | Panc1002 | 11 | 28 | 31 | 30 (96.8%) | 25 | 18 (60.0%) |

| | | Total no. | No. of | No. of PAM | No. of | | No. of | No. of Sanger- | |
|---|---|---|---|---|---|---|---|---|---|
| | | of somatic | initial | confirmed | PAM with | | good | validated | |
| Method | Cell line | mutations | novel PAM^{&} | in IGV | VAF >95% | | sgRNAs^{#} | good sgRNAs | |
| WGS | Panc480 | 44311 | 6907 | 494 | 23 | (4.7%) | 13 (56.5%) | 13 | (100%) |
| | Panc504 | 38881 | 6056 | 531 | 76 | (14.3%) | 48 (63.2%) | 47 | (97.9%) |
| | Panc1002 | 48866 | 7901 | 440 | 78 | (17.7%) | 38 (48.7%) | 37 | (97.4%) |

*Each novel PAM was visually inspected and confirmed on IGV. The percentage indicates the proportion of somatic mutations that resulted in novel PAMs that were confirmed on IGV.
^{#}“Good sgRNA” is defined as sgRNAs that have >50 specificity score (prediction of how much the sgRNA sequence may lead to off-target cleavage) in CRISPOR. It includes sgRNAs that are inefficient (low knockout frequencies).
**SVs identified were previously published in Norris et al. (2015) Genes, Chromosomes & Cancer.
^{&}Novel PAM indicates a single base substitution of NGN/NNG sequence to NGG. Only sites with a variant allele frequency (VAF) of at least 5% in tumor and a minimum of 18X read depth in both germline and tumor are counted.

Whole exome sequencing (WES) was performed on both tumor and normal samples for a given cell line. Among an average of 37.3 somatic single base substitutions (SBSs) per cell line, only 4 on average were predicted to create a novel PAM (NGG), and of these only a total of 2 were present at a VAF >95% and produced a good sgRNA based on the specificity score provided by CRISPOR (Table 14) (10). It was concluded that WES provided too few targets compared to the number required to generate toxicity.

SVs were then considered, since they could juxtapose a new target DNA sequence next to an existing NGG PAM (Table 14, FIG. 11B). Somatic SVs were uncovered by using the SV detection software Trellis to analyze WGS data from the three cell lines in comparison to the patient's germline DNA (16). Initially, an average of 35.3 SVs per cell line were detected, and all were confirmed by PCR amplification across the breakpoint and Sanger sequencing (Table 14). A control sample did not amplify using the same set of primers. These SVs contained an average of 23.3 novel targets juxtaposed next to PAMs, which resulted in an average of 16.7 good sgRNAs.

In contrast, using WGS and liberal selection criteria, an average of 44,019 SBSs per cell line in IGV were studied by comparing tumor to normal, and identified an average of 488.3 mutations creating novel PAMs per cell line (Table 14, FIG. 11A). Of these, an average of 59 were present at a VAF>95% and an average of 33 created good sgRNAs. Of the 33 qualifying mutations per line, it was confirmed that all, except 2, of them by Sanger sequencing (Table 14).

From these data, shown below in Table 15, it was concluded that analysis of WGS data for non-coding SBSs was the most productive of the 3 methods and provided hundreds of novel PAMs.

TABLE 15

| | | No. of | No. of | No. of | No. of Sanger- |
|---|---|---|---|---|---|
| | | somatic | novel | good | validated good |
| Method | Cell line | mutations | PAMs* | sgRNAs^{#} | sgRNAs** |
| WES | Panc480 | 44 | 7 | 2 | NA |
| | Panc504 | 38 | 1 | 0 | NA |
| | Panc1002 | 30 | 4 | 0 | NA |
| SV | Panc480 | 38 | 24 | 17 | 17 |
| | Panc504 | 37 | 18 | 15 | 15 |
| | Panc1002 | 31 | 25 | 18 | 18 |
| WGS | Panc480 | 44311 | 494 | 13 | 13 |
| | Panc504 | 38881 | 531 | 48 | 47 |
| | Panc1002 | 48866 | 440 | 38 | 37 |

*For SV approach, the values indicate number of novel junctions flanked by an NGG sequence in which breakpoint sequence has been validated through Sanger sequencing. For WES and WGS approaches, novel PAM indicates a single base substitution of NGN/NNG sequence to NGG. Only sites with a variant allele frequency (VAF) of at least 5% in tumor and a minimum of 18X read depth in both germline and tumor are counted. Each site was visually inspected and confirmed in IGV.
^{#}“Good sgRNA” is defined as sgRNAs that have >50 specificity score (prediction of how much the sgRNA sequence may lead to off-target cleavage) in CRISPOR. It includes sgRNAs that are inefficient (low knockout frequencies). For SVs all VAFs included. For WES and WGS, only VAF >95% included.
**For WES, Sanger sequencing wasn't performed due to low number of good sgRNAs.
Selective cancer cell death in mixed cell cultures

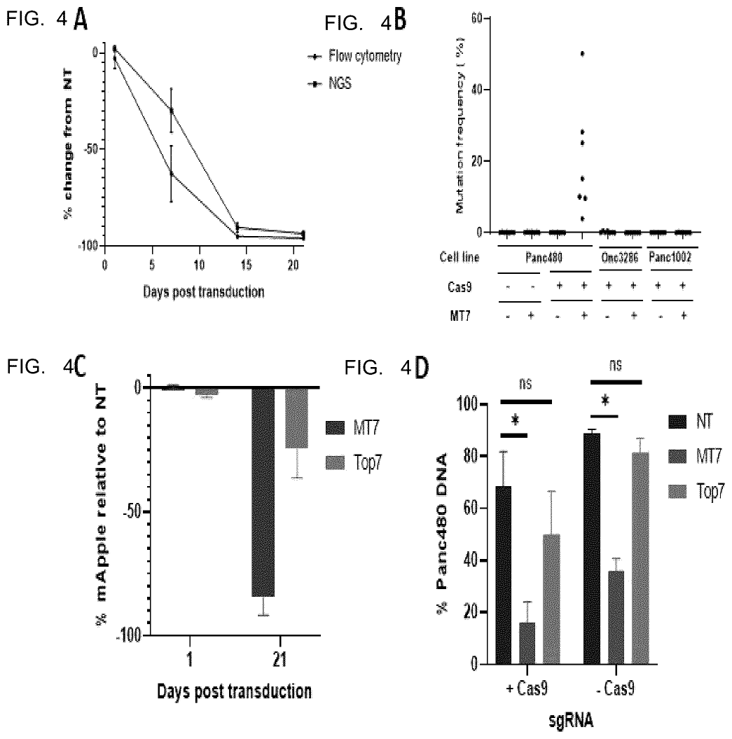

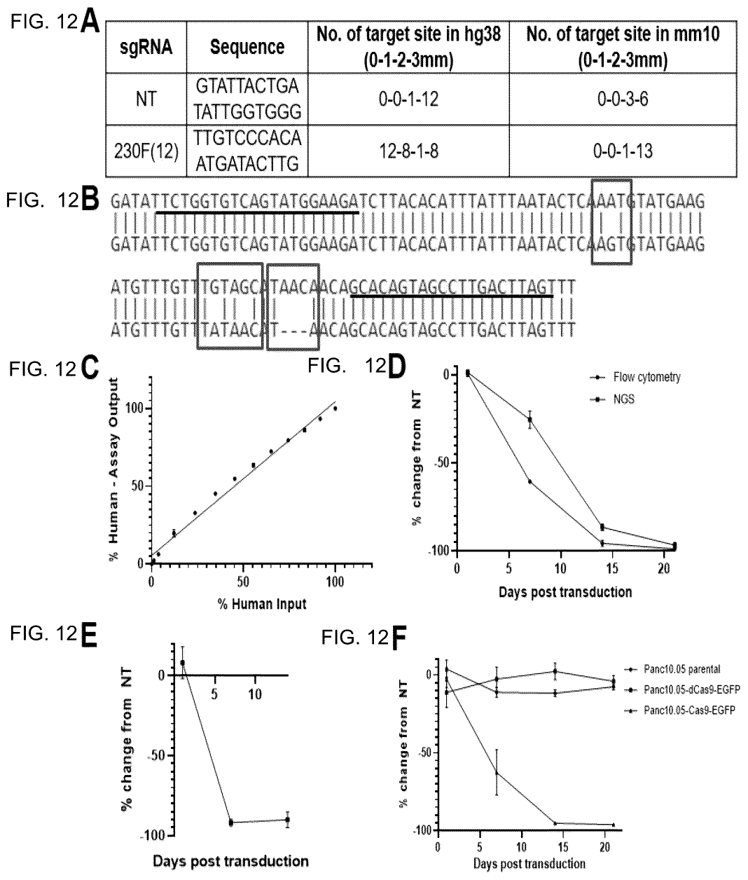

Based on the toxicity seen with the multi-target sgRNAs, the hypothesis that an individual patient's target could selectively be targeted was studied. To show proof-of-concept of CRISPR-Cas9 selectivity, cultures were seeded with Panc10.05-mApple human PC cells mixed with NIH3T3-GFP non-malignant mouse cells, both of which stably expressed Cas9. Co-cultures were transduced with a multi-target sgRNA with 12 target sites in the human genome but none in the mouse genome (FIG. 12A). The co-cultures were monitored at weekly intervals and compared the 12-cutter to the NT control sgRNA. Using flow cytometry, greater than 50% reduction in the PC cells was observed by 7 days and greater than 95% reduction by 21 days after transduction (FIG. 4A). A human-mouse NGS assay was also developed and validated based on a previously reported species-specific length polymorphism in the RC3H2 gene (FIG. 12B-12C), and confirmed >95% reduction in the human cancer cells using this independent assay (FIG. 4A)(17). Further, it was confirmed that the same level of selective cell elimination using a second human PC cell line (TS0111/NIH3T3 cells, FIG. 12D), and with a second mouse cell line derived from a genetically engineered KPC mouse model (Panc10.05/Panc02 mouse cells, FIG. 12E(18)). The human specific cell killing was dependent on both functional Cas9 and the human-specific sgRNA (FIG. 12F), showing that CRISPR-Cas9 is capable of cancer-specific selective toxicity.

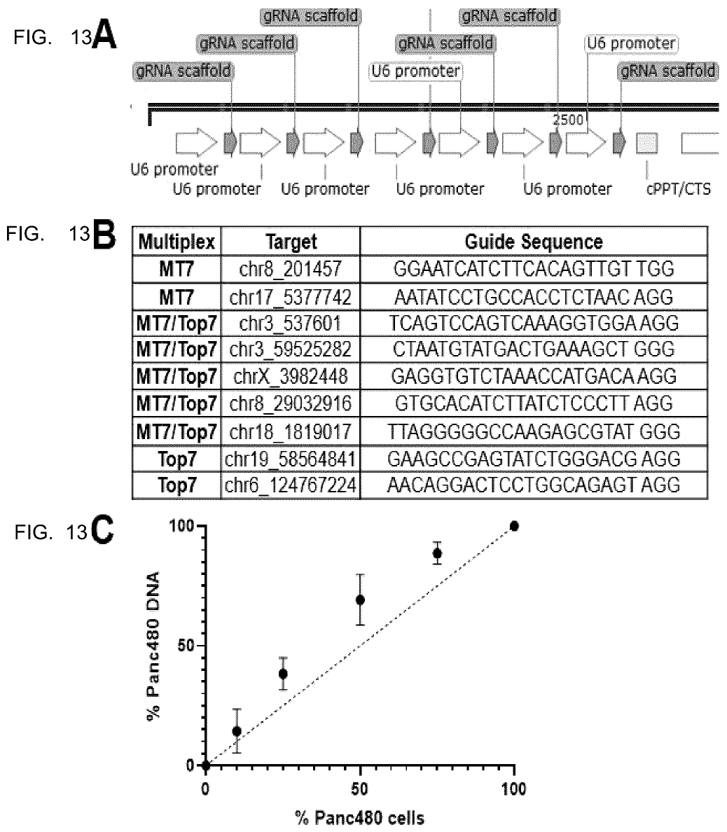

To test selective targeting of a patient's cancer cells while leaving normal cells intact, 7 of the 13 targets that were identified in Panc480 were selected using the novel PAM approach, and cloned the corresponding sgRNAs into a multiplex sgRNA expression vector with a lentiGuide-puro backbone (designated MT7 FIG. 13A-13B). After transduction into Panc480 Cas9-expressing cells, cutting activity of all 7 sgRNAs were detected by deep sequencing at the targeted loci (FIG. 4B). Importantly, cutting did not occur in Panc480 cells not expressing Cas9, normal lymphoblasts from the same patient, or in a different PC cell line lacking the PAMs adjacent to the targets (FIG. 4B). To demonstrate selective elimination in human-human PC co-cultures, Panc480 Cas9-expressing cells labeled with mApple (Panc480-Cas9-mApple) were co-cultured along with Panc10.05-Cas9-EGFP cells and transduced with MT7. Cells were cultured and selected over 21 days. Flow cytometry showed >80% selective reduction of Panc480 cells on day 21 (FIG. 4C). Cell elimination was also corroborated with an independent assay, STR profiling (FIG. 4D, FIG. 13C), which showed that the MT7 expression vector itself was somewhat toxic, but that functional Cas9 is needed to produce the full observed toxicity. A second vector (Top7) was constructed using the sgRNAs that showed the highest functional cutting activity (FIG. 13B), however this produced only 24% reduction in targeted cells. (FIG. 4C-4D). These results demonstrated that the sgRNAs designed via the target identification approach described herein were able to yield significant yet selective toxicity to targeted cells in a co-culture system. However, the differences in activity reflect the complexity of predicting sgRNA-specific cell elimination.

### Novel PAMs are Maintained in Regional Lymph Node Metastases

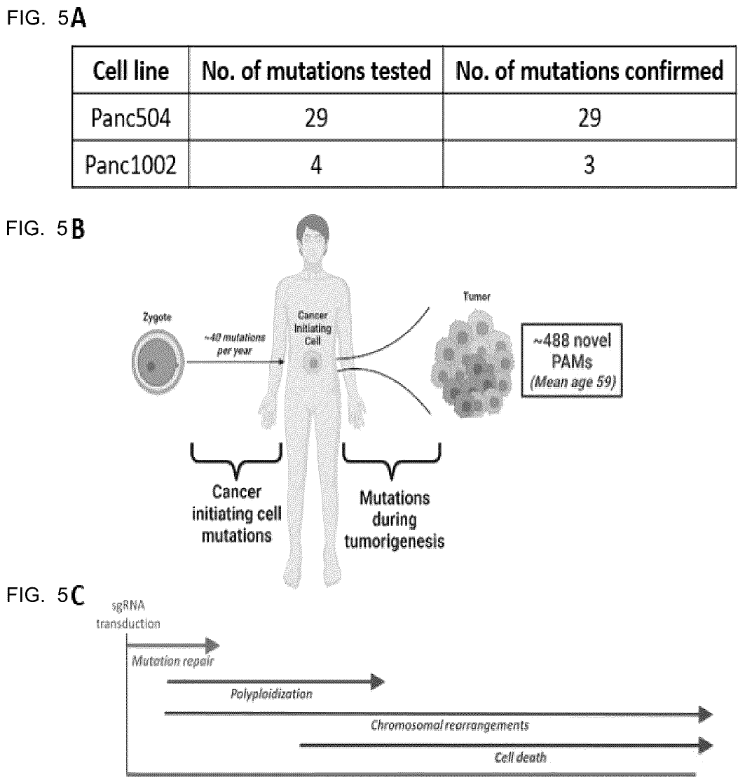

Having demonstrated selective toxicity against cancer cell lines, it was asked whether the target mutations identified in a primary tumor were maintained in metastases from the patient. For the patient from whom the cell line Panc504 was generated, a 6×5 mm focus of cancer in one of the regional lymph nodes was studied and the presence of all (29 out of 29) mutations tested (FIG. 5A) documented. A second patient, from whom the cell line Panc1002 was generated, had a very small focus (2×1 mm) of cancer in one lymph node and after careful macrodissection, we were able to demonstrate the presence of 3 out of 4 mutations tested (FIG. 5A). Archived material for the third patient (origin of Panc480) was unavailable. While available samples limited our analysis, the data showed that the majority of mutations that created novel PAM were maintained in regional lymph node metastases.

### Discussion

Mutations are one of the hallmarks of cancer (19). Most investigators naturally focus on the few driver mutations within cancers that increase the replication rate, prevent apoptosis, promote invasion or produce genomic instability (20). Far less attention has been paid to the larger set of passenger mutations, the majority of which likely arose in the patient prior to the initiation of carcinogenesis (4, 21). By definition, mutations in the cancer initiating cell must be present in all daughter cells, unless they are deleted during clonal expansion (FIG. 5B). Additional passenger mutations may arise during carcinogenesis, invasion and metastasis, allowing them to serve as a molecular clock to time these events (22).

While the concept of genetically targeting cancer cells is not new, the CRISPR-Cas9 system allows one to rapidly customize the targeting (5, 23). A variety of cancer-specific targets have been leveraged for CRISPR-based anti-cancer therapy in other laboratories, including gene fusions (24), HPV-E7 (25), insertion-deletion mutations (26), and mutant KRAS(27).

These results demonstrate that targeting 12 sites in the human genome is sufficient to eliminate >99% of cancer cells, consistent with the findings of others (26, 28). These results also show that the toxicity results from the accumulation of genomic instability (chromosomal instability, CIN) events in a TP53 mutant background (FIG. 5C). Although CIN is a key hallmark of cancer, many therapies are based on increasing this instability, such as radiation and some chemotherapeutic drugs. However, the implications of CIN have been contradictory, as some studies associated higher CIN with better therapeutic response while others have linked it to therapeutic resistance (29). As most of the target regions described herein are located near telomeres, the multitarget sgRNA treated PC cells seemed to have followed a trajectory similar to a telomere crisis, in which cells undergo massive chromosomal rearrangements and endoreduplication, resulting in high rates of cell death (30, 31).

The approach described herein presents a unique opportunity as a new precision medicine-based therapeutic tool that possesses the specificity of a targeted therapy, but without the restriction of a targetable protein. If sufficient toxicity can be achieved and delivery solved, genetically targeting a cancer's somatic mutations should provide an additional anti-cancer therapeutic approach.

### Example 3: Materials and Methods for Use in Example 4

WGS-Based PAM Discovery and sgRNA Design

DNA from tumors and corresponding normals of Panc480, Panc504, and Panc1002 were whole genome sequenced and FASTQ files were aligned to hg19 using bwa v0.7.7 (mem, https://github.com/lh3/bwa) (73) to create BAM files. The default parameters were used. Picard-tools1.119 (http://broadinstitute.github.jo/picard/) was used to add read groups as well as to remove duplicate reads. GATK v3.6.0 (67) base call recalibration steps were used to create a final alignment file. MuTect2 v3.6.0 (67) was used to call somatic variants between the tumor-normal pairs. The default parameters and SnpEff (v4.1) (74) were used to annotate the passed variant calls and to create a clean tab separated table of variants. PAMfinder (perl) was written to process VCFs based on their genome builds (hg19 or hg38) to identify somatic variants that produced novel PAMs. Tumor (arrayT) and normal (arrayN) were specified based on column number, read depth was set at 18× (75), and VAF cutoff could be modified based on the tumor purity (30% cutoff for 100% tumor purity). For somatic variants that passed through the read depth and VAF filters, the 5′ and 3′ genomic sequences flanking the somatic variants were obtained from the FASTA of individual chromosomes to inspect whether novel Cs were adjacent to an existing C or novel Gs were adjacent to an existing G. The output contained information about the somatic variant, the potential sgRNA sequence along with the novel PAM, and specified whether the novel PAM was located on the plus or minus strand of the genome. Script is available on https://github.com/selinateh/PAMfinder. Somatic mutations with VAF >95% were then chosen to put through CRISPOR (76). Somatic mutations that produced sgRNAs with >50 specificity score in CRISPOR were subsequently validated by PCR and Sanger sequencing (Table 2

### PAM Discovery on ICGC Samples

VCFs containing raw SNV calls from WGS data via the GATK Mutect2 variant calling workflow were downloaded from the ICGC-ARGO Data Portal (77). These VCFs were sourced from four projects: APGI-AU (Australian Pancreatic Cancer Genome Initiative; N=44), LUCA-KR (Personalised Genomic Characterisation of Korean Lung Cancers; N=29), PACA-CA (Pancreatic Cancer Harmonized “Omics” analysis for Personalized Treatment; N=130), and OCCAMS-GB (Oesophageal Cancer Clinical and Molecular Stratification; N=388). Clinical data corresponding to each patient was also downloaded.

VCFs were subjected to PAMfinder to identify base substitutions that produced novel PAMs. % novel PAM was calculated by dividing the number of novel PAM by the total number of base substitutions.

### Co-Culture Assays

Cells that expressed either mApple or mNeon-Green fluorescence were co-cultured at different ratios. Proportion of mApple-expressing cells post-transduction of sgRNAs were measured at different time points using Attune NxT Flow Cytometer (ThermoFisher). FCS Express 7 (De Novo Software) was used to analyze the flow cytometry data.

### CRISPR Multiplex Plasmid Functional Testing

To test the efficacy of multiplex CRISPR arrays expressing multiple sgRNA cassettes, the targeted cell line Panc480 was transduced at a 10:1 MOI with lentivirus expressing a non-targeting sgRNA (NT) or the multiplexed CRISPR array in a lentiGuide-puro backbone. 14 days after transduction and selection with puromycin, cells were harvested and gDNA extracted. The targeted loci were PCR amplified (see “Panc480 mutation validation primers” under Table 2 with NGS adaptors and sent for amplicon sequencing. The sequencing data was analyzed for the percent of edited reads by CRISPResso2 (78). Functional testing was performed in parallel for a non-targeted cell line, Panc1002, and a patient-matched EBV lymph normal cell line for Panc480, Onc3286.

### STR Analysis

Mixed human DNA samples were PCR amplified using the AmpFLSTR Identifiler PCR Amplification Kit that amplifies 15 microsatellites (Applied Biosystems, Foster City, CA) per manufacturer's instructions, and amplicons resolved on a 3130 capillary electrophoresis instrument (Applied Biosystems). Percentage of a given individual was calculated from on-scale informative peak heights using Chimeranalyzer (https://github.com/young-jon/chimeranalyzer).

### Statistical Analysis

The appropriate statistical tests were performed in GraphPad Prism (Version 9.2.0). The statistical models used were stated in results and in the Brief Description of the Figures. For all statistically significant results, * indicates P<0.05, ** indicates P<0.01, *** indicates P<0.001, and * indicates P<0.0001.

SV Target Validation and sgRNA Design

DNA from tumor and corresponding normal tissue for Panc480, Panc504, and Panc1002 were used for high-density SNP microarray and whole genome sequencing (WGS) as previously described (32, 79). A list of SVs were compiled from SVs previously published in Norris et al. (2015) (79). Additional SVs were discovered by using Trellis (16), an SV caller on WGS data via tumor-normal subtraction. SVs that were present in normal based on IGV (39) visual inspection were further eliminated from the list. Primers were designed to PCR amplify across breakpoints and sent for Sanger sequencing (Table 1). Among the validated ones, we selected for potential sgRNA sequences in which either the PAM spanned across the breakpoint junction or at least 4 bases of the sgRNA sequence crossed the junction. Then, we entered the sequence into CRISPOR (35) and selected candidates that have >50 specificity score.

WES Target Identification and sgRNA Design

DNA from tumor and corresponding normal tissue for Panc480, Panc504, and Panc1002 were whole exome sequenced and variants called as previously described (32). Mutations were inspected to include novel Cs that were adjacent to an existing C or novel Gs that were adjacent to an existing G after tumor-normal subtraction. The resulting list of mutations was put through CRISPOR and the ones that produced sgRNAs with >50 specificity score in CRISPOR were subsequently examined for their VAFs.

SBS filter

A perl script was written to process VCFs to identify somatic variants that pass through a predetermined set of read depth and VAF filters. Tumor (arrayT) and normal (arrayN) were specified based on column number, read depth were set at 18× (50), and VAF cutoff could be modified based on the purpose of the analysis. Script is available on https:/Mfinder.

### Cas9-mApple Plasmid Construction

mApple-N1 (54) was a gift from Michael Davidson (Addgene plasmid #54567). Primers were designed to amplify the vector from pLentiCas9-T2A-GFP and mApple insert from mApple-N1 using Q5 Hot Start High-Fidelity polymerase (NEB) according to the manufacturer's protocol (Table 5). PCR products were subjected to gel electrophoresis with 0.8% agorose gel at 150V for 2 hours. Gel extraction was performed with QIAquick Gel Extraction Kit (QIAGEN) according to the manufacturer's protocol to purify the vectors and inserts. Then, Gibson assembly was performed with a 2:1 ratio of insert:vector using Gibson Assembly Master Mix (NEB) and an incubation time of 1 hour at 50° C. The Gibson product was transformed into NEB 5-alpha Competent E. coli according to the manufacturer's protocol and were selected by both carbenicillin and ampicillin. Plasmids were extracted from ampicillin-resistant clones using QIAprep Spin Miniprep kit (QIAGEN) according to the manufacturer's protocol. Analytical digestion with restriction enzymes (NEB) was performed to verify the identity of the plasmid. Primers were designed to confirm insertion (Table 5). The plasmid was then transfected into 293T cells with Invitrogen Lipofectamine 3000 reagent and P3000 reagent (ThermoFisher) according to manufacturer's protocol, and observed under fluorescence microscope for functional validation.

dCas9 Plasmid Construction

pLentiCas9-T2A-GFP was a gift from Roderic Guigo & Rory Johnson (52) (Addgene plasmid #78548) and pZLCv2-3×FLAG-dCas9-HA-2×NLS was a gift from Stephen Tapscott (53) (Addgene plasmid #106357). Primers were designed to amplify the vector from pLentiCas9-T2A-GFP and dCas9 insert from pZLCv2-3×FLAG-dCas9-HA-2×NLS using Q5 Hot Start High-Fidelity polymerase (NEB) according to the manufacturer's protocol (Table 4). PCR products were subjected to gel electrophoresis with 0.8% agarose gel at 150V for 2 hours. Gel extraction was performed with QIAquick Gel Extraction Kit (QIAGEN) according to the manufacturer's protocol to purify the vectors and inserts. Then, Gibson assembly was performed with a 3:1 ratio of insert:vector using Gibson Assembly Master Mix (NEB) and an incubation time of 1 hour at 50° C. The Gibson product was transformed into NEB 5-alpha Competent E. coli according to the manufacturer's protocol and were selected by both carbenicillin and ampicillin. Plasmids were extracted from ampicillin-resistant clones using QIAprep Spin Miniprep kit (QIAGEN) according to the manufacturer's protocol. Analytical digestion with restriction enzymes (NEB) was performed to verify the identity of the plasmid. Primers were designed to PCR and Sanger sequence regions spanning D10 and H840 of dCas9 to validate the mutations on dCas9 (Table 4).

Non-Targeting and 12-Cutter sgRNA Design

Chromosome range was entered into CRISPOR (5) 2 kb at a time starting at chr1:0-2000 and ending at chr1:100,248,000-100,250,000 based on hg19 and hg38, respectively. sgRNAs that have 12 perfect target sites were selected from the pool of sgRNA options generated by CRISPOR based on the following criteria: (1) none of the perfect target sites and potential off-target sites target exons; (2) Doench′16 (36) efficiency score is >50%, and (3) the number of off-targets that have no mismatches in the 12 bp adjacent to the PAM (SEED region) is <10. The sequence of the sgRNA selected, 230F(12), is TTGTCCCACAATGATACTTG (SEQ ID NO:11). Sequence of non-targeting control (NT: GTATTACTGATATTGGTGGG (SEQ ID NO:1) sgRNA was obtained from Doench et al (36).

sgRNA-Expressing Plasmid Construction

lentiGuide-Puro (55) was a gift from Feng Zhang (Addgene plasmid #52963) and lentiCRISPRv2 puro (56) was a gift from Brett Stringer (Addgene plasmid #98290). Oligonucleotides of sgRNA sequences were ordered from IDT for cloning into both lentiGuide-Puro and lentiCRISPRv2 puro backbones according to Feng Zhang's Lab Target Guide Sequence Cloning protocol (55, 13). The resulting product was transformed into One Shot Stb13 chemically competent E. coli (ThermoFisher) according to the manufacturer's protocol and selected with both carbenicillin and ampicillin. Plasmids were extracted from ampicillin-resistant clones using QIAprep Spin Miniprep kit (QIAGEN) according to the manufacturer's protocol. Analytical digestion with restriction enzymes (NEB) was performed to verify the identity of the plasmids and Sanger sequencing was performed to validate the insertion of sgRNA sequence.

### Lentivirus Titer Preparation and Quantification

pCMV-VSV-G (17) was a gift from Dr. Bob Weinberg (Addgene plasmid #8454), pMDLg/pRRE and pRSV-Rev were gifts from Dr. Didier Trono (58) (Addgene plasmid #12251 & #12253). 2.5 ug pCMV-VSV-G, 5 ug pMDLg/pRRE, 5 ug pRSV-Rev, and 7.5 ug transfer plasmids were used along with 50 uL Invitrogen Lipofectamine 3000 reagent and 40 uL P3000 reagent (ThermoFisher) for transfection into 293T cells on a 10-cm plate (95-99% confluent at transfection). Cell culture and transfection workflows were the same as the manufacturer's protocol. Upon harvesting and pooling the lenvirus-containing supernatant, the clarified supernatant was concentrated with Lenti-X Concentrator (Takara Bio) by following the manufacturer's protocol. Lenti-X qRT-PCR titration kit (Takara Bio) was used to quantify an aliquot of the clarified lentiviral supernatant according to the manufacturer's protocol.

### Cell Culture

Panc10.05, TS0111, Panc480, Panc1002, NIH3T3, Panc02, Onc3286, and their derivative cell lines were STR profiled and mycoplasma tested before the start of experiments. All cells, except for Onc3286, were maintained in monolayer cultures at 37° C. and 5% CO₂. The culture medium consisted of 1×DMEM, 10% fetal bovine serum, 2 mM L-glutamine, and 1× antibiotic antimycotic solution (Sigma; contains 100u penicillin, 100 ug streptomycin, and 0.25 ug amphotericin B). Onc3286 was maintained in a suspension culture at 37° C. and 5% CO₂. The culture medium consisted of 1×RPMI 1640, 20% heat-inactivated bovine calf serum, 2 mM L-glutamine, and 1× antibiotic antimycotic solution (Sigma).

### Fluorescent Cas9-Expressing Cell Line Construction

Cells were seeded at 50% confluence for 24 hours before the media was replaced to contain 10 ug/mL of polybrene. Lentivirus of Cas9-expressing plasmids, either pLentiCas9-T2A-GFP or pLentiCas9-T2A-mApple, were added into the media at MOI 0.01 and transduction took place for 18-20 hours. The media was then removed, washed once with PBS, and replaced with normal media. After 24 hours, the media was replaced with media that contained 5 ug/mL blasticidin for a 7-day selection. The cells were then sent to the SKCCC Flow Cytometry Core or SKCCC High Parameter Flow Core for fluorescence activated cell sorting using BD FACSAria II or BD Fusion sorter, respectively, to sort for cells with the optimal fluorescence intensity. The sorted cells were cultured in the presence of blasticidin selection and subjected to STR profiling and mycoplasma testing. Fluorescence microscopy was performed to verify the presence of fluorescent markers before experiments were carried out on these cell lines.

### Cas9 Activity Assay

Cells were transduced with sgRNAs targeting HPRT1 gene to induce mutations, which could be functionally screened via 6-thioguanine (6-TG) positive selection. For human, the sgRNA used was HPRTc.465 (designed via CRISPOR) and non-targeting control was NT2 (37); for mouse, it was mchrX:52M with mchrX:53M as an off-target control, both designed via CRISPOR (Table 6). Target site was PCR amplified and sent for NGS (see Methods below; Table 6). Mutation frequency of target site was quantified using CRISPResso2 pipeline (59).

### Next Generation Sequencing (NGS) of Amplicons

PCR was performed with primers containing partial Illumina adapter sequences to generate amplicons. Either NEBNext High-Fidelity 2×PCR Master Mix (NEB) or Platinum SuperFi II PCR Master Mix (Thermo Fisher) was used for PCR preparations, and thermocycling conditions were set based on manufacturers' suggestions. Amplicons were purified using QIAGEN MinElute PCR purification kit based on manufacturer's protocol. Purified PCR products were sent to Azenta for Amplicon-EZ service, in which 2×250 bp sequencing was performed to provide ˜50,000 reads per sample. FASTQ files were obtained for further analysis.

### Mouse-Human NGS Assay

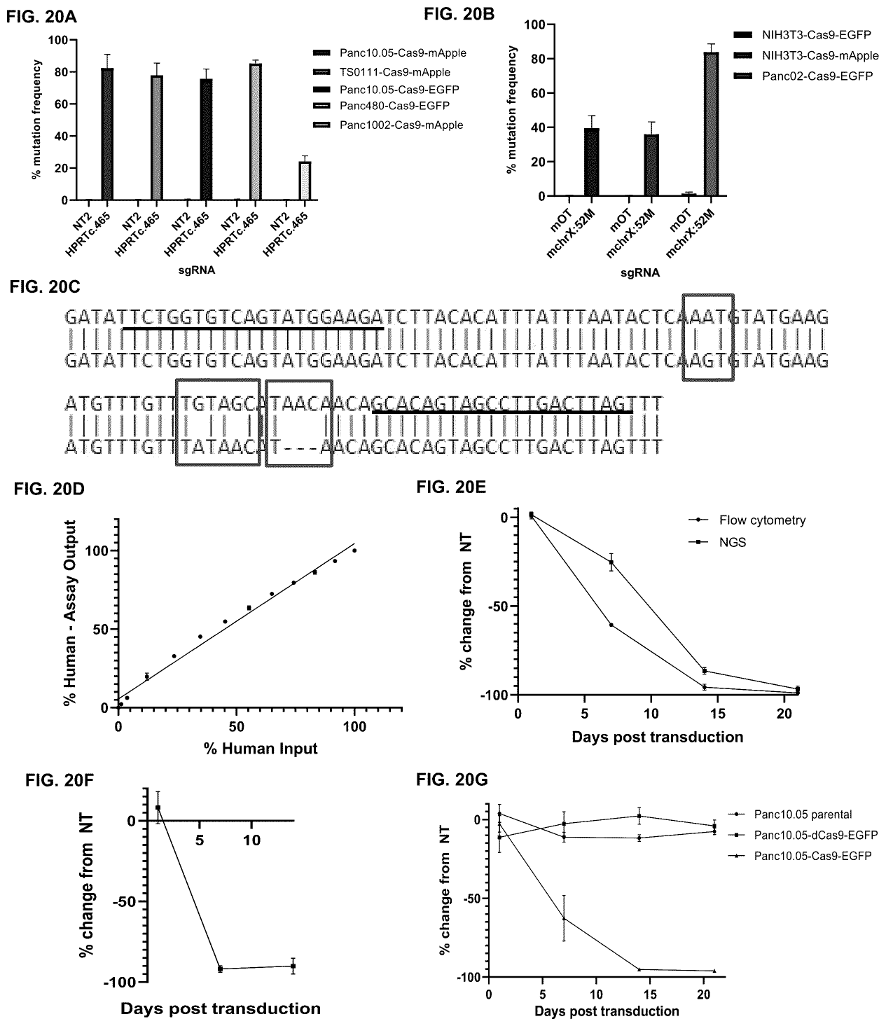

The RC3H2 gene was selected as the mouse and human orthologs differ by a 3 bp indel followed by 3 SNPs (FIG. 20C). Primers for unbiased PCR amplification of the locus in mouse and human DNA were previously developed by Lin et. al. (17), designated as primer pair 45 (Table 3). For this assay, a 101 bp amplicon in the RC3H2 gene was amplified with primers containing Illumina adaptor sequences. Amplicons were subjected to NGS, and FASTQ files were aligned to the hg19 genome using bwa 0.7.17 (51) and visualized in IGV. Human and mouse reads were quantified as reads, and deletions, respectively, as the 3 bp-shorter mouse sequence maps as a deletion in the human genome. For validation, mouse DNA was obtained from the liver of a nude mouse, and human DNA from human splenic tissue.

### Multiplex Cloning

Individual sgRNA targeting novel PAMs were obtained as ssDNA oligos from IDT and cloned into lentiGuide-puro (Addgene #52963) and lentiCRISPRv2-puro (Addgene #98290) lentiviral expression vectors per the protocol previously published by the Zhang Lab (55, 13). The U6 promoter, guide sequence, and sgRNA scaffold, referred to here as cassettes, were then PCR amplified off each lentiGuide-puro-sgRNA construct for each locus targeted (Table 8). For multiplexing, the lentiGuide-puro construct containing the first guide was linearized by PpuMI digestion (NEB) and cassettes were serially added by Gibson assembly with PpuMI linearization of the growing array for each cycle (Table 8). The final multitarget-7 (MT7) construct was then back-cloned into the original species of lentiGuide-puro and verified by analytical digestion and Sanger sequencing (Table 8).

### WGS Analyses for Potential Off-Target Sites on Panc1002 Control

MuTect2 v3.6.0 (38) was used to call somatic variants between the sample-control pair. The default parameters were used. From the list of results generated, we looked for loci within the VCF that closely matched our sgRNA sequence. Two independent approaches were performed for subsequent analyses. For the first approach, this was performed with R script that performed the following steps: 1) Read in an Excel file containing one mutation per row. 2) Obtain the forward and reverse strand sequences from the hg19 genome between the start −50 bp and stop +50 bp positions of the locus. 3) Align each locus's forward and reverse sequences to the target sgRNA with no gaps using the Smith-Waterman algorithm. 4) Determine the number of mismatches between the sgRNA and the nearest matching piece of DNA within each junctions. Output the original information along with new columns displaying the mismatches between each junction and the sgRNA into a new Excel file. From the list of outputs, we only considered potential target sites that have <5 bp mismatch to the sgRNA sequence.

As an orthogonal method to check for off-target editing, a second investigator manually reviewed all the indel mutations from the VCF on IGV. This was done according to the following steps: 1) Screen the original 212 calls to see if the mutation detected is present in IGV, the pre-treatment sample (T0) as well as the post-treatment sample (T14), or a result of polymerase slippage or mapping error in a repetitive region. 2) For the remaining potential new indel mutations, 50 bp upstream and downstream are analyzed for >5 bp homology with any of the 7 sgRNAs in MT7 using NCBI Blast2Seq.

### Example 4: Development of PAM Discovery Approach

Two approaches were tested with the potential to lead to highly selective target cell killing with minimal off-target risk. S. pyogenes NGG PAM were selected due to its smaller PAM size (61). As pancreatic cancer (PC) is one of the most lethal cancers with a dismal five-year survival rate of only 11.5% (62), whole genome sequencing (WGS) data from three PC cell lines and their corresponding normal DNA (normal cell line available) was used to perform tumor-normal subtraction for identification of somatic mutations (Table S1). All three PC samples harbored deleterious mutations in KRAS, CDKN2A, SMAD4, and TP53, which are the most common driver mutations in PCs (Table 16).

TABLE 16
Source of genomic DNA and mutation profile of the
driver genes of three pancreatic cancer cell lines.

| | Source of | Source of | Tumor | Tumor | Tumor | Tumor |
|---|---|---|---|---|---|---|
| Sample | tumor DNA | normal DNA | KRAS | CDKN2A | SMAD4 | TP53 |
| Panc480 | Primary | Lymph | G12D | Frameshift | Homozygous | V274A |
| | | | | | deletion | |
| Panc504 | Primary | Duodenum | G12V | Homozygous | Homozygous | Frame- |
| | | | | deletion | deletion | shift |
| Panc1002 | Primary | Lymph | Q61H | Homozygous | Homozygous | R248Q |
| | | | | deletion | deletion | |

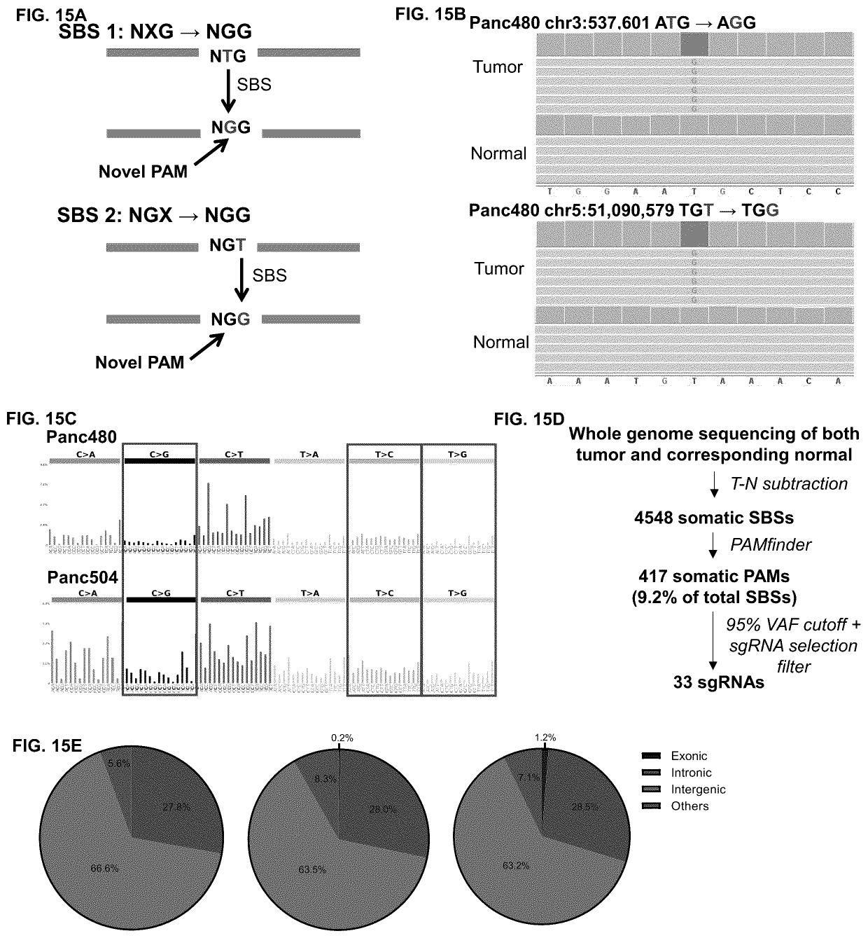

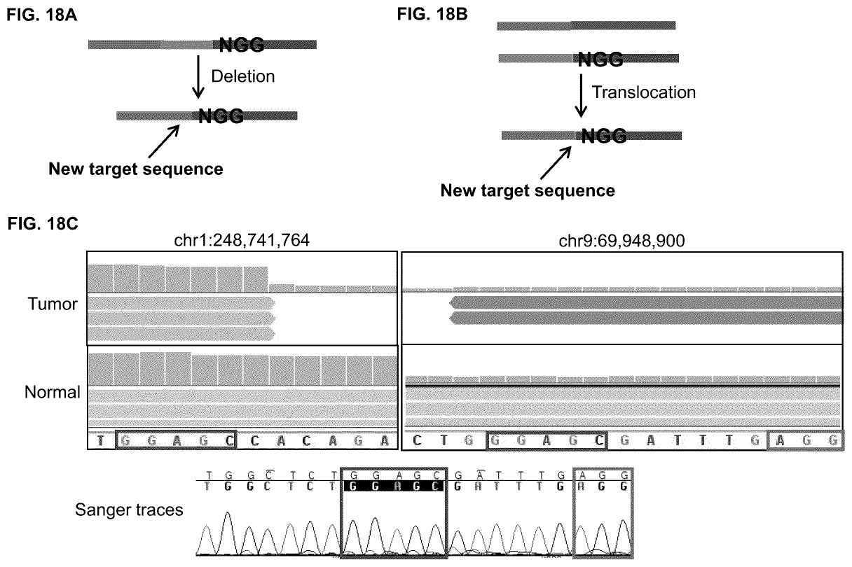

Structural variants (SVs) were considered first, since they could juxtapose a new target DNA sequence next to an existing NGG PAM (FIG. 15A-15B). This could theoretically decrease the risk of off-target effects, as the resulting breakpoint is significantly different from the original sequence in the human genome (FIG. 18C). A SV detection software, Trellis (24), was used to identify SVs comprehensively from WGS data. An average of 35 SVs per cell line was confirmed by comparing tumor to normal, and validated 84.9% of them by PCR amplification across the breakpoint and Sanger sequencing (Table 17, FIG. 18C). An average of 22 novel SVs juxtaposed next to an existing PAM per cell line were found (Table 17). Using the sgRNA selection criteria (see Example 3 above), an average of 17 good sgRNAs per cell line were obtained (Table 17).

TABLE 17
Novel SVs discovered for sgRNA design.

| | No. of | | | | | |
|---|---|---|---|---|---|---|
| | somatic SVs | No. of | | No. of | | |
| | discovered | somatic SVs | Total no. | Sanger- | No. of | No. of |
| | via SNP | discovered | of somatic | validated | SVs with | good |
| Cell line | microarray* | via WGS | SVs | SVs | PAM | sgRNAs^{#} |
| Panc480 | 7 | 37 | 38 | 31 | 24 | 17 |
| Panc504 | 8 | 33 | 37 | 29 | 18 | 15 |
| Panc1002 | 11 | 28 | 31 | 30 | 25 | 18 |
| Average | 9 | 33 | 35 | 30 | 22 | 17 |

*SVs identified were previously published in Norris et al. (2015) Genes, Chromosomes & Cancer.
^{#}“Good sgRNA” is defined as sgRNAs that have >50 specificity score (prediction of how much the sgRNA sequence may lead to off-target cleavage) in CRISPOR. It includes sgRNAs that are inefficient (low knockout frequencies).

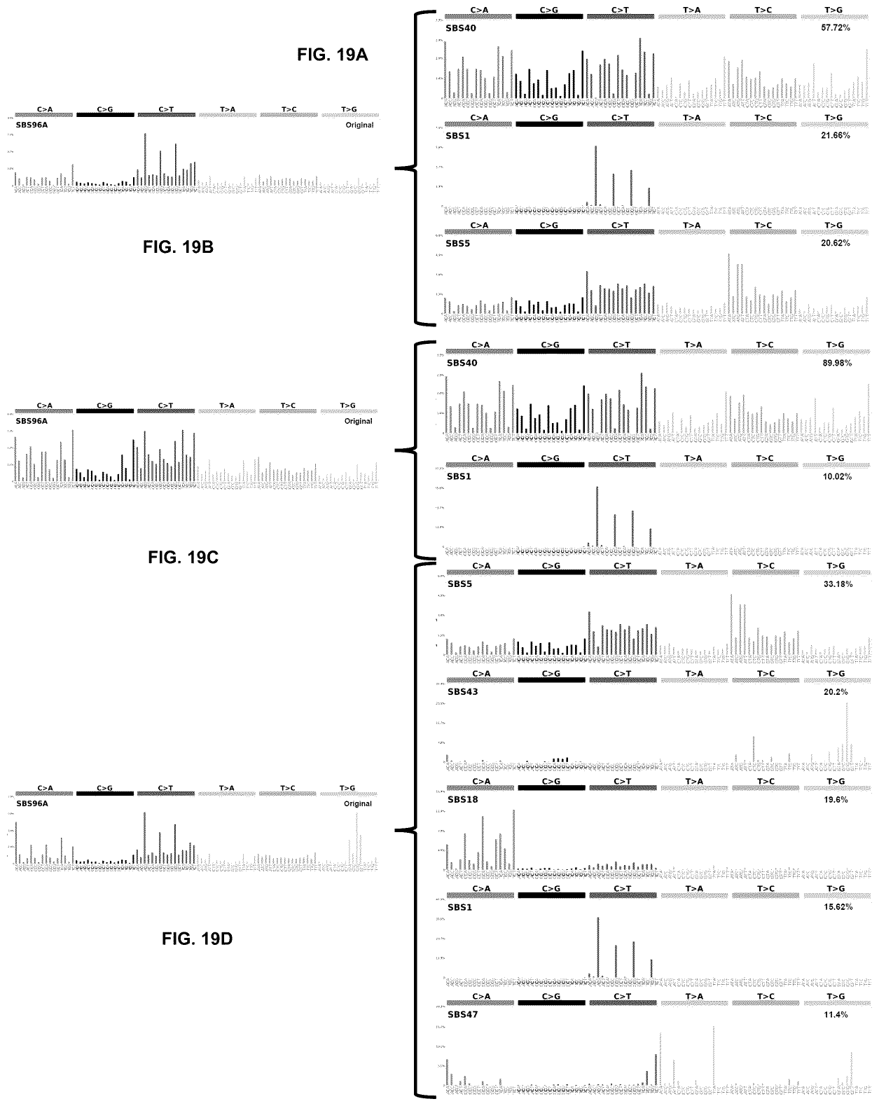

Next, an attempt was made to discover novel PAMs created from SBSs (FIG. 15A-15B). Somatic NGG PAMs can arise through SBS that creates a novel G from A/T/C, and this novel G is adjacent to an existing G one nucleotide upstream or downstream of the novel G (FIG. 15A-15B). The same concept applies to the complementary strand which would use the CCN sequence. Mutational signature analyses of the PC samples also showed that somatic mutations that produced novel Cs and Gs were evident in the samples (FIG. 15C). The most common signatures were SBS1, 5, and 40, which are all clock-like signatures (63-65), suggesting that aging itself could give rise to novel PAMs (FIG. 19). A program, PAMfinder, was developed, to discover somatic base substitutions that produced novel PAMs in a given tumor sample.

An average of 4548 SBSs per sample were identified, in which 9.2% of them created somatic PAMs (mean=417; FIG. 15D, Table 18).

TABLE 18
Novel PAMs discovered from SBSs using WGS.

| | | | | | | No. of |
|---|---|---|---|---|---|---|
| | | | | | | Sanger- |
| | No. | No. of | | No. of | No. of | validated |
| Cell | of | somatic | % | PAM with | good | good |
| line | SBS | PAM^{&} | PAM | VAF >95% | sgRNAs^{#} | sgRNAs |
| Panc480 | 4576 | 385 | 8.4 | 23 | 13 | 13 |
| Panc504 | 4502 | 417 | 9.3 | 76 | 48 | 47 |
| Panc1002 | 4566 | 448 | 9.8 | 78 | 38 | 37 |
| Average | 4548 | 417 | 9.2 | 63 | 33 | 32 |

^{&}Somatic PAM indicates a SBS of NGN/NNG sequence to NGG (both + and − strands). Only mutations with a variant allele frequency (VAF) of at least 30% in tumor (to account for subclonal mutations that potentially arose from in vitro culture) and a minimum of 18X read depth in both normal and tumor were included.
^{#}“Good sgRNA” is defined as sgRNAs that have >50 specificity score (prediction of how much the sgRNA sequence may lead to off-target cleavage) in CRISPOR. It includes sgRNAs that are inefficient (low knockout frequencies).

A variant allele frequency (VAF) cutoff of 30% was used to exclude mutations that might be subclonal or have arisen through in vitro culture of these cell lines. For initial functional testing of sgRNAs, novel PAMs with VAFs >95% (mean=63) were selected as intuitively, targeting them should produce the highest toxicity; and of them, an average of 33 good sgRNAs could be designed using the sgRNA selection criteria (FIG. 15D, Table 19). It was possible to confirm all the qualifying mutations, except two, using Sanger sequencing (Table 19). A similar approach using whole exome sequencing (WES) data failed to yield sufficient targets (mean=1; Table 19).

TABLE 19
Novel PAMs discovered from SBSs using WES.

| | | | | No. of good |
|---|---|---|---|---|
| | Total no. | No. of | No. of | sgRNAs with |
| | of somatic | novel | good | PAM of |
| Cell line | mutations | PAM | sgRNAs^{#} | VAF >95% |
| Panc480 | 44 | 8 | 5 | 2 |
| Panc504 | 38 | 3 | 0 | 0 |
| Panc1002 | 30 | 4 | 2 | 0 |
| Average | 37 | 5 | 2 | 1 |

#“Good sgRNA” is defined as sgRNAs that have >50 specificity score (prediction of how much the sgRNA sequence may lead to off-target cleavage) in CRISPOR. It includes sgRNAs that are inefficient (low knockout frequencies).

This was because the majority of the novel PAMs were located in noncoding regions, as 64.4% of all somatic PAMs were located in intergenic regions, 28.1% in introns, 0.5% in exons, and the remaining 7.0% in regions such as non-coding RNAs (FIG. 15E). Thus, it was concluded that the WGS-based PAM discovery approach using SBSs was more productive than the SV and WES approaches, and provided hundreds of novel PAMs per cancer as potential CRISPR-Cas9 target sites.

### High Prevalence of Novel PAMs in Different Tumor Types

To determine the prevalence of novel PAM in different tumor types, VCFs from the ICGC Data Portal (66) were analyzed using PAMfinder and identified a large number of PAMs in lung cancers (LUCA-KR), esophageal cancers (OCCAMS-GB), and additional PCs (APGI-AU and PACA-CA). To briefly describe the data in these VCFs, WGS data were aligned to GRCh38 reference genome to produce aligned CRAM files, and these CRAM files were processed through the GATK Mutect2 variant calling (67) workflow as tumor-normal pairs to identify somatic base substitutions. As the WGS on tumors were performed on primary tumor samples, the tumor purity was calculated for each sample and varied the VAF cutoffs for each to filter out mutations that were likely subclonal or background (see Example 3, Table 20).

TABLE 20
Summary of tumor purity, base substitutions, and somatic
PAMs obtained from different ICGC projects.

| | | % tumor purity | | No. of base substitutions | | No. of somatic PAM | | % PAM* | |
|---|---|---|---|---|---|---|---|---|---|
| Project | N | Median | IQR^{#} | Median | IQR^{#} | Median | IQR^{#} | Median | IQR^{#} |
| APGI- | 44 | 29.7 | 29.2- | 5890.5 | 4058.8- | 478.5 | 344.8- | 8.9 | 8.1- |
| AU | | | 40.1 | | 8390.3 | | 844.0 | | 10.5 |
| PACA- | 130 | 38.2 | 29.8- | 5354.5 | 4232.8- | 430.5 | 340.5- | 8.4 | 7.7- |
| CA | | | 47.8 | | 7942.0 | | 711.5 | | 9.8 |
| LUCA- | 29 | 36.3 | 30.8- | 30553.0 | 19081.5- | 2790.0 | 2211.5- | 8.5 | 7.8- |
| KR | | | 47.3 | | 45893.0 | | 3675.0 | | 9.2 |
| OCCA | 388 | 32.8 | 29.5- | 20106.0 | 13542.5- | 3235.5 | 1741.3- | 16.1 | 12.3- |
| MS-GB | | | 40.0 | | 31705.0 | | 6167.3 | | 20.5 |
| All | 591 | 34.4 | 29.5- | 15552.0 | 7091.0- | 2131.0 | 662.0- | 12.9 | 9.0- |
| | | | 41.0 | | 26989.0 | | 4535.0 | | 18.2 |

^{#}IQR indicates interquartile range (25^{th}-75^{th} percentile).
*% PAM = No. of somatic PAM/No. of base substitutions

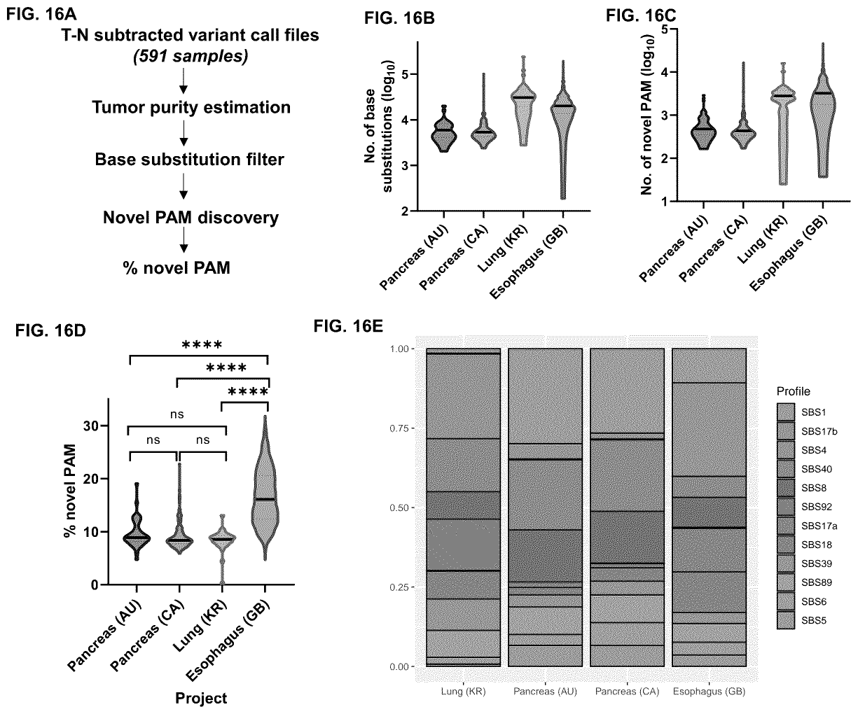

Overall, it was found that the number of base substitutions and number of somatic PAM from the two PC projects, APGI-AU (N=44) and PACA-CA (N=130), were comparable to findings from the discovery PC lines, in which a median of 478.5 and 430.5 somatic PAMs were identified, respectively (FIG. 16C, Table 20). Regarding the 29 lung cancer samples (LUCA-KR) and 388 esophageal cancer samples (OCCAMS-GB), the number of PAMs identified was >5 fold higher than that of PCs, with a median of 2790 and 3235.5, respectively (FIG. 16C, Table 21). Since the number of base substitutions were also higher in lung cancers (median=30553) and esophageal cancers (median=20106) compared to PCs (median=5890.5 and 5354.5), these results indicate tissue specificity in which different mechanisms contributed to the varying number of mutations present (FIG. 16B, Table 20).

Notably, while the percentage of base substitutions that gave rise to somatic PAMs (% novel PAM) were similar among PCs and lung cancers with medians at 8.8% (APGI-AU), 8.4% (PACA-CA), and 8.5% (LUCA-KR), esophageal cancers had significantly higher % novel PAM of 16.1% (interquartile range=12.3-20.5%; P<0.0001; FIG. 16D, Table 20). To investigate the potential mechanism contributing to the higher % novel PAM, mutational signature analysis was performed of all samples. It was found that the two cohorts of PC samples showed similar mutational signatures that were consistent with previous findings using the discovery PC cell lines (SBS1 and SBS40), while the top mutational signature for lung cancers, SBS4, is associated with tobacco smoking (26,30) (FIG. 16E). More importantly, the top ranked mutational signature of esophageal cancer samples, SBS17b, distinguished itself from the other tumor types (FIG. 16E). It was characterized primarily by a T>G transversion with an unknown etiology, but previous studies have associated it with fluorouracil (5FU) treatment and possibly damage by reactive oxygen species (68, 69). This finding was also consistent with previous studies published with these samples (70, 71). Based on the analyses of different large tumor cohorts, it was concluded that somatic base substitutions in the tumor types examined yielded hundreds, if not thousands, of novel PAMs in each tumor, and these findings are tissue, and potentially, treatment-dependent.

Selective Cell Killing with CRISPR-Cas9

Finally, the hypothesis was tested that an individual patient's cancer could selectively be targeted using sgRNAs designed from the PAM discovery approach. To show proof-of-concept of CRISPR-Cas9 selectivity, Cas9-expressing mouse and human cell lines were generated and Cas9 activity documented (FIG. 20A-20B). Then, mouse-human cell line co-cultures were seeded, and transduced with a multi-target sgRNA with 12 target sites in the human genome but none in the mouse genome (Table 21).

TABLE 21
Number of target sites of NT and 230F(12) sgRNAs in both mouse (mm10)
and human (hg38) genomes.

| | | No. of target | No. of target |
|---|---|---|---|
| | | site in hg38 | site in mm10 |
| sgRNA | Sequence | (0-1-2-3 mismatches) | (0-1-2-3 mismatches) |
| NT | GTATTACTGATATTGGTGGG | 0-0-1-12 | 0-0-3-6 |
| | (SEQ ID NO: 1) | | |
| 230F(12) | TTGTCCCACAATGATACTTG | 12-8-1-8 | 0-0-1-13 |
| | (SEQ ID NO: 11) | | |

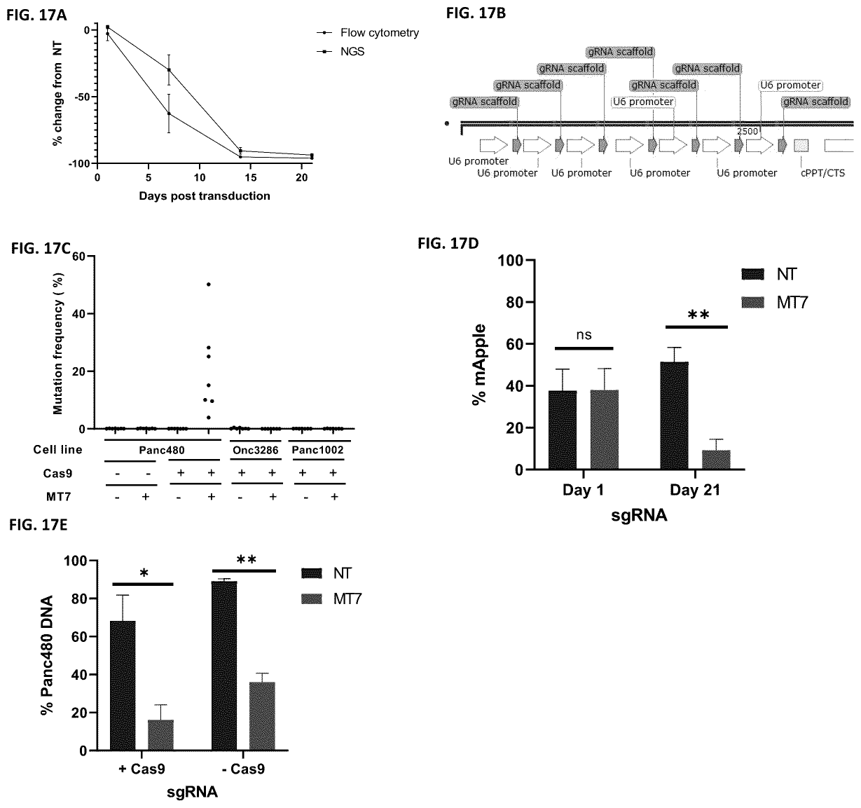

Using both flow cytometry and a human-mouse NGS assay (see Supplementary methods, FIG. 20C-20D), a >95% reduction of the human cancer cells in different co-cultures was observed (FIG. 17A, FIG. 20E-20F). The human-specific cell killing was dependent on both functional Cas9 and the human-specific sgRNA, showing that the CRISPR-Cas9 system is capable of selectively eliminating cancer cells (FIG. 20G).

To test selective targeting of a patient's cancer cells while leaving normal cells intact, 7 of the 13 targets were selected that were identified in Panc480 using the novel PAM discovery approach, confirmed targeting efficiency of individual sgRNAs, and cloned the corresponding sgRNAs into a multiplex sgRNA expression vector (designated MT7; FIG. 17B; Table 22).

TABLE 22
Cutting efficiency and off-target activity tests of the list of sgRNAs in
Panc480-MT7.

| | | | | | Lowest |
|---|---|---|---|---|---|
| | | | | Mutation | number of |
| | | | Mutation type | frequency | mismatch |
| Target | sgRNA sequence | PAM | (copy number) | (%)^{&} | in T14* |
| chr8:201457 | GGAATCATCTTCACAGTTGT | TGG | D-LOH^{#} (1) | 22.6 | 7 |
| | (SEQ ID NO: 448) | | | | |
| chr17:5377742 | AATATCCTGCCACCTCTAAC | AGG | D-LOH (1) | 36.4 | 7 |
| | (SEQ ID NO: 464) | | | | |
| chr3:537601 | TCAGTCCAGTCAAAGGTGGA | AGG | D-LOH (1) | 87.3 | 7 |
| | (SEQ ID NO: 465) | | | | |
| chr3:59525282 | CTAATGTATGACTGAAAGCT | GGG | D-LOH (1) | 71.1 | 5 |
| | (SEQ ID NO: 450) | | | | |
| chrX:3982448 | GAGGTGTCTAAACCATGACA | AGG | D-LOH (1) | 67.8 | 7 |
| | (SEQ ID NO: 452) | | | | |
| chr8:29032916 | GTGCACATCTTATCTCCCTT | AGG | D-LOH (1) | 57.6 | 6 |
| | (SEQ ID NO: 466) | | | | |
| chr18:1819017 | TTAGGGGGCCAAGAGCGTAT | GGG | D-LOH (1) | 68.7 | 7 |
| | (SEQ ID NO: 467) | | | | |

^{#}D-LOH: deletion-based loss of heterozygosity
^{&}Individual sgRNAs were transduced into Panc480 cells separately and puromycin-selected for 7 days. Cells were harvested for NGS and mutation frequency was quantified using CRISPResso2.
*WGS analyses were performed for T14. For each indel detected by Mutect2, the original sequence on the reference genome was compared to the sgRNA sequence to determine the homology between both using an in-house R script (see Supplementary methods). The lowest number of sequence mismatch was shown.

After transduction into Panc480 Cas9-expressing cells, we detected cutting activity of all 7 sgRNAs, and not in its controls (Panc1002 Cas9-expressing cell line) or corresponding normal cells from the patient (Onc3286), by deep sequencing at the targeted loci (FIG. 17C). As another negative control to check for potential Cas9 off-target activity, Panc1002 Cas9-expressing cells lacking the targets were seeded in cell culture and transduced with Panc480-MT7 which targets mutations unique to Panc480. WGS was performed before transduction (TO) and 14 days post-transduction of MT7 (T14). Using two independent approaches for objective assessment (see Supplementary methods), it was found that the indels novel to T14 did not exhibit homology to any of the 7 sgRNAs in 480-MT7 (Tables 22-23). These indels, present at low VAF, likely represent background heterogeneity in a bulk cell population or ongoing genomic instability.

TABLE 23
Analysis of indels that were present in T14 from WGS analyses.

| | | Sequencing | | | Reference sequence |
|---|---|---|---|---|---|
| Total number | | artifact in | Mutation | Novel | shared >5 bp |
| detected by | Present | repetitive | not present | indel | homology with |
| Mutect2 | in T0 | regions | in IGV | in T14 | sgRNA |
| 212 | 132/212 | 49/212 | 6/212 | 25/212 | 0/25 |

Panc480-Cas9-mApple cells were co-cultured along with Panc10.05-Cas9-EGFP cells and transduced them with MT7. Flow cytometry showed >80% selective reduction of Panc480 cells on day 21 (FIG. 17D; paired t test, P=0.003), and this finding was corroborated with STR profiling (FIG. 17E; paired t test, P=0.03). Although selective reduction was also seen in Panc480 parental cell line lacking Cas9 (FIG. 17E; paired t test, P=0.009), the magnitude of reduction in the presence of Cas9 was larger (76.4% vs 59.6%). This suggests the MT7 expression vector itself was somewhat toxic, but that functional Cas9 was needed to produce the full observed toxicity (FIG. 17D-17E). These results demonstrated that the sgRNAs designed via PAM discovery approach were able to yield significant cell death of targeted cells.

### Results

The above demonstrates a highly efficient cancer-specific PAM discovery approach that allows selective killing of cancer cells. This data demonstrates that in PCs which generally have low mutational burden, >400 novel PAMs could be identified as candidates for CRISPR-Cas9 targeting, significantly expanding the repertoire of targetable mutations in a given solid tumor. Since point mutations increase as a function of age (72, 66) and this mutational signature analyses revealed that most of these mutations showed clock-like signatures, these findings suggest that adult solid tumors, in general, would produce hundreds of novel PAMs, more than enough for subsequent screening and selection of sgRNAs. This was corroborated by studies in esophageal and lung cancers which revealed thousands of somatic PAMs, indicating that additional tissue-dependent factors, likely environmental, could increase the number of somatic PAMs. While it is conceivable that pediatric tumors might not contain as many somatic PAMs as adult patients, it was found that <10 sgRNAs are required to achieve significant toxicity, demonstrating that not many sgRNAs would be needed to achieve selective killing and provide therapeutic window for other modalities.

The approach described above exploits the vast number of novel PAMs located in noncoding regions, it requires WGS analyses of both tumor and normal. The approach described herein is cancer- and, patient-specific. This approach presents a unique opportunity as a new precision medicine-based therapeutic tool that possesses the specificity of a targeted therapy, but without the restriction of a targetable protein. As cancer is a clonal disease, the distinct set of mutations found in the cancer initiating cell should be present in all primary tumor and metastatic sites, thus making this approach a potential solution to multi-site cancer killing.

### Clauses

Clause 1. A CRISPR-Cas9 system for treating a disease, disorder, or condition associated with one or more somatic mutations in a subject in need of treatment thereof, the system comprising a sgRNA, wherein the sgRNA targets between about 1 to about 50 mutations in a target cell.

Clause 2. The CRISPR-Cas9 system of clause 1, wherein the sgRNA is designed as a multi-target sgRNA which is both patient-specific and cancer-specific.

Clause 3. The CRISPR-Cas9 system of clause 1, wherein the sgRNA is selected from the group consisting of NT, NT2, HPRTc.80, HPRTc.465, 531F(2), 52F(3), 715F(5), 451F(6), 176R(7), 551R(8), 230F(12), 164R(14), 676F(16), AGGn, L1.4_209F, and ALU_112a. Clause 4. The CRISPR-Cas9 system of clause 3, wherein the NT has the sequence of SEQ ID NO:1.

Clause 5. The CRISPR-Cas9 system of clause 3, wherein the NT2 has the sequence of SEQ ID NO:2.

Clause 6. The CRISPR-Cas9 system of clause 3, wherein the HPRTc.80 has the sequence of SEQ ID NO:3.

Clause 7. The CRISPR-Cas9 system of clause 3, wherein the HPRTc.465 has the sequence of SEQ ID NO:4.

Clause 8. The CRISPR-Cas9 system of clause 3, wherein the 531F(2) has the sequence of SEQ ID NO:5.

Clause 9. The CRISPR-Cas9 system of clause 3, wherein the 52F(3) has the sequence of SEQ ID NO:6.

Clause 10. The CRISPR-Cas9 system of clause 3, wherein the 715F(5) has the sequence of SEQ ID NO:7.

Clause 11. The CRISPR-Cas9 system of clause 3, wherein the 451F(6) has the sequence of SEQ ID NO:8.

Clause 12. The CRISPR-Cas9 system of clause 3, wherein the 176R(7) has the sequence of SEQ ID NO:9.

Clause 13. The CRISPR-Cas9 system of clause 3, wherein the 551R(8) has the sequence of SEQ ID NO:10.

Clause 14. The CRISPR-Cas9 system of clause 3, wherein the 230F(12) has the sequence of SEQ ID NO:11.

Clause 15. The CRISPR-Cas9 system of clause 3, wherein the 164R(14) has the sequence of SEQ ID NO:12.

Clause 16. The CRISPR-Cas9 system of clause 3, wherein the 676F has the sequence of SEQ ID NO:13.

Clause 17. The CRISPR-Cas9 system of clause 3, wherein the AGGn has the sequence of SEQ ID NO:14.

Clause 18. The CRISPR-Cas9 system of clause 3, wherein the L1.4_209F has the sequence of SEQ ID NO:15.

Clause 19. The CRISPR-Cas9 system of clause 3, wherein the ALU_112a has the sequence of SEQ ID NO:16.

Clause 20. The CRISPR-Cas9 system of clause 1, wherein the sgRNA targets at least 12 mutations in the target cell.

Clause 21. The CRISPR-Cas9 system of clause 1, wherein the mutation is in the non-coding region of the target cell.

Clause 22. The CRISPR-Cas9 system of clause 1, wherein the disease, disorder, or condition associated with one or more somatic mutations is a cancer, an autoimmune disease, or a neurodegenerative disease.

Clause 23. The CRISPR-Cas9 system of clause 22, wherein the cancer is pancreatic cancer.

Clause 24. The CRISPR-Cas9 system of clause 22, wherein the cancer is metastatic cancer.

Clause 25. An sgRNA of clauses 3-19.

Clause 26. The sgRNA of clause 25, wherein the sgRNA is designed as a multi-target sgRNA which is both patient-specific and cancer-specific.

Clause 27. A method for treating a disease, disorder, or condition associated with one or more somatic mutations in a subject in need of treatment thereof, the method comprising administering an effective amount of the CRISPR-Cas9 system of any one of clauses 1-24 to a target cell of the subject in need of treatment thereof.

Clause 28. The method of clause 27, wherein the disease, disorder, or condition comprises a cancer, an autoimmune disease, or a neurodegenerative disease.

Clause 29. The method of clause 28, wherein the cancer is pancreatic cancer.

Clause 30. The method of clause 28, wherein the cancer is metastatic cancer.

Clause 31. The method of clause 27, wherein administering the CRISPR-Cas9 system to the target cell induces multiple double-strand breaks.

Clause 32. The method of clause 27, wherein the CRISPR-Cas9 system is delivered via a viral vector.

Clause 33. The method of clause 32, wherein the viral vector is selected from an adenovirus, adeno-associated virus, retrovirus, lentivirus, Newcastle disease virus (NDV), and lymphocytic choriomeningitis virus (LCMV).

Clause 34. The method of clause 27, wherein the subject is a mammalian subject.

Clause 35. The method of clause 34, wherein the mammalian subject is a human subject.

Clause 36. A kit comprising the CRISPR-Cas9 system of any one of clauses 1-24.

Clause 37. A method for identifying novel protospacer adjacent motifs (PAMs), novel target sites, or novel PAMs and novel target sites in cells of a sample obtained from a subject, the method comprising:
a) analyzing sequencing data from one or more cells obtained from the subject for one or more somatic single base substitutions (SBS), one or more structural variants (SV), or one or more SBS and SVs that produce a PAM, a target site, or a PAM and a target site; and
b) identifying one or more PAMs, target sites, or PAMs and target sites in the cells based on the analysis in step a).

Clause 38. The method of clause 37, wherein the one or more cells is a cancer cell.

Clause 39. The method of clause 38, wherein the cancer cell is a cancer initiating cell.

Clause 40. The method of clause 37, wherein the sequencing data is whole genome sequencing data.

Clause 41. The method of any of clauses 37 to 40, wherein the subject has cancer.

Clause 42. A method of treating a disease, disorder or a condition in a subject, the method comprising:
a) analyzing sequencing data from one or more cells of a sample obtained from a subject suffering from a disease, disorder, or a condition, for one or more somatic single base substitutions (SBS), one or more structural variants (SV), or one or more SBS and SVs that produce a PAM, a target site, or a PAM and a target site;
b) identifying one or more PAMs, target sites, or PAMs and target sites in the cells based on the analysis in step a); and
c) administering to the subject an effective amount of a CRISPR-Cas9 system comprising a sgRNA, wherein the sgRNA targets (i) a sequence adjacent to the PAM; (ii) the target site; or (iii) combinations of (i) and (ii).

Clause 43. The method of clause 42, wherein the one or more cells is a cancer cell.

Clause 44. The method of clause 43, wherein the cancer cell is a cancer initiating cell.

Clause 45. The method of clause 42, wherein the sequencing data is whole genome sequencing data.

Clause 46. A method of treating a subject suffering from a disease, disorder or a condition, the method comprising:
a) identifying one or more single somatic single base substitutions (SBS), one or more structural variants (SV), or one or more SBS and SVs that produce a PAM, a target site, or a PAM and a target site in one or more cells of a sample obtained from a subject suffering from a disease, disorder, or a condition; and
b) administering to the subject an effective amount of a CRISPR-Cas9 system comprising a sgRNA, wherein the sgRNA targets (i) a sequence adjacent to the PAM; (ii) the target site; or (iii) combinations of (i) and (ii).

Clause 47. The method of clause 46, wherein the one or more cells is a cancer cell.

Clause 48. The method of clause 47, wherein the cancer cell is a cancer initiating cell.

Clause 49. The method of any of clauses 46-48, wherein the disease is cancer.

Clause 50. The method of any of clauses 46-49, wherein the method further comprises monitoring the subject receiving treatment with the CRISPR-Cas9 system.

Clause 51. A method of treating a subject suffering from a disease, disorder, or condition, the method comprising:
a) obtaining a sample from a subject suffering from a disease, disorder, or condition that is receiving treatment with a CRISPR-Cas system comprising a sgRNA that has developed resistance to said treatment;
b) identifying one or more single somatic single base substitutions (SBS), one or more structural variants (SV), or one or more SBS and SVs that were not previously identified in the subject and that produce a PAM, a target site, or a PAM and a target site in one or more cells of a sample obtained from the subject and that is different than the PAM and/or target site previously identified in the subject; and
c) administering to the subject an effective amount of a CRISPR-Cas9 system comprising a sgRNA, wherein the sgRNA targets (i) a sequence adjacent to the PAM; (ii) the target site; or (iii) combinations of (i) and (ii) identified in step b).

Clause 52. The method of clause 51, wherein the one or more cells is a cancer cell.

Clause 53. The method of clause 51, wherein the cancer cell is a cancer initiating cell.

Clause 54. The method of any of clauses 51-53, wherein the disease is cancer.

Clause 55. The method of any of clauses 51-54, wherein the method further comprises monitoring the subject receiving treatment with the CRISPR-Cas9 system.

Clause 56. A method of identifying somatic mutations in a tumor that produce a protospacer adjacent motif (PAM) in a subject, the method comprising the steps of:
a. obtaining from a subject having at least one tumor: i) at least one sample from the tumor; and ii) at least one non-tumor sample;
b. obtaining DNA from the tumor sample and from the non-tumor sample;
c. performing next generation sequencing of DNA obtained from the tumor sample and the normal sample to produce a tumor sequence and a normal sequence;
d. aligning the tumor sequence and the normal sequence; and
e. identifying one or more somatic mutations in the tumor sequence that produce one or more PAMs.

Clause 57. The method of clause 56, wherein the tumor sample is a tissue sample, a blood sample, a plasma sample, a serum sample, an urine sample, cerebrospinal fluid, stool or feces, saliva, ascites fluid, sputum, synovial fluid, or any combination thereof.

Clause 58. The method of clause 56 or clause 57, wherein the non-tumor sample is a tissue sample, a blood sample, a plasma sample, a serum sample, an urine sample, cerebrospinal fluid, stool or feces, saliva, ascites fluid, sputum, synovial fluid, or any combination thereof.

Clause 59. The method of any of causes 56-58, wherein the identifying of one or more somatic mutations in the tumor sequence involves identifying one or more single somatic base substitutions (BS), one or more structural variants (SV), or one or more BS and SVs that produce one or more PAMs.

Clause 60. The method of any of clauses 56-59, wherein the tumor is cancer.

Clause 61. The method of any of clauses 56-60, wherein the cancer is pancreatic cancer, lung cancer, esophageal cancer, or any combinations thereof.

Clause 62. The method of any of clauses 56-61, wherein the next generation sequencing is whole genome sequencing.

Clause 63. A method of designing a CRISPR-Cas 9 system to target protospacer adjacent motifs (PAMs) identified in a tumor sample obtained from a subject, the method comprising:
a. obtaining from a subject having a tumor: i) at least one sample from the tumor; and ii) at least one non-tumor sample;
b. obtaining DNA from the tumor sample and from the non-tumor sample;
c. performing next generation sequencing of DNA obtained from the tumor cell line and the normal cell line to produce a tumor sequence and a normal sequence;
d. aligning the tumor sequence and the normal sequence;
e. identifying one or more somatic mutations in the tumor sequence that produce one or more PAMs;
f. designing one or more CRISPR-Cas9 systems, wherein the CRISPR-Cas9 system comprises one or more sgRNAs that target a sequence adjacent to one or more PAMs.

Clause 64. The method of clause 63, wherein the tumor sample is a tissue sample, a blood sample, a plasma sample, a serum sample, an urine sample, cerebrospinal fluid, stool or feces, saliva, ascites fluid, sputum, synovial fluid, or any combination thereof.

Clause 65. The method of clause 63 or clause 64, wherein the non-tumor sample is a tissue sample, a blood sample, a plasma sample, a serum sample, an urine sample, cerebrospinal fluid, stool or feces, saliva, ascites fluid, sputum, synovial fluid, or any combination thereof.

Clause 66. The method of any of clauses 63-65, wherein the identifying of one or more somatic mutations in the tumor sequence involves identifying one or more single somatic base substitutions (BS), one or more structural variants (SV), or one or more BS and SVs that produce one or more PAMs.

Clause 67. The method of any of clauses 63-66, wherein the tumor is cancer.

Clause 68. The method of any of clauses 63-67, wherein the cancer is pancreatic cancer, lung cancer, esophageal cancer, or any combinations thereof.

Clause 69. The method of any of clauses 63-68, wherein the method further comprises confirming that the sgRNA of step f) target somatic mutations contained in the tumor.

Clause 70. The method of any of clauses 63-69, wherein the next generation sequencing is whole genome sequencing.

Clause 71. A method of treating a subject suffering from pancreatic cancer, lung cancer, esophageal cancer, or any combination thereof, the method comprising administering to the subject a therapeutically effective amount of the CRISPR-Cas9 system designed according to any of clauses 63-70.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
1. F. Blokzijl et al., Tissue-specific mutation accumulation in human adult stem cells during life. Nature 538, 260-264 (2016).
2. P. C. Nowell, The clonal evolution of tumor cell populations. Science 194, 23-28 (1976).
3. E. R. Fearon, B. Vogelstein, A genetic model for colorectal tumorigenesis. Cell 61, 759-767 (1990).
4. C. Tomasetti, B. Vogelstein, G. Parmigiani, Half or more of the somatic mutations in cancers of self-renewing tissues originate prior to tumor initiation. Proc Natl Acad Sci USA 110, 1999-2004 (2013).
5. M. Jinek et al., A programmable dual-RNA-guided DNA endonuclease in adaptive bacterial immunity. Science 337, 816-821 (2012).
6. L. Cong et al., Multiplex genome engineering using CRISPR/Cas systems. Science 339, 819-823 (2013).
7. P. Mali et al., RNA-guided human genome engineering via Cas9. Science 339, 823-826 (2013).
8. Y. Fu et al., High-frequency off-target mutagenesis induced by CRISPR-Cas nucleases in human cells. Nat Biotechnol 31, 822-826 (2013).
9. G. Alanis-Lobato et al., Frequent loss of heterozygosity in CRISPR-Cas9-edited early human embryos. Proc Natl Acad Sci USA 118, (2021).
10. M. Haeussler et al., Evaluation of off-target and on-target scoring algorithms and integration into the guide RNA selection tool CRISPOR. Genome Biol 17, 148 (2016).
11. R. Graf, X. Li, V. T. Chu, K. Rajewsky, sgRNA Sequence Motifs Blocking Efficient CRISPR/Cas9-Mediated Gene Editing. Cell Rep 26, 1098-1103 e1093 (2019).
12. T. Wang, J. J. Wei, D. M. Sabatini, E. S. Lander, Genetic screens in human cells using the CRISPR-Cas9 system. Science 343, 80-84 (2014).
13. O. Shalem et al., Genome-scale CRISPR-Cas9 knockout screening in human cells. Science 343, 84-87 (2014).
14. R. S. Zou et al., Massively parallel genomic perturbations with multi-target CRISPR interrogates Cas9 activity and DNA repair at endogenous sites. Nat Cell Biol 24, 1433-1444 (2022).
15. X. Chen et al., Manta: rapid detection of structural variants and indels for germline and cancer sequencing applications. Bioinformatics 32, 1220-1222 (2016).
16. E. Papp et al., Integrated Genomic, Epigenomic, and Expression Analyses of Ovarian Cancer Cell Lines. Cell Rep 25, 2617-2633 (2018).
17. M. T. Lin et al., Quantifying the relative amount of mouse and human DNA in cancer xenografts using species-specific variation in gene length. Biotechniques 48, 211-218 (2010).
18. S. R. Hingorani et al., Trp53R172H and KrasG12D cooperate to promote chromosomal instability and widely metastatic pancreatic ductal adenocarcinoma in mice. Cancer Cell 7, 469-483 (2005).
19. D. Hanahan, R. A. Weinberg, Hallmarks of cancer: the next generation. Cell 144, 646-674 (2011).
20. C. J. Tokheim, N. Papadopoulos, K. W. Kinzler, B. Vogelstein, R. Karchin, Evaluating the evaluation of cancer driver genes. Proc Natl Acad Sci USA 113, 14330-14335 (2016).
21. M. Gerstung et al., The evolutionary history of 2,658 cancers. Nature 578, 122-128 (2020).
22. S. Yachida et al., Distant metastasis occurs late during the genetic evolution of pancreatic cancer. Nature 467, 1114-1117 (2010).
23. C. Shi et al., Anti-gene padlocks eliminate Escherichia coli based on their genotype. J Antimicrob Chemother 61, 262-272 (2008).
24. Z. H. Chen et al., Targeting genomic rearrangements in tumor cells through Cas9-mediated insertion of a suicide gene. Nat Biotechnol 35, 543-550 (2017).
25. L. Jubair, A. K. Lam, S. Fallaha, N. A. J. McMillan, CRISPR/Cas9-loaded stealth liposomes effectively cleared established HPV16-driven tumours in syngeneic mice. PLOS One 16, e0223288 (2021).
26. T. Kwon et al., Precision targeting tumor cells using cancer-specific InDel mutations with CRISPR-Cas9. Proc Natl Acad Sci USA 119, (2022).
27. W. Kim et al., Targeting mutant KRAS with CRISPR-Cas9 controls tumor growth. Genome Res, (2018).
28. D. M. Munoz et al., CRISPR Screens Provide a Comprehensive Assessment of Cancer Vulnerabilities but Generate False-Positive Hits for Highly Amplified Genomic Regions. Cancer Discov 6, 900-913 (2016).
29. L. Sansregret, B. Vanhaesebroeck, C. Swanton, Determinants and clinical implications of chromosomal instability in cancer. Nat Rev Clin Oncol 15, 139-150 (2018).
30. S. M. Dewhurst, Chromothripsis and telomere crisis: engines of genome instability. Curr Opin Genet Dev 60, 41-47 (2020).
31. T. Davoli, T. de Lange, Telomere-driven tetraploidization occurs in human cells undergoing crisis and promotes transformation of mouse cells. Cancer Cell 21, 765-776 (2012).
32. A. L. Norris et al., Familial and sporadic pancreatic cancer share the same molecular pathogenesis. Fam Cancer 14, 95-103 (2015).
33. T. T. Seppala et al., Patient-derived Organoid Pharmacotyping is a Clinically Tractable Strategy for Precision Medicine in Pancreatic Cancer. Ann Surg 272, 427-435 (2020).
34. J. D. Gillmore et al., CRISPR-Cas9 In Vivo Gene Editing for Transthyretin Amyloidosis. N Engl J Med 385, 493-502 (2021).
35. J. P. Concordet, M. Haeussler, CRISPOR: intuitive guide selection for CRISPR/Cas9 genome editing experiments and screens. Nucleic Acids Res 46, W242-W245 (2018).
36. J. G. Doench et al., Optimized sgRNA design to maximize activity and minimize off-target effects of CRISPR-Cas9. Nat Biotechnol 34, 184-191 (2016).
37. S. H. Chiou et al., Pancreatic cancer modeling using retrograde viral vector delivery and in vivo CRISPR/Cas9-mediated somatic genome editing. Genes Dev 29, 1576-1585 (2015).
38. G. v. d. Auwera, B. D. O'Connor, Genomics in the cloud: using Docker, GATK, and WDL in Terra. (O'Reilly Media, Sebastopol, CA, ed. First edition., 2020), pp. xxiv, 467 pages.
39. J. T. Robinson et al., Integrative genomics viewer. Nat Biotechnol 29, 24-26 (2011).
40. P. Cingolani et al., A program for annotating and predicting the effects of single nucleotide polymorphisms, SnpEff: SNPs in the genome of Drosophila melanogaster strain w1118; iso-2; iso-3. Fly (Austin) 6, 80-92 (2012).
41. L. Jiang et al., Clinical Utility of Targeted Next-Generation Sequencing Assay to Detect Copy Number Variants Associated with Myelodysplastic Syndrome in Myeloid Malignancies. J Mol Diagn 23, 467-483 (2021).
42. J. Joung et al., Genome-scale CRISPR-Cas9 knockout and transcriptional activation screening. Nat Protoc 12, 828-863 (2017).
43. W. Li et al., MAGeCK enables robust identification of essential genes from genome-scale CRISPR/Cas9 knockout screens. Genome Biol 15, 554 (2014).
44. B. Daniel, M. A. DeCoster, Quantification of sPLA2-induced early and late apoptosis changes in neuronal cell cultures using combined TUNEL and DAPI staining. Brain Res Brain Res Protoc 13, 144-150 (2004).
45. Y. Jiao et al., DAXX/ATRX, MEN1, and mTOR pathway genes are frequently altered in pancreatic neuroendocrine tumors. Science 331, 1199-1203 (2011).
46. N. J. Roberts et al., Whole Genome Sequencing Defines the Genetic Heterogeneity of Familial Pancreatic Cancer. Cancer Discov 6, 166-175 (2016).
47. A. R. Quinlan, I. M. Hall, BEDTools: a flexible suite of utilities for comparing genomic features. Bioinformatics 26, 841-842 (2010).
48. K. Wang, M. Li, H. Hakonarson, ANNOVAR: functional annotation of genetic variants from high-throughput sequencing data. Nucleic Acids Res 38, e164 (2010).
49. D. Karolchik et al., The UCSC Table Browser data retrieval tool. Nucleic Acids Res 32, D493-496 (2004).
50. A. M. Meynert, M. Ansari, D. R. FitzPatrick, M. S. Taylor, Variant detection sensitivity and biases in whole genome and exome sequencing. BMC Bioinformatics 15, 247 (2014).
51. H. Li, R. Durbin, Fast and accurate short read alignment with Burrows-Wheeler transform. Bioinformatics 25, 1754-1760 (2009).
52. C. Pulido-Quetglas et al., Scalable Design of Paired CRISPR Guide RNAs for Genomic Deletion. PLOS Comput Biol 13, e1005341 (2017).
53. A. E. Campbell et al., NuRD and CAF-1-mediated silencing of the D4Z4 array is modulated by DUX4-induced MBD3L proteins. Elife 7, (2018).
54. N. C. Shaner et al., Improving the photostability of bright monomeric orange and red fluorescent proteins. Nat Methods 5, 545-551 (2008).
55. N. E. Sanjana, O. Shalem, F. Zhang, Improved vectors and genome-wide libraries for CRISPR screening. Nat Methods 11, 783-784 (2014).
56. B. W. Stringer et al., A reference collection of patient-derived cell line and xenograft models of proneural, classical and mesenchymal glioblastoma. Sci Rep 9, 4902 (2019).
57. S. A. Stewart et al., Lentivirus-delivered stable gene silencing by RNAi in primary cells. RNA 9, 493-501 (2003).
58. T. Dull et al., A third-generation lentivirus vector with a conditional packaging system. J Virol 72, 8463-8471 (1998).
59. K. Clement et al., CRISPResso2 provides accurate and rapid genome editing sequence analysis. Nat Biotechnol 37, 224-226 (2019).
60. B. Langmead, S. L. Salzberg, Fast gapped-read alignment with Bowtie 2. Nat Methods 9, 357-359 (2012).
61. Mojica F J M, Díez-Villaseñor C, García-Martínez J, Almendros C. Short motif sequences determine the targets of the prokaryotic CRISPR defence system. Microbiology. 2009; 155:733-40.
62. Cancer of the Pancreas-Cancer Stat Facts [Internet]. SEER. [cited 2023 Feb. 7]. Available from: https://seer.cancer.gov/statfacts/html/pancreas.html
63. Alexandrov L B, Kim J, Haradhvala N J, Huang M N, Tian Ng A W, Wu Y, et al. The repertoire of mutational signatures in human cancer. Nature. 2020; 578:94-101.
64. Alexandrov L B, Nik-Zainal S, Wedge D C, Aparicio S A J R, Behjati S, Biankin A V, et al. Signatures of mutational processes in human cancer. Nature. 2013; 500:415-21.
65. Nik-Zainal S, Alexandrov L B, Wedge D C, Van Loo P, Greenman C D, Raine K, et al. Mutational processes molding the genomes of 21 breast cancers. Cell. 2012; 149:979-93.
66. ICGC/TCGA Pan-Cancer Analysis of Whole Genomes Consortium. Pan-cancer analysis of whole genomes. Nature. 2020; 578:82-93.
67. Van der Auwera G A, O'Connor B D. Genomics in the Cloud. O'Reilly Media, Inc.
68. Christensen S, Van der Roest B, Besselink N, Janssen R, Boymans S, Martens J W M, et al. 5-Fluorouracil treatment induces characteristic T>G mutations in human cancer. Nat Commun. 2019; 10:4571.
69. Secrier M, Li X, de Silva N, Eldridge M D, Contino G, Bornschein J, et al. Mutational signatures in esophageal adenocarcinoma define etiologically distinct subgroups with therapeutic relevance. Nat Genet. 2016; 48:1131-41.
70. Noorani A, Bornschein J, Lynch A G, Secrier M, Achilleos A, Eldridge M, et al. A comparative analysis of whole genome sequencing of esophageal adenocarcinoma pre- and post-chemotherapy. Genome Res. 2017; 27:902-12.
71. Kris A. Wetterstrand M S. DNA Sequencing Costs: Data [Internet]. Genome.gov. NHGRI; 2019 [cited 2023 Feb. 14]. Available from: https://www.genome.gov/about-genomics/fact-sheets/DNA-Sequencing-Costs-Data
72. Blokzijl F, de Ligt J, Jager M, Sasselli V, Roerink S, Sasaki N, et al. Tissue-specific mutation accumulation in human adult stem cells during life. Nature. 2016; 538:260-4.
73. Li H. Aligning sequence reads, clone sequences and assembly contigs with BWA-MEM [Internet]. arXiv [q-bio.GN]. 2013. Available from: http://arxiv.org/abs/1303.3997
74. Cingolani P, Platts A, Wang L L, Coon M, Nguyen T, Wang L, et al. A program for annotating and predicting the effects of single nucleotide polymorphisms, SnpEff: SNPs in the genome of Drosophila melanogaster strain w1118; iso-2; iso-3. Fly. 2012; 6:80-92.
75. Meynert A M, Ansari M, FitzPatrick D R, Taylor M S. Variant detection sensitivity and biases in whole genome and exome sequencing. BMC Bioinformatics. 2014; 15:247.
76. Concordet J-P, Haeussler M. CRISPOR: intuitive guide selection for CRISPR/Cas9 genome editing experiments and screens. Nucleic Acids Res. 2018; 46: W242-5.
77. International Cancer Genome Consortium, Hudson T J, Anderson W, Artez A, Barker A D, Bell C, et al. International network of cancer genome projects. Nature. 2010; 464:993-8.
78. Clement K, Rees H, Canver M C, Gehrke J M, Farouni R, Hsu J Y, et al. CRISPResso2 provides accurate and rapid genome editing sequence analysis. Nat Biotechnol. 2019; 37:224-6.
79. Norris A L, Kamiyama H, Makohon-Moore A, Pallavajjala A, Morsberger L A, Lee K, et al. Transflip mutations produce deletions in pancreatic cancer. Genes Chromosomes Cancer. 2015; 54:472-81.

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A method of identifying somatic mutations in a tumor that produce a protospacer adjacent motif (PAM) in a subject, the method comprising the steps of:
a. obtaining from a subject having at least one tumor: i) at least one sample from the tumor; and ii) at least one non-tumor sample;
b. obtaining DNA from the tumor sample and from the non-tumor sample;
c. performing next generation sequencing of DNA obtained from the tumor sample and the normal sample to produce a tumor sequence and a normal sequence;
d. aligning the tumor sequence and the normal sequence; and
e. identifying one or more somatic mutations in the tumor sequence that produce one or more PAMs.

2. The method of claim 1, wherein the tumor sample is a tissue sample, a blood sample, a plasma sample, a serum sample, an urine sample, cerebrospinal fluid, stool or feces, saliva, ascites fluid, sputum, synovial fluid, or any combination thereof.

3. The method of claim 1, wherein the non-tumor sample is a tissue sample, a blood sample, a plasma sample, a serum sample, an urine sample, cerebrospinal fluid, stool or feces, saliva, ascites fluid, sputum, synovial fluid, or any combination thereof.

4. The method of claim 1, wherein the identifying of one or more somatic mutations in the tumor sequence involves identifying one or more single somatic base substitutions (BS), one or more structural variants (SV), or one or more BS and SVs that produce one or more PAMs.

5. The method of claim 1, wherein the tumor is cancer.

6. The method of claim 1, wherein the cancer is pancreatic cancer, lung cancer, esophageal cancer, or any combinations thereof.

7. The method of claim 1, wherein the next generation sequencing is whole genome sequencing.

8. A method of designing a CRISPR-Cas 9 system to target protospacer adjacent motifs (PAMs) identified in a tumor sample obtained from a subject, the method comprising:
a. obtaining from a subject having a tumor: i) at least one sample from the tumor; and ii) at least one non-tumor sample;
b. obtaining DNA from the tumor sample and from the non-tumor sample;
c. performing next generation sequencing of DNA obtained from the tumor cell line and the normal cell line to produce a tumor sequence and a normal sequence;
d. aligning the tumor sequence and the normal sequence;
e. identifying one or more somatic mutations in the tumor sequence that produce one or more PAMs;
f. designing one or more CRISPR-Cas9 systems, wherein the CRISPR-Cas9 system comprises one or more sgRNAs that target a sequence adjacent to one or more PAMs.

9. The method of claim 8, wherein the tumor sample is a tissue sample, a blood sample, a plasma sample, a serum sample, an urine sample, cerebrospinal fluid, stool or feces, saliva, ascites fluid, sputum, synovial fluid, or any combination thereof.

10. The method of claim 8, wherein the non-tumor sample is a tissue sample, a blood sample, a plasma sample, a serum sample, an urine sample, cerebrospinal fluid, stool or feces, saliva, ascites fluid, sputum, synovial fluid, or any combination thereof.

11. The method of claim 8, wherein the identifying of one or more somatic mutations in the tumor sequence involves identifying one or more single somatic base substitutions (BS), one or more structural variants (SV), or one or more BS and SVs that produce one or more PAMs.

12. The method of claim 8, wherein the tumor is cancer.

13. The method of claim 8, wherein the cancer is pancreatic cancer, lung cancer, esophageal cancer, or any combinations thereof.

14. The method of claim 8, wherein the method further comprises confirming that the sgRNA of step f) target somatic mutations contained in the tumor.

15. The method of claim 8, wherein the next generation sequencing is whole genome sequencing.

16. A method of treating a subject suffering from pancreatic cancer, lung cancer, esophageal cancer, or any combination thereof, the method comprising administering to the subject a therapeutically effective amount of the CRISPR-Cas9 system designed according to claim 8.


SEQUENCE LISTING (474 sequences)

SEQ ID NO: 1 - RNA, 20 nt, Synthetic construct
  source 1..20: mol_type = other RNA
gtattactgatattggtggg

SEQ ID NO: 2 - RNA, 20 nt, Synthetic construct
  source 1..20: mol_type = other RNA
gcgaggtattcggctccgcg

SEQ ID NO: 3 - RNA, 20 nt, Synthetic construct
  source 1..20: mol_type = other RNA
attatgctgaggatttggaa

SEQ ID NO: 4 - RNA, 20 nt, Synthetic construct
  source 1..20: mol_type = other RNA
tggattatactgcctgacca

SEQ ID NO: 5 - RNA, 20 nt, Synthetic construct
  source 1..20: mol_type = other RNA
cactcagcatcgacttacga

SEQ ID NO: 6 - RNA, 20 nt, Synthetic construct
  source 1..20: mol_type = other RNA
taattactgcacgatgcgca

SEQ ID NO: 7 - RNA, 20 nt, Synthetic construct
  source 1..20: mol_type = other RNA
atatatatgcgatcgagccc

SEQ ID NO: 8 - RNA, 20 nt, Synthetic construct
  source 1..20: mol_type = other RNA
actagtgtgcgtatgatttg

SEQ ID NO: 9 - RNA, 20 nt, Synthetic construct
  source 1..20: mol_type = other RNA
tcgatgttctacatcgatgt

SEQ ID NO: 10 - RNA, 20 nt, Synthetic construct
  source 1..20: mol_type = other RNA
ttgaattgagttgcaaccga

SEQ ID NO: 11 - RNA, 20 nt, Synthetic construct
  source 1..20: mol_type = other RNA
ttgtcccacaatgatacttg

SEQ ID NO: 12 - RNA, 20 nt, Synthetic construct
  source 1..20: mol_type = other RNA
ggatatttcactacagactt

SEQ ID NO: 13 - RNA, 20 nt, Synthetic construct
  source 1..20: mol_type = other RNA
ctccgaacttaacttgccct

SEQ ID NO: 14 - RNA, 20 nt, Synthetic construct
  source 1..20: mol_type = other RNA
aggaggaggaggaggaggag

SEQ ID NO: 15 - RNA, 20 nt, Synthetic construct
  source 1..20: mol_type = other RNA
tgcctcacctgggaagcgca

SEQ ID NO: 16 - RNA, 20 nt, Synthetic construct
  source 1..20: mol_type = other RNA
ttgcccaggctggagtgcag

SEQ ID NO: 17 - skipped

SEQ ID NO: 18 - RNA, 34 nt, Synthetic construct
  source 1..34: mol_type = other RNA
gtaaaacgacggccagctctttggctgatgttcc

SEQ ID NO: 19 - RNA, 36 nt, Synthetic construct
  source 1..36: mol_type = other RNA
gtaaaacgacggccagaagaatcgcctgaacctggg

SEQ ID NO: 20 - RNA, 34 nt, Synthetic construct
  source 1..34: mol_type = other RNA
gtaaaacgacggccagtctcaaagttacacgtca

SEQ ID NO: 21 - RNA, 36 nt, Synthetic construct
  source 1..36: mol_type = other RNA
gtaaaacgacggccagactaccactccttcatcccc

SEQ ID NO: 22 - RNA, 37 nt, Synthetic construct
  source 1..37: mol_type = other RNA
gtaaaacgacggccaggttcaccatcttagccacagg

SEQ ID NO: 23 - RNA, 34 nt, Synthetic construct
  source 1..34: mol_type = other RNA
gtaaaacgacggccagaaagagtgtgacggaggg

SEQ ID NO: 24 - RNA, 36 nt, Synthetic construct
  source 1..36: mol_type = other RNA
gtaaaacgacggccagtatttgatgagggccagtgc

SEQ ID NO: 25 - RNA, 34 nt, Synthetic construct
  source 1..34: mol_type = other RNA
gtaaaacgacggccagagtggcatggaacagatt

SEQ ID NO: 26 - RNA, 36 nt, Synthetic construct
  source 1..36: mol_type = other RNA
gtaaaacgacggccagttaccaaagttccccaggtg

SEQ ID NO: 27 - RNA, 36 nt, Synthetic construct
  source 1..36: mol_type = other RNA
gtaaaacgacggccaggaggcaggcatggaaagtta

SEQ ID NO: 28 - RNA, 36 nt, Synthetic construct
  source 1..36: mol_type = other RNA
gtaaaacgacggccagagcaggctttatgccacatc

SEQ ID NO: 29 - RNA, 36 nt, Synthetic construct
  source 1..36: mol_type = other RNA
gtaaaacgacggccaggccacatctttcccattcaa

SEQ ID NO: 30 - RNA, 34 nt, Synthetic construct
  source 1..34: mol_type = other RNA
gtaaaacgacggccagttaaagcttttggacttt

SEQ ID NO: 31 - RNA, 36 nt, Synthetic construct
  source 1..36: mol_type = other RNA
gtaaaacgacggccagattctacctgttcagggccc

SEQ ID NO: 32 - RNA, 34 nt, Synthetic construct
  source 1..34: mol_type = other RNA
gtaaaacgacggccagttcaactaggtaggtctc

SEQ ID NO: 33 - RNA, 36 nt, Synthetic construct
  source 1..36: mol_type = other RNA
gtaaaacgacggccagtgaaagatgcaatgctcctg

SEQ ID NO: 34 - RNA, 34 nt, Synthetic construct
  source 1..34: mol_type = other RNA
gtaaaacgacggccagctgagcttattctcagac

SEQ ID NO: 35 - RNA, 34 nt, Synthetic construct
  source 1..34: mol_type = other RNA
gtaaaacgacggccagcgatctcaaatcaaactc

SEQ ID NO: 36 - RNA, 37 nt, Synthetic construct
  source 1..37: mol_type = other RNA
gtaaaacgacggccaggggcatacagggacaattcac

SEQ ID NO: 37 - RNA, 36 nt, Synthetic construct
  source 1..36: mol_type = other RNA
gtaaaacgacggccagggttccacagtaacccagca

SEQ ID NO: 38 - RNA, 37 nt, Synthetic construct; identical to SEQ ID NO: 36
  source 1..37: mol_type = other RNA

SEQ ID NO: 39 - RNA, 34 nt, Synthetic construct
  source 1..34: mol_type = other RNA
gtaaaacgacggccagttttctgctgataatttc

SEQ ID NO: 40 - RNA, 34 nt, Synthetic construct
  source 1..34: mol_type = other RNA
gtaaaacgacggccagagaaagaaaaggtaggaa

SEQ ID NO: 41 - RNA, 36 nt, Synthetic construct
  source 1..36: mol_type = other RNA
gtaaaacgacggccagccactcctggcttcaagaac

SEQ ID NO: 42 - RNA, 37 nt, Synthetic construct
  source 1..37: mol_type = other RNA
gtaaaacgacggccagagggacatgtcataagcctct

SEQ ID NO: 43 - RNA, 36 nt, Synthetic construct
  source 1..36: mol_type = other RNA
gtaaaacgacggccagtagcttgatggggatggcat

SEQ ID NO: 44 - RNA, 34 nt, Synthetic construct
  source 1..34: mol_type = other RNA
gtaaaacgacggccagaaagaaggaaggaaccac

SEQ ID NO: 45 - RNA, 38 nt, Synthetic construct
  source 1..38: mol_type = other RNA
gtaaaacgacggccagggaacctcacaaagtaactctg

SEQ ID NO: 46 - RNA, 36 nt, Synthetic construct
  source 1..36: mol_type = other RNA
gtaaaacgacggccagacacattcgaaggaggctca

SEQ ID NO: 47 - RNA, 37 nt, Synthetic construct
  source 1..37: mol_type = other RNA
gtaaaacgacggccagccactagcctggcatatctga

SEQ ID NO: 48 - RNA, 38 nt, Synthetic construct
  source 1..38: mol_type = other RNA
gtaaaacgacggccagttcatctatgtctttggtggct

SEQ ID NO: 49 - RNA, 36 nt, Synthetic construct
  source 1..36: mol_type = other RNA
gtaaaacgacggccagacacccccaccaactgtaga

SEQ ID NO: 50 - RNA, 37 nt, Synthetic construct
  source 1..37: mol_type = other RNA
gtaaaacgacggccagaggatatgtggaaagcgctct

SEQ ID NO: 51 - RNA, 36 nt, Synthetic construct
  source 1..36: mol_type = other RNA
gtaaaacgacggccagcacatcacatttgcagggga

SEQ ID NO: 52 - RNA, 37 nt, Synthetic construct
  source 1..37: mol_type = other RNA
gtaaaacgacggccagcgtttcccaactaaatgcaga

SEQ ID NO: 53 - RNA, 36 nt, Synthetic construct
  source 1..36: mol_type = other RNA
gtaaaacgacggccagtggcccttatcccttctttt

SEQ ID NO: 54 - RNA, 36 nt, Synthetic construct
  source 1..36: mol_type = other RNA
gtaaaacgacggccagggggacttggctatttcaca

SEQ ID NO: 55 - RNA, 37 nt, Synthetic construct
  source 1..37: mol_type = other RNA
gtaaaacgacggccagacttcccagtcagtgtgtaca

SEQ ID NO: 56 - RNA, 36 nt, Synthetic construct
  source 1..36: mol_type = other RNA
gtaaaacgacggccagaagcccaggaattcaagacc

SEQ ID NO: 57 - RNA, 39 nt, Synthetic construct
  source 1..39: mol_type = other RNA
gtaaaacgacggccagtggtacagttggttgataacaca

SEQ ID NO: 58 - RNA, 36 nt, Synthetic construct
  source 1..36: mol_type = other RNA
gtaaaacgacggccaggagtccgggcattgtacaag

SEQ ID NO: 59 - RNA, 36 nt, Synthetic construct
  source 1..36: mol_type = other RNA
gtaaaacgacggccagtgcatttgacgcgcttgata

SEQ ID NO: 60 - RNA, 36 nt, Synthetic construct
  source 1..36: mol_type = other RNA
gtaaaacgacggccagcccctgatcagcgtcaaatt

SEQ ID NO: 61 - RNA, 36 nt, Synthetic construct
  source 1..36: mol_type = other RNA
gtaaaacgacggccaggggaggacgcttcagagaaa

SEQ ID NO: 62 - RNA, 33 nt, Synthetic construct
  source 1..33: mol_type = other RNA
gtaaaacgacggccagactttagtaatatgttt

SEQ ID NO: 63 - RNA, 36 nt, Synthetic construct
  source 1..36: mol_type = other RNA
gtaaaacgacggccaggtctgtgctgtccctcctgt

SEQ ID NO: 64 - RNA, 36 nt, Synthetic construct
  source 1..36: mol_type = other RNA
gtaaaacgacggccagaaccccaacgatcaattcac

SEQ ID NO: 65 - RNA, 36 nt, Synthetic construct
  source 1..36: mol_type = other RNA
gtaaaacgacggccagcacaaagcccacaccatgaa

SEQ ID NO: 66 - RNA, 36 nt, Synthetic construct
  source 1..36: mol_type = other RNA
gtaaaacgacggccagggctcattcgactcacttcc

SEQ ID NO: 67 - RNA, 36 nt, Synthetic construct
  source 1..36: mol_type = other RNA
gtaaaacgacggccaggccacacattgtctcatcca

SEQ ID NO: 68 - RNA, 36 nt, Synthetic construct
  source 1..36: mol_type = other RNA
gtaaaacgacggccaggggttcaagcagttctcctg

SEQ ID NO: 69 - RNA, 36 nt, Synthetic construct
  source 1..36: mol_type = other RNA
gtaaaacgacggccagaatgcagtggggtgaacaac

SEQ ID NO: 70 - RNA, 36 nt, Synthetic construct
  source 1..36: mol_type = other RNA
gtaaaacgacggccagcacccatttctagtgctgcc

SEQ ID NO: 71 - RNA, 34 nt, Synthetic construct
  source 1..34: mol_type = other RNA
gtaaaacgacggccagagtaggggtagaggacag

SEQ ID NO: 72 - RNA, 36 nt, Synthetic construct
  source 1..36: mol_type = other RNA
gtaaaacgacggccagggaagtgcaggcaaaatgat

SEQ ID NO: 73 - RNA, 38 nt, Synthetic construct
  source 1..38: mol_type = other RNA
gtaaaacgacggccagtggtcttctttcaaggtttgcc

SEQ ID NO: 74 - RNA, 34 nt, Synthetic construct
  source 1..34: mol_type = other RNA
gtaaaacgacggccagaagctcttgaagacataa

SEQ ID NO: 75 - RNA, 34 nt, Synthetic construct
  source 1..34: mol_type = other RNA
gtaaaacgacggccagagtcaaaggccctcctct

SEQ ID NO: 76 - RNA, 34 nt, Synthetic construct
  source 1..34: mol_type = other RNA
gtaaaacgacggccagtaccataggatgcttaac

SEQ ID NO: 77 - RNA, 34 nt, Synthetic construct
  source 1..34: mol_type = other RNA
gtaaaacgacggccaggtcccagctacttgggag

SEQ ID NO: 78 - RNA, 34 nt, Synthetic construct
  source 1..34: mol_type = other RNA
gtaaaacgacggccagggaaacttcataaacatt

SEQ ID NO: 79 - RNA, 36 nt, Synthetic construct
  source 1..36: mol_type = other RNA
gtaaaacgacggccagttagggaggcaaatcaacca

SEQ ID NO: 80 - RNA, 35 nt, Synthetic construct
  source 1..35: mol_type = other RNA
gtaaaacgacggccagaattgtgccctgaccatgc

SEQ ID NO: 81 - RNA, 34 nt, Synthetic construct
  source 1..34: mol_type = other RNA
gtaaaacgacggccagggcgttccttggggttca

SEQ ID NO: 82 - RNA, 34 nt, Synthetic construct
  source 1..34: mol_type = other RNA
gtaaaacgacggccaggaaatgatgtctggagga

SEQ ID NO: 83 - RNA, 34 nt, Synthetic construct
  source 1..34: mol_type = other RNA
gtaaaacgacggccagttctctgttgaggttgac

SEQ ID NO: 84 - RNA, 34 nt, Synthetic construct
  source 1..34: mol_type = other RNA
gtaaaacgacggccaggtcaatattgaaagaagg

SEQ ID NO: 85 - RNA, 34 nt, Synthetic construct
  source 1..34: mol_type = other RNA
gtaaaacgacggccagtatagccatcatagcata

SEQ ID NO: 86 - RNA, 34 nt, Synthetic construct
  source 1..34: mol_type = other RNA
gtaaaacgacggccagaagctgcagatcttcacg

SEQ ID NO: 87 - RNA, 34 nt, Synthetic construct
  source 1..34: mol_type = other RNA
gtaaaacgacggccaggtaatatatttaaagatt

SEQ ID NO: 88 - RNA, 34 nt, Synthetic construct
  source 1..34: mol_type = other RNA
gtaaaacgacggccagctctagatctggatgagg

SEQ ID NO: 89 - RNA, 34 nt, Synthetic construct
  source 1..34: mol_type = other RNA
gtaaaacgacggccagaatatggaagatactaat

SEQ ID NO: 90 - RNA, 36 nt, Synthetic construct
  source 1..36: mol_type = other RNA
gtaaaacgacggccagccaaatatgaaagccccaaa

SEQ ID NO: 91 - RNA, 34 nt, Synthetic construct
  source 1..34: mol_type = other RNA
gtaaaacgacggccagtcttctgtgtcgctcacg

SEQ ID NO: 92 - RNA, 36 nt, Synthetic construct
  source 1..36: mol_type = other RNA
gtaaaacgacggccaggtgaatttcctggggttcag

SEQ ID NO: 93 - RNA, 34 nt, Synthetic construct
  source 1..34: mol_type = other RNA
gtaaaacgacggccagtgatggcacaaaggaaaa

SEQ ID NO: 94 - RNA, 36 nt, Synthetic construct
  source 1..36: mol_type = other RNA
gtaaaacgacggccaggggttgcagtcttccttgtc

SEQ ID NO: 95 - RNA, 34 nt, Synthetic construct
  source 1..34: mol_type = other RNA
gtaaaacgacggccagtaccaattacatgtgagg

SEQ ID NO: 96 - RNA, 34 nt, Synthetic construct
  source 1..34: mol_type = other RNA
gtaaaacgacggccagtgtgataggctaaataat

SEQ ID NO: 97 - RNA, 36 nt, Synthetic construct
  source 1..36: mol_type = other RNA
gtaaaacgacggccagagatggagaagggaatgcaa

SEQ ID NO: 98 - RNA, 36 nt, Synthetic construct
  source 1..36: mol_type = other RNA
gtaaaacgacggccagattagcccctggaaagcagt

SEQ ID NO: 99 - RNA, 36 nt, Synthetic construct
  source 1..36: mol_type = other RNA
gtaaaacgacggccagattgtgcagaagccagtcct

SEQ ID NO: 100 - RNA, 36 nt, Synthetic construct
  source 1..36: mol_type = other RNA
gtaaaacgacggccagcccattgcaagcctacagtt

SEQ ID NO: 101 - RNA, 34 nt, Synthetic construct
  source 1..34: mol_type = other RNA
gtaaaacgacggccagatctttctcttaccctac

SEQ ID NO: 102 - RNA, 36 nt, Synthetic construct
  source 1..36: mol_type = other RNA
gtaaaacgacggccaggggacagtagaggcatcaga

SEQ ID NO: 103 - RNA, 34 nt, Synthetic construct
  source 1..34: mol_type = other RNA
gtaaaacgacggccagaaatgtttttgaagttca

SEQ ID NO: 104 - RNA, 34 nt, Synthetic construct
  source 1..34: mol_type = other RNA
gtaaaacgacggccagatcattttatcttcaatt

SEQ ID NO: 105 - RNA, 36 nt, Synthetic construct
  source 1..36: mol_type = other RNA
gtaaaacgacggccaggcttgtgggaaatgcagaat

SEQ ID NO: 106 - RNA, 36 nt, Synthetic construct
  source 1..36: mol_type = other RNA
gtaaaacgacggccagcttcccctccctagttgacc

SEQ ID NO: 107 - RNA, 34 nt, Synthetic construct
  source 1..34: mol_type = other RNA
gtaaaacgacggccaggcattgtagattcataca

SEQ ID NO: 108 - RNA, 35 nt, Synthetic construct
  source 1..35: mol_type = other RNA
caggaaacagctatgactctgcacataacggtgga

SEQ ID NO: 109 - RNA, 20 nt, Synthetic construct
  source 1..20: mol_type = other RNA
gcctgtcccttgtttccttg

SEQ ID NO: 110 - RNA, 35 nt, Synthetic construct
  source 1..35: mol_type = other RNA
caggaaacagctatgacagtagagaagcttgaaat

SEQ ID NO: 111 - RNA, 23 nt, Synthetic construct
  source 1..23: mol_type = other RNA
tgcacacatcacaaagaagtttc

SEQ ID NO: 112 - RNA, 20 nt, Synthetic construct
  source 1..20: mol_type = other RNA
cccaggctgttctcgaaaac

SEQ ID NO: 113 - RNA, 35 nt, Synthetic construct
  source 1..35: mol_type = other RNA
caggaaacagctatgacatgaaaacagtgaaatat

SEQ ID NO: 114 - RNA, 21 nt, Synthetic construct
  source 1..21: mol_type = other RNA
ggaacctctgctcttcatgac

SEQ ID NO: 115 - RNA, 35 nt, Synthetic construct
  source 1..35: mol_type = other RNA
caggaaacagctatgactgaaaatcaaaagtatct

SEQ ID NO: 116 - RNA, 20 nt, Synthetic construct
  source 1..20: mol_type = other RNA
cacttgattgggatgaatcg

SEQ ID NO: 117 - RNA, 20 nt, Synthetic construct
  source 1..20: mol_type = other RNA
cccagaaggaatgaagtcca

SEQ ID NO: 118 - RNA, 20 nt, Synthetic construct
  source 1..20: mol_type = other RNA
gggaaaagtctccctggttc

SEQ ID NO: 119 - RNA, 20 nt, Synthetic construct
  source 1..20: mol_type = other RNA
atctgacacaaaggcccaag

SEQ ID NO: 120 - RNA, 35 nt, Synthetic construct
  source 1..35: mol_type = other RNA
caggaaacagctatgacctgtactctgaaaggatg

SEQ ID NO: 121 - RNA, 22 nt, Synthetic construct
  source 1..22: mol_type = other RNA
tgttcagagaagtctttgctca

SEQ ID NO: 122 - RNA, 35 nt, Synthetic construct
  source 1..35: mol_type = other RNA
caggaaacagctatgactagctggatctagggatt

SEQ ID NO: 123 - RNA, 20 nt, Synthetic construct
  source 1..20: mol_type = other RNA
cctcctcctgaattcctcct

SEQ ID NO: 124 - RNA, 35 nt, Synthetic construct
  source 1..35: mol_type = other RNA
caggaaacagctatgacttccaacttctttacatc

SEQ ID NO: 125 - RNA, 35 nt, Synthetic construct
  source 1..35: mol_type = other RNA
caggaaacagctatgacgctacacatatttcataa

SEQ ID NO: 126 - RNA, 20 nt, Synthetic construct
  source 1..20: mol_type = other RNA
cccaccaaccagagagaact

SEQ ID NO: 127 - RNA, 20 nt, Synthetic construct
  source 1..20: mol_type = other RNA
ctgtgtggctgctttcactg

SEQ ID NO: 128 - RNA, 20 nt, Synthetic construct
  source 1..20: mol_type = other RNA
tgtaagatggagcagggacc

SEQ ID NO: 129 - RNA, 35 nt, Synthetic construct
  source 1..35: mol_type = other RNA
caggaaacagctatgaccctggatgacatatttgt

SEQ ID NO: 130 - RNA, 35 nt, Synthetic construct
  source 1..35: mol_type = other RNA
caggaaacagctatgacctgaatttacaaattcgt

SEQ ID NO: 131 - RNA, 20 nt, Synthetic construct
  source 1..20: mol_type = other RNA
gtatgagggccaatttgtgg

SEQ ID NO: 132 - RNA, 20 nt, Synthetic construct
  source 1..20: mol_type = other RNA
tgcgcgtgttttaagagagg

SEQ ID NO: 133 - RNA, 20 nt, Synthetic construct
  source 1..20: mol_type = other RNA
taacaggagaattgggcggt

SEQ ID NO: 134 - RNA, 35 nt, Synthetic construct
  source 1..35: mol_type = other RNA
caggaaacagctatgacccaacaagagtaaaggtt

SEQ ID NO: 135 - RNA, 21 nt, Synthetic construct
  source 1..21: mol_type = other RNA
aggctccttttgaacaccttc

SEQ ID NO: 136 - RNA, 20 nt, Synthetic construct
  source 1..20: mol_type = other RNA
aatgaaccaccctgtcccat

SEQ ID NO: 137 - RNA, 20 nt, Synthetic construct
  source 1..20: mol_type = other RNA
ggcccagatgtctcactaca

SEQ ID NO: 138 - RNA, 20 nt, Synthetic construct
  source 1..20: mol_type = other RNA
ctcccatccgaagagacagc

SEQ ID NO: 139 - RNA, 22 nt, Synthetic construct
  source 1..22: mol_type = other RNA
ggctatacatacctgcacagca

SEQ ID NO: 140 - RNA, 22 nt, Synthetic construct
  source 1..22: mol_type = other RNA
tgcatggcttcttctacaagtg

SEQ ID NO: 141 - RNA, 20 nt, Synthetic construct
  source 1..20: mol_type = other RNA
ggaacattgctccccattcc

SEQ ID NO: 142 - RNA, 20 nt, Synthetic construct
  source 1..20: mol_type = other RNA
tcttgggatcatccttgaca

SEQ ID NO: 143 - RNA, 20 nt, Synthetic construct
  source 1..20: mol_type = other RNA
cgacctccttccaatccagt

SEQ ID NO: 144 - RNA, 20 nt, Synthetic construct
  source 1..20: mol_type = other RNA
ctcgtcagaaccaacggtct

SEQ ID NO: 145 - RNA, 20 nt, Synthetic construct
  source 1..20: mol_type = other RNA
gcaggcaaacaggaacagaa

SEQ ID NO: 146 - RNA, 20 nt, Synthetic construct
  source 1..20: mol_type = other RNA
gtgacagcgagtcagacgtt

SEQ ID NO: 147 - RNA, 20 nt, Synthetic construct
  source 1..20: mol_type = other RNA
aagtggaagaggtgaagggt

SEQ ID NO: 148 - RNA, 20 nt, Synthetic construct
  source 1..20: mol_type = other RNA
ggttttgtggcttcttgcat

SEQ ID NO: 149 - RNA, 20 nt, Synthetic construct
  source 1..20: mol_type = other RNA
aagacgatcgagaccatccc

SEQ ID NO: 150 - RNA, 20 nt, Synthetic construct
  source 1..20: mol_type = other RNA
gctttgttttccagtgcctg

SEQ ID NO: 151 - RNA, 22 nt, Synthetic construct
  source 1..22: mol_type = other RNA
tcttgaggaagggagaaacaca

SEQ ID NO: 152 - RNA, 35 nt, Synthetic construct
  source 1..35: mol_type = other RNA
caggaaacagctatgacctaaggcaaacaacactg

SEQ ID NO: 153 - RNA, 20 nt, Synthetic construct
  source 1..20: mol_type = other RNA
tccatgggcactagaagagc

SEQ ID NO: 154 - RNA, 20 nt, Synthetic construct
  source 1..20: mol_type = other RNA
gccctgagcaatcctatctg

SEQ ID NO: 155 - RNA, 20 nt, Synthetic construct
  source 1..20: mol_type = other RNA
acgggttgaatggattggtg

SEQ ID NO: 156 - RNA, 22 nt, Synthetic construct
  source 1..22: mol_type = other RNA
ggaggaatcagtctacccaatt

SEQ ID NO: 157 - RNA, 21 nt, Synthetic construct
  source 1..21: mol_type = other RNA
ccagaaaggtgaatgctgtca

SEQ ID NO: 158 - RNA, 20 nt, Synthetic construct
  source 1..20: mol_type = other RNA
tcaaacttcagctgggaacc

SEQ ID NO: 159 - RNA, 20 nt, Synthetic construct
  source 1..20: mol_type = other RNA
catggagaaacaggcgagtg

SEQ ID NO: 160 - RNA, 20 nt, Synthetic construct
  source 1..20: mol_type = other RNA
ctggagaggcatggagagtt

SEQ ID NO: 161 - RNA, 35 nt, Synthetic construct
  source 1..35: mol_type = other RNA
caggaaacagctatgactgtgtggttcagtatatc

SEQ ID NO: 162 - RNA, 20 nt, Synthetic construct
  source 1..20: mol_type = other RNA
tagcaagcaccacctcctct

SEQ ID NO: 163 - RNA, 20 nt, Synthetic construct
  source 1..20: mol_type = other RNA
ataggtggtcattcgagggc

SEQ ID NO: 164 - RNA, 35 nt, Synthetic construct
  source 1..35: mol_type = other RNA
caggaaacagctatgacattccaaagccatgctaa

SEQ ID NO: 165 - RNA, 35 nt, Synthetic construct
  source 1..35: mol_type = other RNA
caggaaacagctatgactccagcctcagacagaac

SEQ ID NO: 166 - RNA, 35 nt, Synthetic construct
  source 1..35: mol_type = other RNA
caggaaacagctatgacttcagcccagatccctaa

SEQ ID NO: 167 - RNA, 35 nt, Synthetic construct
  source 1..35: mol_type = other RNA
caggaaacagctatgacaagtcagatcaccttcat

SEQ ID NO: 168 - RNA, 35 nt, Synthetic construct
  source 1..35: mol_type = other RNA
caggaaacagctatgacgtatttctccaacctata

SEQ ID NO: 169 - RNA, 20 nt, Synthetic construct
  source 1..20: mol_type = other RNA
tttgctgcagctagccattt

SEQ ID NO: 170 - RNA, 21 nt, Synthetic construct
  source 1..21: mol_type = other RNA
gagagacagagacagaggtga

SEQ ID NO: 171 - RNA, 35 nt, Synthetic construct
  source 1..35: mol_type = other RNA
caggaaacagctatgactcatccaaatctactttc

SEQ ID NO: 172 - RNA, 35 nt, Synthetic construct
  source 1..35: mol_type = other RNA
caggaaacagctatgactgaggaagtgaaaacatt

SEQ ID NO: 173 - RNA, 35 nt, Synthetic construct
  source 1..35: mol_type = other RNA
caggaaacagctatgacgctcttttctttttcttt

SEQ ID NO: 174 - RNA, 35 nt, Synthetic construct
  source 1..35: mol_type = other RNA
caggaaacagctatgacacccagttaacatcacaa

SEQ ID NO: 175 - RNA, 35 nt, Synthetic construct
  source 1..35: mol_type = other RNA
caggaaacagctatgacgcacctacctcacctgca

SEQ ID NO: 176 - RNA, 35 nt, Synthetic construct
  source 1..35: mol_type = other RNA
caggaaacagctatgacttctgtaattctacaaga

SEQ ID NO: 177 - RNA, 35 nt, Synthetic construct
  source 1..35: mol_type = other RNA
caggaaacagctatgacaagatggtgaagaattag

SEQ ID NO: 178 - RNA, 35 nt, Synthetic construct
  source 1..35: mol_type = other RNA
caggaaacagctatgacgaagcagggttttctgca

SEQ ID NO: 179 - RNA, 35 nt, Synthetic construct
  source 1..35: mol_type = other RNA
caggaaacagctatgacgtaaatgtcatattgtga

SEQ ID NO: 180 - RNA, 20 nt, Synthetic construct
  source 1..20: mol_type = other RNA
ggggttcagaacttcagtgg

SEQ ID NO: 181 - RNA, 35 nt, Synthetic construct
  source 1..35: mol_type = other RNA
caggaaacagctatgactatgatcaccttgtataa

SEQ ID NO: 182 - RNA, 20 nt, Synthetic construct
  source 1..20: mol_type = other RNA
atggattgggtgtccagaaa

SEQ ID NO: 183 - RNA, 35 nt, Synthetic construct
  source 1..35: mol_type = other RNA
caggaaacagctatgacaatgggaaagatatataa

SEQ ID NO: 184 - RNA, 20 nt, Synthetic construct
  source 1..20: mol_type = other RNA
tgggagaagacccagctaaa

SEQ ID NO: 185 - RNA, 35 nt, Synthetic construct
  source 1..35: mol_type = other RNA
caggaaacagctatgaccctccaaataccatccca

SEQ ID NO: 186 - RNA, 35 nt, Synthetic construct
  source 1..35: mol_type = other RNA
caggaaacagctatgacttcctgtccagcattcac

SEQ ID NO: 187 - RNA, 21 nt, Synthetic construct
  source 1..21: mol_type = other RNA
tgcgttgttatcatactgtgc

SEQ ID NO: 188 - RNA, 20 nt, Synthetic construct
  source 1..20: mol_type = other RNA
acatgccgtacaagtcatcc

SEQ ID NO: 189 - RNA, 20 nt, Synthetic construct
  source 1..20: mol_type = other RNA
gggatggggaaagagaagtc

SEQ ID NO: 190 - RNA, 21 nt, Synthetic construct
  source 1..21: mol_type = other RNA
tccctgagaaagtcctggttt

SEQ ID NO: 191 - RNA, 32 nt, Synthetic construct
  source 1..32: mol_type = other RNA
caggaaacagctatgactgttaactagaataa

SEQ ID NO: 192 - RNA, 20 nt, Synthetic construct
  source 1..20: mol_type = other RNA
gacaaagtggcatggcatga

SEQ ID NO: 193 - RNA, 35 nt, Synthetic construct
  source 1..35: mol_type = other RNA
caggaaacagctatgacttccctgcaatggagggc

SEQ ID NO: 194 - RNA, 35 nt, Synthetic construct
  source 1..35: mol_type = other RNA
caggaaacagctatgacgaaaaggcaaaaccacaa

SEQ ID NO: 195 - RNA, 20 nt, Synthetic construct
  source 1..20: mol_type = other RNA
caccaagccattcatgaggg

SEQ ID NO: 196 - RNA, 20 nt, Synthetic construct
  source 1..20: mol_type = other RNA
gaagggggaaaagggtgata

SEQ ID NO: 197 - RNA, 35 nt, Synthetic construct
  source 1..35: mol_type = other RNA
caggaaacagctatgacattggctggtgggcacac

SEQ ID NO: 198 - RNA, 21 nt, Synthetic construct
  source 1..21: mol_type = other RNA
tgagactgtatttgtgggcca

SEQ ID NO: 199 - RNA, 20 nt, Synthetic construct
  source 1..20: mol_type = other RNA
ggccctcaccatgtaaaagg

SEQ ID NO: 200 - RNA, 20 nt, Synthetic construct
  source 1..20: mol_type = other RNA
actgggaagttgggtcttca

SEQ ID NO: 201 - RNA, 23 nt, Synthetic construct
  source 1..23: mol_type = other RNA
tggaggtaggatattacagggaa

SEQ ID NO: 202 - RNA, 20 nt, Synthetic construct
  source 1..20: mol_type = other RNA
gccatccactcactacaggt

SEQ ID NO: 203 - RNA, 20 nt, Synthetic construct
  source 1..20: mol_type = other RNA
tggaaggctagaggaagctg

SEQ ID NO: 204 - RNA, 20 nt, Synthetic construct
  source 1..20: mol_type = other RNA
tgtgtgccttcaaaatgggg

SEQ ID NO: 205 - RNA, 22 nt, Synthetic construct
  source 1..22: mol_type = other RNA
tgaagcatacattctggaggtt

SEQ ID NO: 206 - RNA, 20 nt, Synthetic construct
  source 1..20: mol_type = other RNA
tggatgaactggatggatga

SEQ ID NO: 207 - RNA, 20 nt, Synthetic construct
  source 1..20: mol_type = other RNA
tgcctagtccagtaatgcga

SEQ ID NO: 208 - RNA, 21 nt, Synthetic construct
  source 1..21: mol_type = other RNA
acaccatggcctcatctatca

SEQ ID NO: 209 - RNA, 20 nt, Synthetic construct
  source 1..20: mol_type = other RNA
tgctctcaactttccctgga

SEQ ID NO: 210 - RNA, 20 nt, Synthetic construct
  source 1..20: mol_type = other RNA
gggggatggtcatgagattt

SEQ ID NO: 211 - RNA, 20 nt, Synthetic construct
  source 1..20: mol_type = other RNA
cctgccccagtgaaatcagt

SEQ ID NO: 212 - RNA, 20 nt, Synthetic construct
  source 1..20: mol_type = other RNA
aggcagctagagttcacagg

SEQ ID NO: 213 - RNA, 20 nt, Synthetic construct
  source 1..20: mol_type = other RNA
gcagaggggagctcttttct

SEQ ID NO: 214 - RNA, 20 nt, Synthetic construct
  source 1..20: mol_type = other RNA
ccattcctctccacactcca

SEQ ID NO: 215 - RNA, 22 nt, Synthetic construct
  source 1..22: mol_type = other RNA
agcacgcaatattactgggaac

SEQ ID NO: 216 - RNA, 19 nt, Synthetic construct
  source 1..19: mol_type = other RNA
tgaccaccacatccaggat

SEQ ID NO: 217 - RNA, 20 nt, Synthetic construct
  source 1..20: mol_type = other RNA
cactcccaagaacgcagaat

SEQ ID NO: 218 - RNA, 22 nt, Synthetic construct
  source 1..22: mol_type = other RNA
accatcgttttaaaaggtgcaa

SEQ ID NO: 219 - RNA, 20 nt, Synthetic construct
  source 1..20: mol_type = other RNA
gctcgagatcacagtccctt

SEQ ID NO: 220 - RNA, 20 nt, Synthetic construct
  source 1..20: mol_type = other RNA
atgtgcggtggtaggagaag

SEQ ID NO: 221 - RNA, 23 nt, Synthetic construct
  source 1..23: mol_type = other RNA
agcaatatggaggaacaaaagca

SEQ ID NO: 222 - RNA, 22 nt, Synthetic construct
  source 1..22: mol_type = other RNA
gtcatccacttcatccacttca

SEQ ID NO: 223 - RNA, 20 nt, Synthetic construct
  source 1..20: mol_type = other RNA
aggagtggctgcaaattgtt

SEQ ID NO: 224 - RNA, 21 nt, Synthetic construct
  source 1..21: mol_type = other RNA
cggtatagtttccacagcagg

SEQ ID NO: 225 - RNA, 20 nt, Synthetic construct
  source 1..20: mol_type = other RNA
cagtttgccagtggttcctc

SEQ ID NO: 226 - RNA, 20 nt, Synthetic construct
  source 1..20: mol_type = other RNA
caccgagtttgagatgcctg

SEQ ID NO: 227 - RNA, 20 nt, Synthetic construct
  source 1..20: mol_type = other RNA
tgatccagtgtgggtgagaa

SEQ ID NO: 228 - RNA, 20 nt, Synthetic construct
  source 1..20: mol_type = other RNA
ggagagtgtaccctgttgct

SEQ ID NO: 229 - RNA, 20 nt, Synthetic construct
  source 1..20: mol_type = other RNA
gccccgctactgagagaata

SEQ ID NO: 230 - RNA, 20 nt, Synthetic construct
  source 1..20: mol_type = other RNA
acctcatctccctgctatgc

SEQ ID NO: 231 - RNA, 20 nt, Synthetic construct
  source 1..20: mol_type = other RNA
tcagcctcatctttctccca

SEQ ID NO: 232 - RNA, 20 nt, Synthetic construct
  source 1..20: mol_type = other RNA
acttgacatgtatggtgggg

SEQ ID NO: 233 - RNA, 23 nt, Synthetic construct
  source 1..23: mol_type = other RNA
ggattttacagctggaagggatc

SEQ ID NO: 234 - RNA, 21 nt, Synthetic construct
  source 1..21: mol_type = other RNA
gcaacattgcatgttcagaaa

SEQ ID NO: 235 - RNA, 20 nt, Synthetic construct
  source 1..20: mol_type = other RNA
cggtagcttggatgacagaa

SEQ ID NO: 236 - RNA, 20 nt, Synthetic construct
  source 1..20: mol_type = other RNA
ggccctacccatacctactg

SEQ ID NO: 237 - RNA, 20 nt, Synthetic construct
  source 1..20: mol_type = other RNA
taggactacagcagcacacc

SEQ ID NO: 238 - RNA, 21 nt, Synthetic construct
  source 1..21: mol_type = other RNA
tccattccttgttcttgccac

SEQ ID NO: 239 - RNA, 20 nt, Synthetic construct
  source 1..20: mol_type = other RNA
ccaagcaacataaagcagca

SEQ ID NO: 240 - RNA, 23 nt, Synthetic construct
  source 1..23: mol_type = other RNA
tctttctcctagatctggacact

SEQ ID NO: 241 - RNA, 20 nt, Synthetic construct
  source 1..20: mol_type = other RNA
gctgcctttcttccagtgat

SEQ ID NO: 242 - RNA, 20 nt, Synthetic construct
  source 1..20: mol_type = other RNA
aggctccactgcttctgtgt

SEQ ID NO: 243 - RNA, 21 nt, Synthetic construct
  source 1..21: mol_type = other RNA
tcctggggcaattttactttt

SEQ ID NO: 244 - RNA, 20 nt, Synthetic construct
  source 1..20: mol_type = other RNA
gctcacccactttccattca

SEQ ID NO: 245 - RNA, 20 nt, Synthetic construct
  source 1..20: mol_type = other RNA
tctgccccaacatgagactt

SEQ ID NO: 246 - RNA, 21 nt, Synthetic construct
  source 1..21: mol_type = other RNA
tgtccacacctacaatcctga

SEQ ID NO: 247 - RNA, 22 nt, Synthetic construct
  source 1..22: mol_type = other RNA
tcagttgtttcacagatctgca

SEQ ID NO: 248 - RNA, 20 nt, Synthetic construct
  source 1..20: mol_type = other RNA
gtgcagcttcactcctcaca

SEQ ID NO: 249 - RNA, 20 nt, Synthetic construct
  source 1..20: mol_type = other RNA
caattgcaacgggaattctt

SEQ ID NO: 250 - RNA, 23 nt, Synthetic construct
  source 1..23: mol_type = other RNA
gcagataatgaccttcctattgc

SEQ ID NO: 251 - RNA, 21 nt, Synthetic construct
  source 1..21: mol_type = other RNA
ggtagagaaaagccctgagga

SEQ ID NO: 252 - RNA, 19 nt, Synthetic construct
  source 1..19: mol_type = other RNA
gagaacgggaggattctgg

SEQ ID NO: 253 - RNA, 20 nt, Synthetic construct
  source 1..20: mol_type = other RNA
tgcatcacaagggttattgc

SEQ ID NO: 254 - RNA, 20 nt, Synthetic construct
  source 1..20: mol_type = other RNA
atgcaaccttttgtgttcca

SEQ ID NO: 255 - RNA, 23 nt, Synthetic construct
  source 1..23: mol_type = other RNA
tgtggcacagatttattagcaga

SEQ ID NO: 256 - RNA, 20 nt, Synthetic construct
  source 1..20: mol_type = other RNA
acaggcacaaccatccattt

SEQ ID NO: 257 - RNA, 20 nt, Synthetic construct
  source 1..20: mol_type = other RNA
atgcctgcatttaccaccat

SEQ ID NO: 258 - RNA, 20 nt, Synthetic construct
  source 1..20: mol_type = other RNA
ccaattttaggcatgcaggt

SEQ ID NO: 259 - RNA, 20 nt, Synthetic construct
  source 1..20: mol_type = other RNA
ggcaaatgttccctgatgtt

SEQ ID NO: 260 - RNA, 20 nt, Synthetic construct
  source 1..20: mol_type = other RNA
gccaatcatgtgcctctctt

SEQ ID NO: 261 - RNA, 19 nt, Synthetic construct
  source 1..19: mol_type = other RNA
tttcccaggcttcgtcgat

SEQ ID NO: 262 - RNA, 20 nt, Synthetic construct
  source 1..20: mol_type = other RNA
gcggggatttgcacagaatt

SEQ ID NO: 263 - RNA, 18 nt, Synthetic construct
  source 1..18: mol_type = other RNA
gcactcgaaggcttctcc

SEQ ID NO: 264 - RNA, 21 nt, Synthetic construct
  source 1..21: mol_type = other RNA
tcaatcaagtgagacagggct

SEQ ID NO: 265 - RNA, 20 nt, Synthetic construct
  source 1..20: mol_type = other RNA
catgggaggctggattcatt

SEQ ID NO: 266 - RNA, 20 nt, Synthetic construct
  source 1..20: mol_type = other RNA
ctccccatagctaaggacca

SEQ ID NO: 267 - RNA, 22 nt, Synthetic construct
  source 1..22: mol_type = other RNA
gtcaagatgtggactactagca

SEQ ID NO: 268 - RNA, 20 nt, Synthetic construct
  source 1..20: mol_type = other RNA
gccaaatcggaaacaaagaa

SEQ ID NO: 269 - RNA, 20 nt, Synthetic construct
  source 1..20: mol_type = other RNA
caatgtaagtgggcagcaga

SEQ ID NO: 270 - RNA, 22 nt, Synthetic construct
  source 1..22: mol_type = other RNA
accaaggctaaagatcagtgat

SEQ ID NO: 271 - RNA, 22 nt, Synthetic construct
  source 1..22: mol_type = other RNA
tcattggtatttggagctttgc

SEQ ID NO: 272 - RNA, 20 nt, Synthetic construct
  source 1..20: mol_type = other RNA
ccagcctctagaactgtgga

SEQ ID NO: 273 - RNA, 20 nt, Synthetic construct
  source 1..20: mol_type = other RNA
atggtgtgtcagacgctgtt

SEQ ID NO: 274 - RNA, 23 nt, Synthetic construct
  source 1..23: mol_type = other RNA
ggtaaataactttgtcctgggtg

SEQ ID NO: 275 - RNA, 20 nt, Synthetic construct
  source 1..20: mol_type = other RNA
gaaattcttcctgccagcac

SEQ ID NO: 276 - RNA, 20 nt, Synthetic construct
  source 1..20: mol_type = other RNA
tggtggtgttggtgattcag

SEQ ID NO: 277 - RNA, 20 nt, Synthetic construct
  source 1..20: mol_type = other RNA
tggtggtgttggtgattcag

SEQ ID NO: 278 - RNA, 21 nt, Synthetic construct
  source 1..21: mol_type = other RNA
tgcttgcttaaactcctcagt

SEQ ID NO: 279 - RNA, 20 nt, Synthetic construct
  source 1..20: mol_type = other RNA
gggtgacaatcttcctgtgg

SEQ ID NO: 280 - RNA, 20 nt, Synthetic construct
  source 1..20: mol_type = other RNA
gttccttcaattgccgatgt

SEQ ID NO: 281 - RNA, 20 nt, Synthetic construct
  source 1..20: mol_type = other RNA
cagctcatccagaacccaga

SEQ ID NO: 282 - RNA, 20 nt, Synthetic construct
  source 1..20: mol_type = other RNA
atgctgctgtgatcgttttg

SEQ ID NO: 283 - RNA, 20 nt, Synthetic construct
  source 1..20: mol_type = other RNA
ggaaagtggtgtccaggatg

SEQ ID NO: 284 - RNA, 21 nt, Synthetic construct
  source 1..21: mol_type = other RNA
catgaattacaagggcagcaa

SEQ ID NO: 285 - RNA, 20 nt, Synthetic construct
  source 1..20: mol_type = other RNA
ataggcgtacccctgaatcc

SEQ ID NO: 286 - RNA, 21 nt, Synthetic construct
  source 1..21: mol_type = other RNA
aaagacctttgaaggatgcaa

SEQ ID NO: 287 - RNA, 23 nt, Synthetic construct
  source 1..23: mol_type = other RNA
tggattccagaaattgtttttga

SEQ ID NO: 288 - RNA, 20 nt, Synthetic construct
  source 1..20: mol_type = other RNA
gctattcattagcggggaca

SEQ ID NO: 289 - RNA, 23 nt, Synthetic construct
  source 1..23: mol_type = other RNA
aaaggcttagtgctgaccttaca

SEQ ID NO: 290 - RNA, 20 nt, Synthetic construct
  source 1..20: mol_type = other RNA
catgggcagtttgctttacc

SEQ ID NO: 291 - RNA, 20 nt, Synthetic construct
  source 1..20: mol_type = other RNA
tttccaaggtgatgaccaca

SEQ ID NO: 292 - RNA, 20 nt, Synthetic construct
  source 1..20: mol_type = other RNA
agaaggccctttcatcatca

SEQ ID NO: 293 - RNA, 20 nt, Synthetic construct
  source 1..20: mol_type = other RNA
aactggattggttgctgctt

SEQ ID NO: 294 - RNA, 23 nt, Synthetic construct
  source 1..23: mol_type = other RNA
acactgtatttcgcttacatgca

SEQ ID NO: 295 - RNA, 19 nt, Synthetic construct
  source 1..19: mol_type = other RNA
tgggtgacagagcaagact

SEQ ID NO: 296 - RNA, 20 nt, Synthetic construct
  source 1..20: mol_type = other RNA
ggctcctcctccctacaaat

SEQ ID NO: 297 - RNA, 20 nt, Synthetic construct
  source 1..20: mol_type = other RNA
tcatccctttgtccagcaga

SEQ ID NO: 298 - RNA, 20 nt, Synthetic construct
  source 1..20: mol_type = other RNA
tgtcctcatttccctgtgca

SEQ ID NO: 299 - RNA, 20 nt, Synthetic construct
  source 1..20: mol_type = other RNA
agacacgtaacggcagatgt

SEQ ID NO: 300 - RNA, 20 nt, Synthetic construct
  source 1..20: mol_type = other RNA
tctttgtcttgtgcatggcg

SEQ ID NO: 301 - RNA, 22 nt, Synthetic construct
  source 1..22: mol_type = other RNA
cttagaaaaggcacagcatagg

SEQ ID NO: 302 - RNA, 20 nt, Synthetic construct
  source 1..20: mol_type = other RNA
gctccagggtttaacaggga

SEQ ID NO: 303 - RNA, 19 nt, Synthetic construct
  source 1..19: mol_type = other RNA
gccagccttgaagtgtgtc

SEQ ID NO: 304 - RNA, 23 nt, Synthetic construct
  source 1..23: mol_type = other RNA
gcacatccaaatttattcacacg

SEQ ID NO: 305 - RNA, 21 nt, Synthetic construct
  source 1..21: mol_type = other RNA
gaacaacaccaggcacatagt

SEQ ID NO: 306 - RNA, 21 nt, Synthetic construct
  source 1..21: mol_type = other RNA
ggaattcctgactccaaaaca

SEQ ID NO: 307 - RNA, 23 nt, Synthetic construct
  source 1..23: mol_type = other RNA
ctggtgcttttgttttgattagg

SEQ ID NO: 308 - RNA, 20 nt, Synthetic construct
  source 1..20: mol_type = other RNA
aggcaacaggacatttcagg

SEQ ID NO: 309 - RNA, 20 nt, Synthetic construct
  source 1..20: mol_type = other RNA
gctgttccaatacaagcccc

SEQ ID NO: 310 - RNA, 20 nt, Synthetic construct
  source 1..20: mol_type = other RNA
tctggtcccataactgcaca

SEQ ID NO: 311 - RNA, 21 nt, Synthetic construct
  source 1..21: mol_type = other RNA
tctggagaacaaaggcattcc

SEQ ID NO: 312 - RNA, 20 nt, Synthetic construct
  source 1..20: mol_type = other RNA
ggttcctgacttccatacgg

SEQ ID NO: 313 - RNA, 20 nt, Synthetic construct
  source 1..20: mol_type = other RNA
gggagggagggaagaaacaa

SEQ ID NO: 314 - RNA, 23 nt, Synthetic construct
  source 1..23: mol_type = other RNA
tgcatttcttatttcccagcaac

SEQ ID NO: 315 - RNA, 21 nt, Synthetic construct
  source 1..21: mol_type = other RNA
agctgtgcaggattgaattct

SEQ ID NO: 316 - RNA, 25 nt, Synthetic construct
  source 1..25: mol_type = other RNA
atgaccaaaatgagaaattattagc

SEQ ID NO: 317 - RNA, 19 nt, Synthetic construct
  source 1..19: mol_type = other RNA
gtatgccaggagccaggtt

SEQ ID NO: 318 - RNA, 20 nt, Synthetic construct
  source 1..20: mol_type = other RNA
cttgggtatgtgccttgctc

SEQ ID NO: 319 - RNA, 21 nt, Synthetic construct
  source 1..21: mol_type = other RNA
gcatgtgtttacctggcctac

SEQ ID NO: 320 - RNA, 20 nt, Synthetic construct
  source 1..20: mol_type = other RNA
cctaagggtgtgactccaga

SEQ ID NO: 321 - RNA, 20 nt, Synthetic construct
  source 1..20: mol_type = other RNA
catcacgcccggctaatttt

SEQ ID NO: 322 - RNA, 22 nt, Synthetic construct
  source 1..22: mol_type = other RNA
gagcttttgaatggtgactgga

SEQ ID NO: 323 - RNA, 20 nt, Synthetic construct
  source 1..20: mol_type = other RNA
caagcgcctatggagttgtc

SEQ ID NO: 324 - RNA, 21 nt, Synthetic construct
  source 1..21: mol_type = other RNA
agaaaccagtgaaggatctcc

SEQ ID NO: 325 - RNA, 23 nt, Synthetic construct
  source 1..23: mol_type = other RNA
gggcaataaacatgaaaagtggt

SEQ ID NO: 326 - RNA, 20 nt, Synthetic construct
  source 1..20: mol_type = other RNA
acagccccaatctgtttcac

SEQ ID NO: 327 - RNA, 20 nt, Synthetic construct
  source 1..20: mol_type = other RNA
tagaggagttgggggaaggt

SEQ ID NO: 328 - RNA, 22 nt, Synthetic construct
  source 1..22: mol_type = other RNA
tcatcccgagagttatatcccc

SEQ ID NO: 329 - RNA, 20 nt, Synthetic construct
  source 1..20: mol_type = other RNA
aagatcaagccagccacaat

SEQ ID NO: 330 - RNA, 21 nt, Synthetic construct
  source 1..21: mol_type = other RNA
catcaactcactcacaggcag

SEQ ID NO: 331 - RNA, 19 nt, Synthetic construct
  source 1..19: mol_type = other RNA
gatgtgctggcatgtggac

SEQ ID NO: 332 - RNA, 20 nt, Synthetic construct
  source 1..20: mol_type = other RNA
gctgcgggacatagaactgt

SEQ ID NO: 333 - RNA, 20 nt, Synthetic construct
  source 1..20: mol_type = other RNA
tgaccctggaattcacctgc

SEQ ID NO: 334 - RNA, 20 nt, Synthetic construct
  source 1..20: mol_type = other RNA
tgtatctgcgccaagggaaa

SEQ ID NO: 335 - RNA, 20 nt, Synthetic construct
  source 1..20: mol_type = other RNA
ttttgagtgaacgtggcagc

SEQ ID NO: 336 - RNA, 21 nt, Synthetic construct
  source 1..21: mol_type = other RNA
aggattactgattgggccact

SEQ ID NO: 337 - RNA, 22 nt, Synthetic construct
  source 1..22: mol_type = other RNA
aggtttgttctcccatagttga

SEQ ID NO: 338 - RNA, 21 nt, Synthetic construct
  source 1..21: mol_type = other RNA
agatgtttgctccaagaacct

SEQ ID NO: 339 - RNA, 23 nt, Synthetic construct
  source 1..23: mol_type = other RNA
acaaagacatgcaacagatcaca

SEQ ID NO: 340 - RNA, 23 nt, Synthetic construct
  source 1..23: mol_type = other RNA
agcaaacaaaagaaccactagct

SEQ ID NO: 341 - RNA, 21 nt, Synthetic construct
  source 1..21: mol_type = other RNA
agggtgtcgtactaaatggga

SEQ ID NO: 342 - RNA, 20 nt, Synthetic construct
  source 1..20: mol_type = other RNA
ccaaggttaggtgtggggaa

SEQ ID NO: 343 - RNA, 23 nt, Synthetic construct
  source 1..23: mol_type = other RNA
gctaaggtgatcaacaagtttcc

SEQ ID NO: 344 - RNA, 22 nt, Synthetic construct
  source 1..22: mol_type = other RNA
agatctcccttttgttggttga

SEQ ID NO: 345 - RNA, 20 nt, Synthetic construct
  source 1..20: mol_type = other RNA
cagggatgtgtgggagatga

SEQ ID NO: 346 - RNA, 20 nt, Synthetic construct
  source 1..20: mol_type = other RNA
ggtacgcactcaatagctgg

SEQ ID NO: 347 - RNA, 19 nt, Synthetic construct
  source 1..19: mol_type = other RNA
gggtgatagaggcaggtcc

SEQ ID NO: 348 - RNA, 22 nt, Synthetic construct
  source 1..22: mol_type = other RNA
cctttaccctcaagtgctttcc

SEQ ID NO: 349 - RNA, 23 nt, Synthetic construct
  source 1..23: mol_type = other RNA
tgagtgtctattaagtgccagtg

SEQ ID NO: 350 - RNA, 22 nt, Synthetic construct
  source 1..22: mol_type = other RNA
cagaccacctatgactagagca

SEQ ID NO: 351 - RNA, 20 nt, Synthetic construct
  source 1..20: mol_type = other RNA
gtcccccttcctcaatcaat

SEQ ID NO: 352 - RNA, 26 nt, Synthetic construct
  source 1..26: mol_type = other RNA
tgttttcagaaataaactttttcacc

SEQ ID NO: 353 - RNA, 20 nt, Synthetic construct
  source 1..20: mol_type = other RNA
ctctgggaattgtggtcgtt

SEQ ID NO: 354 - RNA, 20 nt, Synthetic construct
  source 1..20: mol_type = other RNA
ggtgctacttgtctctcagc

SEQ ID NO: 355 - RNA, 20 nt, Synthetic construct
  source 1..20: mol_type = other RNA
catgaaatgggaacggtagg

SEQ ID NO: 356 - RNA, 21 nt, Synthetic construct
  source 1..21: mol_type = other RNA
ccacagacagagtaggacaga

SEQ ID NO: 357 - RNA, 20 nt, Synthetic construct
  source 1..20: mol_type = other RNA
cagcacatcctccttcctcc

SEQ ID NO: 358 - RNA, 20 nt, Synthetic construct
  source 1..20: mol_type = other RNA
tcccaccgttctctgatcat

SEQ ID NO: 359 - RNA, 20 nt, Synthetic construct
  source 1..20: mol_type = other RNA
agttgctgtggagaccttca

SEQ ID NO: 360 - RNA, 22 nt, Synthetic construct
  source 1..22: mol_type = other RNA
acttgtttcatgcccttgtttt

SEQ ID NO: 361 - RNA, 21 nt, Synthetic construct
  source 1..21: mol_type = other RNA
ttggttgtgcttcttgttgaa

SEQ ID NO: 362 - RNA, 20 nt, Synthetic construct
  source 1..20: mol_type = other RNA
tcgagaagggaaagattgga

SEQ ID NO: 363 - RNA, 20 nt, Synthetic construct
  source 1..20: mol_type = other RNA
tggtgatggaggcaatgact

SEQ ID NO: 364 - RNA, 20 nt, Synthetic construct
  source 1..20: mol_type = other RNA
ataggagggaggcacaagtg

SEQ ID NO: 365 - RNA, 21 nt, Synthetic construct
  source 1..21: mol_type = other RNA
ggtgataaagcgaccttgagt

SEQ ID NO: 366 - RNA, 22 nt, Synthetic construct
  source 1..22: mol_type = other RNA
gtacagagtctcggatgctttt

SEQ ID NO: 367 - RNA, 21 nt, Synthetic construct
  source 1..21: mol_type = other RNA
cacaccactccatttgtctgt

SEQ ID NO: 368 - RNA, 23 nt, Synthetic construct
  source 1..23: mol_type = other RNA
tgctcagtgattaaattccaagg

SEQ ID NO: 369 - RNA, 20 nt, Synthetic construct
  source 1..20: mol_type = other RNA
atgctatcatcatgggcccc

SEQ ID NO: 370 - RNA, 20 nt, Synthetic construct
  source 1..20: mol_type = other RNA
tggacagacatttggggtga

SEQ ID NO: 371 - RNA, 21 nt, Synthetic construct
  source 1..21: mol_type = other RNA
tccaggtgacttgatgtagca

SEQ ID NO: 372 - RNA, 20 nt, Synthetic construct
  source 1..20: mol_type = other RNA
cagcagcaaaagatgagcag

SEQ ID NO: 373 - RNA, 20 nt, Synthetic construct
  source 1..20: mol_type = other RNA
tctgtcctaatgcccttcca

SEQ ID NO: 374 - RNA, 21 nt, Synthetic construct
  source 1..21: mol_type = other RNA
agctctggaagtaggcattga

SEQ ID NO: 375 - RNA, 20 nt, Synthetic construct
  source 1..20: mol_type = other RNA
ccactgagggtattgggaca

SEQ ID NO: 376 - RNA, 20 nt, Synthetic construct
  source 1..20: mol_type = other RNA
tgagttggtgtggagaggaa

SEQ ID NO: 377 - RNA, 20 nt, Synthetic construct
  source 1..20: mol_type = other RNA
tagcaccccagatctcagtg

SEQ ID NO: 378 - RNA, 22 nt, Synthetic construct
  source 1..22: mol_type = other RNA
gattgaaccctcatcatttgcc

SEQ ID NO: 379 - RNA, 19 nt, Synthetic construct
  source 1..19: mol_type = other RNA
ccccgctgcactcaataac

SEQ ID NO: 380 - RNA, 21 nt, Synthetic construct
  source 1..21: mol_type = other RNA
gcattctctcactcaagcaca

SEQ ID NO: 381 - RNA, 23 nt, Synthetic construct
  source 1..23: mol_type = other RNA
tggctgttcagatattggattca

SEQ ID NO: 382 - RNA, 23 nt, Synthetic construct
  source 1..23: mol_type = other RNA
ggggaaagagatgagaagagaga

SEQ ID NO: 383 - RNA, 22 nt, Synthetic construct
  source 1..22: mol_type = other RNA
agagtcattgtctacgatccca

SEQ ID NO: 384 - RNA, 22 nt, Synthetic construct
  source 1..22: mol_type = other RNA
tgctcttcacatttcctgaaca

SEQ ID NO: 385 - RNA, 22 nt, Synthetic construct
  source 1..22: mol_type = other RNA
gccatttccagaattgagacca

SEQ ID NO: 386 - RNA, 21 nt, Synthetic construct
  source 1..21: mol_type = other RNA
tgcctccttgaatgaactgtg

SEQ ID NO: 387 - RNA, 23 nt, Synthetic construct
  source 1..23: mol_type = other RNA
agagagaaacactagtagcctga

SEQ ID NO: 388 - RNA, 22 nt, Synthetic construct
  source 1..22: mol_type = other RNA
gcgtaactgctagaagaagaga

SEQ ID NO: 389 - RNA, 20 nt, Synthetic construct
  source 1..20: mol_type = other RNA
aagtcactgggaagcagtca

SEQ ID NO: 390 - RNA, 87 nt, Synthetic construct
  source 1..87: mol_type = other RNA
aatgatacggcgaccaccgagatctacactctttccctacacgacgctcttccgatcttaagtagagactaagtcaaggctactgtg

SEQ ID NO: 391 - RNA, 88 nt, Synthetic construct
  source 1..88: mol_type = other RNA
aatgatacggcgaccaccgagatctacactctttccctacacgacgctcttccgatctatcatgcttaactaagtcaaggctactgtg

SEQ ID NO: 392 - RNA, 89 nt, Synthetic construct
  source 1..89: mol_type = other RNA
aatgatacggcgaccaccgagatctacactctttccctacacgacgctcttccgatctgatgcacatctactaagtcaaggctactgtg

SEQ ID NO: 393 - RNA, 90 nt, Synthetic construct
  source 1..90: mol_type = other RNA
aatgatacggcgaccaccgagatctacactctttccctacacgacgctcttccgatctcgattgctcgacactaagtcaaggctactgtg

SEQ ID NO: 394 - RNA, 91 nt, Synthetic construct
  source 1..91: mol_type = other RNA
aatgatacggcgaccaccgagatctacactctttccctacacgacgctcttccgatcttcgatagcaattcactaagtcaaggctactgtg

SEQ ID NO: 395 - RNA, 86 nt, Synthetic construct
  source 1..86: mol_type = other RNA
caagcagaagacggcatacgagattcgccttggtgactggagttcagacgtgtgctcttccgatctttctggtgtcagtatggaag

SEQ ID NO: 396 - RNA, 86 nt, Synthetic construct
  source 1..86: mol_type = other RNA
caagcagaagacggcatacgagatatagcgtcgtgactggagttcagacgtgtgctcttccgatctttctggtgtcagtatggaag

SEQ ID NO: 397 - RNA, 86 nt, Synthetic construct
  source 1..86: mol_type = other RNA
caagcagaagacggcatacgagatgaagaagtgtgactggagttcagacgtgtgctcttccgatctttctggtgtcagtatggaag

SEQ ID NO: 398 - RNA, 86 nt, Synthetic construct
  source 1..86: mol_type = other RNA
caagcagaagacggcatacgagatattctagggtgactggagttcagacgtgtgctcttccgatctttctggtgtcagtatggaag

SEQ ID NO: 399 - RNA, 86 nt, Synthetic construct
  source 1..86: mol_type = other RNA
caagcagaagacggcatacgagatcgttaccagtgactggagttcagacgtgtgctcttccgatctttctggtgtcagtatggaag

SEQ ID NO: 400 - RNA, 60 nt, Synthetic construct
  source 1..60: mol_type = other RNA
gtacgagacacggatcgacctgtctcagctgggaggcgacaagcgacctgccgccacaaa

SEQ ID NO: 401 - RNA, 60 nt, Synthetic construct
  source 1..60: mol_type = other RNA
ctgtgttctggcggcaaacccgttgcgaaaaagaacgttcacggcgactactgcacttat

SEQ ID NO: 402 - RNA, 60 nt, Synthetic construct
  source 1..60: mol_type = other RNA
gaacgttctttttcgcaacgggtttgccgccagaacacaggaccggtgccgcccaccatg

SEQ ID NO: 403 - RNA, 60 nt, Synthetic construct
  source 1..60: mol_type = other RNA
gtcgcctcccagctgagacaggtcgatccgtgtctcgtacaggccggtgatgctctggtg

SEQ ID NO: 404 - RNA, 20 nt, Synthetic construct
  source 1..20: mol_type = other RNA
tggctccgcctttttcccga

SEQ ID NO: 405 - RNA, 20 nt, Synthetic construct
  source 1..20: mol_type = other RNA
ctcggctgtttctccgctgt

SEQ ID NO: 406 - RNA, 20 nt, Synthetic construct
  source 1..20: mol_type = other RNA
gagctgggcagccagatcct

SEQ ID NO: 407 - RNA, 20 nt, Synthetic construct
  source 1..20: mol_type = other RNA
cttggcattcagcagctggc

SEQ ID NO: 408 - RNA, 40 nt, Synthetic construct
  source 1..40: mol_type = other RNA
ctccaccggcggcatggacgagctgtacaagcatcatcac

SEQ ID NO: 409 - RNA, 40 nt, Synthetic construct
  source 1..40: mol_type = other RNA
ccatgttattctcctcgcccttgctcaccatggtggcgac

SEQ ID NO: 410 - RNA, 40 nt, Synthetic construct
  source 1..40: mol_type = other RNA
gggcgaggagaataacatggccatcatcaaggagttcatg

SEQ ID NO: 411 - RNA, 40 nt, Synthetic construct
  source 1..40: mol_type = other RNA
cgtccatgccgccggtggagtggcggccctcggcgcgttc

SEQ ID NO: 412 - RNA, 20 nt, Synthetic construct
  source 1..20: mol_type = other RNA
ccccgtaatgcagaagaaga

SEQ ID NO: 413 - RNA, 21 nt, Synthetic construct
  source 1..21: mol_type = other RNA
catagcgtaaaaggagcaaca

SEQ ID NO: 414 - RNA, 21 nt, Synthetic construct
  source 1..21: mol_type = other RNA
tgctccactttgaaacagctg

SEQ ID NO: 415 - RNA, 21 nt, Synthetic construct
  source 1..21: mol_type = other RNA
ggggactgacattacctctgc

SEQ ID NO: 416 - RNA, 91 nt, Synthetic construct
  source 1..91: mol_type = other RNA
aatgatacggcgaccaccgagatctacactctttccctacacgacgctcttccgatctatcatgcttagagggccagatgatatagattcc

SEQ ID NO: 417 - RNA, 90 nt, Synthetic construct
  source 1..90: mol_type = other RNA
caagcagaagacggcatacgagatatagcgtcgtgactggagttcagacgtgtgctcttccgatctggcaaggaagtgactgtaattatg

SEQ ID NO: 418 - RNA, 88 nt, Synthetic construct
  source 1..88: mol_type = other RNA
aatgatacggcgaccaccgagatctacactctttccctacacgacgctcttccgatcttaagtagagtgctccactttgaaacagctg

SEQ ID NO: 419 - RNA, 87 nt, Synthetic construct
  source 1..87: mol_type = other RNA
caagcagaagacggcatacgagattcgccttggtgactggagttcagacgtgtgctcttccgatctacacatgcctctcctctctct

SEQ ID NO: 420 - RNA, 87 nt, Synthetic construct
  source 1..87: mol_type = other RNA
aatgatacggcgaccaccgagatctacactctttccctacacgacgctcttccgatcttaagtagaggggatcatcaccggaccttt

SEQ ID NO: 421 - RNA, 87 nt, Synthetic construct; identical to SEQ ID NO: 420
  source 1..87: mol_type = other RNA

SEQ ID NO: 422 - RNA, 88 nt, Synthetic construct
  source 1..88: mol_type = other RNA
aatgatacggcgaccaccgagatctacactctttccctacacgacgctcttccgatctatcatgcttatcaccagaccttcggctttt

SEQ ID NO: 423 - RNA, 87 nt, Synthetic construct
  source 1..87: mol_type = other RNA
aatgatacggcgaccaccgagatctacactctttccctacacgacgctcttccgatcttaagtagagcaccacgcctgcctaatttt

SEQ ID NO: 424 - RNA, 87 nt, Synthetic construct; identical to SEQ ID NO: 423
  source 1..87: mol_type = other RNA

SEQ ID NO: 425 - RNA, 88 nt, Synthetic construct
  source 1..88: mol_type = other RNA
aatgatacggcgaccaccgagatctacactctttccctacacgacgctcttccgatctatcatgcttagatcatcaccggacctttgg

SEQ ID NO: 426 - RNA, 87 nt, Synthetic construct; identical to SEQ ID NO: 423
  source 1..87: mol_type = other RNA

SEQ ID NO: 427 - RNA, 86 nt, Synthetic construct
  source 1..86: mol_type = other RNA
caagcagaagacggcatacgagattcgccttggtgactggagttcagacgtgtgctcttccgatctcaccacgcctgcctaatttt

SEQ ID NO: 428 - RNA, 86 nt, Synthetic construct
  source 1..86: mol_type = other RNA
caagcagaagacggcatacgagattcgccttggtgactggagttcagacgtgtgctcttccgatcttttcatcatgttggccaggc

SEQ ID NO: 429 - RNA, 86 nt, Synthetic construct
  source 1..86: mol_type = other RNA
caagcagaagacggcatacgagatatagcgtcgtgactggagttcagacgtgtgctcttccgatctcaccacgcctgcctaatttt

SEQ ID NO: 430 - RNA, 86 nt, Synthetic construct
  source 1..86: mol_type = other RNA
caagcagaagacggcatacgagattcgccttggtgactggagttcagacgtgtgctcttccgatctgggatcatcaccggaccttt

SEQ ID NO: 431 - RNA, 86 nt, Synthetic construct; identical to SEQ ID NO: 430
  source 1..86: mol_type = other RNA

SEQ ID NO: 432 - RNA, 86 nt, Synthetic construct
  source 1..86: mol_type = other RNA
caagcagaagacggcatacgagatatagcgtcgtgactggagttcagacgtgtgctcttccgatctgcctcagcctcctaagtagc

SEQ ID NO: 433 - RNA, 86 nt, Synthetic construct; identical to SEQ ID NO: 430
  source 1..86: mol_type = other RNA

SEQ ID NO: 434 - RNA, 42 nt, Synthetic construct
  source 1..42: mol_type = other RNA
cccacctcccaaccccgaggggacccagagagggcctatttc

SEQ ID NO: 435 - RNA, 50 nt, Synthetic construct
  source 1..50: mol_type = other RNA
gggaaataggccctctctgggtcgaaaaaagcaccgactcggtgccactt

SEQ ID NO: 436 - RNA, 34 nt, Synthetic construct
  source 1..34: mol_type = other RNA
tatcgttgtgtacaaggcagggatattcaccatt

SEQ ID NO: 437 - RNA, 36 nt, Synthetic construct
  source 1..36: mol_type = other RNA
tatcgttgcgccggcgaattgtggatgaatactgcc

SEQ ID NO: 438 - RNA, 37 nt, Synthetic construct
  source 1..37: mol_type = other RNA
tatcgttgcgccggcggaattcgctagctaggtcttg

SEQ ID NO: 439 - RNA, 31 nt, Synthetic construct
  source 1..31: mol_type = other RNA
tatcgttgtgtacaccaaactggatctctgc

SEQ ID NO: 440 - RNA, 38 nt, Synthetic construct
  source 1..38: mol_type = other RNA
tatcgttgcgccggcggagacaaatggcagtattcatc

SEQ ID NO: 441 - RNA, 57 nt, Synthetic construct
  source 1..57: mol_type = other RNA
tatcgttgtgtacactctattcactatagaaagtacagcaaaaactattcttaaacc

SEQ ID NO: 442 - RNA, 37 nt, Synthetic construct
  source 1..37: mol_type = other RNA
agggatattcaccattatcgtcgtttcagacccacct

SEQ ID NO: 443 - RNA, 44 nt, Synthetic construct
  source 1..44: mol_type = other RNA
gggttgggaggtgggtctgactcaagatctagttacgccaagct

SEQ ID NO: 444 - RNA, 40 nt, Synthetic construct
  source 1..40: mol_type = other RNA
tggcgtaactagatcttgagtcagacccacctcccaaccc

SEQ ID NO: 445 - RNA, 33 nt, Synthetic construct
  source 1..33: mol_type = other RNA
gggaggtgggtctgaaacgacgataatggtgaa

SEQ ID NO: 446 - RNA, 20 nt, Synthetic construct
  source 1..20: mol_type = other RNA
aggcagggatattcaccatt

SEQ ID NO: 447 - RNA, 20 nt, Synthetic construct
  source 1..20: mol_type = other RNA
aattgtggatgaatactgcc

SEQ ID NO: 448 - RNA, 20 nt, Synthetic construct
  source 1..20: mol_type = other RNA
ggaatcatcttcacagttgt

SEQ ID NO: 449 - RNA, 20 nt, Synthetic construct
  source 1..20: mol_type = other RNA
acaactgtgaagatgattcc

SEQ ID NO: 450 - RNA, 20 nt, Synthetic construct
  source 1..20: mol_type = other RNA
ctaatgtatgactgaaagct

SEQ ID NO: 451 - RNA, 20 nt, Synthetic construct
  source 1..20: mol_type = other RNA
agctttcagtcatacattag

SEQ ID NO: 452 - RNA, 20 nt, Synthetic construct
  source 1..20: mol_type = other RNA
gaggtgtctaaaccatgaca

SEQ ID NO: 453 - RNA, 20 nt, Synthetic construct
  source 1..20: mol_type = other RNA
tgtcatggtttagacacctc

SEQ ID NO: 454 - RNA, 20 nt, Synthetic construct
  source 1..20: mol_type = other RNA
ctgcaggtcgaccatatggg

SEQ ID NO: 455 - RNA, 23 nt, Synthetic construct
  source 1..23: mol_type = other RNA
ggaatcatcttcacagttgttgg

SEQ ID NO: 456 - RNA, 23 nt, Synthetic construct
  source 1..23: mol_type = other RNA
aatatcctgccacctctaacagg

SEQ ID NO: 457 - RNA, 23 nt, Synthetic construct
  source 1..23: mol_type = other RNA
tcagtccagtcaaaggtggaagg

SEQ ID NO: 458 - RNA, 23 nt, Synthetic construct
  source 1..23: mol_type = other RNA
ctaatgtatgactgaaagctggg

SEQ ID NO: 459 - RNA, 23 nt, Synthetic construct
  source 1..23: mol_type = other RNA
gaggtgtctaaaccatgacaagg

SEQ ID NO: 460 - RNA, 23 nt, Synthetic construct
  source 1..23: mol_type = other RNA
gtgcacatcttatctcccttagg

SEQ ID NO: 461 - RNA, 23 nt, Synthetic construct
  source 1..23: mol_type = other RNA
ttagggggccaagagcgtatggg

SEQ ID NO: 462 - RNA, 23 nt, Synthetic construct
  source 1..23: mol_type = other RNA
gaagccgagtatctgggacgagg

SEQ ID NO: 463 - RNA, 23 nt, Synthetic construct
  source 1..23: mol_type = other RNA
aacaggactcctggcagagtagg

SEQ ID NO: 464 - RNA, 20 nt, Synthetic construct
  source 1..20: mol_type = other RNA
aatatcctgccacctctaac

SEQ ID NO: 465 - RNA, 20 nt, Synthetic construct
  source 1..20: mol_type = other RNA
tcagtccagtcaaaggtgga

SEQ ID NO: 466 - RNA, 20 nt, Synthetic construct
  source 1..20: mol_type = other RNA
gtgcacatcttatctccctt

SEQ ID NO: 467 - RNA, 20 nt, Synthetic construct
  source 1..20: mol_type = other RNA
ttagggggccaagagcgtat

SEQ ID NO: 468 - RNA, 12 nt, Synthetic construct
  source 1..12: mol_type = other RNA
tggagccacaga

SEQ ID NO: 469 - RNA, 17 nt, Synthetic construct
  source 1..17: mol_type = other RNA
ctgggagcgatttgagg

SEQ ID NO: 470 - RNA, 21 nt, Synthetic construct
  source 1..21: mol_type = other RNA
tggctctggagcgatttgagg

SEQ ID NO: 471 - RNA, 60 nt, Synthetic construct
  source 1..60: mol_type = other RNA
gatattctggtgtcagtatggaagatcttacacatttatttaatactcaaatgtatgaag

SEQ ID NO: 472 - RNA, 60 nt, Synthetic construct
  source 1..60: mol_type = other RNA
gatattctggtgtcagtatggaagatcttacacatttatttaatactcaagtgtatgaag

SEQ ID NO: 473 - RNA, 47 nt, Synthetic construct
  source 1..47: mol_type = other RNA
atgtttgtttgtagcataacaacagcacagtagccttgacttagttt

SEQ ID NO: 474 - RNA, 44 nt, Synthetic construct
  source 1..44: mol_type = other RNA
atgtttgtttataacataacagcacagtagccttgacttagttt